(19) United States
(12) Patent Application Publication
(10) Pub. No.: US 2025/0270246 A1
(43) Pub. Date: Aug. 28, 2025

(54) LINCOSAMIDES AND USES THEREOF

(71) Applicant: President and Fellows of Harvard College, Cambridge, MA (US)

(72) Inventors: Andrew G. Myers, Cambridge, MA (US); Kelvin Wu, Cambridge, MA (US); Ben Calhoun Tresco, Cambridge, MA (US)

(73) Assignee: President and Fellows of Harvard College, Cambridge, MA (US)

(21) Appl. No.: 18/858,278

(22) PCT Filed: Apr. 19, 2023
(86) PCT No.: PCT/US2023/019059

§ 371 Date: Oct. 18, 2024

Related U.S. Application Data
(60) Provisional application No. 63/332,993, filed on Apr. 20, 2022.

(51) Int. Cl. C07H 15/26; A61K 31/7056; A61K 31/706; A61P 31/04

(52) U.S. Cl. CPC C07H 15/26; A61K 31/7056; A61K 31/706; A61P 31/04

(57) ABSTRACT

Provided are compounds useful for the treatment and prevention of infectious diseases. The compound structures are lincosamide analogs modified at the aminooctose (northern) and amino acid (southern) regions. Also provided are methods for preparing the lincosamide compounds, pharmaceutical compositions comprising the lincosamide compounds, and methods of treating infectious diseases using the disclosed lincosamide compounds.

### RELATED APPLICATIONS

This patent application claims priority under 35 U.S.C. 119 (e) to U.S. Provisional Patent Application U.S. Ser. No. 63/332,993, filed Apr. 20, 2022, which is incorporated herein by reference in its entirety.

### BACKGROUND

Emerging resistance to existing antibiotics is rapidly developing as a crisis of global proportions, especially for infections originating from drug-resistant Gram-negative bacteria. Pathogenic bacteria can transmit genes coding for antibiotic resistance both vertically (to their progeny) and horizontally (to neighboring bacteria of different lineages), and as a result antibiotic resistance can evolve quickly, particularly in nosocomial (hospital) settings. See, e.g., Wright, Chem. Commun. (2011) 47:4055-4061. More than 99,000 people die annually in the U.S. from healthcare-associated infections, more than all casualties from car accidents, HIV, and breast cancer combined, creating an estimated burden of up to $45 billion in U.S. healthcare costs. See, e.g., Klevens et al., Public Health Rep (2007) 122:160-166. The current crisis is exacerbated by decreased research in the development of new antibiotics by most major pharmaceutical companies. See, e.g., Projan, Curr. Opin. Microbiol. (2003) 6:427-430. The current rate of introduction of new antibiotics does not adequately address growing resistance, and with the ease of international travel and increasing population densities, the need for innovation in the field has never been higher.

The lincosamides are a class of antibiotics that prevent bacteria growth by interfering with the synthesis of proteins. They bind to the 23s portion of the 50S subunit of bacterial ribosomes and cause premature dissociation of the peptidyl-tRNA from the ribosome. Lincosamides do not interfere with protein synthesis in human cells (or those of other eukaryotes) because human ribosomes are structurally different from those of bacteria.

The first lincosamide to be discovered was lincomycin, but the use of lincomycin as an antibiotic has been largely superseded by clindamycin, which exhibits improved antibacterial activity. Clindamycin also exhibits some activity against parasitic protozoa and has been used to treat toxoplasmosis and malaria. Lincosamides are typically used to treat Staphylococcus and Streptococcus infections but have also proved to be useful in treating Bacteroides fragilis and other anaerobic infections. They are used in the treatment of toxic shock syndrome and thought to directly block the M protein production that leads to the severe inflammatory response.

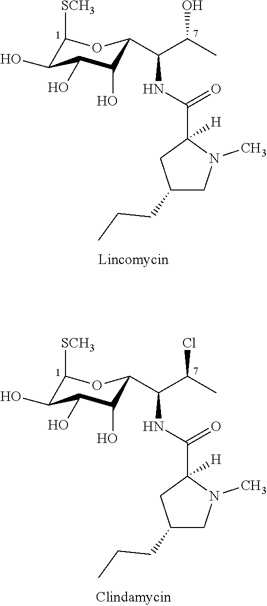

Target bacteria may alter the drug's binding site leading to resistance (similar to resistance found with macrolides and streptogramins). The resistance mechanism is methylation of the 23s binding site. If this occurs, then the bacteria are resistant to both macrolides and lincosamides. In rare instances, enzymatic inactivation of clindamycin has also been reported.

Lincosamide antibiotics are also associated with pseudomembranous colit is caused by Clostridium difficile (C. difficile). Pseudomembranous colit is is inflammation of the colon associated with an overgrowth of C. difficile. This overgrowth of C. difficile is most often related to recent lincosamide antibiotic use. For example, clindamycin, currently the only lincosamide in clinical use, carries a black-box warning for its tendency to promote C. difficile-associated diarrhea (CDAD).

Accordingly, the discovery and development of new antibiotics effective against drug-resistant bacteria, particularly new lincosamides, represents a currently unmet medical need.

### SUMMARY

Clindamycin's primary degradation pathway in vivo is the “first-pass” oxidation of its methyl thioglycoside by hepatic cytochrome P450 enzymes to form comparatively inactive clindamycin sulfoxide diastereomers. In general, reducing the metabolic degradation of an antibiotic can improve its therapeutic index by achieving the same inhibitory effect with a lower dosage. The present disclosure stems from the recognition that there is significant unrealized potential to improve therapeutic efficacy of the lincosamides against MDR ESKAPE pathogens in vivo by targeted glycosidic modifications, a contention supported by preliminary data. In particular, crystal structure analysis of lincosamide analog iboxamycin bound to the bacterial ribosome suggested that formation of a macrocyclic moiety constraining the aminooctose (northern) region would promote the prearrangement of the analog into its preferred binding conformation, thus lowering entropy loss upon binding and increasing target engagement.

Thus, disclosed are compounds that are analogs of lincosamides. The compounds have modified northern and amino-acid (southern) regions. The compounds include a novel heterocyclic moiety constraining the northern region. The disclosed synthetic lincosamides demonstrate potent activity against both Gram-positive and Gram-negative pathogens.

In one aspect, the present disclosure provides compounds of Formula (I):

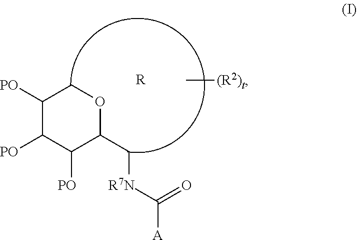

and pharmaceutically acceptable salts, co-crystals, tautomers, stereoisomers, solvates, hydrates, polymorphs, isotopically enriched derivatives, or prodrugs thereof, wherein:
each occurrence of P is independently hydrogen or a protecting group;
A is substituted or unsubstituted heteroaliphatic, substituted or unsubstituted heteroaryl, or substituted or unsubstituted heteroaralkyl;
R⁷ is hydrogen or unsubstituted alkyl;
R is heterocyclyl;
each occurrence of R² is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0-12; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

The disclosed compounds have anti-microbial activity and may be used to treat and/or prevent infectious diseases. Pharmaceutical compositions of the compounds, kits comprising the compounds and/or compositions, and methods of treatment using the compounds or compositions thereof are provided herein. Infectious diseases which may be treated with compounds of the invention include, but are not limited to, bacterial infections caused by Staphylococcus, Streptococcus, Enterococcus, Acinetobacter, Clostridium, Bacterioides, Klebsiella, Escherichia, Pseudomonas, and Haemophilus species.

Methods of preparing the disclosed compounds are also provided herein. In certain embodiments, the disclosed compounds are prepared by a ring closing metathesis reaction (see, e.g., Scheme 1).

The details of certain embodiments of the invention are set forth in the Detailed Description of Certain Embodiments, as described below. Other features, objects, and advantages of the invention will be apparent from the Definitions, Examples, and Claims.

### DETAILED DESCRIPTION OF CERTAIN EMBODIMENTS

The compounds disclosed herein include lincosamide analogues. The disclosed compounds have increased structural diversity over known lincosamides, such as lincomycin and clindamycin. The compounds have modified northern and amino-acid (southern) regions, including a novel heterocyclic moiety constraining the northern region. The disclosed compounds provide unexpected and potent activity against various microorganisms, including Gram negative bacteria. Also disclosed are methods for the preparation of the disclosed compounds, pharmaceutical compositions comprising the compounds, uses of the compounds, and methods of using the compounds (e.g., treatment of an infectious disease, prevention of an infectious disease).

In one aspect, provided are compounds of Formula (I):

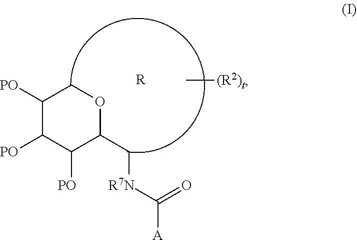

and pharmaceutically acceptable salts, co-crystals, tautomers, stereoisomers, solvates, hydrates, polymorphs, isotopically enriched derivatives, or prodrugs thereof, wherein:
each occurrence of P is independently hydrogen or a protecting group;
A is substituted or unsubstituted heteroaliphatic, substituted or unsubstituted heteroaryl, or substituted or unsubstituted heteroaralkyl;
R is heterocyclyl;
R⁷ is hydrogen or unsubstituted alkyl;
each occurrence of R² is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0-12; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

Unless otherwise stated, any formulae described herein are also meant to include salts, solvates, hydrates, polymorphs, co-crystals, tautomers, stereoisomers, and isotopically labeled derivatives thereof. In certain embodiments, the provided compound is a salt of any of the formulae described herein. In certain embodiments, the provided compound is a pharmaceutically acceptable salt of any of the formulae described herein. In certain embodiments, the provided compound is a solvate of any of the formulae described herein. In certain embodiments, the provided compound is a hydrate of any of the formulae described herein. In certain embodiments, the provided compound is a polymorph of any of the formulae described herein. In certain embodiments, the provided compound is a co-crystal of any of the formulae described herein. In certain embodiments, the provided compound is a tautomer of any of the formulae described herein. In certain embodiments, the provided compound is a stereoisomer of any of the formulae described herein. In certain embodiments, the provided compound is of an isotopically labeled form of any of the formulae described herein. For example, compounds having the present structures except for the replacement of hydrogen by deuterium or tritium, replacement of ¹⁹F with ¹⁸F, or the replacement of a ¹²C by a ¹³C or ¹⁴C are within the scope of the disclosure. In certain embodiments, the provided compound is a deuterated form of any of the formulae or compounds described herein.

In certain embodiments of the compound of Formula (I), each occurrence of P is hydrogen.

### Group R⁷

As generally defined herein, R⁷ is hydrogen or unsubstituted alkyl.

In certain embodiments, R⁷ is hydrogen or unsubstituted alkyl. In certain embodiments, R⁷ is unsubstituted alkyl. In certain embodiments, R⁷ is unsubstituted C₁₋₆ alkyl. In certain embodiments, R⁷ is unsubstituted C₁₋₄ alkyl. In certain embodiments, R⁷ is unsubstituted C₁₋₃ alkyl. In certain embodiments, R⁷ is unsubstituted C₁₋₂ alkyl. In certain embodiments, R⁷ is ethyl. In certain embodiments, R⁷ is methyl. In certain embodiments, R⁷ is hydrogen.

### Group A

As generally defined herein, A is substituted or unsubstituted heteroaliphatic, substituted or unsubstituted heteroaryl, or substituted or unsubstituted heteroaralkyl.

In certain embodiments, A is substituted or unsubstituted heteroaliphatic.

In certain embodiments, A is substituted or unsubstituted heterocyclyl.

In certain embodiments, A is substituted or unsubstituted 4-8 membered monocyclic heterocyclyl, or substituted or unsubstituted fused bicyclic heterocyclyl. In certain embodiments, A is substituted or unsubstituted 4-8 membered monocyclic heterocyclyl, or substituted or unsubstituted 8-12 membered fused bicyclic heterocyclyl. In certain embodiments, A is substituted or unsubstituted 5-8 membered monocyclic heterocyclyl, or substituted or unsubstituted 8-12 membered fused bicyclic heterocyclyl. In certain embodiments, A is substituted or unsubstituted 5-8 membered monocyclic heterocyclyl, or substituted or unsubstituted 9-11 membered fused bicyclic heterocyclyl. In certain embodiments, A is substituted or unsubstituted 5-7 membered monocyclic heterocyclyl, or substituted or unsubstituted 9-11 membered fused bicyclic heterocyclyl. In certain embodiments, A is substituted or unsubstituted 5-7 membered monocyclic heterocyclyl, or substituted or unsubstituted 9-10 membered fused bicyclic heterocyclyl. In certain embodiments, A is substituted or unsubstituted 5-7 membered monocyclic heterocyclyl, or substituted or unsubstituted 10-membered fused bicyclic heterocyclyl.

In certain embodiments, A is substituted or unsubstituted 4-8 membered monocyclic heterocyclyl. In certain embodiments, A is substituted or unsubstituted 5-8 membered monocyclic heterocyclyl. In certain embodiments, A is substituted or unsubstituted 5-7 membered monocyclic heterocyclyl. In certain embodiments, A is substituted or unsubstituted 5-membered monocyclic heterocyclyl. In certain embodiments, A is substituted or unsubstituted 6-membered monocyclic heterocyclyl. In certain embodiments, A is substituted or unsubstituted 7-membered monocyclic heterocyclyl.

In certain embodiments, A is of formula:

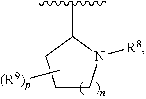

wherein:
n is 1, 2, or 3;
R⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaliphatic, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —S(O)₂R^{A}, or a nitrogen protecting group;
each occurrence of R⁹ is independently, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, OC(═O)N(R^{A})₂, —NR^{A}S(O)₂R^{A}, —OS(O)₂R^{A}, or —S(O)₂R^{A}; and
p is 0-4.

In certain embodiments, A is of formula:

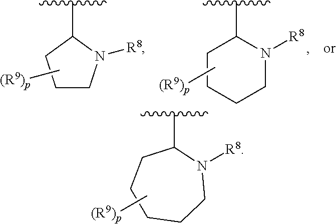

In certain embodiments, A is of formula:

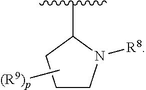

In certain embodiments, A is of formula:

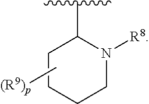

In certain embodiments, A is of formula:

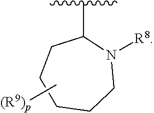

In certain embodiments, A is of formula:

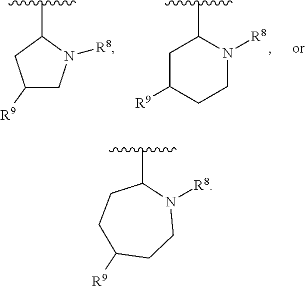

In certain embodiments, A is of formula:

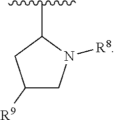

In certain embodiments, A is of formula:

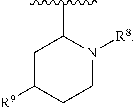

In certain embodiments, A is of formula:

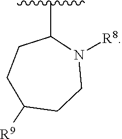

In certain embodiments, A is substituted or unsubstituted fused bicyclic heterocyclyl. In certain embodiments, A is substituted or unsubstituted 8-12 membered fused bicyclic heterocyclyl. In certain embodiments, A is substituted or unsubstituted 9-11 membered fused bicyclic heterocyclyl. In certain embodiments, A is substituted or unsubstituted 9-10 membered fused bicyclic heterocyclyl. In certain embodiments, A is substituted or unsubstituted 10-membered fused bicyclic heterocyclyl.

In certain embodiments, A is of formula:

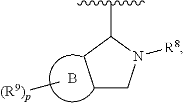

wherein:
B is carbocyclyl or heterocyclyl;
R⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaliphatic, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —S(O)₂R^{A}, or a nitrogen protecting group;
each occurrence of R⁹ is independently, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, —SR^{A}, —CN, —SCN, —C(═NR^{A})R^{A}, C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring; and
p is 0-4.

In certain embodiments, B is cycloalkyl, cycloalkenyl, or heterocyclyl. In certain embodiments, B is heterocyclyl. In certain embodiments, B is a 5-8 membered cycloalkyl, cycloalkenyl, or heterocyclyl ring. In certain embodiments, B is a 5-8 membered heterocyclyl ring. In certain embodiments, B is a 5-7 membered cycloalkyl, cycloalkenyl, or heterocyclyl ring. In certain embodiments, B is a 5-7 membered heterocyclyl ring. In certain embodiments, B is a 6-7 membered cycloalkyl, cycloalkenyl, or heterocyclyl ring. In certain embodiments, B is a 6-7 membered heterocyclyl ring. In certain embodiments, B is a 6-membered cycloalkyl, cycloalkenyl, or heterocyclyl ring. In certain embodiments, B is a 6-membered heterocyclyl ring. In certain embodiments, B is a 7-membered cycloalkyl, cycloalkenyl, or heterocyclyl ring. In certain embodiments, B is a 7-membered heterocyclyl ring.

In certain embodiments, A is of formula:

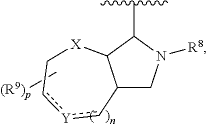

wherein:
X is O, S, NR⁹ or C(R⁹)₂;
Y is O, S, N, NR⁹, C(R⁹)₂, CR⁹, CH₂, or CH;
n is 0 or 1;
p is 0-4; and [symbol] represents a single or double bond.

In certain embodiments, X is O, S, NR⁹ or C(R⁹)₂. In certain embodiments, X is O, S, or NR⁹. In certain embodiments, X is O or S. In certain embodiments, X is O.

In certain embodiments, Y is O, S, NR⁹, C(R⁹)₂, CR⁹, CHR⁹, CH₂, or CH. In certain embodiments, Y is O, NR⁹, C(R⁹)₂, CR⁹, CHR⁹, CH₂, or CH. In certain embodiments, Y is NR⁹, C(R⁹)₂, CR⁹, CHR⁹, CH₂, or CH. In certain embodiments, Y is C(R⁹)₂, CR⁹, CHR⁹, CH₂, or CH. In certain embodiments, Y is C(R⁹)₂, CR⁹, CHR⁹, CH₂, or CH. In certain embodiments, Y is C(R⁹)₂, CHR⁹, or CH₂. In certain embodiments, Y is C(R⁹)₂. In certain embodiments, Y is CHR⁹. In certain embodiments, Y is CH₂. In certain embodiments, Y is CR⁹ or CH. In certain embodiments, Y is CR⁹. In certain embodiments, Y is CH.

In certain embodiments, n is 0. In certain embodiments, n is 1.

In certain embodiments, p is 0-3. In certain embodiments, p is 0-2. In certain embodiments, p is 0-1. In certain embodiments, p is 1-2. In certain embodiments, p is 0 or 2. In certain embodiments, p is 0. In certain embodiments, p is 1. In certain embodiments, p is 2.

In certain embodiments, X is O; Y is NR⁹, C(R⁹)₂, CR⁹, CHR⁹, CH₂, or CH; n is 1; and p is 0-2. In certain embodiments, X is O; Y is C(R⁹)₂, CR⁹, CHR⁹, CH₂, or CH; n is 1; and p is 0-2. In certain embodiments, X is O; Y is CR⁹ or CH; n is 1; and p is 1. In certain embodiments, X is O; Y is C(R⁹)₂, CHR⁹, or CH₂; n is 1; and p is 0-2.

In certain embodiments, A is of formula:

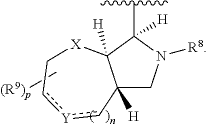

In certain embodiments, A is of formula:

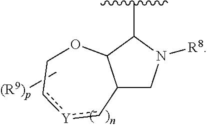

In certain embodiments, A is of formula:

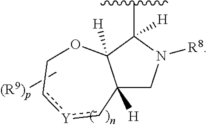

In certain embodiments, A is of formula:

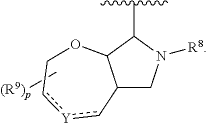

In certain embodiments, A is of formula:

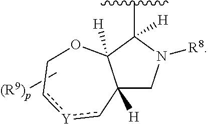

In certain embodiments, A is of formula:

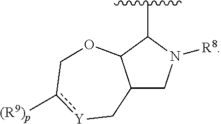

In certain embodiments, A is of formula:

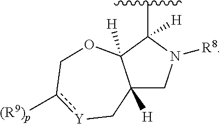

In certain embodiments, A is of formula:

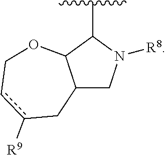

In certain embodiments, A is of formula:

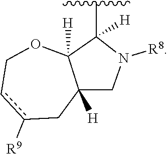

In certain embodiments, A is of formula:

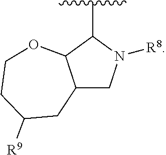

In certain embodiments, A is of formula:

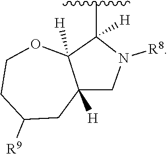

In certain embodiments, A is of formula:

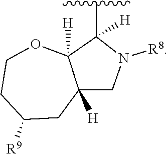

In certain embodiments, A is of formula:

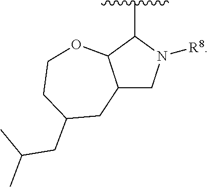

In certain embodiments, A is of formula:

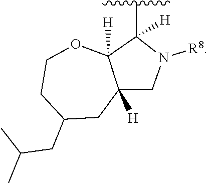

In certain embodiments, A is of formula:

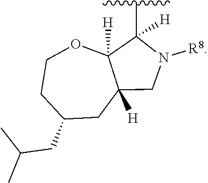

In certain embodiments, A is of formula:

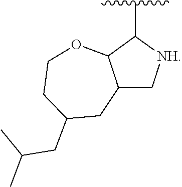 [SMILES: *C1NCC2CC(CC(C)C)CCOC21]

In certain embodiments, A is of formula:

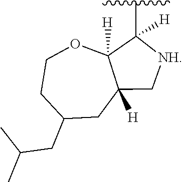 [SMILES: *[C@H]1NC[C@@H]2CC(CC(C)C)CCO[C@H]21]

In certain embodiments, A is of formula:

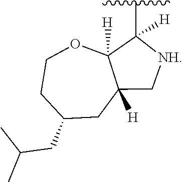 [SMILES: *[C@H]1NC[C@@H]2C[C@H](CC(C)C)CCO[C@H]21]

In certain embodiments, A is of formula:

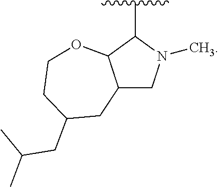 [SMILES: *C1C2OCCC(CC(C)C)CC2CN1C]

In certain embodiments, A is of formula:

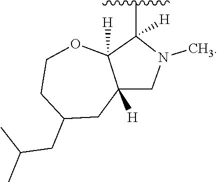 [SMILES: *[C@@H]1[C@@H]2OCCC(CC(C)C)C[C@H]2CN1C]

In certain embodiments, A is of formula:

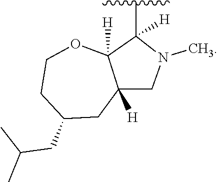 [SMILES: *[C@@H]1[C@@H]2OCC[C@@H](CC(C)C)C[C@H]2CN1C]

### Group R⁸

As generally defined herein, R⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaliphatic, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —S(O)₂R^{A}, or a nitrogen protecting group.

In certain embodiments, R⁸ is hydrogen, substituted or unsubstituted alkyl, or —C(═O)R^{A}. In certain embodiments, R⁸ is hydrogen or substituted or unsubstituted alkyl. In certain embodiments, R⁸ is hydrogen or unsubstituted alkyl. In certain embodiments, R⁸ is hydrogen or unsubstituted C₁₋₆ alkyl. In certain embodiments, R⁸ is hydrogen or unsubstituted C₁₋₄ alkyl. In certain embodiments, R⁸ is hydrogen or unsubstituted C₁₋₃ alkyl. In certain embodiments, R⁸ is hydrogen or unsubstituted C₁₋₂ alkyl. In certain embodiments, R⁸ is hydrogen or ethyl. In certain embodiments, R⁸ is hydrogen or methyl. In certain embodiments, R⁸ is hydrogen.

In certain embodiments, R⁸ is unsubstituted C₁₋₆ alkyl. In certain embodiments, R⁸ is unsubstituted C₁₋₄ alkyl. In certain embodiments, R⁸ is unsubstituted C₁₋₃ alkyl. In certain embodiments, R⁸ is unsubstituted C₁₋₂ alkyl. In certain embodiments, R⁸ is ethyl. In certain embodiments, R⁸ is methyl.

### Group R⁹

As generally defined herein, each occurrence of R⁹ is independently, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring. In certain embodiments, the R⁹ substituent is the(S) stereocenter (i.e., the carbon to which it is attached is of the(S) configuration). In certain embodiments, the R⁹ substituent is the (R) stereocenter (i.e., the carbon to which it is attached is of the (R) configuration).

In certain embodiments, R⁹ is halogen, substituted or unsubstituted alkyl, substituted or unsubstituted alkenyl, substituted or unsubstituted alkynyl, substituted or unsubstituted heterocyclyl, substituted or unsubstituted aryl, substituted or unsubstituted aralkyl, substituted or unsubstituted heteroaryl, substituted or unsubstituted heteroaralkyl, —OR^{A}, or —N(R^{A})₂; or two R⁹ groups are joined to form a substituted or unsubstituted heterocyclyl ring, or a substituted or unsubstituted carbocyclyl ring.

In certain embodiments, R⁹ is halogen, substituted or unsubstituted alkyl, substituted or unsubstituted alkenyl, substituted or unsubstituted alkynyl, substituted or unsubstituted aryl, substituted or unsubstituted aralkyl, substituted or unsubstituted heteroaryl, substituted or unsubstituted heteroaralkyl, —C(═O)OR^{A}, —C(═O)N(R^{A})₂, —C(═NR^{A})N(R^{A})₂, —OR^{A}, or —N(R^{A})₂; or two R⁹ groups are joined to form a substituted or unsubstituted heterocyclyl ring, or a substituted or unsubstituted carbocyclyl ring.

In certain embodiments, R⁹ is halogen, substituted or unsubstituted alkenyl, substituted alkynyl, substituted or unsubstituted aryl, or substituted or unsubstituted aralkyl; or two R⁹ groups are joined to form a substituted or unsubstituted carbocyclyl ring.

In certain embodiments, R⁹ is halogen, unsubstituted ethenyl, substituted or unsubstituted phenethynyl, substituted or unsubstituted phenyl, substituted or unsubstituted benzyl, or substituted or unsubstituted phenethyl; or two R⁹ groups are joined to form a unsubstituted cycloalkyl ring.

In certain embodiments, R⁹ is substituted or unsubstituted alkyl, substituted or unsubstituted aryl, or substituted or unsubstituted aralkyl. In certain embodiments, R⁹ is substituted or unsubstituted aryl or substituted or unsubstituted aralkyl. In certain embodiments, R⁹ is substituted or unsubstituted phenyl or substituted or unsubstituted phenethyl.

In certain embodiments, R⁹ is substituted or unsubstituted alkyl, substituted or unsubstituted alkenyl, substituted or unsubstituted carbocyclyl, or substituted or unsubstituted heteroalkyl. In certain embodiments, R⁹ is substituted or unsubstituted alkyl, substituted or unsubstituted alkenyl, substituted or unsubstituted carbocyclyl, or unsubstituted heteroalkyl. In certain embodiments, R⁹ is substituted or unsubstituted alkyl, substituted or unsubstituted alkenyl, or substituted or unsubstituted carbocyclyl.

In certain embodiments, R⁹ is substituted or unsubstituted alkenyl. In certain embodiments, R⁹ is unsubstituted alkenyl. In certain embodiments, R⁹ is —CH═CH₂.

In certain embodiments, R⁹ is substituted or unsubstituted carbocyclyl. In certain embodiments, R⁹ is unsubstituted carbocyclyl. In certain embodiments, R⁹ is unsubstituted C₃₋₆ cycloalkyl. In certain embodiments, R⁹ is unsubstituted C₅₋₆ cycloalkyl. In certain embodiments, R⁹ is unsubstituted cyclopentyl. In certain embodiments, R⁹ is unsubstituted cyclohexyl.

In certain embodiments, R⁹ is substituted or unsubstituted alkyl. In certain embodiments, R⁹ is substituted or unsubstituted C₁₋₆ alkyl. In certain embodiments, R⁹ is substituted C₁₋₆ alkyl. In certain embodiments, R⁹ is C₁₋₆ alkyl substituted with halogen, alkenyl, C₃₋₆ cycloalkyl, or —OR^{A}. In certain embodiments, R⁹ is C₁₋₆ alkyl substituted with halogen, alkenyl, C₃₋₆ cycloalkyl, heterocyclyl, —N(R^{A})₂, —SO₂R^{A}, or —OR^{A}. In certain embodiments, R⁹ is —CH₂CH—CH₂. In certain embodiments, R⁹ is —CH₂CH₂F. In certain embodiments, R⁹ is —CH₂CHF₂. In certain embodiments, R⁹ is —CH₂CH₂CH₂F. In certain embodiments, R⁹ is —CH₂CH₂CF₂H. In certain embodiments, R⁹ is —CH₂CH₂CH₂Cl. In certain embodiments, R⁹ is

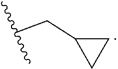 [SMILES: *CC1CC1]

In certain embodiments, R⁹ is —CH₂CH₂CH₂OCH₃. In certain embodiments, R⁹ is —CH₂CH₂CH₂OH. In certain embodiments, R⁹ is —CH₂CH₂CH₂SO₂CH₃. In certain embodiments, R⁹ is

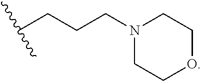 [SMILES: *CCCN1CCOCC1]

In certain embodiments, R⁹ is —CH₂CH₂CH₂NH₂. In certain embodiments, R⁹ is —CH₂CH₂CH₂N(CH₃)₂. In certain embodiments, R⁹ is —CH₂C(CH₃)₂OH. In certain embodiments, R⁹ is —CH₂CF₂CH₃. In certain embodiments, R⁹ is —CH₂CH═CH₂, —CH₂CH₂F, —CH₂CHF₂,

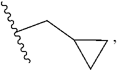 [SMILES: *CC1CC1]

or —CH₂CH₂CH₂OCH₃. In certain embodiments, R⁹ is —CH₂CH═CH₂, —CH₂CH₂F, —CH₂CHF₂, —CH₂CF₂CH₃, —CH₂CH₂CH₂F, —CH₂CH₂CF₂H, —CH₂CH₂CH₂Cl,

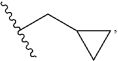 [SMILES: *CC1CC1]

—CH₂CH₂CH₂OCH₃, —CH₂CH₂CH₂OH, —CH₂C(CH₃)₂OH, —CH₂CH₂CH₂SO₂CH₃, CH₂CH₂CH₂NH₂, —CH₂CH₂CH₂N(CH₃)₂, or

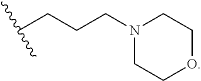 [SMILES: *CCCN1CCOCC1]

In certain embodiments, R⁹ is unsubstituted C₁₋₆ alkyl. In certain embodiments, R⁹ is —CH₃, —CH₂CH₃, —CH₂CH₂CH₃, —CH(CH₃)₂, —CH₂CH(CH₃)₂, or —C(CH₃)₃. In certain embodiments, R⁹ is —CH₃, —CH₂CH₃, —CH₂CH₂CH₃, —CH₂CH₂CH₂CH₃, —CH(CH₃)₂, —CH₂CH(CH₃)₂, —CH₂C(CH₃)₃, or —C(CH₃)₃. In certain embodiments, R⁹ is —CH₃. In certain embodiments, R⁹ is —CH₂CH₃. In certain embodiments, R⁹ is —CH₂CH₂CH₃. In certain embodiments, R⁹ is —CH(CH₃)₂. In certain embodiments, R⁹ is —C(CH₃)₃. In certain embodiments, R⁹ is —CH₂CH(CH₃)₂.

### Ring R

As described herein, R is heterocyclyl. In certain embodiments, R is 6-14 membered heterocyclyl. In certain embodiments, R is 8-12 membered heterocyclyl. In certain embodiments, R is 9-12 membered heterocyclyl. In certain embodiments, R is 9-11 membered heterocyclyl. In certain embodiments, R is 11-membered heterocyclyl. R is 10-membered heterocyclyl.

In certain embodiments, R comprises at least one unsaturated bond in its ring. In certain embodiments, R comprises at least one double bond (e.g., carbon-carbon or carbon-nitrogen). In certain embodiments, R comprises at least one unsaturated carbon-carbon bond in its ring. In certain embodiments, the unsaturated carbon-carbon bond is a double bond (i.e., alkenyl bond). In certain embodiments, the unsaturated carbon-carbon bond is a triple bond (i.e., alkynyl bond). In certain embodiments, R comprises at least one heteroatom in addition to the oxygen in its ring. In certain embodiments, R comprises at least one heteroatom in addition to the oxygen in its ring, wherein the heteroatom is nitrogen, oxygen, or sulfur. In certain embodiments, R comprises at least one heteroatom in addition to the oxygen in its ring, wherein the heteroatom is nitrogen. In certain embodiments, R comprises at least one heteroatom in addition to the oxygen in its ring, wherein the heteroatom is oxygen. In certain embodiments, R comprises at least one heteroatom in addition to the oxygen in its ring, wherein the heteroatom is sulfur. In certain embodiments, R comprises at least one sulfur atom in its ring. In certain embodiments, R comprises one sulfur atom in its ring.

In certain embodiments, R is a heterocyclyl of formula:

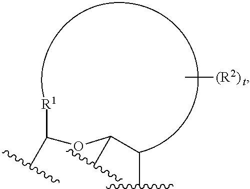

wherein: R¹ is —NR²—, —O—, or —S—. In certain embodiments, R¹ is —NR²—. In certain embodiments, R¹ is —O—. In certain embodiments, R¹ is —S—.

In certain embodiments, R is a heterocyclyl of formula:

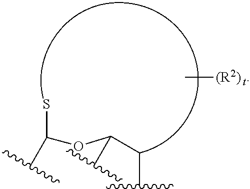

In certain embodiments, R is a heterocyclyl of formula:

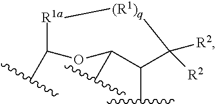

wherein: R^{1a} is —NR²—, —O—, or —S—; each R¹ is independently a bond, —CR²R²—, CR², C, N, —NR²—, —O—, or —S—; and q is 3-6. In certain embodiments, R¹ is —NR²—. In certain embodiments, R¹ is —O—. In certain embodiments, R¹ is —S—. In certain embodiments, R^{1a} is —NR²—. In certain embodiments, R^{1a} is —O—. In certain embodiments, R^{1a} is —S—.

In certain embodiments, R is a heterocyclyl of formula:

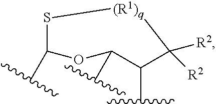

wherein: each R¹ is independently a bond, —CR²R²—, CR², C, N, —NR²—, —O—, or —S—; and q is 3-6. In certain embodiments, each R¹ is independently —CR²R²-, CR², C, N, —NR²—, or —O—. In certain embodiments, each R¹ is independently —CR²R²—, CR², N, —NR²—, or —O—. In certain embodiments, each R¹ is independently —CR²R²—, CR², C, N, or —NR²—. In certain embodiments, each R¹ is independently —CR²R²—, CR², N, or —NR²—. In certain embodiments, each R¹ is independently —CR²R²-, CR², C, or —O—. In certain embodiments, each R¹ is independently —CR²R²-, CR², or —O—. In certain embodiments, each R¹ is independently-CR²R²-, CR², or C. In certain embodiments, each R¹ is independently —CR²R²- or CR². In certain embodiments, each R¹ is independently —CR²R²- or CR². In certain embodiments, each R¹ is independently —CR²R²- or CR²; and q is 3. In certain embodiments, each R¹ is independently —CR²R²- or CR²; and q is 4. In certain embodiments, each R¹ is independently-CR²R²- or CR²; and q is 5. In certain embodiments, each R¹ is independently —CR²R²- or CR²; and q is 6.

In certain embodiments, R is a heterocyclyl of formula:

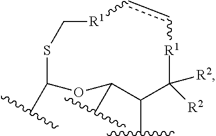

wherein: each R¹ is independently a bond or —CR²R²—; and [symbol] represents a single or double bond.

In certain embodiments, R is a heterocyclyl of formula:

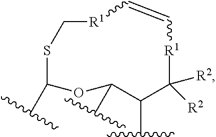

wherein: each R¹ is independently a bond or —CR²R²—.

In certain embodiments, R is a heterocyclyl of formula:

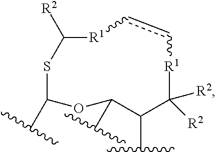

wherein: each R¹ is independently a bond or —CR²R²; and [symbol] represents a single or double bond.

In certain embodiments, R is a heterocyclyl of formula:

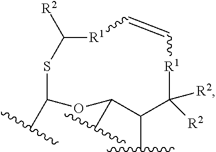

wherein: each R¹ is independently a bond or —CR²R²—.

In certain embodiments, R is a heterocyclyl of formula:

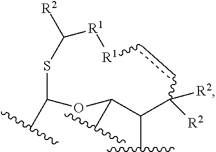

wherein: each R¹ is independently a bond or —CR²R²; and [symbol] represents a single or double bond.

In certain embodiments, R is a heterocyclyl of formula:

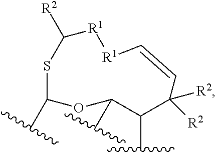

wherein: each R¹ is independently a bond or —CR²R²—.

In certain embodiments, R is a heterocyclyl of formula:

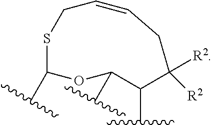

In certain embodiments, R is a heterocyclyl of formula:

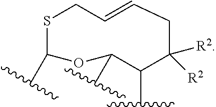

In certain embodiments, R is a heterocyclyl of formula:

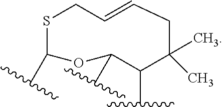 [SMILES: *C1OC(*)C(*)C(C)(C)C/C=C/CS1]

In certain embodiments, R is a heterocyclyl of formula:

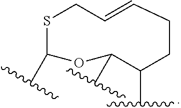 [SMILES: *C1OC(*)C(*)CC/C=C/CS1]

In certain embodiments, R is a heterocyclyl of formula:

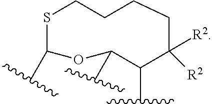

In certain embodiments, R is a heterocyclyl of formula:

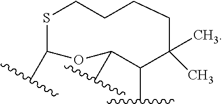 [SMILES: *C1OC(*)C(*)C(C)(C)CCCCS1]

In certain embodiments, R is a heterocyclyl of formula:

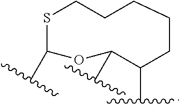 [SMILES: *C1OC(*)C(*)CCCCCS1]

In certain embodiments, R is a heterocyclyl of formula:

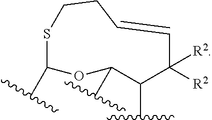

In certain embodiments, R is a heterocyclyl of formula:

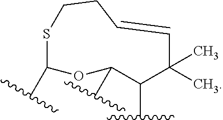 [SMILES: *C1OC(*)C(*)C(C)(C)/C=C/CCS1]

In certain embodiments, R is a heterocyclyl of formula:

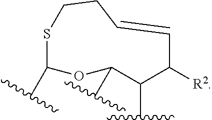

In certain embodiments, R is a heterocyclyl of formula:

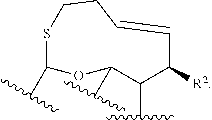

In certain embodiments, R is a heterocyclyl of formula:

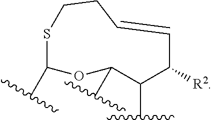

In certain embodiments, R is a heterocyclyl of formula:

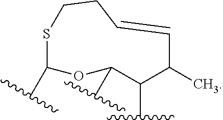 [SMILES: *C1OC(*)C(*)C(C)/C=C/CCS1]

In certain embodiments, R is a heterocyclyl of formula:

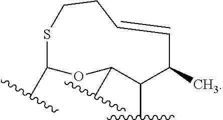 [SMILES: *C1OC(*)C(*)[C@H](C)/C=C/CCS1]

In certain embodiments, R is a heterocyclyl of formula:

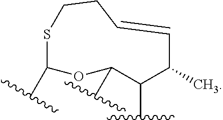 [SMILES: *C1OC(*)C(*)[C@@H](C)/C=C/CCS1]

In certain embodiments, R is a heterocyclyl of formula:

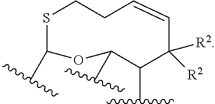

In certain embodiments, R is a heterocyclyl of formula:

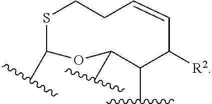

In certain embodiments, R is a heterocyclyl of formula:

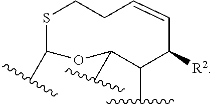

In certain embodiments, R is a heterocyclyl of formula:

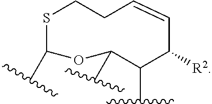

In certain embodiments, R is a heterocyclyl of formula:

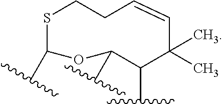 [SMILES: *C1OC(*)C(*)C(C)(C)/C=C\CCS1]

In certain embodiments, R is a heterocyclyl of formula:

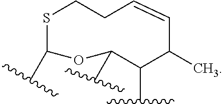 [SMILES: *C1OC(*)C(*)C(C)/C=C\CCS1]

In certain embodiments, R is a heterocyclyl of formula:

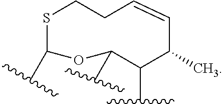 [SMILES: *C1OC(*)C(*)[C@@H](C)/C=C\CCS1]

In certain embodiments, R is a heterocyclyl of formula:

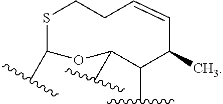 [SMILES: *C1OC(*)C(*)[C@H](C)/C=C\CCS1]

In certain embodiments, R is a heterocyclyl of formula:

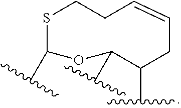 [SMILES: *C1OC(*)C(*)C/C=C\CCS1]

In certain embodiments, R is a heterocyclyl of formula:

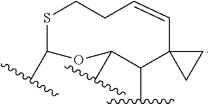 [SMILES: *C1OC(*)C(*)C2(/C=C\CCS1)CC2]

In certain embodiments, R is a heterocyclyl of formula:

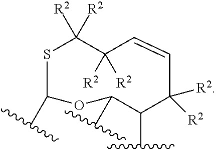

In certain embodiments, R is a heterocyclyl of formula:

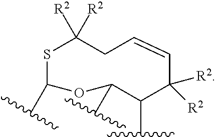

In certain embodiments, R is a heterocyclyl of formula:

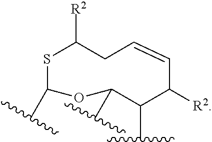

In certain embodiments, R is a heterocyclyl of formula:

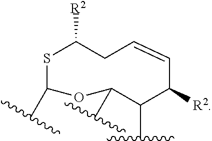

In certain embodiments, R is a heterocyclyl of formula:

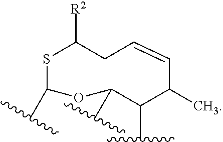

In certain embodiments, R is a heterocyclyl of formula:

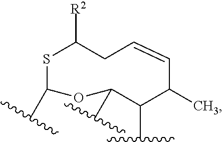

wherein R² is substituted or unsubstituted alkyl or substituted or unsubstituted heteroraryl. In certain embodiments, R² is substituted alkyl or substituted heteroraryl. In certain embodiments, R² is haloalkyl or substituted triazolyl. In certain embodiments, R² is fluoromethyl or triazolyl substituted with 2,4-difluorophenyl.

In certain embodiments, R is a heterocyclyl of formula:

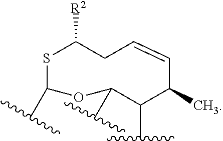

In certain embodiments, R is a heterocyclyl of formula:

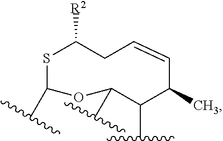

wherein R² is substituted or unsubstituted alkyl or substituted or unsubstituted heteroraryl. In certain embodiments, R² is substituted alkyl or substituted heteroraryl. In certain embodiments, R² is haloalkyl or substituted triazolyl. In certain embodiments, R² is fluoromethyl or triazolyl substituted with 2,4-difluorophenyl.

In certain embodiments, R is a heterocyclyl of formula:

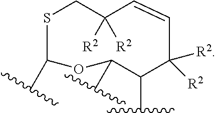

In certain embodiments, R is a heterocyclyl of formula:

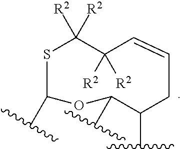

In certain embodiments, R is a heterocyclyl of formula:

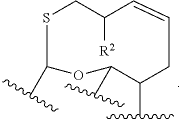

In certain embodiments, R is a heterocyclyl of formula:

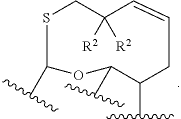

In certain embodiments, R is a heterocyclyl of formula:

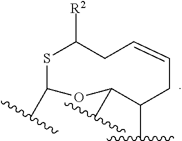

In certain embodiments, R is a heterocyclyl of formula:

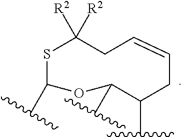

In certain embodiments, R is a heterocyclyl of formula:

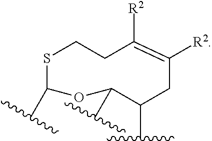

In certain embodiments, the two R² groups are joined to form a substituted or unsubstituted aryl ring, or a substituted or unsubstituted heteroaryl ring. In certain embodiments, the two R² groups are joined to form a substituted or unsubstituted phenyl ring.

In certain embodiments, R is a heterocyclyl of formula:

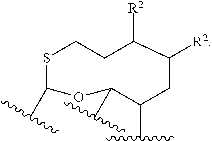

In certain embodiments, the two R² groups are joined to form a substituted or unsubstituted carbocyclyl ring, or a substituted or unsubstituted heterocyclyl ring. In certain embodiments, the two R² groups are each —OH.

In certain embodiments, R is a heterocyclyl of formula:

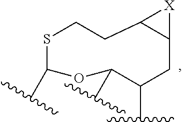

wherein: X is —O—, —NR²—, or —CR²R²—.

In certain embodiments, R is a heterocyclyl of formula:

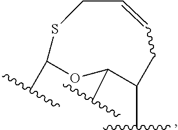

wherein R is optionally substituted with 1-4 independent R² groups.

In certain embodiments, R is a heterocyclyl of formula:

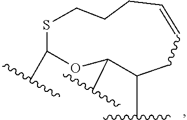

wherein R is optionally substituted with 1-4 independent R² groups.

In certain embodiments, R is a heterocyclyl of formula:

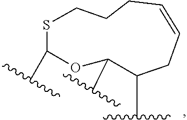

wherein R is optionally substituted with 1-4 independent R² groups.

In certain embodiments, R is a heterocyclyl of formula:

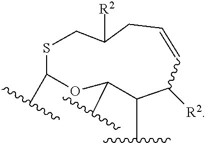

In certain embodiments, R is a heterocyclyl of formula:

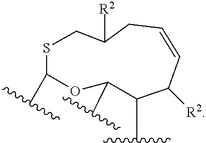

In certain embodiments, R is a heterocyclyl of formula:

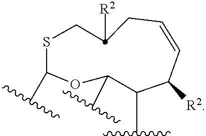

In certain embodiments, R is a heterocyclyl of formula:

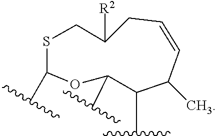

In certain embodiments, R is a heterocyclyl of formula:

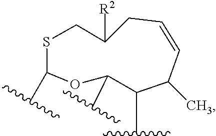

wherein R² is halogen. In certain embodiments, R² is fluoro.

In certain embodiments, R is a heterocyclyl of formula:

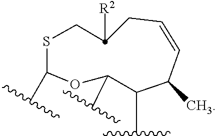

In certain embodiments, R is a heterocyclyl of formula:

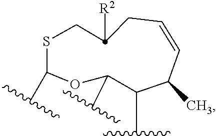

wherein R² is halogen. In certain embodiments, R² is fluoro.

In certain embodiments, R is a heterocyclyl of formula:

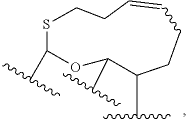

wherein R is optionally substituted with 1-4 independent R² groups.

In certain embodiments, R is a heterocyclyl of formula:

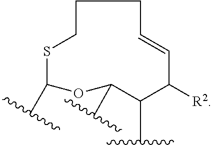

In certain embodiments, R is a heterocyclyl of formula:

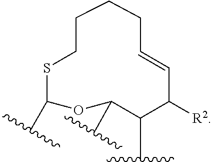

### Group R² and t

As described herein, each occurrence of R² is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring.

In certain embodiments, each occurrence of R² is, independently, hydrogen, halogen, substituted or unsubstituted alkyl, substituted or unsubstituted alkenyl, substituted or unsubstituted alkynyl, substituted or unsubstituted heteroalkyl, a nitrogen protecting group when attached to a nitrogen atom, —OR^{A}, —N(R^{A})₂, —SR^{A}, or —CN; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring.

In certain embodiments, each occurrence of R² is, independently, hydrogen, halogen, substituted or unsubstituted alkyl, substituted or unsubstituted alkenyl, substituted or unsubstituted alkynyl, substituted or unsubstituted heteroalkyl, substituted or unsubstituted aryl, substituted or unsubstituted heteroaryl, —OR^{A}, —N(R^{A})₂, —SR^{A}, or —CN; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring.

In certain embodiments, each occurrence of R² is, independently, hydrogen, halogen, substituted or unsubstituted alkyl, —OR^{A}, —N(R^{A})₂, or —SR^{A}; or two R² groups are joined to form a substituted or unsubstituted carbocyclyl ring, a substituted or unsubstituted aryl ring, or a substituted or unsubstituted heterocyclyl ring.

In certain embodiments, each occurrence of R² is, independently, halogen, substituted or unsubstituted alkyl, or —OR^{A}; or two R² groups are joined to form a substituted or unsubstituted carbocyclyl ring, a substituted or unsubstituted aryl ring, or a substituted or unsubstituted heterocyclyl ring.

In certain embodiments, each occurrence of R² is, independently, hydrogen, halogen, substituted or unsubstituted alkyl, substituted or unsubstituted alkynyl, substituted or unsubstituted aryl, or substituted or unsubstituted heteroaryl. In certain embodiments, each occurrence of R² is, independently, hydrogen, halogen, substituted or unsubstituted alkyl, substituted or unsubstituted alkynyl, or substituted or unsubstituted heteroaryl. In certain embodiments, each occurrence of R² is, independently, hydrogen, halogen, substituted or unsubstituted alkyl, substituted or unsubstituted aryl, or substituted or unsubstituted heteroaryl. In certain embodiments, each occurrence of R² is, independently, hydrogen, halogen, substituted or unsubstituted alkyl, or substituted or unsubstituted heteroaryl. In certain embodiments, each occurrence of R² is, independently, halogen, substituted or unsubstituted alkyl, or substituted or unsubstituted heteroaryl.

In certain embodiments, each occurrence of R² is, independently,

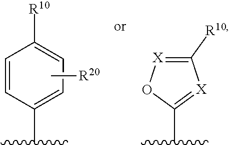

wherein R²⁰ is hydrogen, halogen, substituted or unsubstituted alkyl, —CN, —OR^{A}, —N(R^{A})₂, or —SR^{A}; X is N or CH; and R¹⁰ is hydrogen, substituted or unsubstituted alkyl, substituted or unsubstituted heterocyclyl, substituted or unsubstituted heterocyclylalkyl, substituted or unsubstituted aryl, or substituted or unsubstituted heteroaryl.

In certain embodiments, each occurrence of R² is, independently,

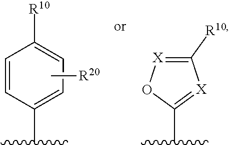

wherein R²⁰ is hydrogen, halogen, substituted or unsubstituted alkyl, —CN, —OR^{A}, —N(R^{A})₂, or —SR^{A}; X is N or CH; and R¹⁰ is hydrogen,

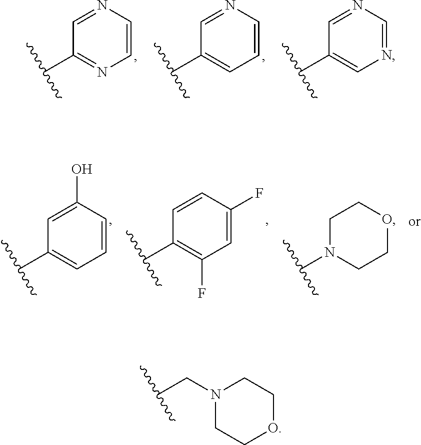

In certain embodiments, each occurrence of R² is, independently, hydrogen, fluoro, fluoromethyl, difluoromethyl, methyl, —OH, —CH₂NH₂, —CH₂OH, —CH₂OAc, —C≡CH,

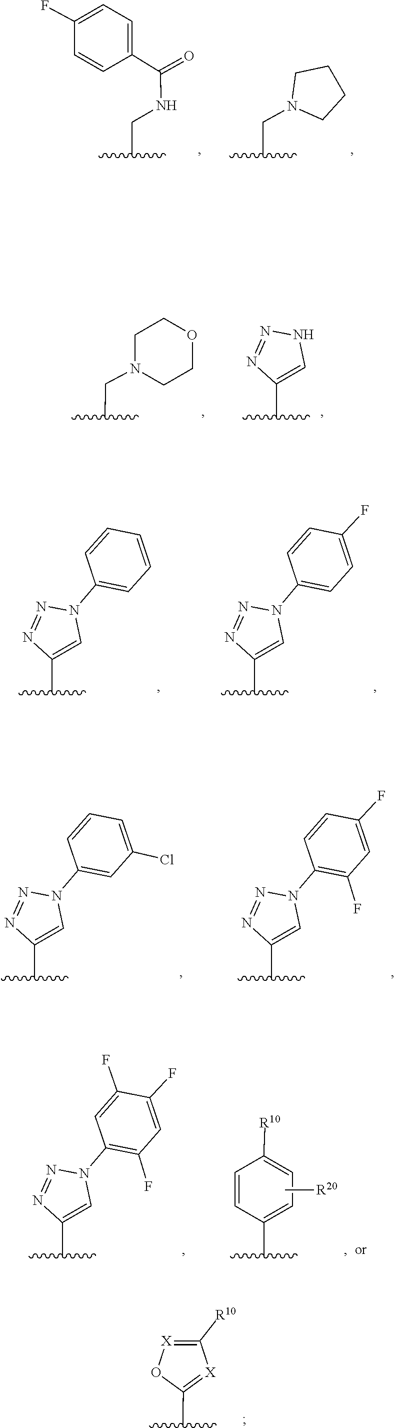

wherein R²⁰ is hydrogen, halogen, substituted or unsubstituted alkyl, —CN, —OR^{A}, —N(R^{A})₂, or —SR^{A}; X is N or CH; and R¹⁰ is hydrogen,

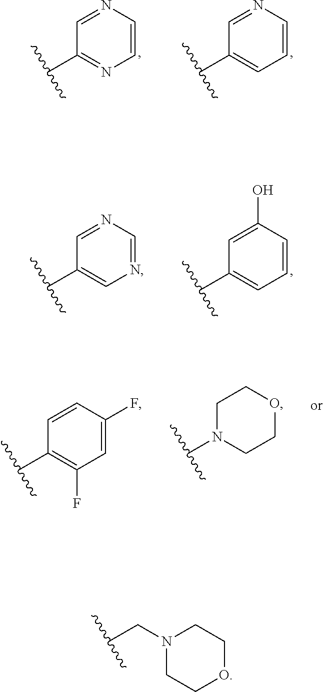

In certain embodiments, each occurrence of R² is, independently, hydrogen, —OH, —CH₂NH₂, —CH₂OH, —CH₂OAc, —C≡CH,

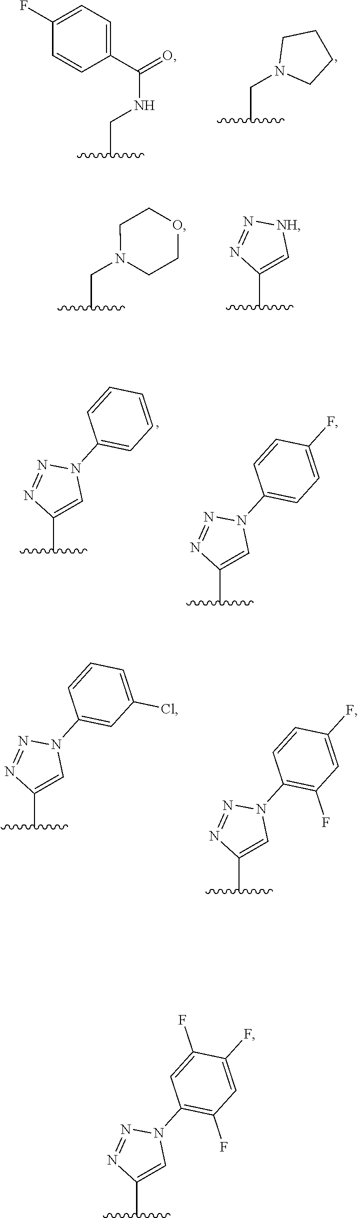

fluoro, fluoromethyl, difluoromethyl, or methyl.

In certain embodiments, R² is hydrogen. In certain embodiments, R² is —OH. In certain embodiments, R² is —CH₂NH₂. In certain embodiments, R² is —CH₂OH. In certain embodiments, R² is —CH₂OAc. In certain embodiments, R² is —C═CH. In certain embodiments, R² is

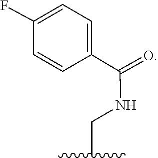 [SMILES: *CNC(=O)c1ccc(F)cc1]

In certain embodiments, R² is

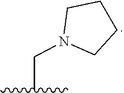 [SMILES: *CN1CCCC1]

In certain embodiments, R² is

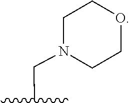 [SMILES: *CN1CCOCC1]

In certain embodiments, R² is

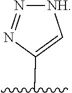 [SMILES: *c1c[nH]nn1]

In certain embodiments, R² is

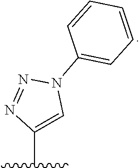 [SMILES: *c1cn(-c2ccccc2)nn1]

In certain embodiments, R² is

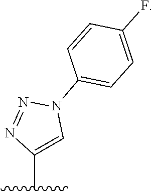 [SMILES: *c1cn(-c2ccc(F)cc2)nn1]

In certain embodiments, R² is

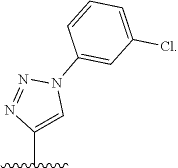 [SMILES: *c1cn(-c2cccc(Cl)c2)nn1]

In certain embodiments, R² is

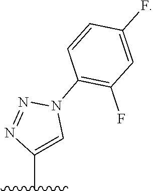 [SMILES: *c1cn(-c2ccc(F)cc2F)nn1]

In certain embodiments, R² is

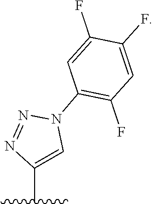 [SMILES: *c1cn(-c2cc(F)c(F)cc2F)nn1]

In certain embodiments, R² is fluoro. In certain embodiments, R² is fluoromethyl. In certain embodiments, R² is difluoromethyl. In certain embodiments, R² is methyl.

In certain embodiments, each occurrence of R² is, independently, fluoro, substituted or unsubstituted alkyl, or substituted or unsubstituted heteroaryl. In certain embodiments, each occurrence of R² is, independently, fluoro, substituted or unsubstituted alkyl, or substituted or unsubstituted 5-membered heteroaryl. In certain embodiments, each occurrence of R² is, independently, fluoro, haloalkyl, unsubstituted alkyl, or substituted or unsubstituted 5-membered heteroaryl. In certain embodiments, each occurrence of R² is, independently, fluoro, C₁₋₆ haloalkyl, unsubstituted C₁₋₆ alkyl, or substituted or unsubstituted 5-membered heteroaryl. In certain embodiments, each occurrence of R² is, independently, fluoro, C₁₋₄ haloalkyl, unsubstituted C₁₋₄ alkyl, or substituted or unsubstituted 5-membered heteroaryl. In certain embodiments, each occurrence of R² is, independently, fluoro, C₁₋₃ haloalkyl, unsubstituted C₁₋₃ alkyl, or substituted or unsubstituted 5-membered heteroaryl. In certain embodiments, each occurrence of R² is, independently, fluoro, C₁₋₂ haloalkyl, unsubstituted C₁₋₂ alkyl, or substituted or unsubstituted 5-membered heteroaryl. In certain embodiments, each occurrence of R² is, independently, fluoro, fluoromethyl, methyl, or substituted or unsubstituted triazolyl. In certain embodiments, each occurrence of R² is, independently, fluoro, fluoromethyl, methyl, or substituted triazolyl. In certain embodiments, each occurrence of R² is, independently, fluoro, fluoromethyl, methyl, or

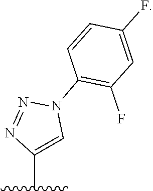 [SMILES: *c1cn(-c2ccc(F)cc2F)nn1]

In certain embodiments, each occurrence of R² is fluoro or methyl. In certain embodiments, each occurrence of R² is fluoromethyl or methyl. In certain embodiments, each occurrence of R² is substituted or unsubstituted triazolyl or methyl. In certain embodiments, each occurrence of R² is substituted triazolyl or methyl. In certain embodiments, each occurrence of R² is methyl or

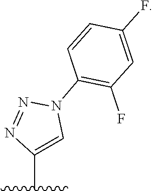 [SMILES: *c1cn(-c2ccc(F)cc2F)nn1]

In certain embodiments, each occurrence of R² is, independently, halogen, substituted or unsubstituted alkyl, or —OR^{A}. In certain embodiments, each occurrence of R² is, independently, fluoro, substituted or unsubstituted alkyl, or —OR^{A}. In certain embodiments, each occurrence of R² is, independently, fluoro, substituted or unsubstituted alkyl, or —OH. In certain embodiments, each occurrence of R² is, independently, fluoro, unsubstituted alkyl, or —OH. In certain embodiments, each occurrence of R² is, independently, fluoro, unsubstituted C₁₋₆ alkyl, or —OH. In certain embodiments, each occurrence of R² is, independently, fluoro, unsubstituted C₁₋₄ alkyl, or —OH. In certain embodiments, each occurrence of R² is, independently, fluoro, unsubstituted C₁₋₃ alkyl, or —OH. In certain embodiments, each occurrence of R² is, independently, fluoro, unsubstituted C₁₋₂ alkyl, or —OH. In certain embodiments, each occurrence of R² is, independently, fluoro, methyl, or —OH.

In certain embodiments, each occurrence of R² is, independently, hydrogen or substituted or unsubstituted alkyl. In certain embodiments, each occurrence of R² is, independently, hydrogen or unsubstituted alkyl. In certain embodiments, each occurrence of R² is, independently, hydrogen or unsubstituted C₁₋₆ alkyl. In certain embodiments, each occurrence of R² is, independently, hydrogen or unsubstituted C₁₋₄ alkyl. In certain embodiments, each occurrence of R² is, independently, hydrogen or unsubstituted C₁₋₃ alkyl. In certain embodiments, each occurrence of R² is, independently, hydrogen or unsubstituted C₁₋₂ alkyl. In certain embodiments, each occurrence of R² is, independently, hydrogen or methyl.

In certain embodiments, each occurrence of R² is, independently, substituted or unsubstituted alkyl. In certain embodiments, each occurrence of R² is, independently, unsubstituted alkyl. In certain embodiments, each occurrence of R² is, independently, unsubstituted C₁₋₆ alkyl. In certain embodiments, each occurrence of R² is, independently, unsubstituted C₁₋₄ alkyl. In certain embodiments, each occurrence of R² is, independently, unsubstituted C₁₋₃ alkyl. In certain embodiments, each occurrence of R² is, independently, unsubstituted C₁₋₂ alkyl. In certain embodiments, each occurrence of R² is, independently, methyl.

As described herein, t is an integer of 0-12. In certain embodiments, t is 0-6. In certain embodiments, t is 1-6. In certain embodiments, t is 0-4. In certain embodiments, t is 1-4. In certain embodiments, t is 0-2. In certain embodiments, t is 1 or 2. In certain embodiments, t is 0 or 1. In certain embodiments, t is 0. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7. In certain embodiments, t is 8. In certain embodiments, t is 9. In certain embodiments, t is 10. In certain embodiments, t is 11. In certain embodiments, t is 12.

### Embodiments of Formula (I)

In certain embodiments, the compound of Formula (I) is a compound of Formula (I-a):

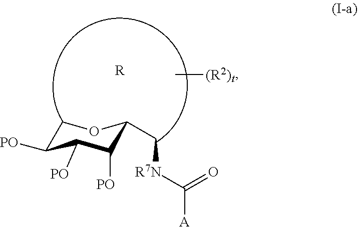

or a pharmaceutically acceptable salt thereof, wherein A, P, R, R², R⁷, and t are as defined herein.

In certain embodiments, the compound of Formula (I) is a compound of Formula (I-b):

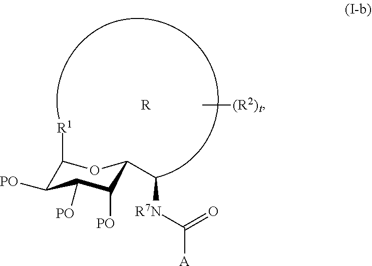

or a pharmaceutically acceptable salt thereof, wherein R¹ is —NR²—, —O—, or —S—; and A, P, R, R², R⁷, and t are as defined herein.

In certain embodiments, the compound of Formula (I) is a compound of Formula (I-c):

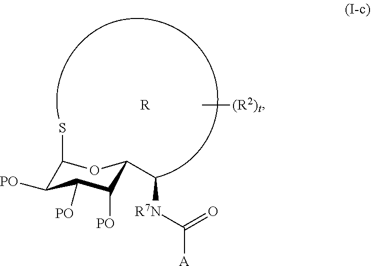

or a pharmaceutically acceptable salt thereof, wherein A, P, R², R⁷, and t are as defined herein.

In certain embodiments, the compound of Formula (I) is a compound of Formula (I-d):

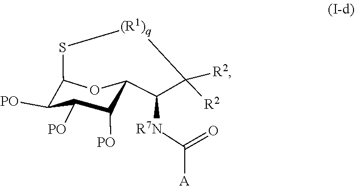

or a pharmaceutically acceptable salt thereof, wherein each R¹ is independently a bond, —CR²R²—, CR², C, N, —NR²—, —O—, or S; q is 3-6; and A, P, R², and R⁷ are as defined herein.

In certain embodiments, the compound of Formula (I) is a compound of Formula (I-e):

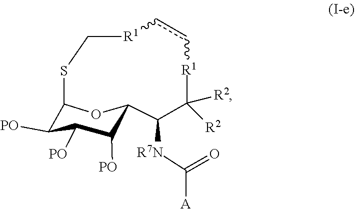

or a pharmaceutically acceptable salt thereof, wherein each R¹ is independently a bond or —CR²R²; [symbol] represents a single or double bond; and A, P, R², and R⁷ are as defined herein.

In certain embodiments, the compound of Formula (I) is a compound of Formula (I-e-1):

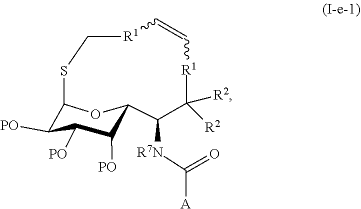

or a pharmaceutically acceptable salt thereof, wherein each R¹ is independently a bond or —CR²R²; and A, P, R², and R⁷ are as defined herein.

In certain embodiments, the compound of Formula (I) is a compound of Formula (I-f):

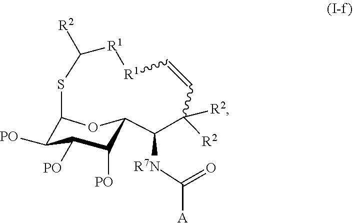

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-f-1):

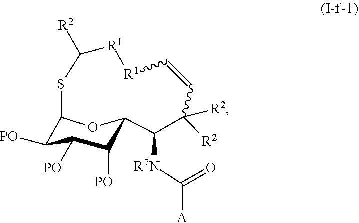

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-f-2):

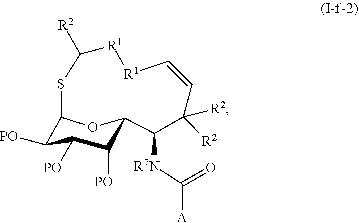

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-f-3):

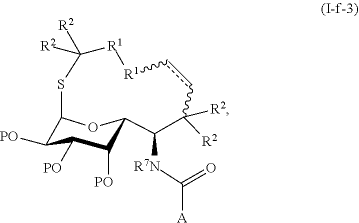

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-f-4):

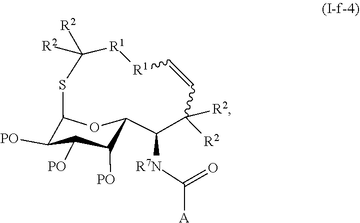

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-f-5):

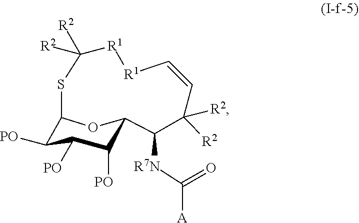

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g):

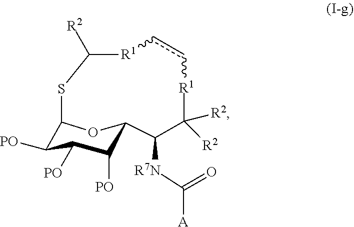

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g-1):

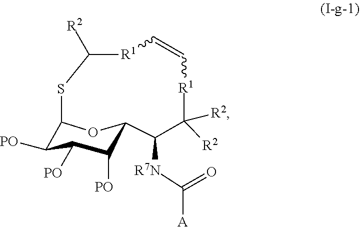

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g-2):

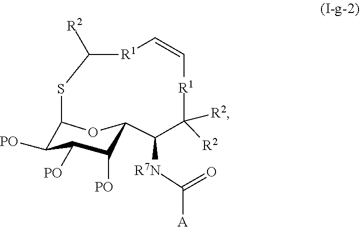

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g-3):

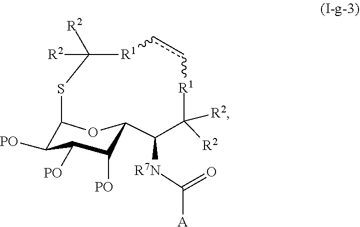

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g-4):

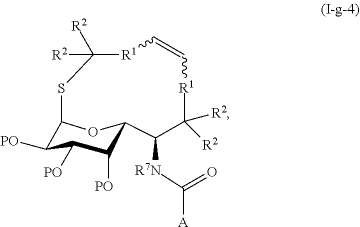

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-g-5):

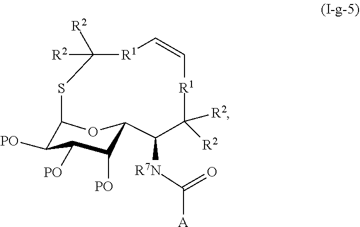

or a pharmaceutically acceptable salt thereof, wherein each R¹ is independently a bond or —CR²R²—; and A, P, R², and R⁷ are as defined herein.

In certain embodiments, the compound of Formula (I) is a compound of Formula (I-h):

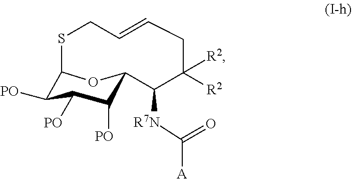

or a pharmaceutically acceptable salt thereof, wherein A, P, R², and R⁷ are as defined herein.

In certain embodiments, the compound of Formula (I) is a compound of Formula (I-h-1):

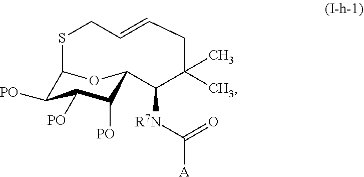

or a pharmaceutically acceptable salt thereof, wherein A, P, and R⁷ are as defined herein.

In certain embodiments, the compound of Formula (I) is a compound of Formula (I-h-2):

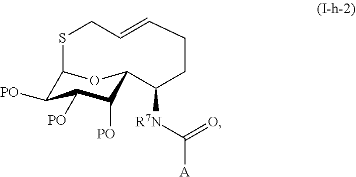

or a pharmaceutically acceptable salt thereof, wherein A, P, and R⁷ are as defined herein.

In certain embodiments, the compound of Formula (I) is a compound of Formula (I-i):

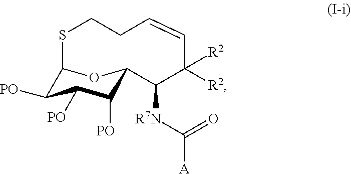

or a pharmaceutically acceptable salt thereof, wherein A, P, R², and R⁷ are as defined herein.

In certain embodiments, the compound of Formula (I) is a compound of Formula (I-i-1):

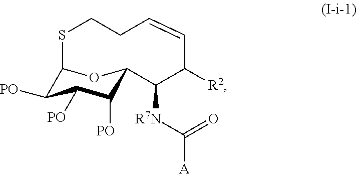

or a pharmaceutically acceptable salt thereof, wherein A, P, R², and R⁷ are as defined herein.

In certain embodiments, the compound of Formula (I) is a compound of Formula (I-i-2):

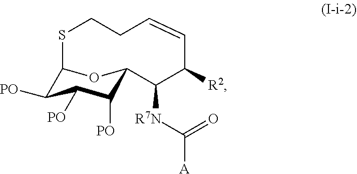

or a pharmaceutically acceptable salt thereof, wherein A, P, R², and R⁷ are as defined herein.

In certain embodiments, the compound of Formula (I) is a compound of Formula (I-i-3):

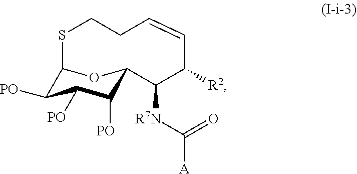

or a pharmaceutically acceptable salt thereof, wherein A, P, R², and R⁷ are as defined herein.

In certain embodiments, the compound of Formula (I) is a compound of Formula (I-i-4):

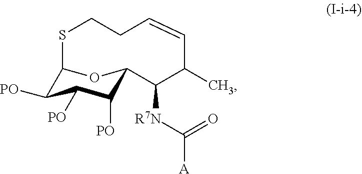

or a pharmaceutically acceptable salt thereof, wherein A, P, and R⁷ are as defined herein.

In certain embodiments, the compound of Formula (I) is a compound of Formula (I-i-5):

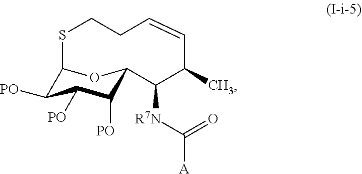

or a pharmaceutically acceptable salt thereof, wherein A, P, and R⁷ are as defined herein.

In certain embodiments, the compound of Formula (I) is a compound of Formula (I-i-6):

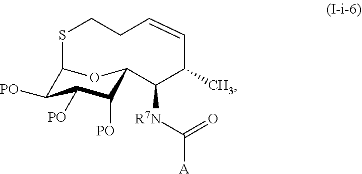

or a pharmaceutically acceptable salt thereof, wherein A, P, and R⁷ are as defined herein.

In certain embodiments, the compound of Formula (I) is a compound of Formula (I-i-7):

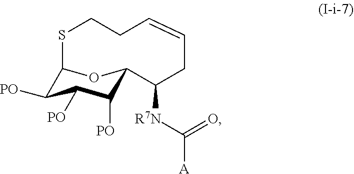

or a pharmaceutically acceptable salt thereof, wherein A, P, and R⁷ are as defined herein.

In certain embodiments, the compound of Formula (I) is a compound of Formula (I-i-8):

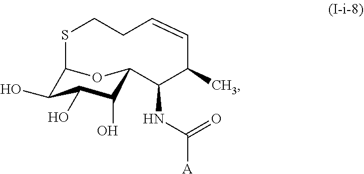

or a pharmaceutically acceptable salt thereof, wherein A is as defined herein.

In certain embodiments, the compound of Formula (I) is a compound of Formula (I-j):

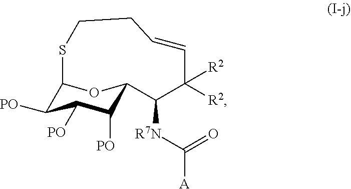

or a pharmaceutically acceptable salt thereof, wherein A, P, R², and R⁷ are as defined herein.

In certain embodiments, the compound of Formula (I) is a compound of Formula (I-j-1):

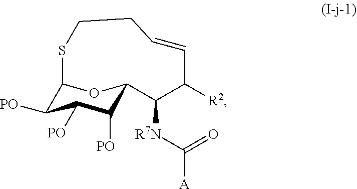

or a pharmaceutically acceptable salt thereof, wherein A, P, R², and R⁷ are as defined herein.

In certain embodiments, the compound of Formula (I) is a compound of Formula (I-j-2):

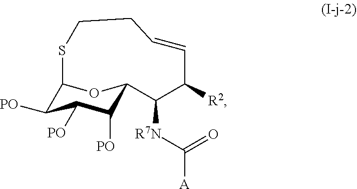

or a pharmaceutically acceptable salt thereof, wherein A, P, R², and R⁷ are as defined herein.

In certain embodiments, the compound of Formula (I) is a compound of Formula (I-j-3):

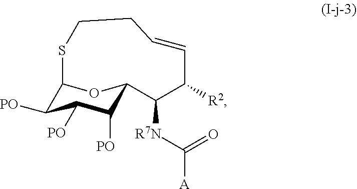

or a pharmaceutically acceptable salt thereof, wherein A, P, R², and R⁷ are as defined herein.

In certain embodiments, the compound of Formula (I) is a compound of Formula (I-j-4):

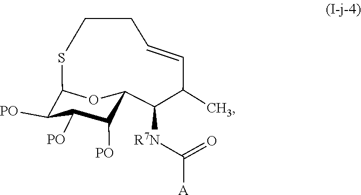

or a pharmaceutically acceptable salt thereof, wherein A, P, and R⁷ are as defined herein.

In certain embodiments, the compound of Formula (I) is a compound of Formula (I-j-5):

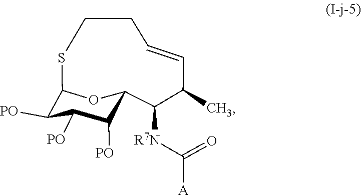

or a pharmaceutically acceptable salt thereof, wherein A, P, and R⁷ are as defined herein.

In certain embodiments, the compound of Formula (I) is a compound of Formula (I-j-6):

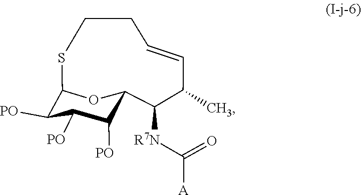

or a pharmaceutically acceptable salt thereof, wherein A, P, and R⁷ are as defined herein.

In certain embodiments, the compound of Formula (I) is a compound of Formula (I-k):

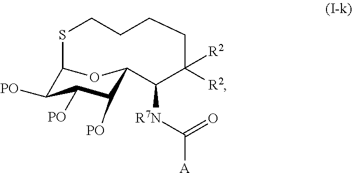

or a pharmaceutically acceptable salt thereof, wherein A, P, R², and R⁷ are as defined herein.

In certain embodiments, the compound of Formula (I) is a compound of Formula (I-k-1):

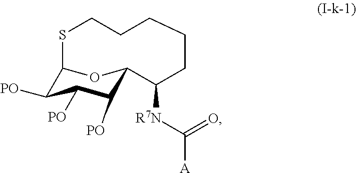

or a pharmaceutically acceptable salt thereof, wherein A, P, and R⁷ are as defined herein.

In certain embodiments, the compound of Formula (I) is a compound of Formula (I-1):

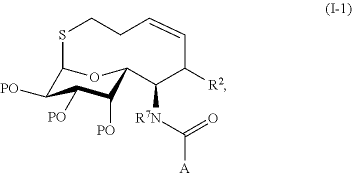

or a pharmaceutically acceptable salt thereof, wherein A, P, R², and R⁷ are as defined herein.

In certain embodiments, the compound of Formula (I) is a compound of Formula (I-1-1):

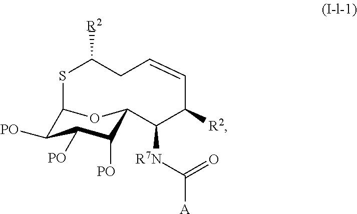

or a pharmaceutically acceptable salt thereof, wherein A, P, R², and R⁷ are as defined herein.

In certain embodiments, the compound of Formula (I) is a compound of Formula (I-1-2):

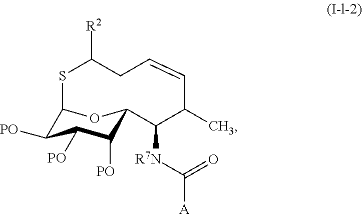

or a pharmaceutically acceptable salt thereof, wherein A, P, R², and R⁷ are as defined herein. In certain embodiments of the compound of Formula (I-1-2), R² is substituted or unsubstituted alkyl or substituted or unsubstituted heteroraryl. In certain embodiments of the compound of Formula (I-1-2), R² is substituted alkyl or substituted heteroraryl. In certain embodiments of the compound of Formula (I-1-2), R² is haloalkyl or substituted triazolyl. In certain embodiments of the compound of Formula (I-1-2), R² is fluoromethyl or triazolyl substituted with 2,4-difluorophenyl.

In certain embodiments, the compound of Formula (I) is a compound of Formula (I-1-3):

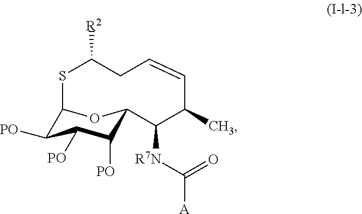

or a pharmaceutically acceptable salt thereof, wherein A, P, R², and R⁷ are as defined herein. In certain embodiments of the compound of Formula (I-1-3), R² is substituted or unsubstituted alkyl or substituted or unsubstituted heteroraryl. In certain embodiments of the compound of Formula (I-1-3), R² is substituted alkyl or substituted heteroraryl. In certain embodiments of the compound of Formula (I-1-3), R² is haloalkyl or substituted triazolyl. In certain embodiments of the compound of Formula (I-1-3), R² is fluoromethyl or triazolyl substituted with 2,4-difluorophenyl.

In certain embodiments, the compound of Formula (I) is a compound of Formula (I-1-4):

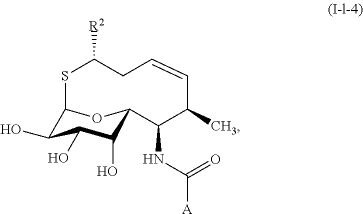

or a pharmaceutically acceptable salt thereof, wherein A and R² are as defined herein. In certain embodiments of the compound of Formula (I-1-4), R² is substituted or unsubstituted alkyl or substituted or unsubstituted heteroraryl. In certain embodiments of the compound of Formula (I-1-4), R² is substituted alkyl or substituted heteroraryl. In certain embodiments of the compound of Formula (I-1-4), R² is haloalkyl or substituted triazolyl. In certain embodiments of the compound of Formula (I-1-4), R² is fluoromethyl or triazolyl substituted with 2,4-difluorophenyl.

In certain embodiments, the compound of Formula (I) is a compound of Formula (I-m):

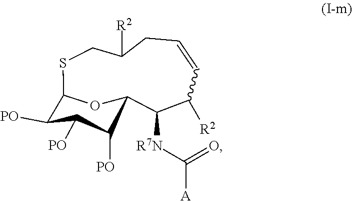

or a pharmaceutically acceptable salt thereof, wherein A, P, R², and R⁷ are as defined herein.

In certain embodiments, the compound of Formula (I) is a compound of Formula (I-m-1):

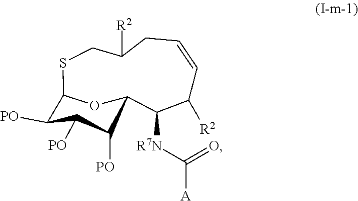

or a pharmaceutically acceptable salt thereof, wherein A, P, R², and R⁷ are as defined herein.

In certain embodiments, the compound of Formula (I) is a compound of Formula (I-m-2):

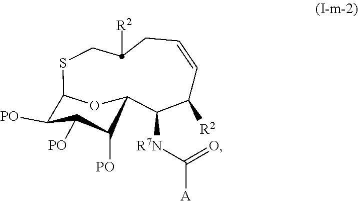

or a pharmaceutically acceptable salt thereof, wherein A, P, R², and R⁷ are as defined herein.

In certain embodiments, the compound of Formula (I) is a compound of Formula (I-m-3):

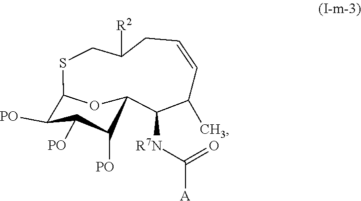

or a pharmaceutically acceptable salt thereof, wherein A, P, R², and R⁷ are as defined herein. In certain embodiments of the compound of Formula (I-m-3), R² is halogen. In certain embodiments of the compound of Formula (I-m-3), R² is fluoro.

In certain embodiments, the compound of Formula (I) is a compound of Formula (I-m-4):

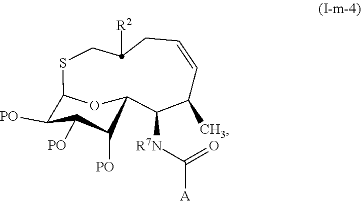

or a pharmaceutically acceptable salt thereof, wherein A, P, R², and R⁷ are as defined herein. In certain embodiments of the compound of Formula (I-m-4), R² is halogen. In certain embodiments of the compound of Formula (I-m-4), R² is fluoro.

In certain embodiments, the compound of Formula (I) is a compound of Formula (I-m-5):

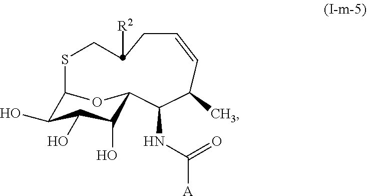

or a pharmaceutically acceptable salt thereof, wherein A and R² are as defined herein. In certain embodiments of the compound of Formula (I-m-5), R² is halogen. In certain embodiments of the compound of Formula (I-m-5), R² is fluoro.

In certain embodiments, the compound of Formula (I) is a compound of Formula (I-n):

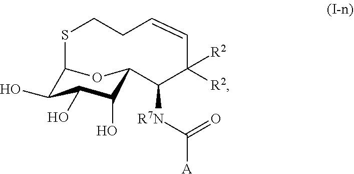

or a pharmaceutically acceptable salt thereof, wherein A, R², and R⁷ are as defined herein.

In certain embodiments, the compound of Formula (I) is a compound of Formula (I-n-1):

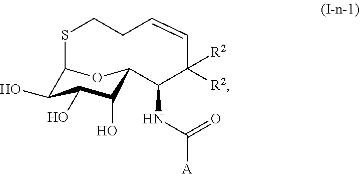

or a pharmaceutically acceptable salt thereof, wherein A and R² are as defined herein.

In certain embodiments, the compound of Formula (I) is a compound of Formula (I-n-2):

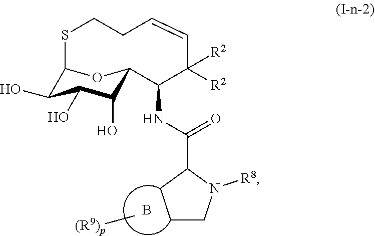

or a pharmaceutically acceptable salt thereof, wherein B, R², R⁸, R⁹, and p are as defined herein.

In certain embodiments, the compound of Formula (I) is a compound of Formula (I-n-3):

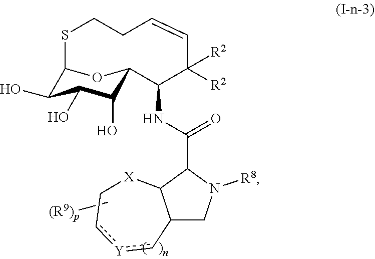

or a pharmaceutically acceptable salt thereof, wherein R², R⁸, R⁹, X, Y, n, and p are as defined herein.

In certain embodiments, the compound of Formula (I) is a compound of Formula (I-n-4):

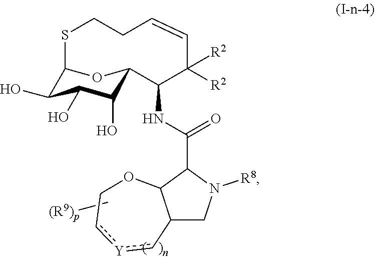

or a pharmaceutically acceptable salt thereof, wherein R², R⁸, R⁹, Y, and p are as defined herein.

In certain embodiments, the compound of Formula (I) is a compound of Formula (I-n-5):

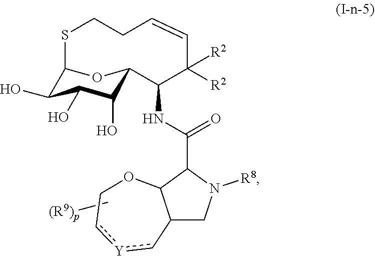

or a pharmaceutically acceptable salt thereof, wherein R², R⁸, R⁹, Y, and p are as defined herein.

In certain embodiments, the compound of Formula (I) is a compound of Formula (I-n-6):

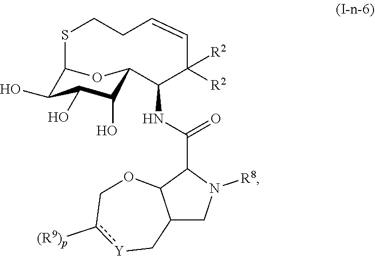

or a pharmaceutically acceptable salt thereof, wherein R², R⁸, R⁹, Y, and p are as defined herein.

In certain embodiments, the compound of Formula (I) is a compound of Formula (I-n-7):

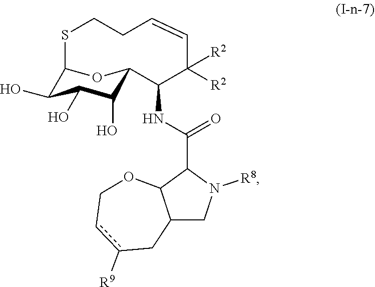

or a pharmaceutically acceptable salt thereof, wherein R², R⁸, and R⁹ are as defined herein.

In certain embodiments, the compound of Formula (I) is a compound of Formula (I-n-8):

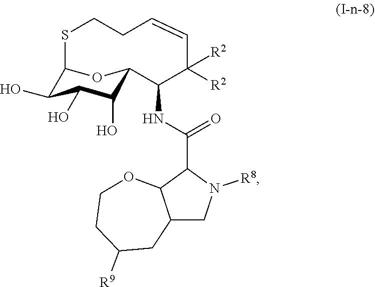

or a pharmaceutically acceptable salt thereof, wherein R², R⁸, and R⁹ are as defined herein.

In certain embodiments, the compound of Formula (I) is a compound of Formula (I-n-9):

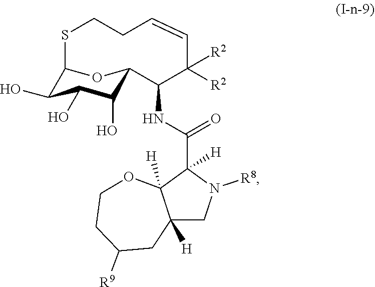

or a pharmaceutically acceptable salt thereof, wherein R², R⁸, and R⁹ are as defined herein.

In certain embodiments, the compound of Formula (I) is a compound of Formula (I-n-10):

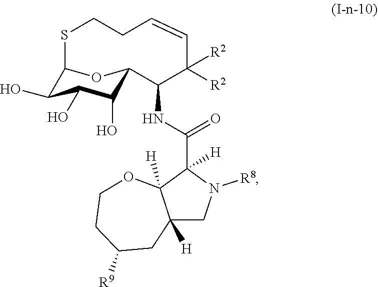

or a pharmaceutically acceptable salt thereof, wherein R², R⁸, and R⁹ are as defined herein.

In certain embodiments, the compound of Formula (I) is a compound of Formula (I-n-11):

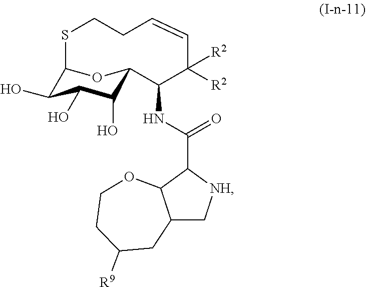

or a pharmaceutically acceptable salt thereof, wherein R² and R⁹ are as defined herein.

In certain embodiments, the compound of Formula (I) is a compound of Formula (I-n-12):

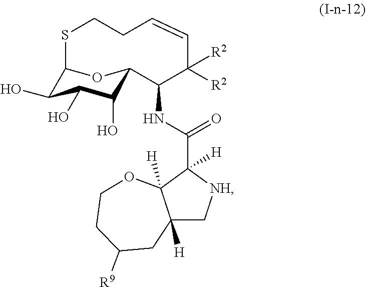

or a pharmaceutically acceptable salt thereof, wherein R² and R⁹ are as defined herein.

In certain embodiments, the compound of Formula (I) is a compound of Formula (I-n-13):

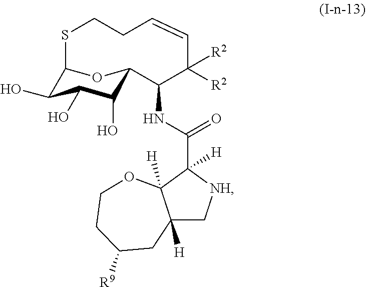

or a pharmaceutically acceptable salt thereof, wherein R² and R⁹ are as defined herein.

In certain embodiments, the compound of Formula (I) is a compound of Formula (I-n-14):

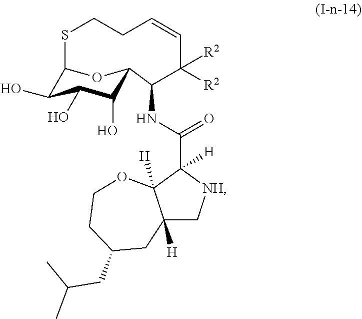

or a pharmaceutically acceptable salt thereof, wherein R² is as defined herein.

In certain embodiments, the compound of Formula (I) is a compound of any of the following formula:

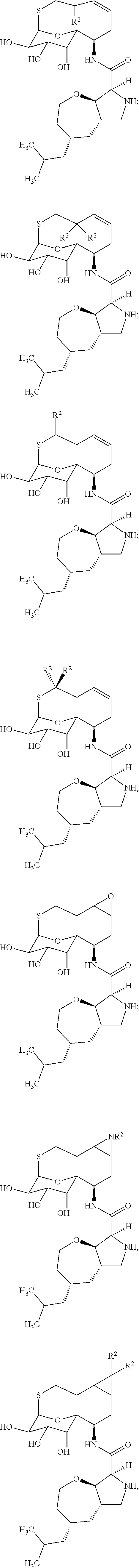

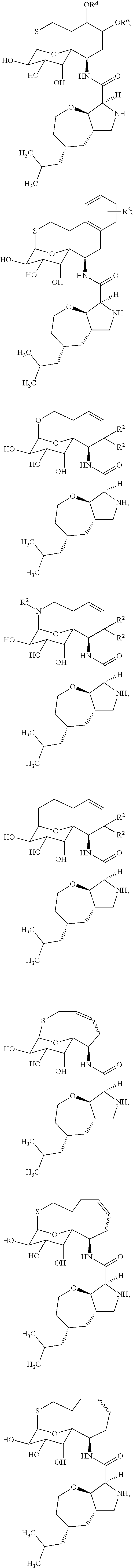

and pharmaceutically acceptable salts thereof.

In certain embodiments, the compound of Formula (I) is a compound of formula:

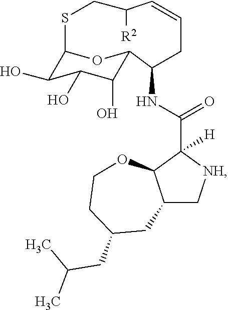

or a pharmaceutically acceptable salt thereof.

In certain embodiments, the compound of Formula (I) is a compound of formula:

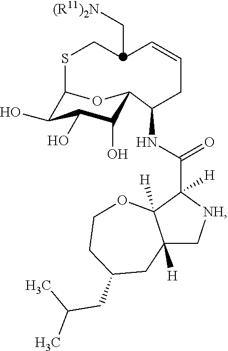

or a pharmaceutically acceptable salt thereof, wherein each R¹¹ is independently hydrogen, substituted or unsubstituted alkyl, substituted or unsubstituted carbocyclyl, substituted or unsubstituted heterocyclyl, or a nitrogen protecting group, or two R¹¹ groups are joined to form a substituted or unsubstituted heterocyclyl ring. In certain embodiments, —N(R¹¹)₂ is

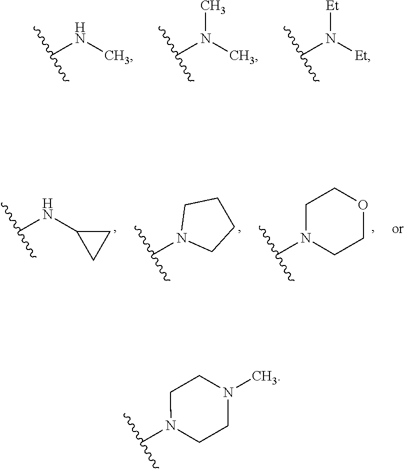

In certain embodiments, the compound of Formula (I) is a compound of formula:

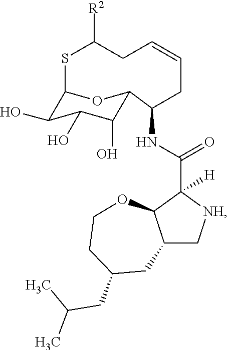

or a pharmaceutically acceptable salt thereof.

In certain embodiments, the compound of Formula (I) is a compound of formula:

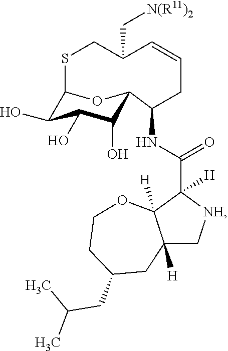

or a pharmaceutically acceptable salt thereof, wherein each R¹¹ is independently hydrogen, substituted or unsubstituted alkyl, substituted or unsubstituted carbocyclyl, substituted or unsubstituted heterocyclyl, a nitrogen protecting group, or two R¹¹ groups are joined to form a substituted or unsubstituted heterocyclyl ring. In certain embodiments, —N(R¹¹)₂ is

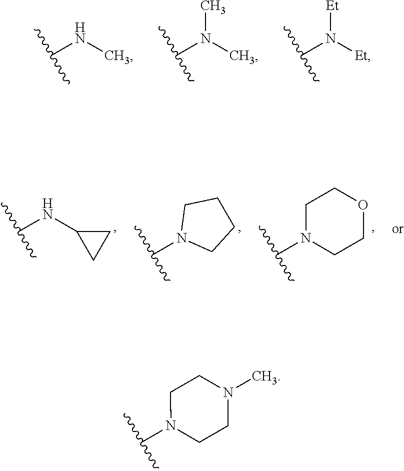

In certain embodiments, the compound of Formula (I) is a compound of formula:

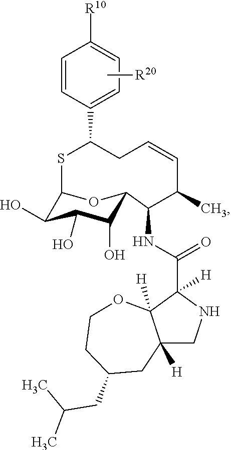

or a pharmaceutically acceptable salt thereof, wherein R²⁰ is hydrogen, halogen, substituted

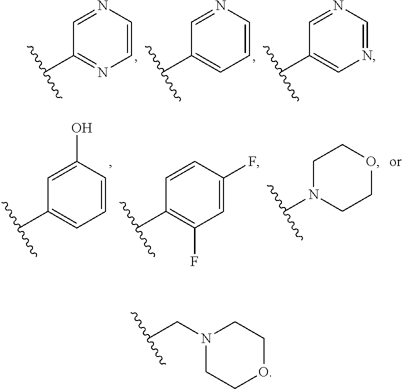

or unsubstituted alkyl, —CN, —OR^{A}, —N(R^{A})₂, or —SR^{A}; and R¹⁰ is hydrogen,

In certain embodiments, the compound of Formula (I) is a compound of formula:

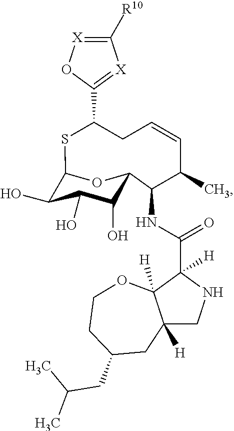

or a pharmaceutically acceptable salt thereof, wherein X is N or CH; and R¹⁰ is hydrogen,

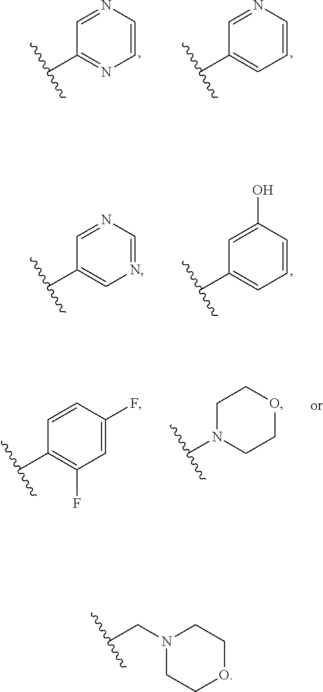

### Exemplary Compounds

Exemplary compounds of Formula (I) include, but are not limited to, the compounds listed in Table 1.

TABLE 1
Exemplary compounds of Formula (I)

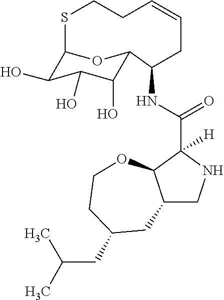 [SMILES: CC(C)C[C@@H]1CCO[C@@H]2[C@H](CN[C@@H]2C(=O)N[C@@H]2C/C=C\CCSC3OC2C(O)C(O)C3O)C1]

FSA-1507013

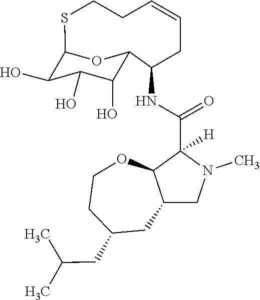 [SMILES: CC(C)C[C@@H]1CCO[C@@H]2[C@@H](C1)CN(C)[C@@H]2C(=O)N[C@@H]1C/C=C\CCSC2OC1C(O)C(O)C2O]

FSA-1508003

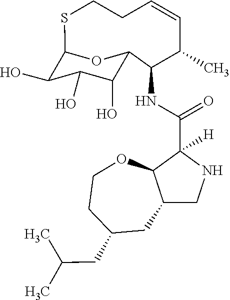 [SMILES: CC(C)C[C@@H]1CCO[C@@H]2[C@H](CN[C@@H]2C(=O)N[C@H]2C3OC(SCC/C=C\[C@@H]2C)C(O)C(O)C3O)C1]

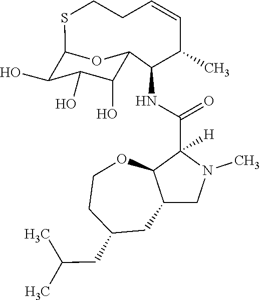 [SMILES: CC(C)C[C@@H]1CCO[C@@H]2[C@@H](C1)CN(C)[C@@H]2C(=O)N[C@H]1C2OC(SCC/C=C\[C@@H]1C)C(O)C(O)C2O]

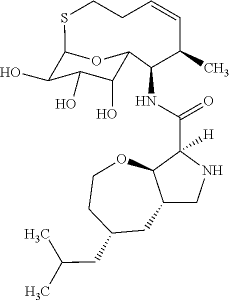 [SMILES: CC(C)C[C@@H]1CCO[C@@H]2[C@H](CN[C@@H]2C(=O)N[C@H]2C3OC(SCC/C=C\[C@H]2C)C(O)C(O)C3O)C1]

FSA-1507066

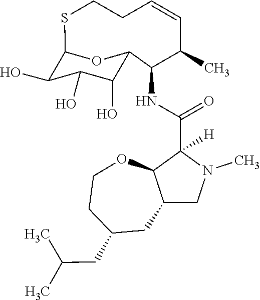 [SMILES: CC(C)C[C@@H]1CCO[C@@H]2[C@@H](C1)CN(C)[C@@H]2C(=O)N[C@H]1C2OC(SCC/C=C\[C@H]1C)C(O)C(O)C2O]

FSA-1508001

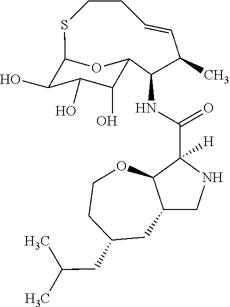 [SMILES: CC(C)C[C@@H]1CCO[C@@H]2[C@H](CN[C@@H]2C(=O)N[C@H]2C3OC(SCC/C=C/[C@H]2C)C(O)C(O)C3O)C1]

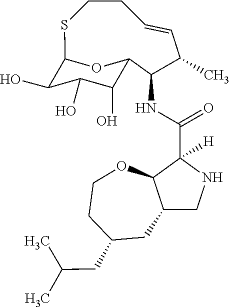 [SMILES: CC(C)C[C@@H]1CCO[C@@H]2[C@H](CN[C@@H]2C(=O)N[C@H]2C3OC(SCC/C=C/[C@@H]2C)C(O)C(O)C3O)C1]

FSA-1507068

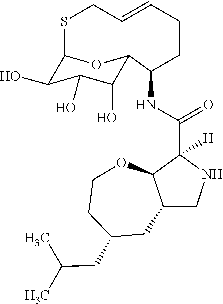 [SMILES: CC(C)C[C@@H]1CCO[C@@H]2[C@H](CN[C@@H]2C(=O)N[C@@H]2CC/C=C/CSC3OC2C(O)C(O)C3O)C1]

FSA-1503054

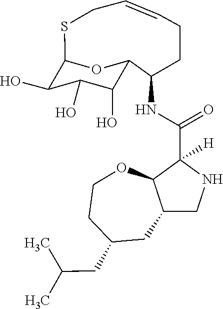 [SMILES: CC(C)C[C@@H]1CCO[C@@H]2[C@H](CN[C@@H]2C(=O)N[C@@H]2CC/C=C\CSC3OC2C(O)C(O)C3O)C1]

FSA-1503055

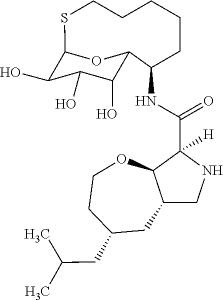 [SMILES: CC(C)C[C@@H]1CCO[C@@H]2[C@H](CN[C@@H]2C(=O)N[C@@H]2CCCCCSC3OC2C(O)C(O)C3O)C1]

FSA-1503058

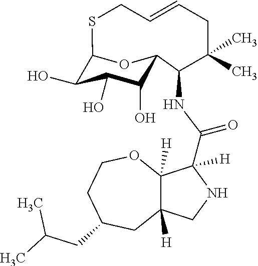

FSA-1504066A

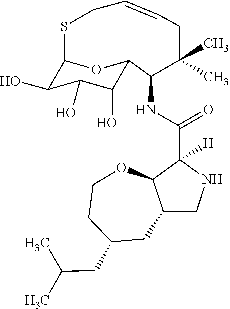

FSA-1504066B

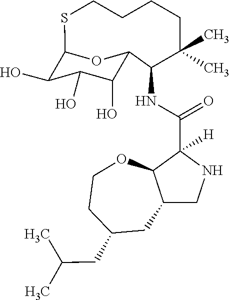

FSA-1504068

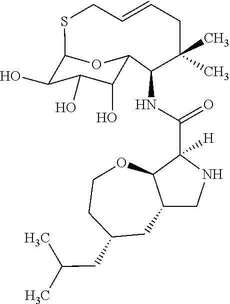

FSA-1503085

Exemplary compounds of Formula (I) include, but are not limited to, the compounds listed in Table 2.

TABLE 2
Exemplary compounds of Formula (I)

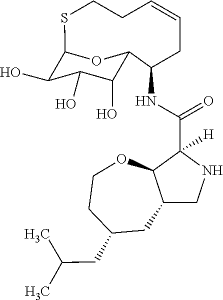 [SMILES: CC(C)C[C@@H]1CCO[C@@H]2[C@H](CN[C@@H]2C(=O)N[C@@H]2C/C=C\CCSC3OC2C(O)C(O)C3O)C1]

FSA-1507013

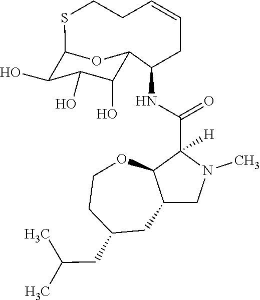 [SMILES: CC(C)C[C@@H]1CCO[C@@H]2[C@@H](C1)CN(C)[C@@H]2C(=O)N[C@@H]1C/C=C\CCSC2OC1C(O)C(O)C2O]

FSA-1508003

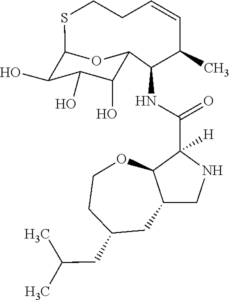 [SMILES: CC(C)C[C@@H]1CCO[C@@H]2[C@H](CN[C@@H]2C(=O)N[C@H]2C3OC(SCC/C=C\[C@H]2C)C(O)C(O)C3O)C1]

FSA-1507066

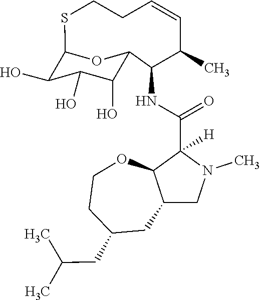 [SMILES: CC(C)C[C@@H]1CCO[C@@H]2[C@@H](C1)CN(C)[C@@H]2C(=O)N[C@H]1C2OC(SCC/C=C\[C@H]1C)C(O)C(O)C2O]

FSA-1508001

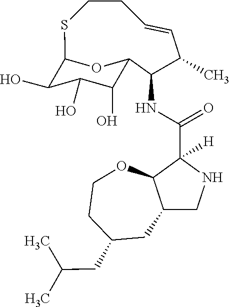 [SMILES: CC(C)C[C@@H]1CCO[C@@H]2[C@H](CN[C@@H]2C(=O)N[C@H]2C3OC(SCC/C=C/[C@@H]2C)C(O)C(O)C3O)C1]

FSA-1507068

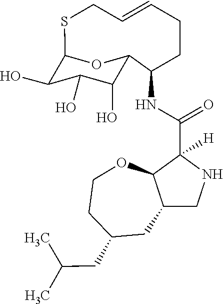 [SMILES: CC(C)C[C@@H]1CCO[C@@H]2[C@H](CN[C@@H]2C(=O)N[C@@H]2CC/C=C/CSC3OC2C(O)C(O)C3O)C1]

FSA-1503054

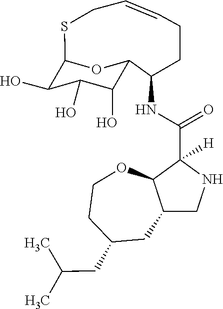 [SMILES: CC(C)C[C@@H]1CCO[C@@H]2[C@H](CN[C@@H]2C(=O)N[C@@H]2CC/C=C\CSC3OC2C(O)C(O)C3O)C1]

FSA-1503055

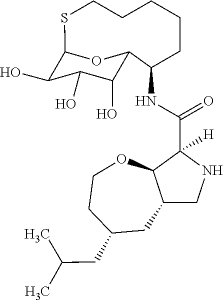 [SMILES: CC(C)C[C@@H]1CCO[C@@H]2[C@H](CN[C@@H]2C(=O)N[C@@H]2CCCCCSC3OC2C(O)C(O)C3O)C1]

FSA-1503058

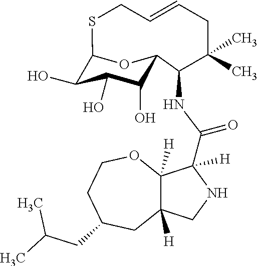

FSA-1504066A

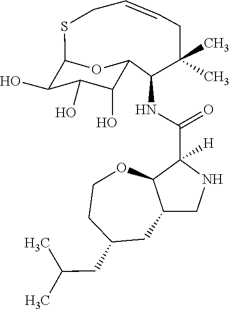

FSA-1504066B

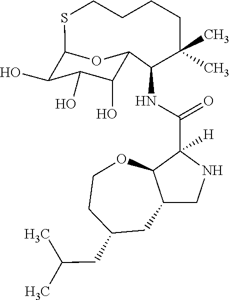

FSA-1504068

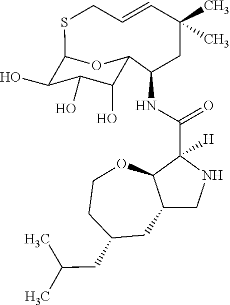

FSA-1503085

Exemplary compounds of Formula (I) include, but are not limited to, the compounds listed in Table 3.

TABLE 3
Exemplary compounds of Formula (I)

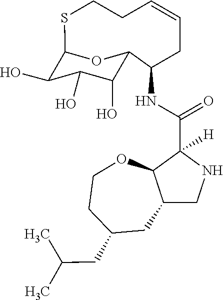 [SMILES: CC(C)C[C@@H]1CCO[C@@H]2[C@H](CN[C@@H]2C(=O)N[C@@H]2C/C=C\CCSC3OC2C(O)C(O)C3O)C1]

FSA-1507013

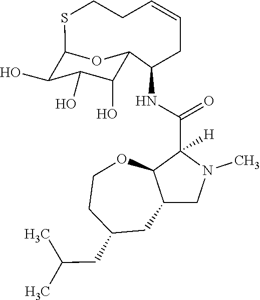 [SMILES: CC(C)C[C@@H]1CCO[C@@H]2[C@@H](C1)CN(C)[C@@H]2C(=O)N[C@@H]1C/C=C\CCSC2OC1C(O)C(O)C2O]

FSA-1508003

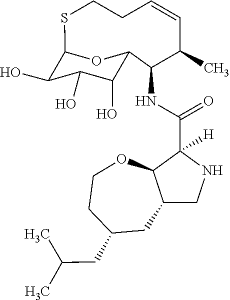 [SMILES: CC(C)C[C@@H]1CCO[C@@H]2[C@H](CN[C@@H]2C(=O)N[C@H]2C3OC(SCC/C=C\[C@H]2C)C(O)C(O)C3O)C1]

FSA-1507066

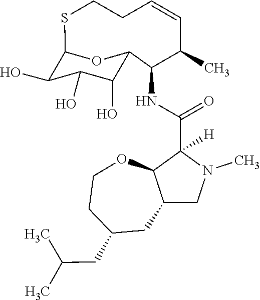 [SMILES: CC(C)C[C@@H]1CCO[C@@H]2[C@@H](C1)CN(C)[C@@H]2C(=O)N[C@H]1C2OC(SCC/C=C\[C@H]1C)C(O)C(O)C2O]

FSA-1508001

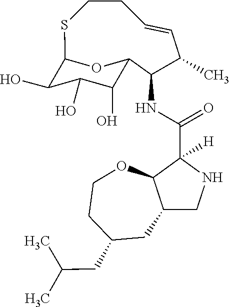 [SMILES: CC(C)C[C@@H]1CCO[C@@H]2[C@H](CN[C@@H]2C(=O)N[C@H]2C3OC(SCC/C=C/[C@@H]2C)C(O)C(O)C3O)C1]

FSA-1507068

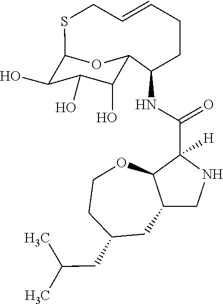 [SMILES: CC(C)C[C@@H]1CCO[C@@H]2[C@H](CN[C@@H]2C(=O)N[C@@H]2CC/C=C/CSC3OC2C(O)C(O)C3O)C1]

FSA-1503054

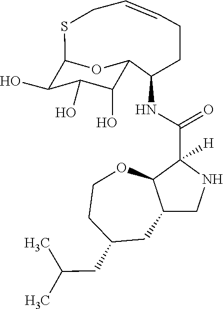 [SMILES: CC(C)C[C@@H]1CCO[C@@H]2[C@H](CN[C@@H]2C(=O)N[C@@H]2CC/C=C\CSC3OC2C(O)C(O)C3O)C1]

FSA-1503055

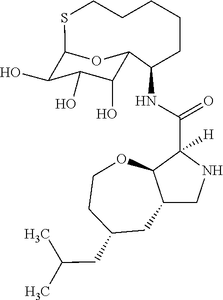 [SMILES: CC(C)C[C@@H]1CCO[C@@H]2[C@H](CN[C@@H]2C(=O)N[C@@H]2CCCCCSC3OC2C(O)C(O)C3O)C1]

FSA-1503058

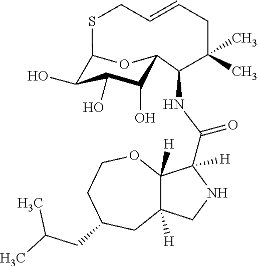

FSA-1504066A

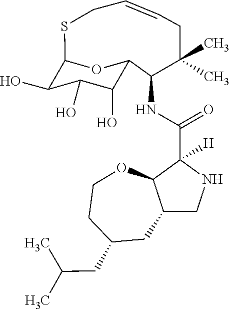

FSA-1504066B

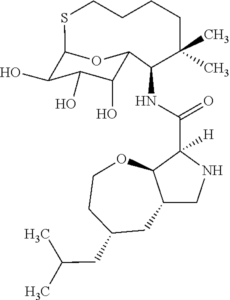

FSA-1504068

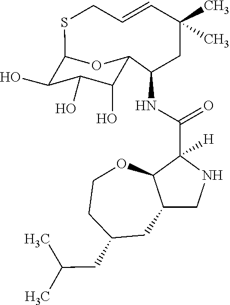

FSA-1503085

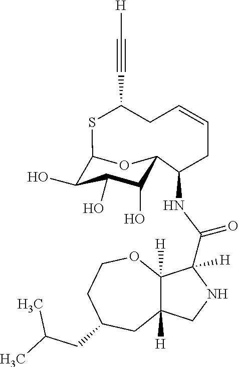 [SMILES: C#C[C@@H]1C/C=C\C[C@@H](NC(=O)[C@H]2NC[C@@H]3C[C@H](CC(C)C)CCO[C@H]32)[C@H]2OC(S1)[C@H](O)[C@H](O)C2O]

FSA-1607077

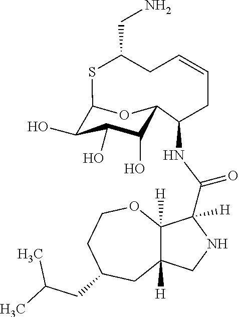 [SMILES: CC(C)C[C@@H]1CCO[C@@H]2[C@H](CN[C@@H]2C(=O)N[C@@H]2C/C=C\C[C@@H](CN)SC3O[C@H]2C(O)[C@@H](O)[C@H]3O)C1]

FSA-1608083

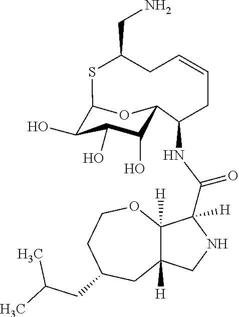 [SMILES: CC(C)C[C@@H]1CCO[C@@H]2[C@H](CN[C@@H]2C(=O)N[C@@H]2C/C=C\C[C@H](CN)SC3O[C@H]2C(O)[C@@H](O)[C@H]3O)C1]

FSA-1608082

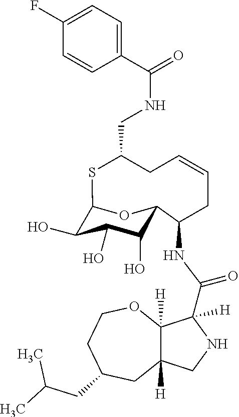 [SMILES: CC(C)C[C@@H]1CCO[C@@H]2[C@H](CN[C@@H]2C(=O)N[C@@H]2C/C=C\C[C@@H](CNC(=O)c3ccc(F)cc3)SC3O[C@H]2C(O)[C@@H](O)[C@H]3O)C1]

FSA-1608088

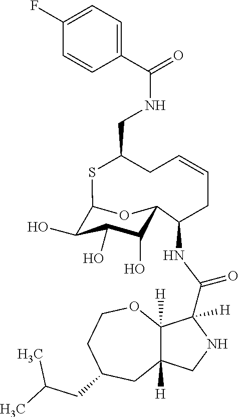 [SMILES: CC(C)C[C@@H]1CCO[C@@H]2[C@H](CN[C@@H]2C(=O)N[C@@H]2C/C=C\C[C@H](CNC(=O)c3ccc(F)cc3)SC3O[C@H]2C(O)[C@@H](O)[C@H]3O)C1]

FSA-1608089

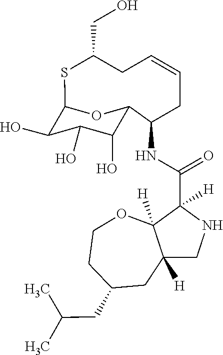 [SMILES: CC(C)C[C@@H]1CCO[C@@H]2[C@H](CN[C@@H]2C(=O)N[C@@H]2C/C=C\C[C@@H](CO)SC3OC2C(O)C(O)C3O)C1]

FSA-1606034

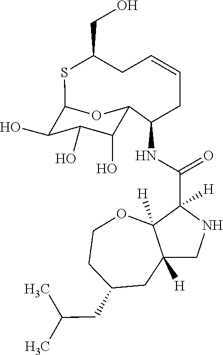 [SMILES: CC(C)C[C@@H]1CCO[C@@H]2[C@H](CN[C@@H]2C(=O)N[C@@H]2C/C=C\C[C@H](CO)SC3OC2C(O)C(O)C3O)C1]

FSA-1606033

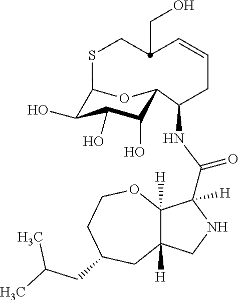 [SMILES: CC(C)C[C@@H]1CCO[C@@H]2[C@H](CN[C@@H]2C(=O)N[C@@H]2C/C=C\[C@@H](CO)CSC3O[C@H]2C(O)[C@@H](O)[C@H]3O)C1]

FSA-1608024

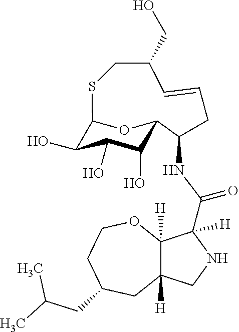 [SMILES: CC(C)C[C@@H]1CCO[C@@H]2[C@H](CN[C@@H]2C(=O)N[C@@H]2C/C=C/[C@@H](CO)CSC3O[C@H]2C(O)[C@@H](O)[C@H]3O)C1]

FSA-1608025A or
FSA-1608025B

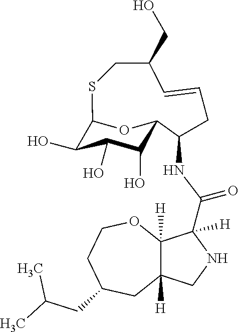 [SMILES: CC(C)C[C@@H]1CCO[C@@H]2[C@H](CN[C@@H]2C(=O)N[C@@H]2C/C=C/[C@H](CO)CSC3O[C@H]2C(O)[C@@H](O)[C@H]3O)C1]

FSA-1608025B or
FSA-1608025A

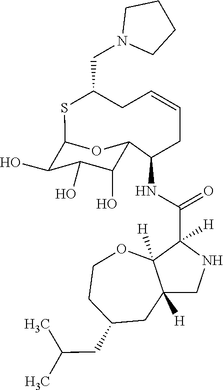 [SMILES: CC(C)C[C@@H]1CCO[C@@H]2[C@H](CN[C@@H]2C(=O)N[C@@H]2C/C=C\C[C@@H](CN3CCCC3)SC3OC2C(O)C(O)C3O)C1]

FSA-1606086

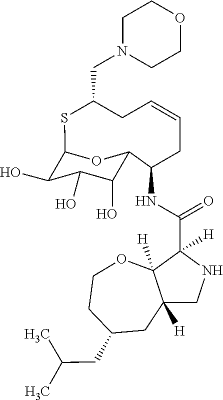 [SMILES: CC(C)C[C@@H]1CCO[C@@H]2[C@H](CN[C@@H]2C(=O)N[C@@H]2C/C=C\C[C@@H](CN3CCOCC3)SC3OC2C(O)C(O)C3O)C1]

FSA-1607019

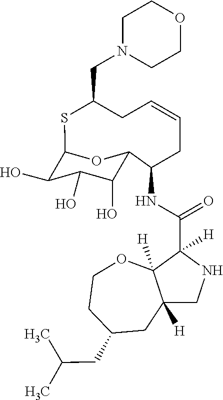 [SMILES: CC(C)C[C@@H]1CCO[C@@H]2[C@H](CN[C@@H]2C(=O)N[C@@H]2C/C=C\C[C@H](CN3CCOCC3)SC3OC2C(O)C(O)C3O)C1]

FSA-1607020

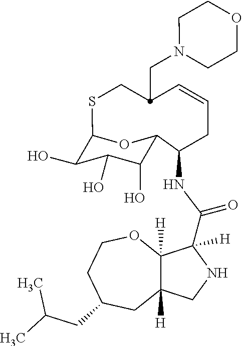 [SMILES: CC(C)C[C@@H]1CCO[C@@H]2[C@H](CN[C@@H]2C(=O)N[C@@H]2C/C=C\[C@@H](CN3CCOCC3)CSC3OC2C(O)C(O)C3O)C1]

FSA-1608036

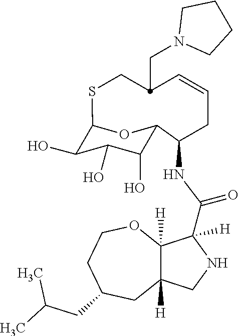 [SMILES: CC(C)C[C@@H]1CCO[C@@H]2[C@H](CN[C@@H]2C(=O)N[C@@H]2C/C=C\[C@@H](CN3CCCC3)CSC3OC2C(O)C(O)C3O)C1]

FSA-1608037

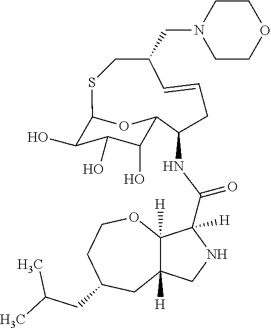 [SMILES: CC(C)C[C@@H]1CCO[C@@H]2[C@H](CN[C@@H]2C(=O)N[C@@H]2C/C=C/[C@@H](CN3CCOCC3)CSC3OC2C(O)C(O)C3O)C1]

FSA-1608056 or
FSA-1608057

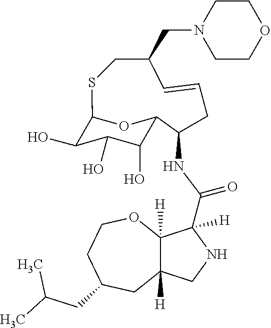 [SMILES: CC(C)C[C@@H]1CCO[C@@H]2[C@H](CN[C@@H]2C(=O)N[C@@H]2C/C=C/[C@H](CN3CCOCC3)CSC3OC2C(O)C(O)C3O)C1]

FSA-1608057 or
FSA-1608056

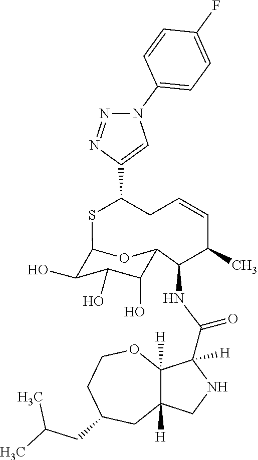 [SMILES: CC(C)C[C@@H]1CCO[C@@H]2[C@H](CN[C@@H]2C(=O)N[C@H]2C3OC(S[C@H](c4cn(-c5ccc(F)cc5)nn4)C/C=C\[C@H]2C)C(O)C(O)C3O)C1]

FSA-1609037

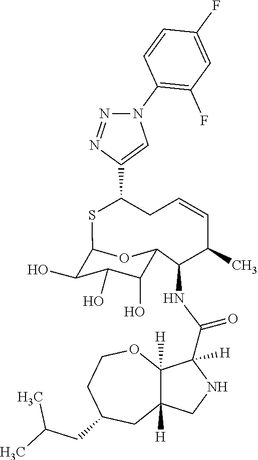 [SMILES: CC(C)C[C@@H]1CCO[C@@H]2[C@H](CN[C@@H]2C(=O)N[C@H]2C3OC(S[C@H](c4cn(-c5ccc(F)cc5F)nn4)C/C=C\[C@H]2C)C(O)C(O)C3O)C1]

FSA-1609052

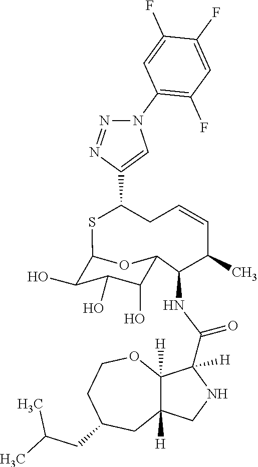 [SMILES: CC(C)C[C@@H]1CCO[C@@H]2[C@H](CN[C@@H]2C(=O)N[C@H]2C3OC(S[C@H](c4cn(-c5cc(F)c(F)cc5F)nn4)C/C=C\[C@H]2C)C(O)C(O)C3O)C1]

FSA-1609051

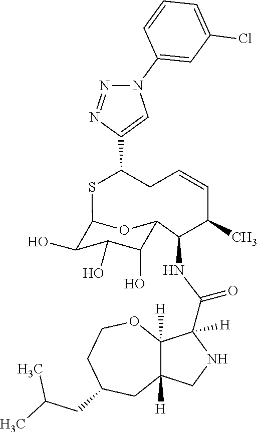 [SMILES: CC(C)C[C@@H]1CCO[C@@H]2[C@H](CN[C@@H]2C(=O)N[C@H]2C3OC(S[C@H](c4cn(-c5cccc(Cl)c5)nn4)C/C=C\[C@H]2C)C(O)C(O)C3O)C1]

FSA-1609015

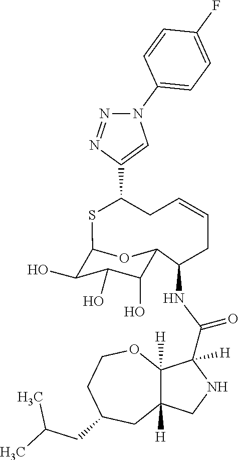 [SMILES: CC(C)C[C@@H]1CCO[C@@H]2[C@H](CN[C@@H]2C(=O)N[C@@H]2C/C=C\C[C@@H](c3cn(-c4ccc(F)cc4)nn3)SC3OC2C(O)C(O)C3O)C1]

FSA-1608021

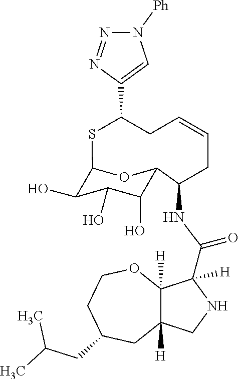 [SMILES: CC(C)C[C@@H]1CCO[C@@H]2[C@H](CN[C@@H]2C(=O)N[C@@H]2C/C=C\C[C@@H](c3cn(-c4ccccc4)nn3)SC3OC2C(O)C(O)C3O)C1]

FSA-1607075

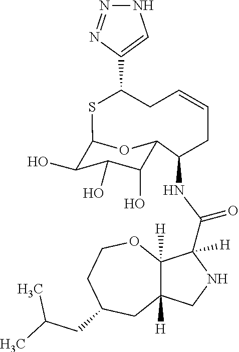 [SMILES: CC(C)C[C@@H]1CCO[C@@H]2[C@H](CN[C@@H]2C(=O)N[C@@H]2C/C=C\C[C@@H](c3c[nH]nn3)SC3OC2C(O)C(O)C3O)C1]

FSA-1607090

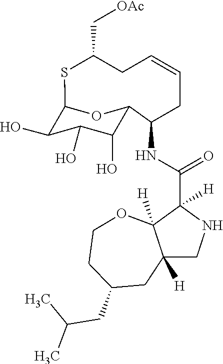 [SMILES: CC(=O)OC[C@@H]1C/C=C\C[C@@H](NC(=O)[C@H]2NC[C@@H]3C[C@H](CC(C)C)CCO[C@H]32)C2OC(S1)C(O)C(O)C2O]

FSA-1607032

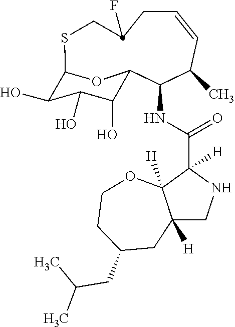 [SMILES: CC(C)C[C@@H]1CCO[C@@H]2[C@H](CN[C@@H]2C(=O)N[C@H]2C3OC(SC[C@H](F)C/C=C\[C@H]2C)C(O)C(O)C3O)C1]

FSA-1609033

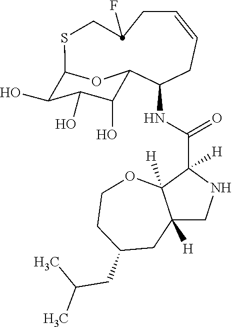 [SMILES: CC(C)C[C@@H]1CCO[C@@H]2[C@H](CN[C@@H]2C(=O)N[C@@H]2C/C=C\C[C@@H](F)CSC3OC2C(O)C(O)C3O)C1]

FSA-1607022

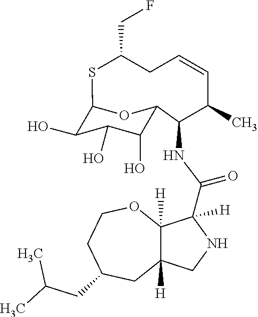 [SMILES: CC(C)C[C@@H]1CCO[C@@H]2[C@H](CN[C@@H]2C(=O)N[C@H]2C3OC(S[C@H](CF)C/C=C\[C@H]2C)C(O)C(O)C3O)C1]

FSA-1609034

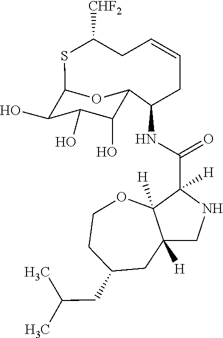 [SMILES: CC(C)C[C@@H]1CCO[C@@H]2[C@H](CN[C@@H]2C(=O)N[C@@H]2C/C=C\C[C@@H](C(F)F)SC3OC2C(O)C(O)C3O)C1]

FSA-1607044

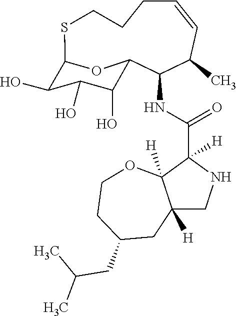 [SMILES: CC(C)C[C@@H]1CCO[C@@H]2[C@H](CN[C@@H]2C(=O)N[C@H]2C3OC(SCCC/C=C\[C@H]2C)C(O)C(O)C3O)C1]

FSA-1609096

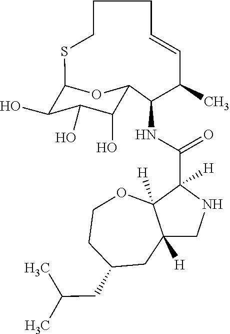 [SMILES: CC(C)C[C@@H]1CCO[C@@H]2[C@H](CN[C@@H]2C(=O)N[C@H]2C3OC(SCCC/C=C/[C@H]2C)C(O)C(O)C3O)C1]

FSA-1609095

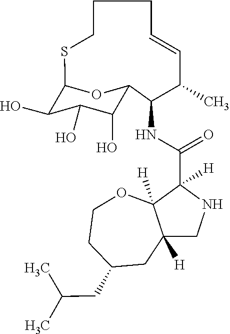 [SMILES: CC(C)C[C@@H]1CCO[C@@H]2[C@H](CN[C@@H]2C(=O)N[C@H]2C3OC(SCCC/C=C/[C@@H]2C)C(O)C(O)C3O)C1]

FSA-1609094

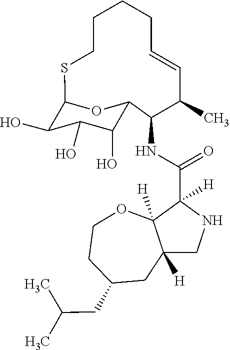 [SMILES: CC(C)C[C@@H]1CCO[C@@H]2[C@H](CN[C@@H]2C(=O)N[C@H]2C3OC(SCCCC/C=C/[C@H]2C)C(O)C(O)C3O)C1]

FSA-1610016

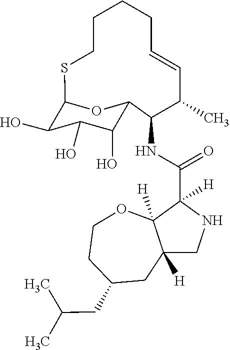 [SMILES: CC(C)C[C@@H]1CCO[C@@H]2[C@H](CN[C@@H]2C(=O)N[C@H]2C3OC(SCCCC/C=C/[C@@H]2C)C(O)C(O)C3O)C1]

FSA-1610014

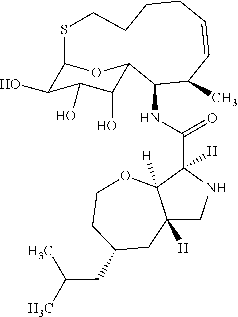 [SMILES: CC(C)C[C@@H]1CCO[C@@H]2[C@H](CN[C@@H]2C(=O)N[C@H]2C3OC(SCCCC/C=C\[C@H]2C)C(O)C(O)C3O)C1]

FSA-1610015

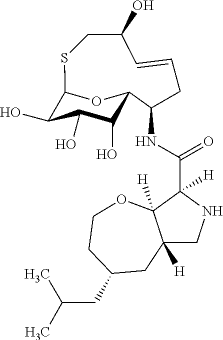 [SMILES: CC(C)C[C@@H]1CCO[C@@H]2[C@H](CN[C@@H]2C(=O)N[C@@H]2C/C=C/[C@H](O)CSC3O[C@H]2C(O)[C@@H](O)[C@H]3O)C1]

FSA-1608009

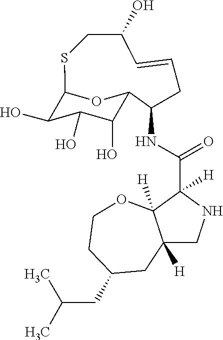 [SMILES: CC(C)C[C@@H]1CCO[C@@H]2[C@H](CN[C@@H]2C(=O)N[C@@H]2C/C=C/[C@@H](O)CSC3OC2C(O)C(O)C3O)C1]

FSA-1608016

### Preparation of Compounds of Formula (I)

Exemplary methods that may be used in the preparation of a compound of the present disclosure are described below and are not to be construed as limiting. The compounds herein may be prepared by other methods of synthesis known in the art, and the procedures described herein may be modified or combined with other known methods.

In another aspect, compounds of the present disclosure are prepared by a method comprising ring closing metathesis of a compound of Formula (A) or (A1) as depicted in Scheme 1.

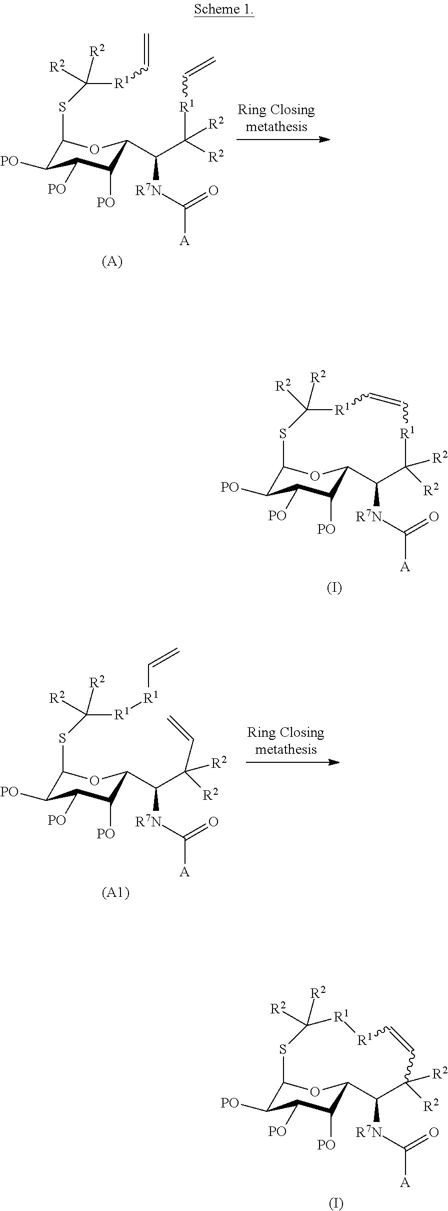

For all intermediates, A, P, R¹, R², and R⁷ are as defined herein for a compound of Formula (I), unless otherwise stated.

In certain embodiments, the ring closing metathesis is achieved through use of a transition metal catalyst. In certain embodiments, the transition metal catalyst is a tungsten (W), molybdenum (Mo), or ruthenium (Ru) catalyst. In certain embodiments, the catalyst is a ruthenium catalyst.

For examples of olefin metathesis reagents, catalysts, and reaction conditions useful in the present methods, see, e.g., Schrodi, Y.; Pederson, R. L. Aldrichimica Acta 2007, 40, 45; Adv. Synth. Catal. 2007, 349, 1-268; Grubbs, R. H. Tetrahedron 2004, 60, 7117; Handbook of Metathesis; Grubbs, R. H., Ed.; Wiley-VCH: Weinheim, 2003; Vols. 1-3; Trnka, T. M.; Grubbs, R. H. Acc. Chem. Res. 2001, 34, 18; Fürstner, A. Angew. Chem., Int. Ed. 2000, 39, 3012; Schuster, M.; Blechert, S. Angew. Chem., Int. Ed. 1997, 36, 2036; Ritter, T. et al. Organometallics 2006, 25, 5740; Chatterjee, A. K. et al. J. Am. Chem. Soc. 2000, 122, 3783; Chatterjee, A. K.; Grubbs, R. H. Org. Lett. 1999, 1, 1751; Murelli, R. P.; Snapper, M. L. Org. Lett. 2007, 9, 1749; Stewart, I. C. et al. Org. Lett. 2007, 9, 1589; Ung, T. et al. Organometallics 2004, 23, 5399; Benitez, D.; Goddard, W. A., III. J. Am. Chem. Soc. 2005, 127, 12218; Love, J. A. et al. Angew. Chem., Int. Ed. 2002, 41, 4035; Sanford, M. S. et al. Organometallics 2001, 20, 5314; Choi, T.-L.; Grubbs, R. H. Angew. Chem. 2003, 115, 1785; Ritter, T. et al. Organometallics 2006, 25, 5740; and references cited therein; each of which is incorporated herein by reference.

In certain embodiments, the metathesis catalyst is a Grubbs catalyst. In certain embodiments, the Grubbs catalyst is of the formula:

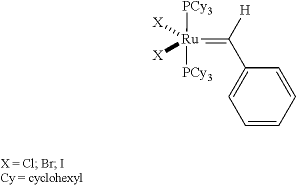

Benzylidenebis-(tricyclohexylphosphine)-dichlororuthenium (X=Cl); Benzylidenebis-(tricyclohexylphosphine)-dibromoruthenium (X=Br); Benzylidenebis-(tricyclohexylphosphine)-diiodoruthenium (X=I);

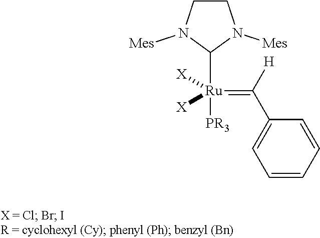

1,3-(Bis(mesityl)-2-imidazolidinylidene)dichloro-(phenylmethylene)(tricyclohexyl-phosphine) ruthenium (X=Cl; R=cyclohexyl); 1,3-(Bis(mesityl)-2-imidazolidinylidene)dibromo-(phenylmethylene)(tricyclohexyl-phosphine) ruthenium (X=Br; R═cyclohexyl); 1,3-(Bis(mesityl)-2-imidazolidinylidene)diiodo-(phenylmethylene) (tricyclohexyl-phosphine) ruthenium (X═I; R═cyclohexyl); 1,3-(Bis(mesityl)-2-imidazolidinylidene)dichloro-(phenylmethylene)(triphenylphosphine) ruthenium (X50 Cl; R═phenyl); 1,3-(Bis(mesityl)-2-imidazolidinylidene)dichloro-(phenylmethylene) (tribenzylphosphine) ruthenium (X═Cl; R═benzyl);

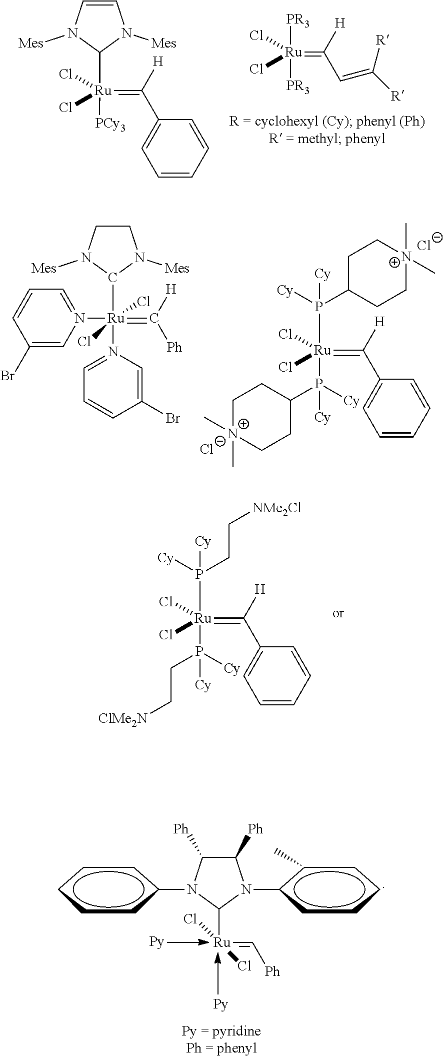

In certain embodiments, the metathesis catalyst is the second generation Grubbs catalyst (Grubbs II) of the formula:

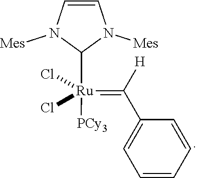

In certain embodiments, the metathesis catalyst is a Grubbs-Hoveyda catalyst. In certain embodiments, the Grubbs-Hoveyda catalyst is of the formula:

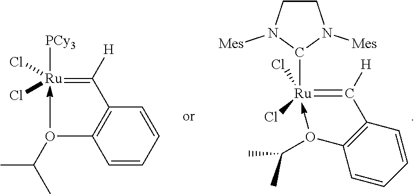

In certain embodiments, the method further comprises coupling a compound of Formula (B) and a compound of Formula (C) to form the compound of Formula (A), or coupling a compound of Formula (B1) and a compound of Formula (C) to form the compound of Formula (A1) as depicted in Scheme 2 below.

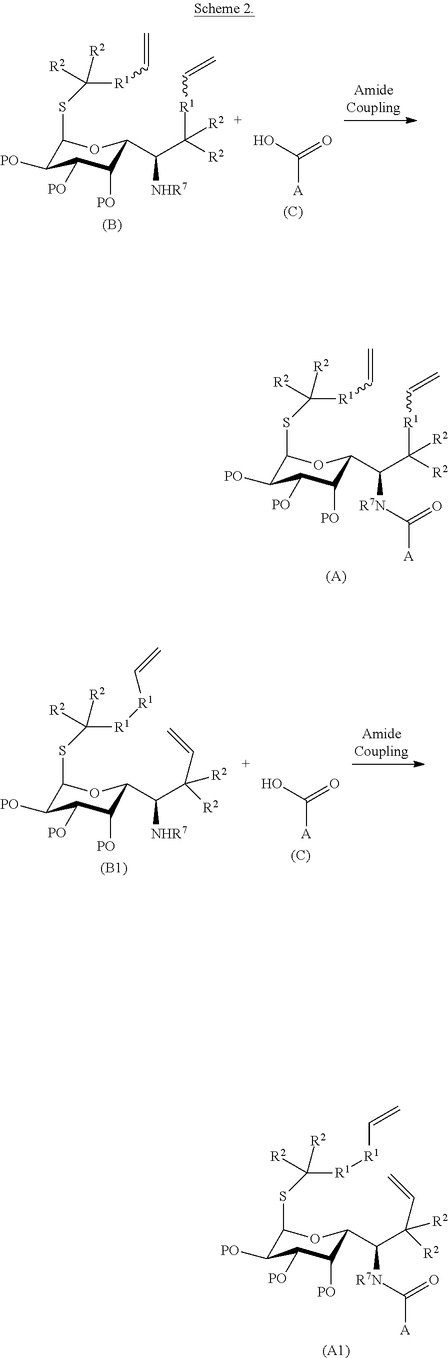

In certain embodiments, the amide bond formation is promoted by an amide coupling reagent (e.g., 1-[bis(dimethylamino)methylene]-1H-1,2,3-triazolo[4,5-b]pyridinium 3-oxid hexafluorophosphate (HATU), 1-ethyl-3-(3-dimethylaminopropyl) carbodiimide (EDC), hydroxybenzotriazole (HOBt), and the like, or a combination thereof). In certain embodiments, the amide coupling reagent (e.g., HATU, EDC, HOBt) is reacted with the compound of Formula (C). In certain embodiments, the amide coupling reagent (e.g., HATU, EDC, HOBt) is reacted with the compound of Formula (C) prior to amide coupling with the compound of Formula (B) or (B1). In certain embodiments, the amide coupling reagent is HATU.

In certain embodiments, the method comprises adding up to 1.0, 1.1, 1.2, 1.3, 1.4, 1.5, 1.6, 1.7, 1.8. 1.9, or 2.0 equivalents of the amide coupling reagent. In certain embodiments, the method comprises performing the coupling reaction at room temperature, ambient temperature, or elevated temperature. In certain embodiments, the method comprises performing the coupling reaction at 20-60° C., 20-50° C., 20-40° C., 20-30° C., 20-25° C., or 25-30° C.

In certain embodiments, an additional reagent may be added to the amide bond forming reaction. In certain embodiments, the additional reagent may facilitate amide coupling by protecting the free hydroxyls of the compound of Formula (B) or (B1). In certain embodiments, the additional reagent is a silylating reagent. In certain embodiments, the silylating reagent reacts with the free hydroxyl groups of the compound of Formula (B) or (B1) to form silyl protecting groups in situ during the reaction. In certain embodiments, the additional reagent is added to the compound of Formula (B) or (B1) before the amide coupling. In certain embodiments, the additional reagent is N,O-bis(trimethylsilyl)trifluoroacetamide. In certain embodiments, the method comprises adding up to 1.0, 1.1, 1.2, 1.3, 1.4, 1.5, 1.6, 1.7, 1.8. 1.9, 2.0, 2.1, 2.2, 2.3, 2.4, 2.5, 2.6, 2.7, 2.8. 2.9, 3.0, or more equivalents of the silylating reagent.

In another aspect, compounds of the present disclosure are prepared by a method comprising ring closing metathesis of a compound of Formula (B) to form a compound of Formula (E), or ring closing metathesis of a compound of Formula (B1) to form a compound of Formula (E1) as depicted in Scheme 3.

In certain embodiments, the ring closing metathesis is achieved through use of any of the catalysts and conditions described herein.

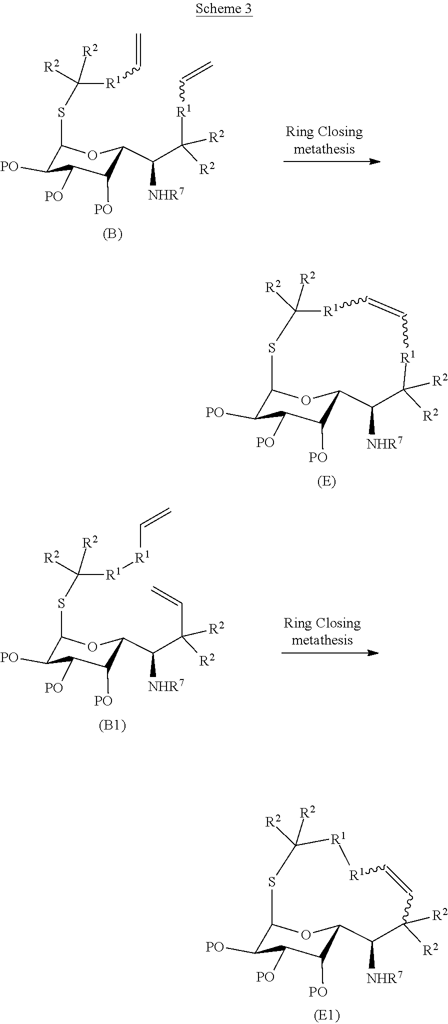

For all intermediates, A, P, R¹, R², and R⁷ are as defined herein for a compound of Formula (I), unless otherwise stated. In certain embodiments, R⁷ is a substituted or unsubstituted acetyl. In certain embodiments, R⁷ is trifluoroacetyl.

In certain embodiments, the method further comprises coupling the compound of Formula (E) and a compound of Formula (C) to form the compound of Formula (I), or coupling a compound of Formula (E1) and a compound of Formula (C) to form the compound of Formula (I) as depicted in Scheme 4 below.

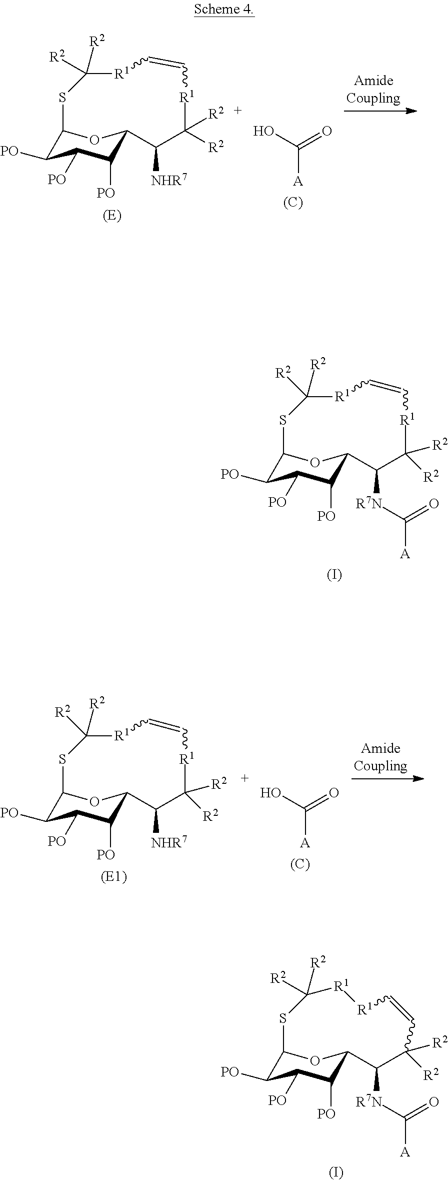

In certain embodiments, the amide bond formation is promoted by an amide coupling reagent (e.g., 1-[bis(dimethylamino)methylene]-1H-1,2,3-triazolo[4,5-b]pyridinium 3-oxid hexafluorophosphate (HATU), 1-ethyl-3-(3-dimethylaminopropyl) carbodiimide (EDC), hydroxybenzotriazole (HOBt), and the like, or a combination thereof). In certain embodiments, the amide coupling reagent (e.g., HATU, EDC, HOBt) is reacted with the compound of Formula (C). In certain embodiments, the amide coupling reagent (e.g., HATU, EDC, HOBt) is reacted with the compound of Formula (C) prior to amide coupling with the compound of Formula (E) or (E1). In certain embodiments, the amide coupling reagent is HATU.

In certain embodiments, the method comprises adding up to 1.0, 1.1, 1.2, 1.3, 1.4, 1.5, 1.6, 1.7, 1.8. 1.9, or 2.0 equivalents of the amide coupling reagent. In certain embodiments, the method comprises performing the coupling reaction at room temperature, ambient temperature, or elevated temperature. In certain embodiments, the method comprises performing the coupling reaction at 20-60° C., 20-50° C., 20-40° C., 20-30° C., 20-25° C., or 25-30° C.

In certain embodiments, an additional reagent may be added to the amide bond forming reaction. In certain embodiments, the additional reagent may facilitate amide coupling by protecting the free hydroxyls of the compound of Formula (E) or (E1). In certain embodiments, the additional reagent is a silylating reagent. In certain embodiments, the silylating reagent reacts with the free hydroxyl groups of the compound of Formula (E) or (E1) to form silyl protecting groups in situ during the reaction. In certain embodiments, the additional reagent is added to the compound of Formula (E) or (E1) before the amide coupling. In certain embodiments, the additional reagent is N,O-bis(trimethylsilyl)trifluoroacetamide. In certain embodiments, the method comprises adding up to 1.0, 1.1, 1.2, 1.3, 1.4, 1.5, 1.6, 1.7, 1.8. 1.9, 2.0, 2.1, 2.2, 2.3, 2.4, 2.5, 2.6, 2.7, 2.8. 2.9, 3.0, or more equivalents of the silylating reagent.

### Pharmaceutical Compositions and Administration

The present disclosure provides pharmaceutical compositions comprising a compound as described herein, or a pharmaceutically acceptable salt thereof, and a pharmaceutically acceptable excipient.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the compound of the present disclosure. The amount of the compound is generally equal to the dosage of the compound which would be administered to a subject and/or a convenient fraction of such a dosage such as, for example, one-half or one-third of such a dosage.

Relative amounts of the compound, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) compound.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Liquid dosage forms for oral and parenteral administration include pharmaceutically acceptable emulsions, microemulsions, solutions, suspensions, syrups and elixirs. In addition to the compounds, the liquid dosage forms may comprise inert diluents commonly used in the art such as, for example, water or other solvents, solubilizing agents, and emulsifiers, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium.

Solid dosage forms for oral administration include capsules, tablets, pills, powders, and granules. In such solid dosage forms, the compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Dosage forms for topical and/or transdermal administration of a compound of this invention may include ointments, pastes, creams, lotions, gels, powders, solutions, sprays, inhalants and/or patches. Generally, the compound is admixed under sterile conditions with a pharmaceutically acceptable carrier and/or any needed preservatives and/or buffers as can be required.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily amount of the compound will be decided by the attending physician within the scope of sound medical judgment. The specific therapeutically effective dose level for any particular subject will depend upon a variety of factors including the disease, disorder, or condition being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent, the therapeutic regimen, and/or the condition of the subject. Oral administration is the preferred mode of administration. However, in certain embodiments, the subject may not be in a condition to tolerate oral administration, and thus intravenous, intramuscular, and/or rectal administration are also preferred alternative modes of administration.

An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein.

It will be also appreciated that a compound or composition, as described herein, can be administered in combination with one or more additional therapeutically active agents. The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In certain embodiments, the levels utilized in combination will be lower than those utilized individually.

Exemplary additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins. Therapeutically active agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the additional therapeutically active agent is an antibiotic. Exemplary antibiotics include, but are not limited to, penicillins (e.g., penicillin, amoxicillin), cephalosporins (e.g., cephalexin), compounds (e.g., erythromycin, clarithromycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, dioxyline, auromomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, and quinupristin/dalfopristin (Syndercid™).

Also encompassed by the invention are kits (e.g., pharmaceutical packs). The kits provided may comprise an inventive pharmaceutical composition or compound and a container (e.g., a vial, ampule, bottle, syringe, and/or dispenser package, or other suitable container). In certain embodiments, provided kits may optionally further include a second container comprising a pharmaceutical excipient for dilution or suspension of an inventive pharmaceutical composition or compound. In certain embodiments, the inventive pharmaceutical composition or compound provided in the container and the second container are combined to form one unit dosage form.

### Methods of Treatment and Uses

The present disclosure contemplates using compounds of the present invention for the treatment of infectious diseases, for example, fungal, bacterial, viral, and/or parasitic infections. Lincosamides are generally known to exhibit anti-bacterial activity.

Thus, as generally described herein, provided is a method of treating an infectious disease comprising administering an effective amount of a compound of the present disclosure, or a pharmaceutically acceptable salt thereof, to a subject in need thereof. Such a method can be conducted in vivo (i.e., by administration to a subject). Treating, as used herein, encompasses therapeutic treatment and prophylactic treatment.

In certain embodiments, the effective amount is a therapeutically effective amount. For example, in certain embodiments, the method slows the progress of an infectious disease in the subject. In certain embodiments, the method improves the condition of the subject suffering from an infectious disease. In certain embodiments, the subject has a suspected or confirmed infectious disease.

In certain embodiments, the effective amount is a prophylactically effective amount. For example, in certain embodiments, the method prevents or reduces the likelihood of an infectious disease, e.g., in certain embodiments, the method comprises administering a compound of the present disclosure to a subject in need thereof in an amount sufficient to prevent or reduce the likelihood of an infectious disease. In certain embodiments, the subject is at risk of an infectious disease (e.g., has been exposed to another subject who has a suspected or confirmed infectious disease or has been exposed or thought to be exposed to a pathogen).

In one aspect, provided is a method of killing a microorganism (e.g., fungus, bacterium, virus, parasite) comprising contacting the microorganism with an effective amount of a compound of the present disclosure. The compound may contact the microorganism in vivo (e.g., in a subject in need thereof) or in vitro.

In another aspect, provided is a method of inhibiting the growth of a microorganism (e.g., fungus, bacterium, virus, parasite) comprising contacting the microorganism with an effective amount of a compound of the present disclosure. The compound may contact the microorganism in vivo (e.g., in a subject in need thereof) or in vitro.

In another aspect, provided is an in vitro method of inhibiting pathogenic growth comprising contacting an effective amount of the compound of the present invention with a pathogen (e.g., a bacteria, virus, fungus, or parasite) in a cell culture.

In another aspect, provided is an in vitro method of inhibiting pathogenic growth comprising contacting a pathogen (e.g., a bacteria, virus, fungus, or parasite) with an effective amount of a compound of the present disclosure. In another aspect, provided is a method of inhibiting protein synthesis (e.g., by interfering with the synthesis of proteins by binding to the 23s portion of the 50S subunit of the bacterial ribosome and causing premature dissociation of the peptidyl-tRNA from the ribosome) with an effective amount of a compound of the present disclosure. In certain embodiments, inhibiting protein synthesis comprises inhibiting the ribosome of bacteria with an effective amount of a compound of the present disclosure. Protein synthesis may be inhibited in vivo or in vitro.

As used herein, “infectious disease” and “microbial infection” are used interchangeably, and refer to an infection with a pathogen, such as a fungus, bacteria, virus, or parasite. In certain embodiments, the infectious disease is caused by a fungus, bacteria, or parasite. In certain embodiments, the infectious disease is caused by a pathogen resistant to other treatments. In certain embodiments, the infectious disease is caused by a pathogen that is multi-drug tolerant or resistant, e.g., the infectious disease is caused by a pathogen that neither grows nor dies in the presence of or as a result of other treatments.

In certain embodiments, the infectious disease is a bacterial infection. For example, in certain embodiments, provided is a method of treating a bacterial infection comprising administering an effective amount of a compound of the present invention, or a pharmaceutically acceptable salt thereof, to a subject in need thereof.

In certain embodiments, the compound has a mean inhibitory concentration (MIC), with respect to a particular bacteria, of less than or equal to 50 μg/mL, less than or equal to 25 μg/mL, less than or equal to 20 μg/mL, less than or equal to 10 μg/mL, less than or equal to 5 μg/mL, less than or equal to 1 μg/mL, less than or equal to 0.5 μg/mL, or less than or equal to 0.25 μg/mL.

In certain embodiments, the bacteria is susceptible (e.g., responds to) or resistant to known commercial compounds, such as azithromycin, lincomycin, clindamycin, telithromycin, erythromycin, spiramycin, and the like. In certain embodiments, the bacteria is resistant to a known compound. For example, in certain embodiments, the bacteria is lincomycin resistant or clindamycin resistant.

In certain embodiments, the bacterial infection is resistant to other antibiotics (e.g., non-compound) therapy. For example, in certain embodiments, the pathogen is vancomycin resistant (VR). In certain embodiments, the pathogen is methicillin-resistant (MR), e.g., in certain embodiments, the bacterial infection is a methicillin-resistant S. aureus infection (a MRSA infection). In certain embodiments, the pathogen is quinolone resistant (QR). In certain embodiments, the pathogen is fluoroquinolone resistant (FR).

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Ntrospirae, phylum

Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacterial infection is an infection with a Gram positive bacterium.

In certain embodiments, the Gram positive bacterium is a bacterium of the phylum Firmicutes.

In certain embodiments, the bacteria is a member of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus.

In certain embodiments, the bacteria is a member of the phylum Firmicutes and the genus Staphylococcus, i.e., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In certain embodiments, the Staphylococcus infection is a S. aureus infection.

In certain embodiments, the S. aureus has an efflux (e.g., mef, msr) genotype. Bacteria of the efflux genotypes actively pump drug out of the cell via efflux pumps.

In certain embodiments, the S. aureus has a methylase (e.g., erm) genotype. In certain embodiments, erm is the bacterial gene class coding for erythromycin ribosomal methylase, which methylates a single adenine in 23S rRNA, itself a component of 50S rRNA.

In certain embodiments, the bacteria is a member of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the Bacillus infection is a B. subtilis infection. In certain embodiments, the B. subtilis has an efflux (e.g., mef, msr) genotype. In certain embodiments, the B. subtilis has a methylase (e.g., erm) genotype.

In certain embodiments, the bacteria is a member of the phylum Firmicutes and the genus Streptococcus, i.e., the bacterial infection is a Streptococcus infection. Exemplary Streptococcus bacteria include, but are not limited to, S. agalactiae, S. anginosus, S. bovis, S. canis, S. constellatus, S. dysgalactiae, S. equinus, S. iniae, S. intermedius, S. mitis, S. mutans, S. oralis, S. parasanguinis, S. peroris, S. pneumoniae, S. pyogenes, S. ratti, S. salivarius, S. thermophilus, S. sanguinis, S. sobrinus, S. suis, S. uberis, S. vestibularis, S. viridans, and S. zooepidemicus. In certain embodiments, the Streptococcus infection is an S. pyogenes infection. In certain embodiments, the Streptococcus infection is an S. pneumoniae infection. In certain embodiments, the S. pneumoniae has an efflux (e.g., mef, msr) genotype. In certain embodiments, the S. pneumoniae has a methylase (e.g., erm) genotype.

In certain embodiments, the bacteria is a member of the phylum Firmicutes and the genus Clostridium, i.e., the bacterial infection is a Clostridium infection. Exemplary Clostridia bacteria include, but are not limited to, C. botulinum, C. difficile, C. perfringens, C. tetani, and C. sordellii.

In certain embodiments, the compounds of the disclosure are a safer alternative to clindamycin, due to reduced incidence of pseudomembranous colitis. In certain embodiments, the compounds of the disclosure have increased activity against Clostridium difficile (C. difficile) in comparison to clindamycin. In certain embodiments, the compounds have a mean inhibitory concentration (MIC), with respect to C. difficile, of less than or equal to 50 μg/mL, less than or equal to 25 μg/mL, less than or equal to 20 μg/mL, less than or equal to 10 μg/mL, less than or equal to 5 μg/mL, less than or equal to 1 μg/mL, less than or equal to 0.5 μg/mL, or less than or equal to 0.25 μg/mL.

In certain embodiments, the bacterial infection is an infection with a Gram negative bacteria.

In certain embodiments, the Gram negative bacteria is a bacteria of the phylum Proteobacteria and the genus Escherichia. i.e., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the Escherichia infection is an E. coli infection.

In certain embodiments, the Gram negative bacteria is a bacteria of the phylum Proteobacteria and the genus Haemophilus. i.e., the bacterial infection is an Haemophilus infection. Exemplary Haemophilus bacteria include, but are not limited to, H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus. In certain embodiments, the Haemophilus infection is an H. influenzae infection.

In certain embodiments, the Gram negative bacteria is a bacteria of the phylum Proteobacteria and the genus Acinetobacter. i.e., the bacterial infection is an Acinetobacter infection. Exemplary Acinetobacter bacteria include, but are not limited to, A. baumanii, A. haemolyticus, and A. lwoffii. In certain embodiments, the Acinetobacter infection is an A. baumanii infection.

In certain embodiments, the Gram negative bacteria is a bacteria of the phylum Proteobacteria and the genus Klebsiella. i.e., the bacterial infection is a Klebsiella infection. Exemplary Klebsiella bacteria include, but are not limited to, K. granulomatis, K. oxytoca, K. michiganensis, K. pneumoniae, K. quasipneumoniae, and K. variicola. In certain embodiments, the Klebsiella infection is a K. pneumoniae infection.

In certain embodiments, the Gram negative bacteria is a bacteria of the phylum Proteobacteria and the genus Pseudomonas. i.e., the bacterial infection is a Pseudomonas infection. Exemplary Pseudomonas bacteria include, but are not limited to, P. aeruginosa, P. oryzihabitans, P. plecoglissicida, P. syringae, P. putida, and P. fluoroscens. In certain embodiments, the Pseudomonas infection is a P. aeruginosa infection.

In certain embodiments, the Gram negative bacteria is a bacteria of the phylum Bacteroidetes and the genus Bacteroides. i.e., the bacterial infection is a Bacteroides infection. Exemplary Bacteroides bacteria include, but are not limited to, B. fragilis, B. distasonis, B. ovatus, B. thetaiotaomicron, and B. vulgatus. In certain embodiments, the Bacteroides infection is a B. fragilis infection.

In certain embodiments, the bacterial infection being treated is a Staphylococcus infection, a Streptococcus infection, an Enterococcus infection, an Acenitobacter infection, a Clostridium infection, a Bacterioides infection, an Escherichia infection, a Pseudomonas infection, a Neisseria infection, a Klebsiella infection, or a Haemophilus infection. In certain embodiments, the bacterial infection being treated is a Staphylococcus infection, a Streptococcus infection, an Enterococcus infection, an Acenitobacter infection, an Escherichia infection, a Pseudomonas infection, or a Klebsiella infection.

In certain embodiments, the bacterial infection is a dental infection, abdominal infection, abscess, pelvic inflammatory disease, or anaerobic infection.

In certain embodiments, the compounds are useful for treating acne, toxic shock syndrome, malaria, and to decrease the risk of premature births in women with bacterial vaginosis. In certain embodiments, the compounds are useful for treating methicillin-resistant S. aureus.

In certain embodiments, the bacteria is an atypical bacteria, i.e., are neither Gram positive nor Gram negative.

In certain embodiments, the infectious disease is an infection with a parasitic infection. Thus, in certain embodiments, provided is a method of treating a parasitic infection comprising administering an effective amount of a compound of the present invention, or a pharmaceutically acceptable salt thereof, to a subject in need thereof.

In certain embodiments, the compound has an IC₅₀ (uM) with respect to a particular parasite, of less than 50 uM, less than 25 uM, less than 20 uM, less than 10 uM, less than 5 uM, or less than 1 uM.

Exemplary parasites include, but are not limited to, Trypanosoma spp. (e.g., Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp. (e.g., P. flaciparum), Crytosporidium spp., Isospora spp., Balantidium spp., Pneumocyst is spp., Babesia, Loa Loa, Ascaris lumbricoides, Dirofilaria immitis, and Toxoplasma ssp. (e.g. T. gondii).

As generally described herein, the present disclosure further provides a method of treating an inflammatory condition comprising administering an effective amount of a compound of the present disclosure, or a pharmaceutically acceptable salt thereof, to a subject in need thereof. Such a method can be conducted in vivo (i.e., by administration to a subject) or in vitro (e.g., upon contact with the pathogen, tissue, or cell culture). Treating, as used herein, encompasses therapeutic treatment and prophylactic treatment.

In certain embodiments, the effective amount is a therapeutically effective amount. For example, in certain embodiments, the method slows the progress of an inflammatory condition in the subject. In certain embodiments, the method improves the condition of the subject suffering from an inflammatory condition. In certain embodiments, the subject has a suspected or confirmed inflammatory condition.

In certain embodiments, the effective amount is a prophylactically effective amount. For example, in certain embodiments, the method prevents or reduces the likelihood of an inflammatory condition, e.g., in certain embodiments, the method comprises administering a compound of the present invention to a subject in need thereof in an amount sufficient to prevent or reduce the likelihood of an inflammatory condition. In certain embodiments, the subject is at risk to an inflammatory condition.

The term “inflammatory condition” refers to those diseases, disorders, or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent). Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

In certain embodiments, the inflammatory condition is an acute inflammatory condition (e.g., for example, inflammation resulting from an infection). In certain embodiments, the inflammatory condition is a chronic inflammatory condition. In certain embodiments, the inflammatory condition is inflammation associated with cancer.

### Definitions

### Chemical Terms

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified (e.g., cis or trans alkene), [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). In certain embodiments, an alkyl group has 2 to 6 carbon atoms (“C₂₋₆ alkyl”). Examples of C₁₋₆ alkyl groups include methyl(C₁), ethyl(C₂), propyl(C₃)(e.g., n-propyl, isopropyl), butyl(C₄)(e.g., n-butyl, tert-butyl, sec-butyl, iso-butyl), pentyl(C₅)(e.g., n-pentyl, 3-pentanyl, amyl, neopentyl, 3-methyl-2-butanyl, tertiary amyl), and hexyl(C₆)(e.g., n-hexyl). Additional examples of alkyl groups include n-heptyl(C₇), n-octyl(C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl(Pr, e.g., unsubstituted n-propyl(n-Pr), unsubstituted isopropyl(i-Pr)), unsubstituted butyl(Bu, e.g., unsubstituted n-butyl(n-Bu), unsubstituted tert-butyl (tert-Bu or t-Bu), unsubstituted sec-butyl(sec-Bu), unsubstituted isobutyl(i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In certain embodiments, the haloalkyl moiety has 1 to 8 carbon atoms (“C₁₋₈ haloalkyl”). In certain embodiments, the haloalkyl moiety has 1 to 6 carbon atoms (“C₁₋₆ haloalkyl”). In certain embodiments, the haloalkyl moiety has 1 to 4 carbon atoms (“C₁₋₄ haloalkyl”). In certain embodiments, the haloalkyl moiety has 1 to 3 carbon atoms (“C₁₋₃ haloalkyl”). In certain embodiments, the haloalkyl moiety has 1 to 2 carbon atoms (“C₁₋₂ haloalkyl”). Examples of haloalkyl groups include-CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In certain embodiments, a heteroalkyl group is a saturated group having 1 to 9 carbon atoms and 1 or more heteroatoms within the parent chain (“heteroC₁₋₉ alkyl”). In certain embodiments, a heteroalkyl group is a saturated group having 1 to 8 carbon atoms and 1 or more heteroatoms within the parent chain (“heteroC₁₋₈ alkyl”). In certain embodiments, a heteroalkyl group is a saturated group having 1 to 7 carbon atoms and 1 or more heteroatoms within the parent chain (“heteroC₁₋₇ alkyl”). In certain embodiments, a heteroalkyl group is a saturated group having 1 to 6 carbon atoms and 1 or more heteroatoms within the parent chain (“heteroC₁₋₆ alkyl”). In certain embodiments, a heteroalkyl group is a saturated group having 1 to 5 carbon atoms and 1 or 2 heteroatoms within the parent chain (“heteroC₁₋₅ alkyl”). In certain embodiments, a heteroalkyl group is a saturated group having 1 to 4 carbon atoms and 1 or 2 heteroatoms within the parent chain (“heteroC₁₋₄ alkyl”). In certain embodiments, a heteroalkyl group is a saturated group having 1 to 3 carbon atoms and 1 heteroatom within the parent chain (“heteroC₁₋₃ alkyl”). In certain embodiments, a heteroalkyl group is a saturated group having 1 to 2 carbon atoms and 1 heteroatom within the parent chain (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl(C₂), 1-propenyl(C₃), 2-propenyl(C₃), 1-butenyl(C₄), 2-butenyl(C₄), butadienyl(C₄), and the like. Examples of C₂₋₆ alkenyl groups include the aforementioned C₂₋₄ alkenyl groups as well as pentenyl(C₅), pentadienyl(C₅), hexenyl(C₆), and the like. Additional examples of alkenyl include heptenyl(C₇), octenyl(C₈), octatrienyl(C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or may

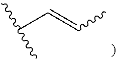

be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In certain embodiments, a heteroalkenyl group has 2 to 9 carbon atoms at least one double bond, and 1 or more heteroatoms within the parent chain (“heteroC₂₋₉ alkenyl”). In certain embodiments, a heteroalkenyl group has 2 to 8 carbon atoms, at least one double bond, and 1 or more heteroatoms within the parent chain (“heteroC₂₋₈ alkenyl”). In certain embodiments, a heteroalkenyl group has 2 to 7 carbon atoms, at least one double bond, and 1 or more heteroatoms within the parent chain (“heteroC₂₋₇ alkenyl”). In certain embodiments, a heteroalkenyl group has 2 to 6 carbon atoms, at least one double bond, and 1 or more heteroatoms within the parent chain (“heteroC₂₋₆ alkenyl”). In certain embodiments, a heteroalkenyl group has 2 to 5 carbon atoms, at least one double bond, and 1 or 2 heteroatoms within the parent chain (“heteroC₂₋₅ alkenyl”). In certain embodiments, a heteroalkenyl group has 2 to 4 carbon atoms, at least one double bond, and 1 or 2 heteroatoms within the parent chain (“heteroC₂₋₄ alkenyl”). In certain embodiments, a heteroalkenyl group has 2 to 3 carbon atoms, at least one double bond, and 1 heteroatom within the parent chain (“heteroC₂₋₃ alkenyl”). In certain embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds)(“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl(C₂), 1-propynyl(C₃), 2-propynyl(C₃), 1-butynyl(C₄), 2-butynyl(C₄), and the like. Examples of C₂₋₆ alkenyl groups include the aforementioned C₂₋₄ alkynyl groups as well as pentynyl(C₅), hexynyl(C₆), and the like. Additional examples of alkynyl include heptynyl(C₇), octynyl(C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In certain embodiments, a heteroalkynyl group has 2 to 9 carbon atoms, at least one triple bond, and 1 or more heteroatoms within the parent chain (“heteroC₂₋₉ alkynyl”). In certain embodiments, a heteroalkynyl group has 2 to 8 carbon atoms, at least one triple bond, and 1 or more heteroatoms within the parent chain (“heteroC₂₋₈ alkynyl”). In certain embodiments, a heteroalkynyl group has 2 to 7 carbon atoms, at least one triple bond, and 1 or more heteroatoms within the parent chain (“heteroC₂-7 alkynyl”). In certain embodiments, a heteroalkynyl group has 2 to 6 carbon atoms, at least one triple bond, and 1 or more heteroatoms within the parent chain (“heteroC₂₋₆ alkynyl”). In certain embodiments, a heteroalkynyl group has 2 to 5 carbon atoms, at least one triple bond, and 1 or 2 heteroatoms within the parent chain (“heteroC₂₋₅ alkynyl”). In certain embodiments, a heteroalkynyl group has 2 to 4 carbon atoms, at least one triple bond, and 1 or 2 heteroatoms within the parent chain (“heteroC₂₋₄ alkynyl”). In certain embodiments, a heteroalkynyl group has 2 to 3 carbon atoms, at least one triple bond, and 1 heteroatom within the parent chain (“heteroC₂₋₃ alkynyl”). In certain embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 7 ring carbon atoms (“C₃₋₇ carbocyclyl”). In certain embodiments, a carbocyclyl group has 3 to 6 ring carbon atoms (“C₃₋₆ carbocyclyl”). In certain embodiments, a carbocyclyl group has 4 to 6 ring carbon atoms (“C₄₋₆ carbocyclyl”). In certain embodiments, a carbocyclyl group has 5 to 6 ring carbon atoms (“C₅₋₆ carbocyclyl”). In certain embodiments, a carbocyclyl group has 5 to 10 ring carbon atoms (“C₅₋₁₀ carbocyclyl”). Exemplary C₃₋₆ carbocyclyl groups include, without limitation, cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), and the like. Exemplary C₃₋₈ carbocyclyl groups include, without limitation, the aforementioned C₃₋₆ carbocyclyl groups as well as cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), and the like. Exemplary C₃₋₁₀ carbocyclyl groups include, without limitation, the aforementioned C₃₋₈ carbocyclyl groups as well as cyclononyl(C₉), cyclononenyl(C₉), cyclodecyl(C₁₀), cyclodecenyl(C₁₀), octahydro-1H-indenyl(C₉), decahydronaphthalenyl(C₁₀), spiro[4.5]decanyl(C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In certain embodiments, “carbocyclyl” is a monocyclic, saturated carbocyclyl group having from 3 to 14 ring carbon atoms (“C₃₋₁₄ cycloalkyl”). In certain embodiments, a cycloalkyl group has 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 4 to 6 ring carbon atoms (“C₄₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl groups include cyclopentyl(C₅) and cyclohexyl(C₅). Examples of C₃₋₆ cycloalkyl groups include the aforementioned C₅₋₆ cycloalkyl groups as well as cyclopropyl(C₃) and cyclobutyl(C₄). Examples of C₃₋₈ cycloalkyl groups include the aforementioned C₃₋₆ cycloalkyl groups as well as cycloheptyl(C₇) and cyclooctyl(C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Carbocyclylalkyl” is a subset of “alkyl” and refers to an alkyl group substituted by a carbocyclyl group, wherein the point of attachment is on the alkyl moiety.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In certain embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In certain embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In certain embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In certain embodiments, the 5-6 membered heterocyclyl has 1-3 ring heteroatoms selected from nitrogen, oxygen, and sulfur. In certain embodiments, the 5-6 membered heterocyclyl has 1-2 ring heteroatoms selected from nitrogen, oxygen, and sulfur. In certain embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Heterocyclylalkyl” is a subset of “alkyl” and refers to an alkyl group substituted by an heterocyclyl group, wherein the point of attachment is on the alkyl moiety.

The term “aryl” refers to a radical of a monocyclic or polycyclic (e.g., bicyclic or tricyclic) 4n+2 aromatic ring system (e.g., having 6, 10, or 14 I electrons shared in a cyclic array) having 6-14 ring carbon atoms and zero heteroatoms provided in the aromatic ring system (“C₆₋₁₄ aryl”). In certain embodiments, an aryl group has 6 ring carbon atoms (“C₆ aryl”; e.g., phenyl). In certain embodiments, an aryl group has 10 ring carbon atoms (“C₁₀ aryl”; e.g., naphthyl such as 1-naphthyl and 2-naphthyl). In certain embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In certain embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In certain embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In certain embodiments, the 5-6 membered heteroaryl has 1-3 ring heteroatoms selected from nitrogen, oxygen, and sulfur. In certain embodiments, the 5-6 membered heteroaryl has 1-2 ring heteroatoms selected from nitrogen, oxygen, and sulfur. In certain embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃+X−; —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(—O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{b})₂, —OP(═O)(NR^{b})₂, —NR^{bb}P(═O)(OR^{cc})₂, —NR^{bb}P(═O)(NR^{b})₂, —P(Rc)₂, —P(R^{cc})₃, —OP(RC)₂, —OP(R^{cc})₃, —B (R^{aa})₂, —B (OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(RC)₂, —CO₂R^{aa}, —SO₂R^{aa}, —C(═NR^{cc})OR^{aa}, —C(═NR^{cc})N(R^{cc})₂, —SO₂N(Rc)₂, —SO₂Rc, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C_{1- 10} perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{o}C. is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X-, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal Rad substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁸⁸ groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X−, —NH(C₁₋₆ alkyl)₂+X−, —NH₂ (C₁₋₆ alkyl)+X−, —NH₃+X−, —N(OC₁₋₆ alkyl)(C₁₋₆ alkyl), —N(OH)(C₁₋₆ alkyl), —NH(OH), —SH, —SC₁₋₆ alkyl, —SS (C₁₋₆ alkyl), —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂ (C₁₋₆ alkyl), —NHC(═O)N(C₁₋₆ alkyl)₂, —NHC(═O)NH(C₁₋₆ alkyl), —NHC(═O)NH₂, —C(═NH) O (C₁₋₆ alkyl), —OC(═NH)(C₁₋₆ alkyl), —OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂ (C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(=S)NH(C₁₋₆ alkyl), C(=S)NH₂, —C(═O)S(C₁₋₆ alkyl), —C(═S) SC₁₋₆ alkyl, —SC(═S) SC₁₋₆ alkyl, —P(═O)₂ (C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂-6alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁸⁸ substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(Rc)₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{o} are as defined herein.

The term “amino” refers to the group-NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and Rec are as defined herein, and wherein R^{bb} of the group-NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and Rec are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃+X″, wherein R^{bb} and X− are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group-S(═O)R^{aa}, wherein R^{aa} is as defined herein.

The term “acyl” refers to a group having the general formula-C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “silyl” refers to the group-Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(Rc)₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{o}C., —SO₂OR^{cc}, —SOR^{aa}, —C(═S)N(Rc)₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C_{1- 10} perhaloalkyl, C₂₋₁₀ alkenyl, C₂₋₁₀ alkynyl, heteroC₁₋₁₀alkyl, heteroC₂₋₁₀alkenyl, heteroC₂-10alkynyl, C₃₋₁₀ carbocyclyl, 3-14 membered heterocyclyl, C₆₋₁₄ aryl, and 5-14 membered heteroaryl, or two Rc groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, Rec and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(Rc)₂, —CO₂R^{aa}, —SO₂R^{aa}, —C(═NR^{cc})R^{aa}, —C(═NR^{cc})OR^{aa}, —C(═NR^{cc})N(Rc)₂, —SO₂N(RC)₂, —SO₂Rc, —SO₂OR^{cc}, —SOR^{aa}, —C(═S)N(Rc)₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10, 10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(Rb)₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{o} are as defined herein. Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR)₂, —P(═O)₂N(R^{bb})₂, and —P(═O)(NR^{bb})₂, wherein R^{aa}, R^{bb}, and Rec are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo), —OR^{aa} (when the O atom is attached to a carbonyl group, wherein R^{aa} is as defined herein), —O (C═O)R^{LG}, or —O (SO)₂R^{LG} (e.g., tosyl, mesyl, besyl), wherein R^{LG} is optionally substituted alkyl, optionally substituted aryl, or optionally substituted heteroaryl. In certain embodiments, the leaving group is a halogen. In certain embodiments, the leaving group is I.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

These and other exemplary substituents are described in more detail in the Detailed Description, Examples, and claims. The invention is not intended to be limited in any manner by the above exemplary listing of substituents.

### Other Definitions

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺ (C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal “Disease,” “disorder,” and “condition” are used interchangeably herein.

The term “administer,” “administering,” or “administration” refers to implanting, absorbing, ingesting, injecting, inhaling, or otherwise introducing a compound described herein, or a composition thereof, in or on a subject.

As used herein, and unless otherwise specified, the terms “treat,” “treating” and “treatment” contemplate an action that occurs while a subject is suffering from the specified infectious disease or inflammatory condition, which reduces the severity of the infectious disease or inflammatory condition, or retards or slows the progression of the infectious disease or inflammatory condition (“therapeutic treatment”), and also contemplates an action that occurs before a subject begins to suffer from the specified infectious disease or inflammatory condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of an infectious disease or inflammatory condition, or to delay or minimize one or more symptoms associated with the infectious disease or inflammatory condition. A therapeutically effective amount of a compound means an amount of therapeutic agent, alone or in combination with other therapies, which provides a therapeutic benefit in the treatment of the infectious disease or inflammatory condition. The term “therapeutically effective amount” can encompass an amount that improves overall therapy, reduces or avoids symptoms or causes of infectious disease or inflammatory condition, or enhances the therapeutic efficacy of another therapeutic agent.

As used herein, and unless otherwise specified, a “prophylactically effective amount” of a compound is an amount sufficient to prevent an infectious disease or inflammatory condition, or one or more symptoms associated with the infectious disease or inflammatory condition, or prevent its recurrence. A prophylactically effective amount of a compound means an amount of a therapeutic agent, alone or in combination with other agents, which provides a prophylactic benefit in the prevention of the infectious disease or inflammatory condition. The term “prophylactically effective amount” can encompass an amount that improves overall prophylaxis or enhances the prophylactic efficacy of another prophylactic agent.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthesis of Lincosamide Analogues

General Experimental Procedures: All reactions were performed in flame-dried round-bottom flasks fitted with rubber septa under positive nitrogen pressure, unless otherwise noted. Air- and moisture-sensitive liquids were transferred via syringe or stainless-steel cannula. Organic solutions were concentrated by rotary evaporation (house vacuum, ca. 25-40 Torr) at ambient temperature, unless otherwise noted. Analytical thin-layer chromatography (TLC) was performed using glass plates precoated with silica gel (0.25 mm, 60 Å pore-size, 230-400 mesh, Merck KGA) impregnated with a fluorescent indicator (254 nm). TLC plates were visualized by exposure to ultraviolet light, then were stained with either an aqueous sulfuric acid solution of ceric ammonium molybdate (CAM), an ethanol-aqueous sulfuric acid solution of 2,4-dinitrophenylhydrazine (DNP), or an aqueous sodium hydroxide-potassium carbonate solution of potassium permanganate (KMnO₄) then briefly heated using a heat gun. Flash-column chromatography was performed as described by Still et al., (Still, W. C.; Khan, M.; Mitra A. J. Org. Chem. 1978, 43, 2923-2925) employing silica gel (60 Å, 15-40 μM, EMD Millipore Corp.).

Materials: Commercial reagents and solvents were used as received.

Instrumentation: Proton magnetic resonance (1H NMR) spectra were recorded on Bruker 400 (400 MHZ)NMR spectrometers at 23° C. Proton chemical shifts are expressed in parts per million (ppm, 8 scale) and are referenced to residual protium in the NMR solvent (CHCl₃, δ 7.26; CD₃OD, δ 3.31). Data are represented as follows: chemical shift, multiplicity (s=singlet, d=doublet, t=triplet, q=quartet, pent=pentent, m=multiplet and/or multiple resonances, br=broad, app=apparent), integration, and coupling constant (J) in Hertz (Hz). Carbon nuclear magnetic resonance spectra (¹³C NMR) were recorded on Bruker 400 (101 MHz)NMR spectrometers at 23° C. Carbon chemical shifts are expressed in parts per million (ppm, δ scale) and are referenced to the carbon resonances of the NMR solvent (CDCl₃, δ 77.16; CD₃OD, δ 49.00). Multiplicity is reported only when ¹³C signals are coupled to magnetically active nuclei (e.g. ¹⁹F). Fluorine nuclear magnetic resonance spectra (¹⁹F NMR) were recorded on Bruker 400 (376 MHz)NMR spectrometers at 23° C. Infrared (IR) spectra were obtained using a Bruker ALPHA FT-IR spectrometer. Data are represented as follows: frequency of absorption (cm⁻¹), intensity of absorption (s=strong, m=medium, w=weak, br=broad). High resolution mass spectra were obtained at the Harvard University Mass Spectrometry Facility using the Thermo Q Exactive Plus Orbitrap mass spectrometer or at the Harvard Laukien-Purcell Instrumentation Center using the Bruker Compact qTOF mass spectrometer via Electrospray Ionization (ESI). High performance liquid chromatography purifications were performed using an Agilent Technologies 1200 series preparative HPLC system.

### Synthesis of FSA1507013, FSA1507066, FSA1507068, FSA1508001, FSA1508003

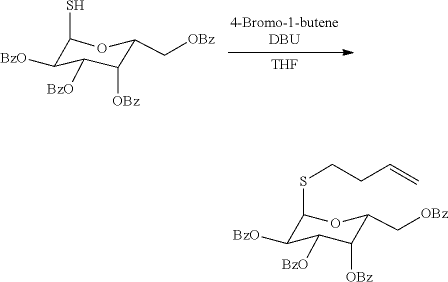

(2R,3S,4S,5R,6R)-2-((benzoyloxy)methyl)-6-(but-3-en-1-ylthio)tetrahydro-2H-pyran-3,4,5-triyl tribenzoate: 4-bromo-1-butene (2.48 mL, 24.5 mmol, 1.5 equiv.) was added to a solution of (2R,3S,4S,5R,6R)-2-((benzoyloxy)methyl)-6-mercaptotetrahydro-2H-pyran-3,4,5-triyl tribenzoate (Prepared according to: Doyle, et al., Org. Lett. 2017, 19, 5802-5805)(10.0 g, 16.3 mmol, 1.0 equiv.) dissolved in distilled THF (60 mL). The solution was cooled to 0° C. and DBU (3.65 mL, 24.5 mmol, 1.5 equiv.) was added, resulting in the immediate formation of a white precipitate. The reaction mixture was stirred for 10 minutes and concentrated under reduced pressure. Purification by flash-column chromatography (10-20% ethyl acetate in hexanes) afforded product (3.0 g, 4.50 mmol, 28% yield) as a white foam. ¹H NMR (400 MHZ, CDCl₃) δ 8.15-8.06 (m, 2H), 8.00 (ddd, J=17.3, 8.3, 1.4 Hz, 4H), 7.85-7.73 (m, 2H), 7.69-7.59 (m, 1H), 7.60-7.43 (m, 4H), 7.46-7.35 (m, 5H), 7.33-7.19 (m, 2H), 6.09-5.97 (m, 2H), 5.90-5.84 (m, 2H), 5.69 (ddt, J=16.9, 10.2, 6.6 Hz, 1H), 5.11-4.90 (m, 3H), 4.61 (dd, J=11.5, 7.5 Hz, 1H), 4.46 (dd, J=11.5, 5.2 Hz, 1H), 2.84-2.53 (m, 2H), 2.53-2.19 (m, 2H). ¹³C NMR (101 MHz, CDCl₃) δ 166.16, 165.84, 165.66, 165.53, 136.19, 133.75, 133.61, 133.36, 133.35, 130.10, 130.07, 129.87, 129.85, 129.61, 129.24, 129.17, 129.14, 128.80, 128.60, 128.57, 128.40, 116.47, 82.78, 69.25, 69.08, 69.06, 67.54, 62.87, 33.78, 29.37. HRMS (ESI+, m/z): [M+Na]⁺ calculated for C₃₈H₃₄O₉S, 689.1816; found 689.1814.

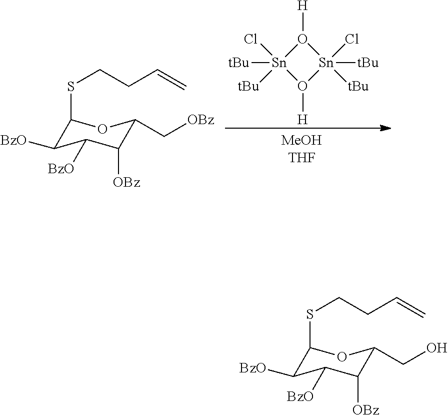

(2R,3R,4S,5S,6R)-2-(but-3-en-1-ylthio)-6-(hydroxymethyl)tetrahydro-2H-pyran-3,4,5-triyl tribenzoate: Dichlorotetrakis(1,1-dimethylethyl)di-μ-hydroxyditin (0.43 g, 0.752 mmol, 0.17 equiv.)(Prepared according to: Ni, M. et al., Carb. Res. 2020, 498, 108172.) was added to (2R,3S,4S,5R,6R)-2-((benzoyloxy)methyl)-6-(but-3-en-1-ylthio)tetrahydro-2H-pyran-3,4,5-triyl tribenzoate (2.95 g, 4.42 mmol, 1.0 equiv.) dissolved in a mixture of anhydrous MeOH (30 mL) and anhydrous THF (9 mL). The reaction mixture was stirred at 65° C. overnight (16 h) and concentrated under reduced pressure. Purification by flash-column chromatography (10-25% ethyl acetate in hexanes) afforded product (2.0 g, 3.25 mmol, 74% yield) as a white foam. ¹H NMR (400 MHZ, CDCl₃) δ 8.11 (dd, J=8.2, 1.4 Hz, 2H), 7.99 (dd, J=8.3, 1.4 Hz, 2H), 7.81 (dd, J=8.2, 1.4 Hz, 2H), 7.64 (t, J=7.4 Hz, 1H), 7.52 (dt, J=9.4, 7.5 Hz, 3H), 7.41 (dt, J=15.6, 7.5 Hz, 3H), 7.23 (m, 2H), 5.99 (d, J=5.1 Hz, 1H), 5.93-5.72 (m, 4H), 5.17-4.98 (m, 2H), 4.73 (t, J=6.8 Hz, 1H), 3.91-3.73 (m, 1H), 3.66 (dd, J=11.7, 6.8 Hz, 1H), 2.82-2.56 (m, 2H), 2.37 (m, 2H). ¹³C NMR (101 MHz, CDCl₃) & 166.73, 165.81, 165.36, 136.18, 133.83, 133.51, 133.28, 130.05, 129.95, 129.70, 129.05, 128.90, 128.71, 128.50, 128.33, 116.46, 82.86, 69.85, 69.55, 69.18, 69.00, 60.78, 33.70, 29.46. HRMS (ESI+, m/z): [M+H]⁺ calculated for C₃₁H₃₀O₈S, 563.1734; found 563.1728.

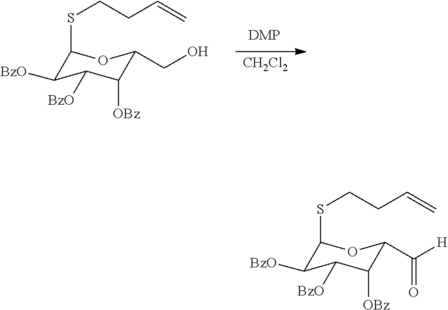

(2R,3R,4S,5R,6S)-2-(but-3-en-1-ylthio)-6-formyltetrahydro-2H-pyran-3,4,5-triyl tribenzoate: To a solution of (2R,3R,4S,5S,6R)-2-(but-3-en-1-ylthio)-6-(hydroxymethyl)tetrahydro-2H-pyran-3,4,5-triyl tribenzoate (2.30 g, 4.09 mmol, 1.00 equiv.) dissolved in dichloromethane (25 mL) was added Dess-Martin Periodinane (2.61 g, 6.14 mmol, 1.50 equiv.) and the resulting white suspension stirred for 1 hour. The reaction mixture was diluted with diethyl ether (100 mL) and treated with saturated aqueous sodium bicarbonate solution (37.5 mL) followed by aqueous sodium thiosulfate solution (50 wt %, 37.5 mL). The resulting bilayer mixture was stirred vigorously at 23° C. for 1 hour and the layers separated. The aqueous layer was extracted with diethyl ether (3×15 mL), and the combined organic extracts were washed sequentially with saturated aqueous sodium bicarbonate solution (37.5 mL) and brine (37.5 mL). The organic solution was dried over sodium sulfate, filtered, and concentrated to yield product (2.27 g, 4.05 mmol, 99% yield) as a white foam, which was used for subsequent sulfinimine condensation without further purification.

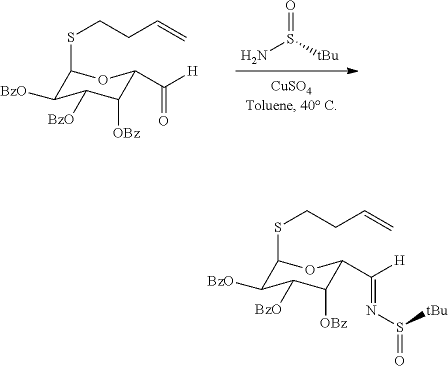

(2R,3R,4S,5S,6R)-2-(but-3-en-1-ylthio)-6-((E)-(((R)-tert-butylsulfinyl)imino)methyl)tetrahydro-2H-pyran-3,4,5-triyl tribenzoate: To a mixture of (2R,3R,4S,5R,6S)-2-(but-3-en-1-ylthio)-6-formyltetrahydro-2H-pyran-3,4,5-triyl tribenzoate (2.27 g, 4.05 mmol, 1.00 equiv.), (R)-(+)-2-methyl-2-propanesulfinamide (983 mg, 8.10 mmol, 2.00 equiv.) and anhydrous copper (II) sulfate (970 mg, 6.08 mmol, 1.50 equiv.) was added anhydrous toluene (13.5 mL). The resulting suspension was heated to 40° C. and stirred for 16 hours. The reaction mixture was allowed to cool to ambience and filtered through celite with washing (3×30 mL of CH₂Cl₂). The combined organic extract was concentrated and purified by flash-column chromatography (10-50% ethyl acetate in hexanes) to yield product (1.88 g, 2.83 mmol, 70% yield) as a white foam. ¹H NMR (400 MHZ, CDCl₃) δ 8.04 (d, J=2.2 Hz, 1H), 7.98 (dd, J=6.9, 1.5 Hz, 3H), 7.78 (dd, J=8.4, 1.4 Hz, 2H), 7.64-7.55 (m, 1H), 7.57-7.48 (m, 1H), 7.45 (t, J=7.6 Hz, 3H), 7.39 (dd, J=8.4, 7.2 Hz, 2H), 7.26 (d, J=2.2 Hz, 3H), 6.30 (dd, J=3.0, 1.5 Hz, 1H), 6.14 (d, J=4.9 Hz, 1H), 5.89-5.71 (m, 3H), 5.53 (t, J=1.8 Hz, 1H), 5.09-4.98 (m, 2H), 2.82-2.63 (m, 2H), 2.42-2.34 (m, 2H), 0.99 (s, 9H). ¹³C NMR (101 MHZ, CDCl₃) δ 165.68, 165.45, 165.30, 164.66, 135.93, 133.59, 133.54, 133.25, 129.95, 129.89, 129.75, 129.14, 129.00, 128.96, 128.61, 128.50, 128.28, 116.72, 83.46, 71.38, 69.45, 69.05, 68.59, 57.11, 33.69, 29.76, 22.08. HRMS (ESI+, m/z): [M+H]⁺ calculated for C₃₅H₃₇NO₈S₂, 664.2033; found 664.2023.

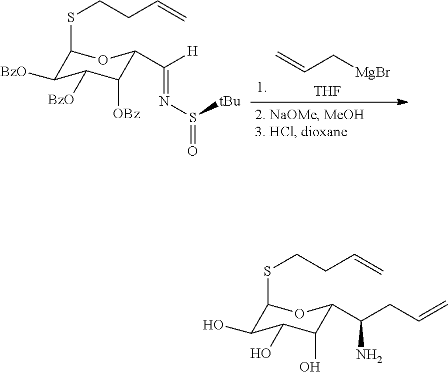

(2R,3R,4S,5R,6R)-2-((R)-1-aminobut-3-en-1-yl)-6-(but-3-en-1-ylthio)tetrahydro-2H-pyran-3,4,5-triol: To a solution of (2R,3R,4S,5S,6R)-2-(but-3-en-1-ylthio)-6-((E)-(((R)-tert-butylsulfinyl)imino)methyl)tetrahydro-2H-pyran-3,4,5-triyl tribenzoate (0.689 g, 1.04 mmol, 1.00 equiv.) dissolved in THF (4 mL) at −78° C. was added allyl magnesium bromide (0.92M in THF, 3.39 mL, 3.12 mmol, 3.00 equiv.) and stirred for 1 hour at −78° C. The solution was allowed to warm to room temperature and 0.5 M NaOMe in MeOH (10 mL, 5.00 mmol, 4.81 equiv.) was added. After stirring for 1 h, 4 M HCl in 1,4-dioxane (2.5 mL, 10.0 mmol, 9.60 equiv.) was added and stirred for 1 h. The reaction mixture was concentrated under reduced pressure and purified by flash-column chromatography (10-20% methanol in DCM with 0.5% sat. aq. NH₄OH) to afford product (0.20 g, 0.691 mmol, 67% yield over 3 steps) as a yellow oil. ¹H NMR (400 MHZ, MeOD) δ 5.92-5.81 (m, 2H), 5.42 (d, J=5.5 Hz, 1H), 5.23-5.15 (m, 2H), 5.12-4.99 (m, 2H), 4.13-4.05 (m, 2H), 3.99 (dd, J=6.9, 1.5 Hz, 1H), 3.57 (dd, J=10.2, 3.4 Hz, 1H), 3.27-3.22 (m, 1H), 2.72-2.48 (m, 3H), 2.46-2.33 (m, 2H), 2.30-2.18 (m, 1H). ¹³C NMR (101 MHZ, MeOD) δ 136.69, 133.93, 117.70, 114.91, 86.67, 70.97, 70.52, 68.91, 67.98, 50.81, 36.18, 33.69, 29.34. HRMS (ESI+, m/z): [M+H]⁺ calculated for C₁₃H₂₃NO₄S, 290.1421; found 290.1423.

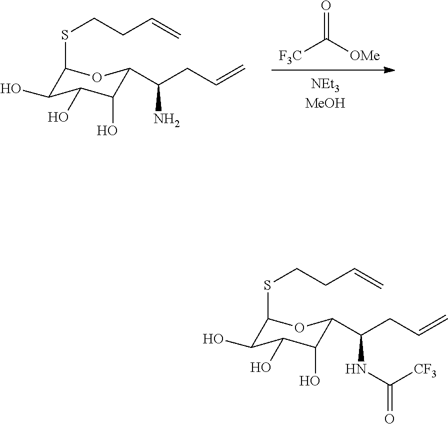 [SMILES: C=CCCSC1OC([C@H](N)CC=C)C(O)C(O)C1O ; COC(=O)C(F)(F)F ; C=CCCSC1OC([C@@H](CC=C)NC(=O)C(F)(F)F)C(O)C(O)C1O]

N—((R)-1-((2R,3R,4S,5R,6R)-6-(but-3-en-1-ylthio)-3,4,5-trihydroxytetrahydro-2H-pyran-2-yl) but-3-en-1-yl)-2,2,2-trifluoroacetamide: To a solution of (2R,3R,4S,5R,6R)-2-((R)-1-aminobut-3-en-1-yl)-6-(but-3-en-1-ylthio)tetrahydro-2H-pyran-3,4,5-triol (0.20 g, 0.691 mmol, 1.00 equiv.) in MeOH (15 mL) was added methyl trifluoroacetate (90 μL, 0.898 mmol, 1.30 equiv.) and triethylamine (289 μL, 2.07 mmol, 3.00 equiv.). After stirring at room temperature overnight (16 h), the reaction mixture was concentrated under reduced pressure. The crude reaction mixture was extracted with ethyl acetate (20 mL) and washed with brine (3×10 mL) to afford product (0.21 g, 0.545 mmol, 79% yield) as a yellow oil. ¹H NMR (400 MHZ, MeOD) δ 5.94-5.70 (m, 2H), 5.41 (d, J=5.7 Hz, 1H), 5.12-4.99 (m, 4H), 4.29 (td, J=8.9, 4.2 Hz, 1H), 4.19 (dd, J=8.4, 1.3 Hz, 1H), 4.09 (dd, J=10.2, 5.6 Hz, 1H), 3.88 (dd, J=3.3, 1.3 Hz, 1H), 3.61-3.54 (m, 1H), 2.71-2.54 (m, 3H), 2.39 (tddt, J=8.1, 6.6, 5.1, 1.4 Hz, 2H), 2.29-2.20 (m, 1H). ¹³C NMR (101 MHz, MeOD) δ 157.53, 157.16, 136.68, 133.89, 116.91, 114.93, 86.66, 70.71, 70.13, 68.76, 67.95, 49.73, 35.35, 33.64, 29.20. HRMS (ESI+, m/z): [M+H]⁺ calculated for C₁₅H₂₂F₃NO₅S, 386.1244; found 386.1244.

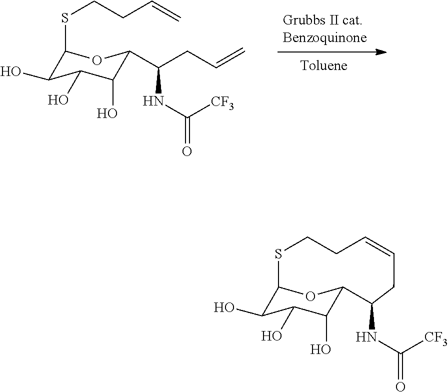 [SMILES: C=CCCSC1OC([C@@H](CC=C)NC(=O)C(F)(F)F)C(O)C(O)C1O ; O=C(N[C@@H]1C/C=C\CCSC2OC1C(O)C(O)C2O)C(F)(F)F]

2,2,2-trifluoro-N-((1R,8R,9R,10R,11S,12R,Z)-10,11,12-trihydroxy-13-oxa-2-thiabicyclo[7.3.1]tridec-5-en-8-yl) acetamide: N—((R)-1-((2R,3R,4S,5R,6R)-6-(but-3-en-1-ylthio)-3,4,5-trihydroxytetrahydro-2H-pyran-2-yl) but-3-en-1-yl)-2,2,2-trifluoroacetamide (0.21 g, 0.545 mmol, 1.00 equiv.) was dissolved in toluene (1360 mL) and the solution was heated to reflux. Freshly sublimed benzoquinone (23.6 mg, 0.218 mmol, 0.40 equiv.) and Grubbs II catalyst (92.6 mg, 0.109 mmol, 0.20 equiv.) dissolved in toluene (5 mL) were added to the reaction mixture. After 20 minutes of stirring at reflux, the reaction mixture was quenched with DMSO (0.77 mL, 10.9 mmol, 20.0 equiv.) and allowed to cool to room temperature. The reaction mixture was concentrated in vacuo, redissolved in ethyl acetate (15 mL) and brine (15 mL), and the layers were separated. The aqueous layer was washed with ethyl acetate (3×15 mL) and the combined organic extract was dried with sodium sulfate and concentrated. Following concentration under reduced pressure, purification by flash-column chromatography (5-10% methanol in DCM) afforded product (75 mg, 0.21 mmol, 39% yield) as a brown oil. ¹H NMR (400 MHZ, MeOD) δ 5.67-5.51 (m, 2H), 5.44 (d, J=5.9 Hz, 1H), 4.50 (dd, J=11.1, 1.0 Hz, 1H), 4.40 (dt, J=11.1, 3.3 Hz, 1H), 4.02 (dd, J=10.2, 5.9 Hz, 1H), 3.77 (d, J=3.3 Hz, 1H), 3.44 (dd, J=10.2, 3.3 Hz, 1H), 3.16 (ddd, J=13.9, 4.2, 2.3 Hz, 1H), 2.99 (tdd, J=13.5, 11.4, 4.2 Hz, 1H), 2.79-2.65 (m, 2H), 2.20-2.13 (m, 1H), 2.05 (dt, J=13.5, 3.7 Hz, 1H). ¹³C NMR (101 MHZ, MeOD) δ 157.49, 157.12, 133.39, 124.10, 117.53, 114.67, 88.97, 71.64, 68.61, 68.51, 67.75, 47.96, 32.88, 27.13, 27.09. HRMS (ESI+, m/z): [M+H]⁺ calculated for C₁₃H₁₈F₃NO₅S, 358.0931; found 358.0932.

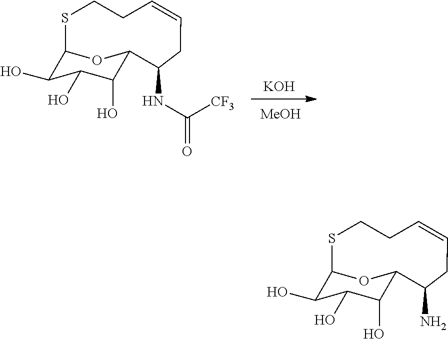

(1R,8R,9R,10R,11S,12R,Z)-8-amino-13-oxa-2-thiabicyclo[7.3.1]tridec-5-ene-10,11,12-triol: To a solution of 2,2,2-trifluoro-N-((1R,8R,9R,10R,11S,12R,Z)-10,11,12-trihydroxy-13-oxa-2-thiabicyclo[7.3.1]tridec-5-en-8-yl) acetamide (40 mg, 0.112 mmol, 1.00 equiv.) dissolved in MeOH (5 mL) was added 20% w/v aqueous KOH (2 mL). The reaction mixture was stirred overnight at room temperature and purification by flash-column chromatography (10-15% methanol in DCM with 0.5% aq. NH₃) afforded product (29 mg, 0.112 mmol, 99% yield) as a yellow oil. ¹H NMR (400 MHZ, MeOD) δ 5.69 (td, J=11.5, 3.4 Hz, 1H), 5.64-5.56 (m, 1H), 5.44 (d, J=5.8 Hz, 1H), 4.36 (dd, J=10.4, 1.1 Hz, 1H), 4.02 (dd, J=10.2, 5.9 Hz, 1H), 3.87 (dd, J=3.4, 1.1 Hz, 1H), 3.56-3.51 (m, 1H), 3.48-3.43 (m, 1H), 3.18-3.13 (m, 1H), 3.04-2.94 (m, 1H), 2.93-2.83 (m, 1H), 2.73 (td, J=13.7, 4.6 Hz, 1H), 2.19 (d, J=13.8 Hz, 1H), 2.08 (d, J=14.1 Hz, 1H). ¹³C NMR (101 MHZ, MeOD) δ 136.08, 123.68, 90.32, 72.74, 70.95, 69.68, 68.87, 49.81, 34.13, 28.56, 28.41. HRMS (ESI+, m/z): [M+H]⁺ calculated for C₁₁H₁₉NO₄S, 262.1108; found 262.1107.

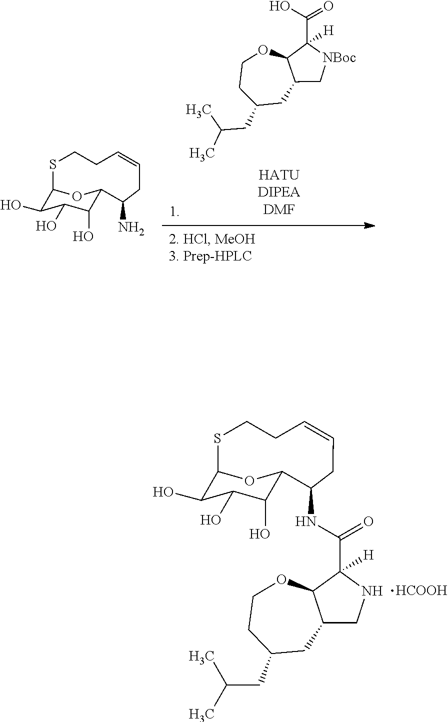

(4S,5aS,8S,8aR)-4-isobutyl-8-(((1R,8R,9R,10R,11S,12R,Z)-10,11,12-trihydroxy-13-oxa-2-thiabicyclo[7.3.1]tridec-5-en-8-yl) carbamoyl) octahydro-2H-oxepino[2,3-c]pyrrol-7-ium formate (FSA1507013·HCOOH): To a solution of (1R,8R,9R,10R,11S,12R,Z)-8-amino-13-oxa-2-thiabicyclo[7.3.1]tridec-5-ene-10,11,12-triol (8.4 mg, 0.032 mmol, 1.00 equiv.) dissolved in DMF (0.5 mL) was added (4S,5aS,8S,8aR)-7-(tert-butoxycarbonyl)-4-isobutyloctahydro-2H-oxepino[2,3-c]pyrrole-8-carboxylic acid (13.2 mg, 0.039 mmol, 1.20 equiv.), HATU (14.7 mg, 0.039 mmol, 1.20 equiv.) and DIPEA (33.6 uL, 0.193 mmol, 6.00 equiv.). The reaction mixture was stirred overnight (16 h) at room temperature. After concentration under reduced pressure, 1 M HCl in MeOH (3 mL, 3.00 mmol, 93.8 equiv.) was added and the reaction mixture was stirred for 1 h. Concentration under reduced pressure, followed by purification via prep-HPLC(5-50% MeCN in H₂O with 0.1% HCOOH over 30 minutes) afforded product FSA1507013·HCOOH (5.0 mg, 0.0094 mmol, 29% yield over two steps) as a thin, transparent film. ¹H NMR (400 MHZ, MeOD) δ 8.35 (s, 2H), 5.74-5.53 (m, 2H), 5.44 (d, J=5.9 Hz, 1H), 4.34 (d, J=5.3 Hz, 4H), 4.01 (td, J=10.3, 4.9 Hz, 2H), 3.85-3.74 (m, 2H), 3.59 (dd, J=11.3, 7.3 Hz, 1H), 3.43 (dd, J=10.2, 3.4 Hz, 1H), 3.16 (ddd, J=14.1, 4.2, 2.3 Hz, 1H), 3.05-2.86 (m, 2H), 2.78-2.61 (m, 2H), 2.32 (d, J=10.7 Hz, 1H), 2.16 (d, J=13.7 Hz, 1H), 2.07 (dd, J=13.3, 4.9 Hz, 1H), 2.01 (d, J=12.8 Hz, 1H), 1.82-1.61 (m, 5H), 1.19 (td, J=6.8, 3.6 Hz, 2H), 1.01-0.93 (m, 1H), 0.90 (d, J=6.5 Hz, 6H). ¹³C NMR (101 MHZ, MeOD) δ 166.62, 166.15, 133.07, 124.72, 88.93, 79.62, 71.62, 69.26, 68.54, 67.92, 67.81, 60.43, 46.68, 42.09, 35.66, 35.17, 34.74, 32.92, 27.14, 24.89, 21.77, 21.53. HRMS (ESI+, m/z): [M+H]⁺ calculated for C₂₄H₄₀N₂O₆S, 485.2680; found 485.2687.

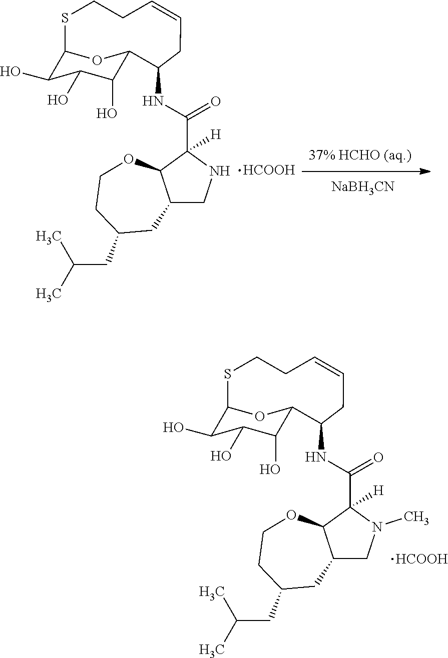

(4S,5aS,8S,8aR)-4-isobutyl-7-methyl-8-(((1R,8R,9R,10R,11S,12R,Z)-10,11,12-trihydroxy-13-oxa-2-thiabicyclo[7.3.1]tridec-5-en-8-yl) carbamoyl) octahydro-2H-oxepino[2,3-c]pyrrol-7-ium formate (FSA1508003·HCOOH): Sodium cyanoborohydride (2.4 mg, 0.038 mmol, 10 equiv.) was added to a solution of FSA1507013·HCOOH (2.0 mg, 0.0038 mmol, 1.0 equiv.) in 37% w/w aqueous formaldehyde (15.4 mg, 0.114 mmol, 50 equiv.) and stirred at ambience for 45 mins. The reaction mixture was quenched with saturated aqueous sodium carbonate (10 mL), extracted with CH₂Cl₂ (2×20 mL), and the combined organic layers dried over sodium sulfate. Concentration under reduced pressure, followed by purification via prep-HPLC(5-50% MeCN in H₂O with 0.1% HCOOH over 30 minutes) afforded product FSA1508003·HCOOH (2.0 mg, 0.0037 mmol, 97% yield) as a thin, transparent film. ¹H NMR (600 MHZ, CD₃OD) δ 8.30 (br s, 1H), 5.66-5.56 (m, 2H), 5.44 (d, J=5.9 Hz, 1H), 4.39 (t, J=9.2 Hz, 1H), 4.36-4.27 (m, 2H), 4.02 (dd, J=10.3, 6.0 Hz, 1H), 4.00-3.97 (m, 1H), 3.85 (d, J=3.5 Hz, 1H), 3.87-3.74 (m, 2H), 3.48 (dd, J=10.3, 6.3 Hz, 1H), 3.42 (dd, J=10.2, 3.3 Hz, 1H), 3.15 (ddd, J=14.1, 4.2, 2.3 Hz, 1H), 2.99 (qd, J=13.7, 4.3 Hz, 1H), 2.74-2.62 (m, 6H), 2.30 (qdd, J=11.7, 8.7, 2.4 Hz, 1H), 2.16 (d, J=13.3 Hz, 1H), 2.03 (d, J=12.6 Hz, 1H), 1.97 (d, J=13.3 Hz, 1H), 1.75-1.61 (m, 4H), 1.24-1.12 (m, 2H), 0.94 (q, J=11.7, 11.0 Hz, 1H), 0.89 (d, J=2.0 Hz, 3H), 0.88 (d, J=1.9 Hz, 3H). ¹³C NMR (101 MHz, CD₃OD) δ 134.78, 125.79, 90.32, 81.52, 72.95, 72.73, 71.00, 70.03, 69.23, 69.02, 59.60, 49.05, 48.09, 45.04, 41.27, 37.71, 37.12, 35.47, 34.27, 28.62, 28.55, 26.30, 23.21, 22.91. HRMS (ESI+, m/z): [M+H]⁺ calculated for C₂₅H₄₂N₂O₆S, 499.2836; found 499.2834.

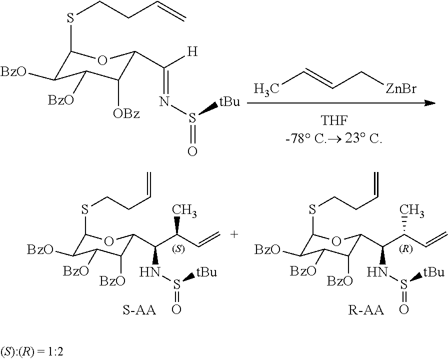

(2R,3R,4S,5S,6R)-2-(but-3-en-1-ylthio)-6-((1R,2S)-1-(((R)-tert-butylsulfinyl)amino)-2-methylbut-3-en-1-yl)tetrahydro-2H-pyran-3,4,5-triyl tribenzoate (S-AA) and (2R,3R,4S,5S,6R)-2-(but-3-en-1-ylthio)-6-((1R,2R)-1-(((R)-tert-butylsulfinyl)amino)-2-methylbut-3-en-1-yl)tetrahydro-2H-pyran-3,4,5-triyl tribenzoate (R-AA): To a solution of (2R,3R,4S,5S,6R)-2-(but-3-en-1-ylthio)-6-((E)-(((R)-tert-butylsulfinyl)imino)methyl)tetrahydro-2H-pyran-3,4,5-triyl tribenzoate (0.320 g, 0.483 mmol, 1.00 equiv.) dissolved in THF (5 mL) at −78° C. was added crotylzinc bromide (Prepared according to: Krasovskiy, A.; Knochel, P. Synthesis 2006, 890-891)(0.20M in THF, 7.2 mL, 1.44 mmol, 3.00 equiv.) and allowed to warm up to room temperature overnight. The mixture was diluted with ethyl acetate (10 mL) and quenched with saturated aqueous ammonium chloride (20 mL). The layers were separated and the aqueous layer further extracted with ethyl acetate (3×20 mL). The combined organic layers were then dried over sodium sulfate, filtered and concentrated in vacuo. Purification via flash-column chromatography (10-40% ethyl acetate in hexanes) afforded product R-AA (129.3 mg, 0.180 mmol, 37% yield) as the major, first-eluting product and S-AA (64.7 mg, 0.090 mmol, 19% yield) as the minor, second-eluting product, both as white foams.

R-AA: ¹H NMR (400 MHZ, CDCl₃) δ 8.15-8.08 (m, 2H), 8.00-7.93 (m, 2H), 7.80-7.73 (m, 2H), 7.66-7.57 (m, 1H), 7.56-7.46 (m, 3H), 7.44-7.34 (m, 3H), 7.23 (t, J=7.8 Hz, 2H), 6.25-6.20 (m, 1H), 6.08-5.92 (m, 2H), 5.83-5.67 (m, 3H), 5.17 (dt, J=10.5, 1.5 Hz, 1H), 5.11 (dt, J=17.3, 1.6 Hz, 1H), 5.04-4.93 (m, 2H), 4.71-4.62 (m, 1H), 3.58-3.50 (m, 2H), 2.90 (pent, J=6.9 Hz, 1H), 2.78-2.62 (m, 2H), 2.44-2.26 (m, 2H), 1.14 (s, 9H), 1.12 (d, J=7.3 Hz, 3H). ¹³C NMR (101 MHz, CDCl₃) δ 165.96, 165.44, 165.22, 138.72, 136.14, 133.56, 133.53, 133.16, 130.10, 130.07, 129.93, 129.72, 129.36, 129.24, 128.78, 128.56, 128.26, 116.68, 116.62, 83.97, 70.49, 69.70, 69.36, 69.10, 60.01, 57.12, 37.80, 33.88, 30.57, 23.03, 16.64. HRMS (ESI+, m/z): [M+H]⁺ calculated for C₃₉H₄₅NO₈S₂, 720.2659; found 720.2655.

S-AA: ¹H NMR (400 MHZ, CDCl₃) δ 8.15-8.06 (m, 2H), 8.01-7.91 (m, 2H), 7.85-7.72 (m, 2H), 7.72-7.57 (m, 1H), 7.55-7.46 (m, 3H), 7.44-7.34 (m, 3H), 7.27-7.18 (m, 2H), 6.20 (d, J=3.2 Hz, 1H), 6.04 (d, J=5.8 Hz, 1H), 5.89-5.71 (m, 3H), 5.67 (dd, J=10.8, 3.2 Hz, 1H), 5.22-5.11 (m, 2H), 5.10-4.94 (m, 2H), 4.74 (d, J=6.9 Hz, 1H), 3.71 (d, J=5.9 Hz, 1H), 3.60-3.51 (m, 1H), 2.79-2.59 (m, 3H), 2.46-2.29 (m, 2H), 1.13 (d, J=7.6 Hz, 3H), 1.09 (s, 9H). ¹³C NMR (101 MHZ, CDCl₃) δ 165.85, 165.44, 165.28, 140.70, 136.22, 133.73, 133.54, 133.22, 130.11, 130.07, 129.87, 129.51, 129.27, 129.24, 128.85, 128.57, 128.32, 116.70, 116.14, 83.91, 69.71, 69.13, 68.96, 68.92, 60.07, 56.82, 39.98, 33.90, 30.39, 22.94, 16.05. HRMS (ESI+, m/z): [M+H]⁺ calculated for C₃₉H₄₅NO₈S₂, 720.2659; found 720.2653.

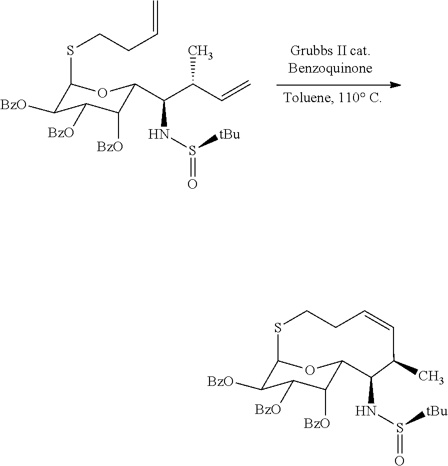

(1R,7R,8R,9R,10S,11S,12R,Z)-8-(((R)-tert-butylsulfinyl)amino)-7-methyl-13-oxa-2-thiabicyclo[7.3.1]tridec-5-ene-10,11,12-triyl tribenzoate: (2R,3R,4S,5S,6R)-2-(but-3-en-1-ylthio)-6-((1R,2R)-1-(((R)-tert-butylsulfinyl)amino)-2-methylbut-3-en-1-yl)tetrahydro-2H-pyran-3,4,5-triyl tribenzoate (129.3 mg, 0.180 mmol, 1.00 equiv.) was dissolved in toluene (895 mL) and the solution was heated to reflux. Freshly sublimed benzoquinone (7.8 mg, 0.072 mmol, 0.40 equiv.) and Grubbs II catalyst (30.6 mg, 0.036 mmol, 0.20 equiv.) dissolved in toluene (5 mL) were added to the reaction mixture. After 20 minutes of stirring at reflux, the reaction mixture was quenched with DMSO (0.25 mL, 3.6 mmol, 20.0 equiv.) and allowed to cool to room temperature. The reaction mixture was concentrated in vacuo, redissolved in ethyl acetate (10 mL) and brine (10 mL), and the layers were separated. The aqueous layer was washed with ethyl acetate (3×10 mL) and the combined organic extract was dried with sodium sulfate and concentrated. Purification by flash-column chromatography (15-45% ethyl acetate in hexanes afforded the product (24 mg, 0.0347 mmol, 19% yield) as a light-yellow film, which was found to exist as a 74:26 mixture of atropisomers by NMR. For clarity, only peaks corresponding to the major atropisomer are presented. ¹H NMR (400 MHZ, CDCl₃) δ 8.14-8.03 (m, 2H), 8.00-7.91 (m, 2H), 7.83-7.72 (m, 2H), 7.66-7.57 (m, 1H), 7.57-7.44 (m, 3H), 7.43-7.33 (m, 3H), 7.26-7.19 (m, 2H), 6.21 (d, J=2.9 Hz, 1H), 6.04 (d, J=5.5 Hz, 1H), 5.75-5.61 (m, 3H), 5.52 (td, J=11.7, 3.6 Hz, 1H), 4.77 (d, J=10.5 Hz, 1H), 3.48 (ddd, J=10.4, 7.0, 3.4 Hz, 1H), 3.34 (d, J=7.2 Hz, 1H), 3.28-3.06 (m, 2H), 2.96 (qd, J=13.5, 4.1 Hz, 1H), 2.67 (td, J=13.7, 4.5 Hz, 1H), 2.26 (d, J=13.9 Hz, 1H), 1.28 (s, 9H), 1.11 (d, J=6.8 Hz, 3H). ¹³C NMR (101 MHz, CDCl₃) δ 166.01, 165.54, 165.06, 133.55, 133.36, 133.06, 131.93, 130.86, 130.11, 130.08, 129.97, 129.87, 129.57, 129.29, 128.70, 128.60, 128.22, 85.39, 70.43, 70.33, 69.31, 68.67, 59.54, 57.49, 33.84, 32.28, 28.10, 23.12, 17.60. HRMS (ESI+, m/z): [M+H]⁺ calculated for C₃₇H₄₁NO: S₂, 692.2346; found 692.2337.

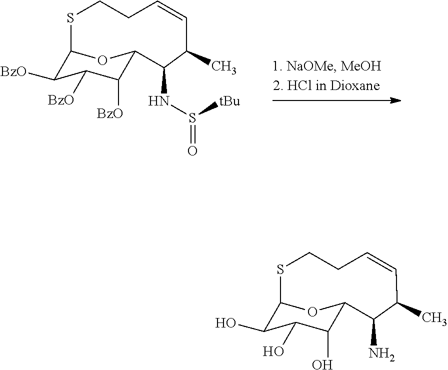

(1R,7S,8R,9R,10R,11S,12R,Z)-8-amino-7-methyl-13-oxa-2-thiabicyclo[7.3.1]tridec-5-ene-10,11,12-triol: NaOMe in MeOH (0.5 M, 1.0 mL, 0.5 mmol, 14.4 equiv.) was added to (1R,7R,8R,9R,10S,11S,12R,Z)-8-(((R)-tert-butylsulfinyl)amino)-7-methyl-13-oxa-2-thiabicyclo[7.3.1]tridec-5-ene-10,11,12-triyl tribenzoate (24 mg, 0.0347 mmol, 1.00 equiv.) and the resulting solution stirred for 1 h. HCl in dioxane (4.0 M, 0.25 mL, 1.0 mmol, 28.8 equiv.) was then added to the mixture, and the resulting white suspension stirred for 1 h. The reaction mixture was concentrated in vacuo and purified via prep-HPLC (5-50% MeCN in H₂O with 0.1% HCOOH over 30 minutes) to yield the product (7.3 mg, 0.0265 mmol, 76% yield over two steps) as a light-yellow film. NMR indicated the product was isolated as its free base rather than its formate salt. ¹H NMR (400 MHZ, MeOD) δ 5.64 (td, J=11.7, 3.7 Hz, 1H), 5.47 (d, J=5.9 Hz, 1H), 5.31 (td, J=11.1, 1.8 Hz, 1H), 4.45 (dd, J=10.5, 1.2 Hz, 1H), 4.04 (dd, J=10.2, 5.9 Hz, 1H), 3.92-3.85 (m, 1H), 3.51 (dd, J=10.4, 3.3 Hz, 1H), 3.47 (dd, J=10.2, 3.4 Hz, 1H), 3.23 (ddd, J=11.1, 7.1 3.5 Hz, 1H), 3.17 (ddd, J=14.0, 4.1, 2.3 Hz, 1H), 2.93 (qd, J=13.2, 4.1 Hz, 1H), 2.72 (td, J=13.6, 4.5 Hz, 1H), 2.33-2.12 (m, 1H), 1.16 (d, J=7.1 Hz, 3H). ¹³C NMR (101 MHZ, MeOD) δ 134.66, 129.25, 90.23, 72.51, 70.68, 69.52, 68.78, 55.20, 33.88, 30.84, 29.06, 16.94. HRMS (ESI+, m/z): [M+H]⁺ calculated for C₁₂H₂₁NO₄S, 276.1264; found 276.1263

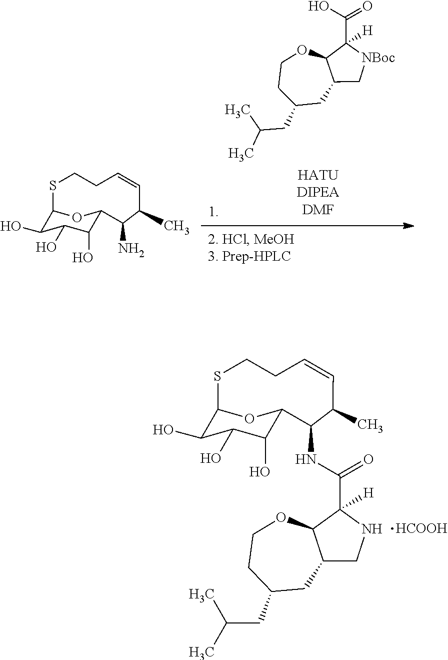

(4S,5aS,8S,8aR)-4-isobutyl-8-(((1R,7R,8R,9R,10R,11S,12R,Z)-10,11,12-trihydroxy-7-methyl-13-oxa-2-thiabicyclo[7.3.1]tridec-5-en-8-yl) carbamoyl) octahydro-2H-oxepino[2,3-c]pyrrol-7-ium formate (FSA1507066·HCOOH): To (1R,7R,8R,9R,10R,11S,12R,Z)-8-amino-7-methyl-13-oxa-2-thiabicyclo[7.3.1]tridec-5-ene-10,11,12-triol (7.3 mg, 0.0265 mmol, 1.00 equiv.) dissolved in DMF (0.5 mL) was added (4S,5aS,8S,8aR)-7-(tert-butoxycarbonyl)-4-isobutyloctahydro-2H-oxepino[2,3-c]pyrrole-8-carboxylic acid (10.8 mg, 0.032 mmol, 1.20 equiv.), HATU (12.0 mg, 0.032 mmol, 1.20 equiv.) and DIPEA (27.7 μL, 0.159 mmol, 6.00 equiv.). The reaction mixture was stirred overnight (16 h) at room temperature. After concentration under reduced pressure, 1M HCl in MeOH (1 mL, 1.00 mmol, 37.7 equiv.) was added and the reaction mixture was stirred for 1 h. Concentration under reduced pressure, followed by purification via prep-HPLC(5-50% MeCN in H₂O with 0.1% HCOOH over 30 minutes) afforded product FSA1507066·HCOOH (5.0 mg, 0.0092 mmol, 35% yield over two steps) as a thin, transparent film. ¹H NMR (400 MHz, MeOD) δ 8.45 (br s, 1H), 5.50 (td, J=11.6, 3.5 Hz, 1H), 5.44 (d, J=5.9 Hz, 1H), 5.38 (td, J=10.9, 1.7 Hz, 1H), 4.43-4.20 (m, 4H), 4.06-3.95 (m, 2H), 3.81 (td, J=11.6, 11.0, 3.7 Hz, 1H), 3.75 (d, J=3.3 Hz, 1H), 3.58 (dd, J=11.3, 7.4 Hz, 1H), 3.41 (dd, J=10.2, 3.4 Hz, 1H), 3.15 (ddd, J=13.9, 4.0, 2.2 Hz, 1H), 3.06 (ddd, J=10.7, 7.1, 3.3 Hz, 1H), 2.98 (dd, J=12.6, 4.1 Hz, 1H), 2.87 (t, J=11.7 Hz, 1H), 2.69 (td, J=13.6, 4.6 Hz, 1H), 2.36-2.24 (m, 1H), 2.23-2.15 (m, 1H), 2.00 (d, J=12.4 Hz, 1H), 1.90-1.58 (m, 4H), 1.19 (td, J=6.8, 3.5 Hz, 2H), 1.03 (d, J=6.9 Hz, 3H), 1.02-0.92 (m, 1H), 0.89 (d, J=6.5 Hz, 6H). ¹³C NMR (101 MHz, MeOD) δ 169.22, 168.65, 132.52, 132.20, 90.19, 80.95, 72.91, 72.14, 70.01, 69.50, 69.11, 62.27, 53.96, 49.50, 48.13, 43.67, 37.31, 36.43, 36.06, 34.11, 32.54, 29.03, 26.29, 23.17, 22.93, 17.45. HRMS (ESI+, m/z): [M+H]⁺ calculated for C₂₅H₄₂N₂O₆S, 499.2836; found 499.2834.

FSA1507066·HCOOH was prepared and isolated as a single diastereomer. The C-7 methyl group was assigned as the R-stereocenter. Moreover, one of ordinary skill would recognize that the compound having either the R or the S-stereocenter at the C-7 methyl group is obtainable by the synthetic route described herein.

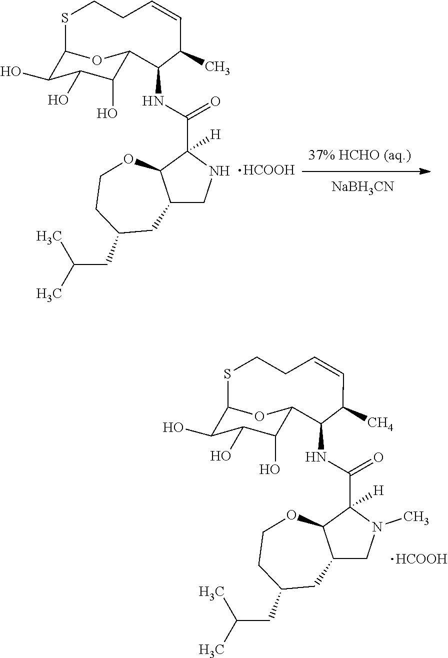

(4S,5aS,8S,8aR)-4-isobutyl-7-methyl-8-(((1R,7R,8R,9R,10R,11S,12R,Z)-10,11,12-trihydroxy-7-methyl-13-oxa-2-thiabicyclo[7.3.1]tridec-5-en-8-yl) carbamoyl) octahydro-2H-oxepino[2,3-c]pyrrol-7-ium formate (FSA1508001·HCOOH): Sodium cyanoborohydride (2.4 mg, 0.038 mmol, 10 equiv.) was added to a solution of FSA1507066·HCOOH (2.1 mg, 0.0038 mmol, 1.0 equiv.) in 37% w/w aqueous formaldehyde (15.4 mg, 0.114 mmol, 50 equiv.) and stirred at ambience for 45 mins. The reaction mixture was quenched with saturated aqueous sodium carbonate (10 mL), extracted with CH₂Cl₂ (2×20 mL), and the combined organic layers dried over sodium sulfate. Concentration under reduced pressure, followed by purification via prep-HPLC(5-50% MeCN in H₂O with 0.1% HCOOH over 30 minutes) afforded product

FSA1508001·HCOOH (1.9 mg, 0.0034 mmol, 89% yield) as a thin, transparent film. ¹H NMR (400 MHZ, MeOD) δ 8.29 (br s, 1H), 5.52 (td, J=11.6, 3.5 Hz, 1H), 5.45 (d, J=5.9 Hz, 1H), 5.34 (td, J=11.0, 1.9 Hz, 1H), 4.37 (dd, J=11.0, 7.2 Hz, 1H), 4.30-4.17 (m, 2H), 4.02 (dd, J=10.2, 5.9 Hz, 1H), 4.02-3.95 (m, 1H), 3.85 (d, J=3.6 Hz, 1H), 3.83-3.66 (m, 2H), 3.48-3.44 (m, 1H), 3.41 (dd, J=10.3, 3.3 Hz, 1H), 3.16 (ddd, J=14.1, 4.2, 2.3 Hz, 1H), 3.13-3.02 (m, 1H), 2.97 (qd, J=13.1, 4.1 Hz, 1H), 2.71 (dd, J=13.7, 4.6 Hz, 1H), 2.68-2.54 (m, f4H), 2.30 (dtt, J=11.9, 5.9, 2.6 Hz, 1H), 2.24-2.15 (m, 1H), 1.97 (d, J=13.4 Hz, 1H), 1.73-1.59 (m, 4H), 1.19 (hept, J=6.7 Hz, 2H), 1.01 (d, J=6.8 Hz, 3H), 0.97-0.90 (m, 1H), 0.894 (d, J=6.5 Hz, 3H), 0.887 (d, J=6.5 Hz, 3H). ¹³C NMR (101 MHZ, MeOD) δ 170.31, 166.89, 132.54, 132.22, 90.18, 81.54, 73.25, 72.88, 72.37, 70.06, 69.39, 68.85, 59.71, 53.49, 48.11, 45.13, 41.51, 37.73, 37.22, 35.36, 34.07, 32.26, 29.07, 26.29, 23.22, 22.91, 17.54. HRMS (ESI+, m/z): [M+H]⁺ calculated for C₂₆H₄₄N₂O₆S, 513.2993; found 513.2992.

FSA1508001·HCOOH was prepared and isolated as a single diastereomer. The C-7 methyl group was assigned as the R-stereocenter. Moreover, one of ordinary skill would recognize that the compound having either the R or the S-stereocenter at the C-7 methyl group is obtainable by the synthetic route described herein.

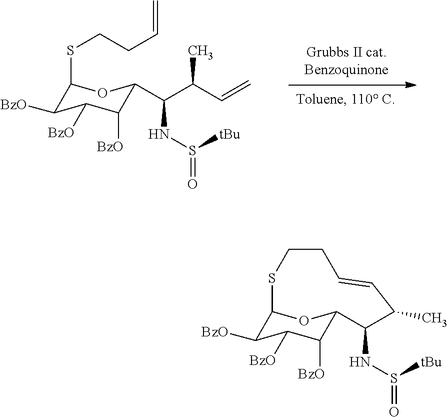

(1R,7S,8R,9R,10S,11S,12R,E)-8-(((R)-tert-butylsulfinyl)amino)-7-methyl-13-oxa-2-thiabicyclo[7.3.1]tridec-5-ene-10,11,12-triyl tribenzoate: (2R,3R,4S,5S,6R)-2-(but-3-en-1-ylthio)-6-((1R,2S)-1-(((R)-tert-butylsulfinyl)amino)-2-methylbut-3-en-1-yl)tetrahydro-2H-pyran-3,4,5-triyl tribenzoate (64.7 mg, 0.090 mmol, 1.00 equiv.) was dissolved in toluene (445 mL) and the solution was heated to reflux. Freshly sublimed benzoquinone (3.9 mg, 0.036 mmol, 0.40 equiv.) and Grubbs II catalyst (15.3 mg, 0.018 mmol, 0.20 equiv.) dissolved in toluene (5 mL) were added to the reaction mixture. After 20 minutes of stirring at reflux, the reaction mixture was quenched with DMSO (0.25 mL, 3.6 mmol, 40.0 equiv.) and allowed to cool to room temperature. The reaction mixture was concentrated in vacuo, redissolved in ethyl acetate (5 mL) and brine (5 mL), and the layers were separated. The aqueous layer was washed with ethyl acetate (3×5 mL) and the combined organic extract was dried with sodium sulfate and concentrated. Purification by flash-column chromatography (15-45% ethyl acetate in hexanes afforded the product (4 mg, 0.0058 mmol, 6% yield) as a light-yellow film. The product was found to exist as a 66:34 mixture of atropisomers by NMR. For clarity, only peaks corresponding to the major atropisomer are presented. ¹H NMR (400 MHZ, CDCl₃) δ 8.14-8.03 (m, 2H), 8.01-7.91 (m, 2H), 7.84-7.72 (m, 2H), 7.67-7.57 (m, 1H), 7.53-7.46 (m, 3H), 7.47-7.33 (m, 3H), 7.25-7.19 (m, 2H), 6.09-5.96 (m, 2H), 5.78-5.56 (m, 3H), 5.31 (t, J=12.9 Hz, 1H), 4.87 (d, J=10.5 Hz, 1H), 3.92 (d, J=4.9 Hz, 1H), 3.34-3.18 (m, 2H), 3.05 (s, 1H), 2.68 (d, J=13.5 Hz, 1H), 2.43 (t, J=13.9 Hz, 1H), 2.10 (q, J=13.5, 12.5 Hz, 1H), 1.31 (s, 9H), 1.26 (d, J=9.8 Hz, 3H). ¹³C NMR (101 MHZ, CDCl₃) δ 166.19, 165.94, 165.53, 133.71, 133.55, 133.28, 133.07, 131.90, 130.14, 130.11, 130.06, 129.97, 129.82, 129.26, 128.79, 128.60, 128.36, 88.29, 70.34, 69.82, 69.50, 69.45, 57.38, 56.61, 38.20, 37.95, 35.69, 22.89, 18.94. HRMS (ESI+, m/z): [M+H]⁺ calculated for C₃₇H₄₁NO: S₂, 692.2346; found 692.2338.

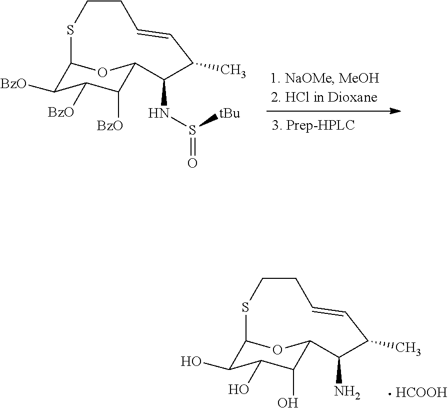

(1R,7S,8R,9R,10R,11S,12R,E)-10,11,12-trihydroxy-7-methyl-13-oxa-2-thiabicyclo[7.3.1]tridec-5-en-8-aminium formate: NaOMe in MeOH (0.5 M, 1.0 mL, 0.5 mmol, 86.2 equiv.) was added to (1R,7S,8R,9R,10S,11S,12R,E)-8-(((R)-tert-butylsulfinyl)amino)-7-methyl-13-oxa-2-thiabicyclo[7.3.1]tridec-5-ene-10,11,12-triyl tribenzoate (4 mg, 0.0058 mmol, 1.00 equiv.) and the resulting solution stirred for 1 h. HCl in dioxane (4.0 M, 0.25 mL, 1.0 mmol, 172.4 equiv.) was then added to the mixture, and the resulting white suspension stirred for 1 h. The reaction mixture was concentrated in vacuo and purified via prep-HPLC(5-50% MeCN in H₂O with 0.1% HCOOH over 30 minutes) to yield the product (1.5 mg, 0.0047 mmol, 80% yield over two steps) as a light-yellow film. The product was found to exist as a 54:46 mixture of atropisomers by NMR, as verified by the method reported by Hu et al. (Hu, D. X.; Grice, P.; Ley, S. V. J. Org. Chem. 2012, 77, 5198-5202). For clarity, only peaks corresponding to the major atropisomer are presented. ¹H NMR (400 MHZ, MeOD) δ 8.34 (s, 1H), 5.76 (s, 1H), 5.30 (dd, J=15.8, 9.6 Hz, 1H), 5.22 (d, J=5.8 Hz, 1H), 4.36 (d, J=7.9 Hz, 1H), 4.15-3.98 (m, 1H), 3.90 (s, 1H), 3.58-3.50 (m, 1H), 3.31-3.17 (m, 1H), 2.94-2.83 (m, 1H), 2.57-2.34 (m, 2H), 2.31-2.17 (m, 1H), 2.11-1.94 (m, 1H), 1.32-1.20 (m, 3H). ¹³C NMR (101 MHz, MeOD) δ 167.92, 135.36, 131.44, 89.91, 71.68, 70.38, 70.23, 69.63, 57.26, 43.88, 36.06, 29.03, 18.09. HRMS (ESI+, m/z): [M+H]⁺ calculated for C₁₂H₂₁NO₄S, 276.1264; found 276.1264.

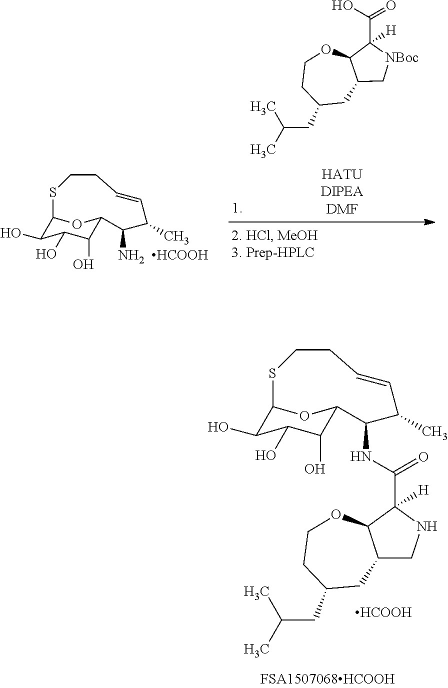

(4S,5aS,8S,8aR)-4-isobutyl-N-((1R,7S,8R,9R,10R,11S,12R,E)-10,11,12-trihydroxy-7-methyl-13-oxa-2-thiabicyclo[7.3.1]tridec-5-en-8-yl) octahydro-2H-oxepino[2,3-c]pyrrole-8-carboxamide formate salt (FSA1507068·HCOOH): To ((1R,7S,8R,9R,10R,11S,12R,E)-10,11,12-trihydroxy-7-methyl-13-oxa-2-thiabicyclo[7.3.1]tridec-5-en-8-aminium formate (1.5 mg, 0.0047 mmol, 1.00 equiv.) dissolved in DMF (0.1 mL) was added (4S,5aS,8S,8aR)-7-(tert-butoxycarbonyl)-4-isobutyloctahydro-2H-oxepino[2,3-c]pyrrole-8-carboxylic acid (1.9 mg, 0.0056 mmol, 1.20 equiv.), HATU (2.1 mg, 0.0056 mmol, 1.20 equiv.) and DIPEA (5.9 μL, 0.0336 mmol, 6.00 equiv.). The reaction mixture was stirred overnight (16 h) at room temperature. After concentration under reduced pressure, 1M HCl in MeOH (0.5 mL, 0.50 mmol, 106 equiv.) was added and the reaction mixture was stirred for 1 h. Concentration under reduced pressure, followed by purification via prep-HPLC(5-50% MeCN in H₂O with 0.1% HCOOH over 30 minutes) afforded product FSA1507068·HCOOH (0.737 mg, 0.00135 mmol, 29% yield over two steps) as a thin, transparent film. ¹H NMR (400 MHZ, MeOD) 8 8.38 (s, 1H), 5.40-5.26 (m, 2H), 5.21 (d, J=5.7 Hz, 1H), 4.41-4.23 (m, 3H), 4.19 (d, J=10.8 Hz, 1H), 4.07-3.93 (m, 2H), 3.84-3.71 (m, 2H), 3.59 (dd, J=11.3, 7.4 Hz, 1H), 3.38 (dd, J=10.3, 3.3 Hz, 1H), 3.21 (dt, J=14.6, 3.5 Hz, 1H), 2.88 (t, J=11.7 Hz, 1H), 2.71-2.47 (m, 3H), 2.34-2.25 (m, 1H), 2.03-1.88 (m, 2H), 1.85-1.57 (m, 4H), 1.31-1.11 (m, 2H), 1.03 (d, J=7.1 Hz, 3H), 1.01-0.92 (m, 1H), 0.89 (d, J=6.5 Hz, 6H). ¹³C NMR (101 MHz, MeOD) δ 168.17, 168.09, 136.37, 131.08, 93.81, 80.95, 72.81, 71.80, 70.59, 69.95, 69.05, 62.51, 52.01, 49.59, 48.11, 43.57, 40.93, 37.86, 37.28, 36.49, 36.20, 36.02, 26.29, 23.16, 22.93, 16.73. HRMS (ESI+, m/z): [M+H]⁺ calculated for C₂₅H₄₂N₂O₆S, 499.2836; found 499.2836.

FSA1507068. HCOOH was prepared and isolated as a single diastereomer. The C-7 methyl group was assigned as the S-stereocenter. Moreover, one of ordinary skill would recognize that the compound having either the R or the S-stereocenter at the C-7 methyl group is obtainable by the synthetic route described herein.

### Synthesis of FSA1503054, FSA1503055, FSA1503058

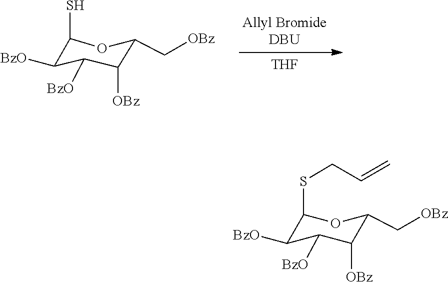

(2R,3R,4S,5S,6R)-2-(allylthio)-6-((benzoyloxy)methyl)tetrahydro-2H-pyran-3,4,5-triyl tribenzoate: Allyl bromide (2.12 mL, 24.5 mmol, 1.5 equiv.) was added to a solution of (2R,3S,4S,5R,6R)-2-((benzoyloxy)methyl)-6-mercaptotetrahydro-2H-pyran-3,4,5-triyl tribenzoate (10.0 g, 16.3 mmol, 1.0 equiv.) dissolved in distilled THF (60 mL). The solution was cooled to 0° C. and DBU (3.65 mL, 24.5 mmol, 1.5 equiv.) was added, resulting in the immediate formation of a white precipitate. The reaction mixture was stirred for 10 minutes and concentrated under reduced pressure. Purification by flash-column chromatography (10-20% ethyl acetate in hexanes) afforded the product (6.38 g, 9.78 mmol, 60% yield) as a white foam. ¹H NMR (500 MHZ, CDCl₃) δ 8.13-8.06 (m, 2H), 8.06-8.01 (m, 2H), 8.00-7.95 (m, 2H), 7.80-7.77 (m, 2H), 7.66-7.59 (m, 1H), 7.59-7.51 (m, 1H), 7.54-7.47 (m, 3H), 7.46-7.35 (m, 5H), 7.28-7.21 (m, 2H), 6.06-6.03 (m, 1H), 5.96 (d, J=4.6 Hz, 1H), 5.96-5.84 (m, 2H), 5.72 (dddd, J=16.9, 9.9, 8.6, 5.6 Hz, 1H), 5.16-5.06 (m, 2H), 5.04-5.00 (m, 1H), 4.64 (dd, J=11.5, 7.3 Hz, 1H), 4.46 (dd, J=11.5, 5.5 Hz, 1H), 3.27 (dd, J=13.6, 8.6 Hz, 1H), 3.16 (dd, J=13.7, 5.7 Hz, 1H). ¹³C NMR (126 MHz, CDCl₃) δ 166.14, 165.71, 165.68, 165.55, 133.75, 133.61, 133.40, 133.36, 132.73, 130.11, 130.06, 129.86, 129.85, 129.63, 129.24, 129.17, 129.14, 128.79, 128.61, 128.60, 128.41, 118.45, 81.21, 69.32, 69.18, 68.81, 67.54, 62.76, 32.15. HRMS (ESI+, m/z): [M+H]⁺ calculated for C₃₇H₃₂O₉S, 653.1840; found 653.1832.

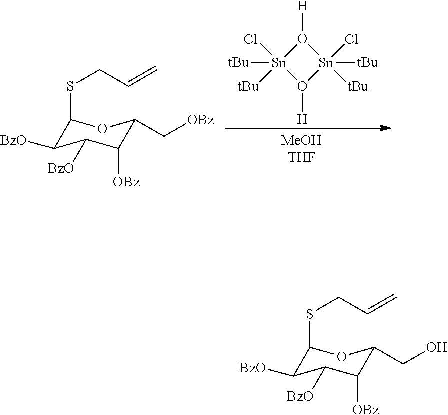

(2R,3R,4S,5S,6R)-2-(allylthio)-6-(hydroxymethyl)tetrahydro-2H-pyran-3,4,5-triyl tribenzoate: Dichlorotetrakis(1,1-dimethylethyl)di-μ-hydroxyditin (0.30 g, 0.521 mmol, 0.17 equiv.) was added to (2R,3R,4S,5S,6R)-2-(allylthio)-6-((benzoyloxy)methyl)tetrahydro-2H-pyran-3,4,5-triyl tribenzoate (2.00 g, 3.06 mmol, 1.0 equiv.) dissolved in a mixture of anhydrous MeOH (20 mL) and anhydrous THF (6 mL). The reaction mixture was stirred at 65° C. overnight (16 h) and concentrated under reduced pressure. Purification by flash-column chromatography (10-25% ethyl acetate in hexanes) afforded the product (1.37 g, 2.50 mmol, 82% yield) as a white foam. ¹H NMR (400 MHZ, CDCl₃) δ 8.16-8.09 (m, 2H), 8.05-7.97 (m, 2H), 7.87-7.79 (m, 2H), 7.67-7.61 (m, 1H), 7.57-7.35 (m, 6H), 7.30-7.21 (m, 2H), 5.99-5.84 (m, 4H), 5.77 (dddd, J=16.9, 9.9, 8.4, 5.8 Hz, 1H), 5.18 (dd, J=17.0, 1.4 Hz, 1H), 5.13 (dd, J=9.9, 1.5 Hz, 1H), 4.75 (td, J=6.6, 1.2 Hz, 1H), 3.86-3.77 (m, 1H), 3.75-3.66 (m, 1H), 3.29 (ddt, J=13.8, 8.5, 1.0 Hz, 1H), 3.19 (ddt, J=13.8, 5.9, 1.3 Hz, 1H). ¹³C NMR (126 MHz, CDCl₃) δ 166.81, 165.79, 165.49, 133.93, 133.62, 133.40, 133.12, 130.17, 130.06, 129.82, 129.16, 129.15, 129.02, 128.82, 128.62, 128.44, 118.27, 81.53, 69.91, 69.79, 69.39, 69.03, 60.93, 32.46. HRMS (ESI+, m/z): [M+H]⁺ calculated for C₃₀H₂₈O₈S, 549.1578; found 549.1570.

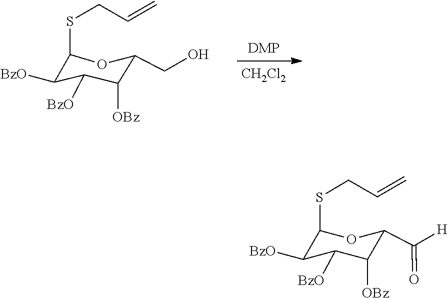

(2R,3R,4S,5R,6S)-2-(allylthio)-6-formyltetrahydro-2H-pyran-3,4,5-triyl tribenzoate: To a solution of (2R,3R,4S,5S,6R)-2-(allylthio)-6-(hydroxymethyl)tetrahydro-2H-pyran-3,4,5-triyl tribenzoate (1.37 g, 2.50 mmol, 1.00 equiv.) dissolved in dichloromethane (15 mL) was added Dess-Martin Periodinane (1.59 g, 3.75 mmol, 1.50 equiv.) and the resulting white suspension stirred for 1 hour. The reaction mixture was diluted with diethyl ether (60 mL) and treated with saturated aqueous sodium bicarbonate solution (22.5 mL) followed by aqueous sodium thiosulfate solution (50 wt %, 22.5 mL). The resulting bilayer mixture was stirred vigorously at 23° C. for 1 hour and the layers separated. The aqueous layer was extracted with diethyl ether (3×10 mL), and the combined organic extracts were washed sequentially with saturated aqueous sodium bicarbonate solution (22.5 mL) and brine (22.5 mL). The organic solution was dried over sodium sulfate, filtered, and concentrated to yield the product (1.37 g, 2.49 mmol, 100% yield) as a white foam, which was used for subsequent sulfinimine condensation without further purification.

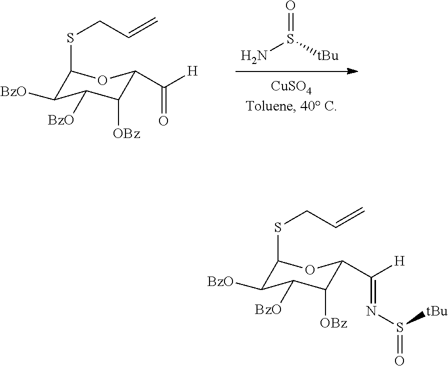

(2R,3R,4S,5S,6R)-2-(allylthio)-6-((E)-(((R)-tert-butylsulfinyl)imino)methyl)tetrahydro-2H-pyran-3,4,5-triyl tribenzoate: To a mixture of (2R,3R,4S,5R,6S)-2-(allylthio)-6-formyltetrahydro-2H-pyran-3,4,5-triyl tribenzoate (1.37 g, 2.49 mmol, 1.00 equiv.), (R)-(+)-2-methyl-2-propanesulfinamide (604 mg, 4.98 mmol, 2.00 equiv.) and anhydrous copper (II) sulfate (596 mg, 3.74 mmol, 1.50 equiv.) was added anhydrous toluene (8 mL). The resulting white suspension was heated to 40° C. and stirred for 16 hours, during which the suspension turned jade green. The reaction mixture was allowed to cool to ambience and filtered through celite with washing (3×15 mL of CH₂Cl₂). The combined organic extract was concentrated and purified by flash-column chromatography (10-50% ethyl acetate in hexanes) to yield the product (1.05 g, 1.62 mmol, 65% yield) as a white foam. ¹H NMR (400 MHZ, CDCl₃) δ 8.05 (d, J=2.1 Hz, 1H), 8.02-7.94 (m, 4H), 7.82-7.74 (m, 2H), 7.63-7.56 (m, 1H), 7.55-7.50 (m, 1H), 7.48-7.34 (m, 5H), 7.30-7.21 (m, 2H), 6.32 (dd, J=3.1, 1.5 Hz, 1H), 6.05 (d, J=5.5 Hz, 1H), 5.89 (dd, J=10.7, 5.5 Hz, 1H), 5.82 (dd, J=10.6, 3.1 Hz, 1H), 5.74 (dddd, J=16.8, 9.8, 8.5, 5.7 Hz, 1H), 5.51 (t, J=1.9 Hz, 1H), 5.22 (dd, J=17.0, 1.3 Hz, 1H), 5.17 (d, J=10.1 Hz, 1H), 3.28 (dd, J=13.8, 8.6 Hz, 1H), 3.20 (dd, J=13.8, 5.8 Hz, 1H), 0.99 (s, 9H). ¹³C NMR (126 MHZ, CDCl₃) 8 165.58, 165.51, 165.37, 133.66, 133.61, 133.32, 132.45, 129.99, 129.95, 129.80, 129.19, 129.03, 128.99, 128.66, 128.57, 128.34, 118.83, 81.81, 71.47, 69.49, 69.40, 68.34, 57.15, 32.46, 22.14. HRMS (ESI+, m/z): [M+H]⁺ calculated for C₃₄H₃₅NO₈S₂, 650.1877; found 650.1867.

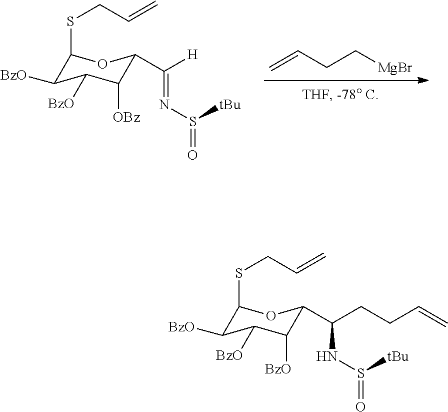

(2R,3R,4S,5S,6R)-2-(allylthio)-6-((R)-1-(((R)-tert-butylsulfinyl)amino)pent-4-en-1-yl)tetrahydro-2H-pyran-3,4,5-triyl tribenzoate: To a solution of (2R,3R,4S,5S,6R)-2-(allylthio)-6-((E)-(((R)-tert-butylsulfinyl)imino)methyl)tetrahydro-2H-pyran-3,4,5-triyl tribenzoate (0.200 g, 0.309 mmol, 1.00 equiv.) dissolved in THF (3 mL) at −78° C. was added butenyl magnesium bromide (Prepared according to: Wolleb, H.; Ogawa, S.; Schneider, M.; Shemet, A.; Muri, J.; Kopf, M.; Carreira, E. M. Org. Lett. 2018, 20, 10, 3014-3016)(0.22M in THF, 1.68 mL, 0.370 mmol, 1.20 equiv.) and stirred for 1 hour at −78° C. The mixture was diluted with ethyl acetate (10 mL) and quenched with saturated aqueous ammonium chloride (15 mL). The layers were separated and the aqueous layer further extracted with ethyl acetate (3×15 mL). The combined organic layers were then dried over sodium sulfate, filtered and concentrated in vacuo. Purification via flash-column chromatography (10-40% ethyl acetate in hexanes) afforded the product (0.188 g, 0.266 mmol, 86% yield) as a white foam. ¹H NMR (400 MHZ, CDCl₃) δ 8.12-8.04 (m, 2H), 8.01-7.93 (m, 2H), 7.79-7.72 (m, 2H), 7.67-7.58 (m, 1H), 7.56-7.45 (m, 3H), 7.45-7.34 (m, 3H), 7.22 (t, J=7.9 Hz, 2H), 6.09 (d, J=3.0 Hz, 1H), 5.95 (d, J=5.7 Hz, 1H), 5.85 (dd, J=10.7, 5.8 Hz, 1H), 5.82-5.66 (m, 3H), 5.20 (dd, J=17.0, 1.4 Hz, 1H), 5.17 (d, J=1.5 Hz, 1H), 5.04 (dq, J=17.1, 1.7 Hz, 1H), 4.97 (dq, J=10.1, 1.4 Hz, 1H), 4.70 (d, J=7.7 Hz, 1H), 3.78 (d, J=5.1 Hz, 1H), 3.58-3.47 (m, 1H), 3.26 (dd, J=13.7, 8.4 Hz, 1H), 3.20 (dd, J=13.7, 5.9 Hz, 1H), 2.35 (ddt, J=14.6, 10.4, 5.8 Hz, 1H), 2.26-2.10 (m, 1H), 2.06-1.83 (m, 2H), 1.19 (s, 9H). ¹³C NMR (101 MHZ, CDCl₃) δ 165.78, 165.74, 165.49, 137.73, 133.81, 133.59, 133.31, 132.67, 130.11, 130.04, 129.80, 129.27, 129.17, 129.17, 128.83, 128.60, 128.38, 118.69, 115.54, 81.68, 70.47, 69.88, 68.91, 68.72, 56.33, 54.75, 32.58, 30.51, 29.76, 22.74. HRMS (ESI+, m/z): [M+H]⁺ calculated for C₃₈H₄₃NO₈S₂, 706.2503; found 706.2500.

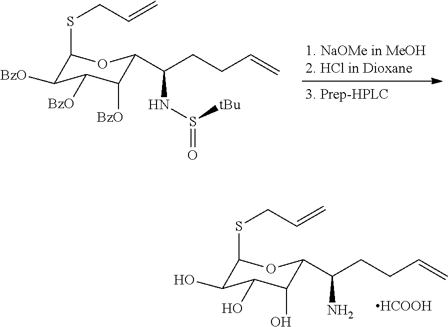

(R)-1-((2R,3R,4S,5R,6R)-6-(allylthio)-3,4,5-trihydroxytetrahydro-2H-pyran-2-yl)pent-4-en-1-aminium formate: NaOMe in MeOH (0.5 M, 4.0 mL, 2.0 mmol, 14.1 equiv.) was added to (2R,3R,4S,5S,6R)-2-(allylthio)-6-((R)-1-(((R)-tert-butylsulfinyl)amino)pent-4-en-1-yl)tetrahydro-2H-pyran-3,4,5-triyl tribenzoate (100 mg, 0.142 mmol, 1.00 equiv.) and the resulting solution stirred for 1 h. HCl in dioxane (4.0 M, 1.00 mL, 4.0 mmol, 28.2 equiv.) was then added to the mixture, and the resulting white suspension stirred for 1 h. The reaction mixture was concentrated in vacuo and purified via prep-HPLC(5-50% MeCN in H₂O with 0.1% HCOOH over 30 minutes) to yield the product (35 mg, 0.104 mmol, 73% yield over two steps) as a light-yellow film. ¹H NMR (400 MHZ, MeOD) δ 8.46 (br s, 1H), 5.93-5.78 (m, 2H), 5.43 (d, J=5.6 Hz, 1H), 5.24-5.03 (m, 4H), 4.30 (dd, J=4.8, 1.6 Hz, 1H), 4.22-4.06 (m, 2H), 3.63 (dd, J=10.1, 3.3 Hz, 1H), 3.43 (td, J=7.1, 4.7 Hz, 1H), 3.23 (dd, J=13.9, 8.1 Hz, 1H), 3.17 (dd, J=13.8, 6.3 Hz, 1H), 2.35-2.16 (m, J=6.8, 6.4 Hz, 2H), 2.00-1.80 (m, 2H). ¹³C NMR (101 MHZ, MeOD) δ 169.28, 137.74, 135.32, 117.73, 116.76, 86.74, 71.55, 70.47, 68.91, 68.70, 54.16, 33.36, 30.63, 29.50. HRMS (ESI+, m/z): [M+H]⁺ calculated for C₁₃H₂₃NO₄S, 290.1421; found 290.1423.

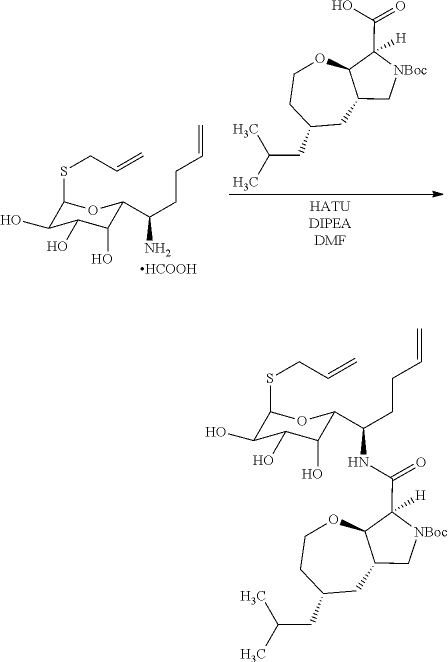 [SMILES: C=CCC[C@@H](N)C1OC(SCC=C)C(O)C(O)C1O ; CC(C)C[C@@H]1CCO[C@@H]2[C@@H](C1)CN(C(=O)OC(C)(C)C)[C@@H]2C(=O)O ; "·HCOOH" ; C=CCC[C@@H](NC(=O)[C@@H]1[C@@H]2OCC[C@@H](CC(C)C)C[C@H]2CN1C(=O)OC(C)(C)C)C1OC(SCC=C)C(O)C(O)C1O]

tert-butyl (4S,5aS,8S,8aR)-8-(((R)-1-((2R,3R,4S,5R,6R)-6-(allylthio)-3,4,5-trihydroxytetrahydro-2H-pyran-2-yl)pent-4-en-1-yl) carbamoyl)-4-isobutyloctahydro-7H-oxepino[2,3-c]pyrrole-7-carboxylate: To (R)-1-((2R,3R,4S,5R,6R)-6-(allylthio)-3,4,5-trihydroxytetrahydro-2H-pyran-2-yl)pent-4-en-1-aminium formate (35 mg, 0.104 mmol, 1.00 equiv.) dissolved in DMF (2.5 mL) was added (4S,5aS,8S,8aR)-7-(tert-butoxycarbonyl)-4-isobutyloctahydro-2H-oxepino[2,3-c]pyrrole-8-carboxylic acid (42.1 mg, 0.125 mmol, 1.20 equiv.), HATU (46.8 mg, 0.125 mmol, 1.20 equiv.) and DIPEA (108 μL, 0.624 mmol, 6.00 equiv.). The reaction mixture was stirred overnight (16 h) at room temperature, concentrated in vacuo, redissolved in ethyl acetate (25 mL) and washed with a 1:1 brine: saturated aqueous NaHCO₃ solution (25 mL). The organic extract was dried with sodium sulfate and concentrated to yield the product (41 mg, 0.0669 mmol, 64% yield) as a yellow oil, which was used in subsequent olefin metathesis without further purification.

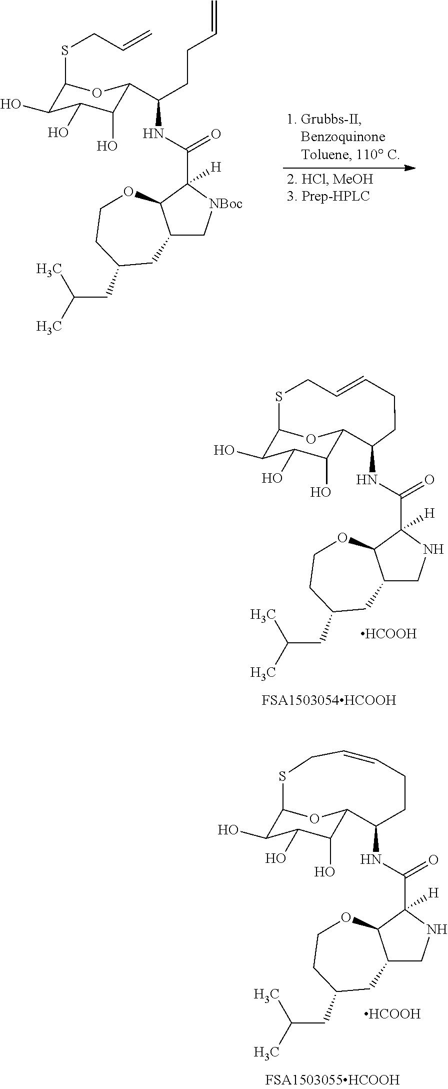 [SMILES: C=CCC[C@@H](NC(=O)[C@@H]1[C@@H]2OCC[C@@H](CC(C)C)C[C@H]2CN1C(=O)OC(C)(C)C)C1OC(SCC=C)C(O)C(O)C1O ; "2. HCl, MeOH3. Prep-HPLC" ; CC(C)C[C@@H]1CCO[C@@H]2[C@H](CN[C@@H]2C(=O)N[C@@H]2CC/C=C/CSC3OC2C(O)C(O)C3O)C1 ; "·HCOOH" ; CC(C)C[C@@H]1CCO[C@@H]2[C@H](CN[C@@H]2C(=O)N[C@@H]2CC/C=C\CSC3OC2C(O)C(O)C3O)C1 ; "·HCOOH"]

(4S,5aS,8S,8aR)-4-isobutyl-N-((1R,8R,9R,10R,11S,12R,E)-10,11,12-trihydroxy-13-oxa-2-thiabicyclo[7.3.1]tridec-4-en-8-yl) octahydro-2H-oxepino[2,3-c]pyrrole-8-carboxamide formate salt (FSA1503054·HCOOH) and (4S,5aS,8S,8aR)-4-isobutyl-N-((1R,8R,9R,10R,11S,12R,Z)-10,11,12-trihydroxy-13-oxa-2-thiabicyclo[7.3.1]tridec-4-en-8-yl) octahydro-2H-oxepino[2,3-c]pyrrole-8-carboxamide formate salt (FSA1503055·HCOOH): tert-butyl (4S,5aS,8S,8aR)-8-(((R)-1-((2R,3R,4S,5R,6R)-6-(allylthio)-3,4,5-trihydroxytetrahydro-2H-pyran-2-yl)pent-4-en-1-yl) carbamoyl)-4-isobutyloctahydro-7H-oxepino[2,3-c]pyrrole-7-carboxylate (41 mg, 0.0669 mmol, 1.00 equiv.) was dissolved in toluene (330 mL) and the solution was heated to reflux. Freshly sublimed benzoquinone (2.9 mg, 0.026 mmol, 0.40 equiv.) and Grubbs II catalyst (11.3 mg, 0.013 mmol, 0.20 equiv.) dissolved in toluene (5 mL) were added to the reaction mixture. After 20 minutes of stirring at reflux, the reaction mixture was quenched with DMSO (0.236 mL, 3.35 mmol, 50 equiv.) and allowed to cool to room temperature. The reaction mixture was concentrated in vacuo, redissolved in ethyl acetate (10 mL) and brine (10 mL), and the layers were separated. The aqueous layer was washed with ethyl acetate (3× 10 mL) and the combined organic extract was dried with sodium sulfate and concentrated. After concentration under reduced pressure, 1M HCl in MeOH (1 mL, 1.00 mmol, 25.6 equiv.) was added and the reaction mixture was stirred for 1 h. Concentration under reduced pressure, followed by purification via prep-HPLC(5-50% MeCN in H₂O with 0.1% HCOOH over 30 minutes) afforded product FSA1503054·HCOOH as the minor product (0.731 mg, 0.00138 mmol, 1.3% yield over three steps) and FSA1503055·HCOOH (0.932 mg, 0.00176 mmol, 1.7% yield over three steps) as the major product, both as thin, transparent films. In addition, a mixed fraction (0.666 mg, 0.00126 mmol, 1.2% yield over three steps) was also obtained and used in subsequent olefin reduction.

FSA1503054·HCOOH: ¹H NMR (400 MHZ, MeOD) δ 8.44 (br s, 1H), 8 5.78 (d, J=5.3 Hz, 1H), 5.54-5.32 (m, 2H), 4.69-4.55 (m, 2H), 4.32 (d, J=5.3 Hz, 2H), 4.20 (d, J=10.3 Hz, 1H), 4.05-3.90 (m, 2H), 3.82-3.72 (m, 2H), 3.56 (dd, J=11.2, 7.2 Hz, 1H), 3.45-3.34 (m, 1H), 3.16 (d, J=13.9 Hz, 1H), 2.85 (t, J=11.7 Hz, 1H), 2.33-2.15 (m, 2H), 2.08-1.96 (m, 2H), 1.89-1.60 (m, 6H), 1.26-1.10 (m, 2H), 1.01-0.91 (m, 1H), 0.89 (d, J=6.5 Hz, 6H). HRMS (ESI+, m/z): [M+H]⁺ calculated for C₂₄H₄₀N₂O₆S, 485.2680; found 485.2682.

FSA1503055·HCOOH: ¹H NMR (400 MHZ, MeOD) δ 8.41 (br s, 1H), 5.56 (br s, 2H), 5.46 (q, J=9.8, 9.3 Hz, 1H), 4.41-4.15 (m, 4H), 4.06-3.93 (m, 2H), 3.88 (br s, 1H), 3.86-3.75 (m, 1H), 3.70 (dd, J=9.0, 3.0 Hz, 1H), 3.56-3.50 (m, 1H), 3.29-3.15 (m, 2H), 2.84-2.76 (m, 1H), 2.35-2.19 (m, 1H), 2.16-1.95 (m, 3H), 1.81-1.57 (m, 5H), 1.36-1.27 (m, 1H), 1.26-1.10 (m, 2H), 1.02-0.91 (m, 1H), 0.90 (d, J=6.5 Hz, 6H). HRMS (ESI+, m/z): [M+H]⁺ calculated for C₂₄H₄₀N₂O₆S, 485.2680; found 485.2684.

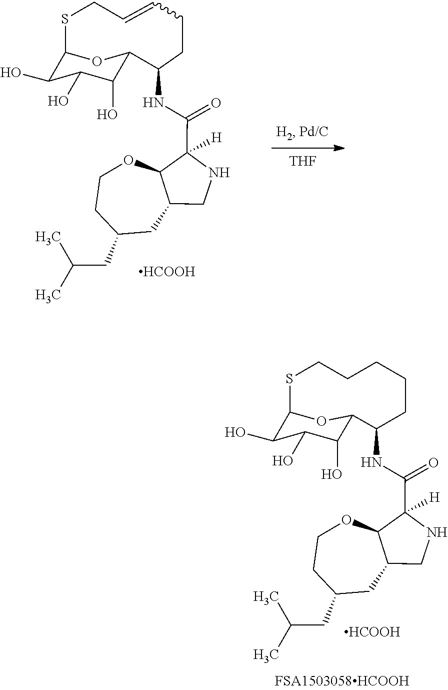 [SMILES: CC(C)C[C@@H]1CCO[C@@H]2[C@H](CN[C@@H]2C(=O)N[C@@H]2CCC=CCSC3OC2C(O)C(O)C3O)C1 ; "·HCOOH" ; CC(C)C[C@@H]1CCO[C@@H]2[C@H](CN[C@@H]2C(=O)N[C@@H]2CCCCCSC3OC2C(O)C(O)C3O)C1 ; "·HCOOH" ; "THF"]

(4S,5aS,8S,8aR)-4-isobutyl-N-((1R,8R,9R,10R,11S,12R)-10,11,12-trihydroxy-13-oxa-2-thiabicyclo[7.3.1]tridecan-8-yl) octahydro-2H-oxepino[2,3-c]pyrrole-8-carboxamide formate salt (FSA1503058·HCOOH): To a mixture of FSA1503054·HCOOH and FSA1503055. HCOOH (0.666 mg, 1.26 μmol, 1.00 equiv.) was added THF (1 mL) and sparged with argon with stirring. While sparging, 10 wt % Pd/C(1 mg, 0.94 μmol, 0.75 equiv.) was added to the mixture. The sparge needle was removed and replaced with a needle connected to a hydrogen balloon. The solution was sparged with hydrogen gas for 5 minutes, then the needle was lifted above the solution and the solution stirred for 5 hours. Pd/C was filtered off using a syringe filter and the filtrate concentrated in vacuo. Purification by prep-HPLC(5-50% MeCN in H₂O with 0.1% HCOOH over 30 minutes) afforded product FSA1503058·HCOOH (0.577 mg, 1.08 μmol, 86% yield) as a thin, transparent film. ¹H NMR (400 MHZ, MeOD) δ 8.49 (br s, 1H), 5.52 (d, J=6.0 Hz, 1H), 4.62-4.42 (m, 2H), 4.28 (t, J=9.2 Hz, 1H), 4.20 (d, J=8.9 Hz, 1H), 4.02 (dd, J=10.1, 5.9 Hz, 1H), 3.97 (dt, J=12.7, 3.6 Hz, 1H), 3.80 (d, J=3.2 Hz, 1H), 3.79-3.73 (m, 1H), 3.55 (dd, J=10.1, 3.3 Hz, 1H), 3.52-3.47 (m, 1H), 3.40-3.32 (m, 1H), 3.19-3.11 (m, 1H), 2.76 (t, J=11.5 Hz, 1H), 2.46 (td, J=13.7, 4.5 Hz, 1H), 2.24 (dtd, J=11.7, 9.8, 8.5, 2.9 Hz, 1H), 2.17-2.05 (m, 2H), 2.03-1.90 (m, 2H), 1.80-1.51 (m, 6H), 1.48-1.33 (m, 2H), 1.25-1.10 (m, 2H), 0.98-0.91 (m, 1H), 0.89 (d, J=6.6 Hz, 6H). HRMS (ESI+, m/z): [M+H]⁺ calculated for C₂₄H₄₂N₂O₆S, 487.2836; found 487.2841.

### Synthesis of FSA1504066A, FSA1504066B, and FSA1504068

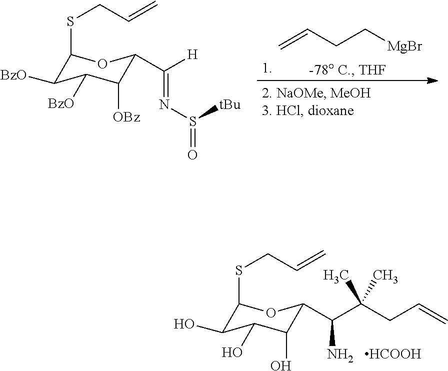

(R)-1-((2R,3R,4S,5R,6R)-6-(allylthio)-3,4,5-trihydroxytetrahydro-2H-pyran-2-yl)-2,2-dimethylpent-4-en-1-aminium formate: A solution of (2R,3R,4S,5S,6R)-2-(allylthio)-6-((E)-(((R)-tert-butylsulfinyl)imino)methyl)tetrahydro-2H-pyran-3,4,5-triyl tribenzoate (0.100 g, 0.154 mmol, 1.00 equiv.) dissolved in THF (1 mL) at −78° C. was added to (2-methylpent-4-en-2-yl) lithium (Prepared according to: Mudryk, B.; Cohen, T. J. Am. Chem. Soc. 1993, 115, 3855-3865.)(0.15M in THF, 3.08 mL, 0.462 mmol, 3.00 equiv.) at −78° C. The reaction was allowed to warm to 0° C. and stirred for 2 hours. The solution was allowed to warm to room temperature and 0.5 M NaOMe in MeOH (2 mL, 1.00 mmol, 6.49 equiv.) was added. After stirring for 1 h, 4 M HCl in 1,4-dioxane (0.5 mL, 2.0 mmol, 12.99 equiv.) was added and the resulting white suspension stirred for 1 h. The reaction mixture was concentrated under reduced pressure and purified via prep-HPLC(5-50% MeCN in H₂O with 0.1% HCOOH over 30 minutes) to yield the product (16 mg, 0.044 mmol, 29% yield over three steps) as a yellow oil. ¹H NMR (400 MHZ, MeOD) δ 8.52 (br s, 1H), 5.97-5.77 (m, 2H), 5.42 (d, J=5.6 Hz, 1H), 5.30-5.09 (m, 4H), 4.47 (dd, J=3.9, 1.5 Hz, 1H), 4.25 (dd, J=3.3, 1.6 Hz, 1H), 4.13 (dd, J=10.1, 5.6 Hz, 1H), 3.64 (dd, J=10.1, 3.3 Hz, 1H), 3.24 (d, J=3.8 Hz, 1H), 3.23-3.11 (m, 2H), 2.31-2.17 (m, 2H), 1.15 (s, 3H), 1.14 (s, 3H). ¹³C NMR (101 MHz, MeOD) δ 169.95, 135.35, 134.28, 119.90, 117.78, 87.04, 72.17, 71.52, 68.74, 67.87, 62.77, 45.39, 36.51, 33.63, 24.85, 23.44. HRMS (ESI+, m/z): [M+H]⁺ calculated for C₁₅H₂₇NO₄S, 318.1734; found 318.1738.

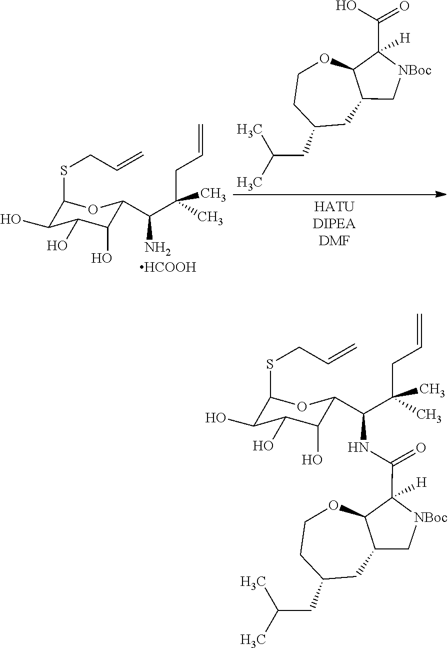

tert-butyl (4S,5aS,8S,8aR)-8-(((R)-1-((2R,3R,4S,5R,6R)-6-(allylthio)-3,4,5-trihydroxytetrahydro-2H-pyran-2-yl)-2,2-dimethylpent-4-en-1-yl) carbamoyl)-4-isobutyloctahydro-7H-oxepino[2,3-c]pyrrole-7-carboxylate: To (R)-1-((2R,3R,4S,5R,6R)-6-(allylthio)-3,4,5-trihydroxytetrahydro-2H-pyran-2-yl)-2,2-dimethylpent-4-en-1-aminium formate (16 mg, 0.044 mmol, 1.00 equiv.) dissolved in DMF (1.0 mL) was added (4S,5aS,8S,8aR)-7-(tert-butoxycarbonyl)-4-isobutyloctahydro-2H-oxepino[2,3-c]pyrrole-8-carboxylic acid (17.8 mg, 0.053 mmol, 1.20 equiv.), HATU (19.8 mg, 0.053 mmol, 1.20 equiv.) and DIPEA (45.7 μL, 0.264 mmol, 6.00 equiv.). The reaction mixture was stirred overnight (16 h) at room temperature, concentrated in vacuo, redissolved in ethyl acetate (10 mL) and washed with a 1:1 brine: saturated aqueous NaHCO₃ solution (10 mL). The organic extract was dried with sodium sulfate and concentrated to yield the product (25 mg, 0.039 mmol, 89% yield) as a yellow oil, which was used in subsequent olefin metathesis without further purification.

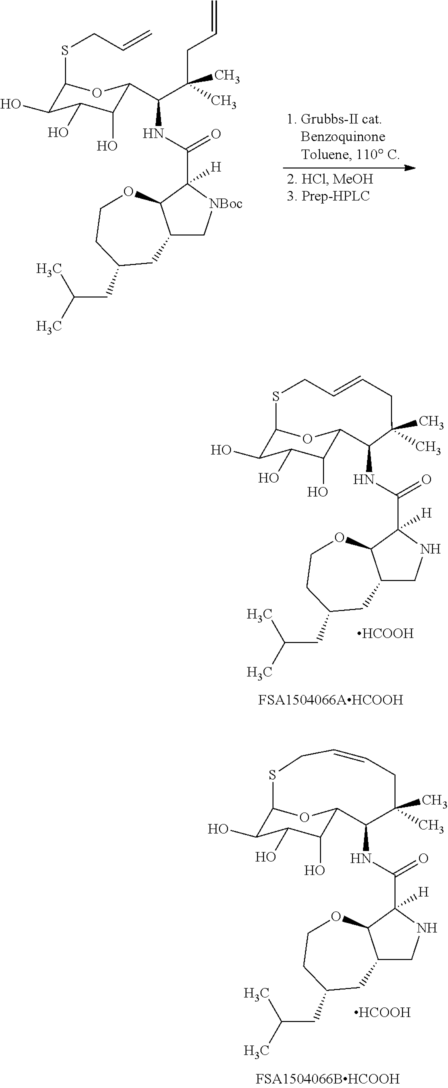

(4S,5aS,8S,8aR)-4-isobutyl-N-((1R,8R,9R,10R,11S,12R,E)-10,11,12-trihydroxy-7,7-dimethyl-13-oxa-2-thiabicyclo[7.3.1]tridec-4-en-8-yl) octahydro-2H-oxepino[2,3-c]pyrrole-8-carboxamide formate salt (FSA1504066A·HCOOH) and (4S,5aS,8S,8aR)-4-isobutyl-N-((1R,8R,9R,10R,11S,12R,Z)-10,11,12-trihydroxy-7,7-dimethyl-13-oxa-2-thiabicyclo[7.3.1]tridec-4-en-8-yl) octahydro-2H-oxepino[2,3-c]pyrrole-8-carboxamide formate salt (FSA1504066B·HCOOH): tert-butyl (4S,5aS,8S,8aR)-8-(((R)-1-((2R,3R,4S,5R,6R)-6-(allylthio)-3,4,5-trihydroxytetrahydro-2H-pyran-2-yl)-2,2-dimethylpent-4-en-1-yl) carbamoyl)-4-isobutyloctahydro-7H-oxepino[2,3-c]pyrrole-7-carboxylate (25 mg, 0.039 mmol, 1.00 equiv.) was dissolved in toluene (190 mL) and the solution was heated to reflux. Freshly sublimed benzoquinone (1.7 mg, 0.015 mmol, 0.40 equiv.) and Grubbs II catalyst (6.6 mg, 0.0078 mmol, 0.20 equiv.) dissolved in toluene (5 mL) were added to the reaction mixture. After 20 minutes of stirring at reflux, the reaction mixture was quenched with DMSO (0.275 mL, 3.9 mmol, 100 equiv.) and allowed to cool to room temperature. The reaction mixture was concentrated in vacuo, redissolved in ethyl acetate (5 mL) and brine (5 mL), and the layers were separated. The aqueous layer was washed with ethyl acetate (3×5 mL) and the combined organic extract was dried with sodium sulfate and concentrated. After concentration under reduced pressure, 1 M HCl in MeOH (1 mL, 1.00 mmol, 25.6 equiv.) was added and the reaction mixture was stirred for 1 h. Concentration under reduced pressure, followed by purification via prep-HPLC(5-50% MeCN in H₂O with 0.1% HCOOH over 30 minutes) afforded product

FSA1504066A·HCOOH as the major, first-eluting product (1.911 mg, 0.00342 mmol, 8% yield over three steps) and FSA1504066B·HCOOH (1.492 mg, 0.00267 mmol, 6% yield over three steps) as the minor, second-eluting product, both as thin, transparent films. In addition, a mixed fraction (1.262 mg, 0.00226 mmol, 5% yield over three steps) was also obtained and used in subsequent olefin reduction.

FSA1504066A·HCOOH: ¹H NMR (400 MHZ, MeOD) δ 8.42 (br s, 1H), 5.80 (d, J=5.3 Hz, 1H), 5.72 (t, J=12.3 Hz, 1H), 5.58 (t, J=12.4 Hz, 1H), 4.43-4.24 (m, 3H), 4.22 (d, J=9.6 Hz, 1H), 4.02-3.90 (m, 2H), 3.78 (td, J=11.2, 10.7, 3.5 Hz, 1H), 3.73-3.63 (m, 2H), 3.58 (dd, J=11.3, 7.6 Hz, 1H), 3.42 (t, J=12.1 Hz, 1H), 3.20-3.12 (m, 1H), 2.88 (t, J=11.7 Hz, 1H), 2.35-2.20 (m, 2H), 2.00 (d, J=13.3 Hz, 1H), 1.79-1.58 (m, 5H), 1.22 (s, 3H), 1.23-1.11 (m, 2H), 1.00-0.92 (m, 1H) 0.97 (s, 3H), 0.89 (d, J=6.6 Hz, 6H). 13C NMR (101 MHz, MeOD) δ 168.77, 167.97, 132.57, 126.77, 89.91, 81.31, 72.96, 72.20, 71.16, 70.15, 69.01, 62.47, 57.66, 49.28, 48.10, 43.63, 40.31, 37.33, 36.51, 35.99, 35.72, 32.58, 26.51, 26.29, 23.17, 22.92. HRMS (ESI+, m/z): [M+H]⁺ calculated for C₂₆H₄₄N₂O₆S, 513.2993; found 513.2992.

FSA1504066B·HCOOH was found to exist as a mixture of two equilibrating atropisomers by NMR, for which the coalescence temperature was close to ambience. This resulted in significant broadening and/or poor visibility of peaks belonging to the aminosugar moiety in both the 1H and 13C spectra, which can be resolved by VT-NMR at −60° C. For ease of comparison, the peaks corresponding to the spectra obtained at 23° C. are presented. ¹H NMR (400 MHZ, MeOD) δ 8.31 (br s, 1H), 5.77 (br s, 1H), 5.53 (pent, J=9.3 Hz, 1H), 4.46-4.26 (m, 3H), 4.15 (br s, 1H), 4.00 (dt, J=12.2, 4.1 Hz, 1H), 3.96 (br s, 1H), 3.87-3.79 (m, 1H), 3.76 (br s, 1H), 3.64 (dd, J=8.6, 2.8 Hz, 1H), 3.59 (dd, J=11.4, 7.7 Hz, 1H), 2.88 (t, J=11.7 Hz, 1H), 2.30-2.15 (m, 1H), 2.00 (d, J=13.5 Hz, 1H), 1.84-1.59 (m, 4H), 1.29-1.16 (m, 2H), 1.16 (s, 3H), 1.06 (s, 3H), 1.03-0.90 (m, 1H), 0.89 (d, J=6.6 Hz, 6H). ¹³C NMR (101 MHZ, MeOD) δ 167.42, 81.18, 71.03, 69.19, 62.00, 49.54, 48.11, 43.45, 38.82, 37.41, 36.16, 36.02, 26.29, 25.67, 23.15, 22.93. HRMS (ESI+, m/z): [M+H]⁺ calculated for C₂₆H₄₄N₂O₆S, 513.2993; found 513.2991.

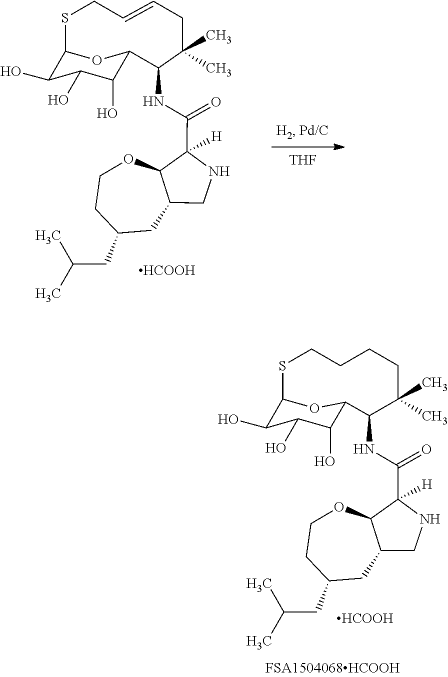

(4S,5aS,8S,8aR)-4-isobutyl-N-((1R,8R,9R,10R,11S,12R)-10,11,12-trihydroxy-7,7-dimethyl-13-oxa-2-thiabicyclo[7.3.1]tridecan-8-yl) octahydro-2H-oxepino[2,3-c]pyrrole-8-carboxamide formate salt: To a mixture of FSA1504066A·HCOOH and FSA1504066B·HCOOH (1.262 mg, 2.26 μmol, 1.00 equiv.) was added THF (1 mL) and sparged with argon with stirring. While sparging, 10 wt % Pd/C(1 mg, 0.94 μmol, 0.42 equiv.) was added to the mixture. The sparge needle was removed and replaced with a needle connected to a hydrogen balloon. The solution was sparged with hydrogen gas for 5 minutes, then the needle was lifted above the solution and the solution stirred for 5 hours. Pd/C was filtered off using a syringe filter and the filtrate concentrated in vacuo. Purification by prep-HPLC(5-50% MeCN in H₂O with 0.1% HCOOH over 30 minutes) afforded product FSA1504068·HCOOH (0.894 mg, 1.59 μmol, 71% yield) as a thin, transparent film. ¹H NMR (400 MHZ, MeOD) δ 8.42 (br s, 1H), 5.39 (d, J=6.2 Hz, 1H), 4.51 (d, J=8.3 Hz, 1H), 4.36-4.18 (m, 2H), 4.06 (dd, J=10.2, 6.2 Hz, 1H), 4.04-4.00 (m, 1H), 3.98 (d, J=11.6 Hz, 1H), 3.87-3.75 (m, 2H), 3.56 (dd, J=10.1, 7.2 Hz, 1H), 3.51 (dd, J=10.3, 3.0 Hz, 1H), 2.94-2.62 (m, 3H), 2.25 (s, 1H), 1.97 (s, 2H), 1.84-1.61 (m, 6H), 1.54 (s, 1H), 1.49-1.30 (m, 2H), 1.24-1.11 (m, 2H), 0.99 (s, 3H), 0.98-0.86 (m, 1H), 0.93 (s, 3H), 0.89 (d, J=6.6 Hz, 6H). HRMS (ESI+, m/z): [M+H]⁺ calculated for C₂₆H₄₆N₂O₆S, 515.3149; found 515.3156.

### Synthesis of FSA1503085

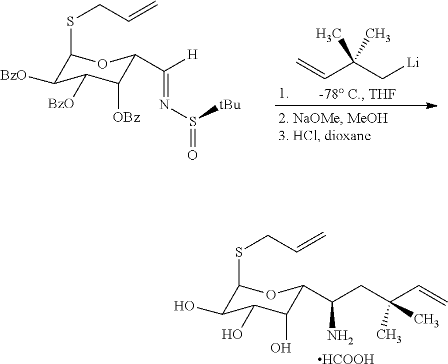

(R)-1-((2R,3R,4S,5R,6R)-6-(allylthio)-3,4,5-trihydroxytetrahydro-2H-pyran-2-yl)-3,3-dimethylpent-4-en-1-aminium formate: A solution of (2R,3R,4S,5S,6R)-2-(allylthio)-6-((E)-(((R)-tert-butylsulfinyl)imino)methyl)tetrahydro-2H-pyran-3,4,5-triyl tribenzoate (0.100 g, 0.154 mmol, 1.00 equiv.) dissolved in THF (1 mL) at −78° C. was added to (2,2-dimethylbut-3-en-1-yl)lithium⁷ (0.15 M in THF, 3.08 mL, 0.462 mmol, 3.00 equiv.) at −78° C. The reaction was allowed to warm to 0° C. and stirred for 2 hours. The solution was allowed to warm to room temperature and 0.5 M NaOMe in MeOH (2 mL, 1.00 mmol, 6.49 equiv.) was added. After stirring for 1 h, 4 M HCl in 1,4-dioxane (0.5 mL, 2.0 mmol, 12.99 equiv.) was added and the resulting white suspension stirred for 1 h. The reaction mixture was concentrated under reduced pressure and purified via prep-HPLC(5-50% MeCN in H₂O with 0.1% HCOOH over 30 minutes) to yield the product (12 mg, 0.033 mmol, 22% yield over three steps) as a yellow oil. ¹H NMR (400 MHZ, MeOD) δ 8.39 (s, 1H), 5.93-5.77 (m, 2H), 5.42 (d, J=5.5 Hz, 1H), 5.17 (dq, J=16.9, 1.5 Hz, 1H), 5.13-5.05 (m, 3H), 4.27 (dd, J=4.5, 1.5 Hz, 1H), 4.20 (dd, J=3.4, 1.5 Hz, 1H), 4.12 (dd, J=10.1, 5.5 Hz, 1H), 3.58 (dd, J=10.2, 3.3 Hz, 1H), 3.36 (dt, J=7.1, 4.7 Hz, 1H), 3.22 (dd, J=13.9, 8.0 Hz, 1H), 3.16 (dd, J=14.0, 6.5 Hz, 1H), 1.92 (dd, J=14.8, 7.0 Hz, 1H), 1.72 (dd, J=14.8, 4.9 Hz, 1H), 1.14 (s, 3H), 1.11 (s, 3H). ¹³C NMR (101 MHZ, MeOD) δ 168.35, 147.67, 135.50, 117.66, 113.40, 86.97, 71.51, 70.36, 69.27, 68.92, 52.73, 42.21, 37.25, 33.49, 28.18, 26.41. HRMS (ESI+, m/z): [M+H]⁺ calculated for C₁₅H₂₇NO₄S, 318.1734; found 318.1735.

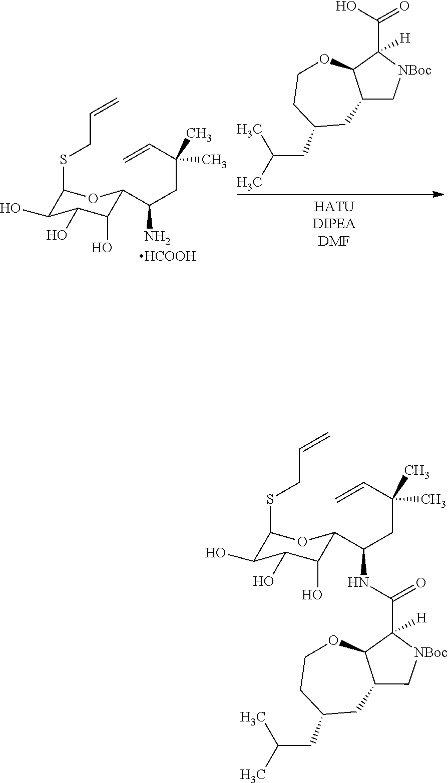

tert-butyl (4S,5aS,8S,8aR)-8-(((R)-1-((2R,3R,4S,5R,6R)-6-(allylthio)-3,4,5-trihydroxytetrahydro-2H-pyran-2-yl)-3,3-dimethylpent-4-en-1-yl) carbamoyl)-4-isobutyloctahydro-7H-oxepino[2,3-c]pyrrole-7-carboxylate: To (R)-1-((2R,3R,4S,5R,6R)-6-(allylthio)-3,4,5-trihydroxytetrahydro-2H-pyran-2-yl)-3,3-dimethylpent-4-en-1-aminium formate (12 mg, 0.033 mmol, 1.00 equiv.) dissolved in DMF (1.0 mL) was added (4S,5aS,8S,8aR)-7-(tert-butoxycarbonyl)-4-isobutyloctahydro-2H-oxepino[2,3-c]pyrrole-8-carboxylic acid (13.4 mg, 0.040 mmol, 1.20 equiv.), HATU (14.9 mg, 0.040 mmol, 1.20 equiv.) and DIPEA (34.3 μL, 0.198 mmol, 6.00 equiv.). The reaction mixture was stirred overnight (16 h) at room temperature, concentrated in vacuo, redissolved in ethyl acetate (10 mL) and washed with a 1:1 brine: saturated aqueous NaHCO₃ solution (10 mL). The organic extract was dried with sodium sulfate and concentrated to yield product (12 mg, 0.019 mmol, 57% yield) as a thin light-yellow film, which was used in subsequent olefin metathesis without further purification. HRMS (ESI+, m/z): [M+H]⁺ calculated for C₃₃H₅₆N₂O₈S, 641.3830; found 641.3829.

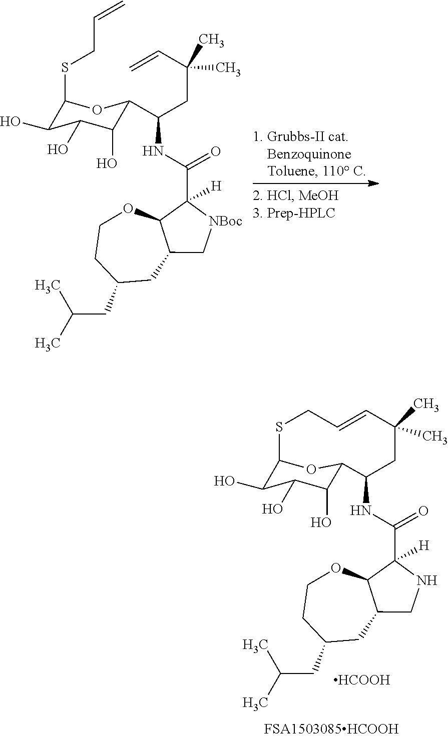

(4S,5aS,8S,8aR)-4-isobutyl-N-((1R,8R,9R,10R,11S,12R,E)-10,11,12-trihydroxy-6,6-dimethyl-13-oxa-2-thiabicyclo[7.3.1]tridec-4-en-8-yl) octahydro-2H-oxepino[2,3-c]pyrrole-8-carboxamide formate salt (FSA1503085·HCOOH): tert-butyl (4S,5aS,8S,8aR)-8-(((R)-1-((2R,3R,4S,5R,6R)-6-(allylthio)-3,4,5-trihydroxytetrahydro-2H-pyran-2-yl)-2,2-dimethylpent-4-en-1-yl) carbamoyl)-4-isobutyloctahydro-7H-oxepino[2,3-c]pyrrole-7-carboxylate (12 mg, 0.019 mmol, 1.00 equiv.) was dissolved in toluene (90 mL) and the solution was heated to reflux. Freshly sublimed benzoquinone (0.83 mg, 0.0076 mmol, 0.40 equiv.) and Grubbs II catalyst (3.2 mg, 0.0038 mmol, 0.20 equiv.) dissolved in toluene (5 mL) were added to the reaction mixture. After 20 minutes of stirring at reflux, the reaction mixture was quenched with DMSO (0.134 mL, 1.9 mmol, 100 equiv.) and allowed to cool to room temperature. The reaction mixture was concentrated in vacuo, redissolved in ethyl acetate (2.5 mL) and brine (2.5 mL), and the layers were separated. The aqueous layer was washed with ethyl acetate (3×2.5 mL) and the combined organic extract was dried with sodium sulfate and concentrated.

After concentration under reduced pressure, 1M HCl in MeOH (1 mL, 1.00 mmol, 52.6 equiv.) was added and the reaction mixture was stirred for 1 h. Concentration under reduced pressure, followed by purification via prep-HPLC(5-50% MeCN in H₂O with 0.1% HCOOH over 30 minutes) afforded product FSA1503085·HCOOH (0.633 mg, 0.00119 mmol, 6% yield over three steps) as a thin, transparent film. ¹H NMR (400 MHZ, MeOD) 8 8.50 (br s, 1H), 5.65 (br s, 1H), 5.58-5.33 (m, 2H), 4.43 (br s, 1H), 4.27 (t, J=9.2 Hz, 1H), 4.12 (d, J=9.0 Hz, 1H), 4.02-3.76 (m, 5H), 3.70 (s, 1H), 3.54-3.42 (m, 1H), 3.40-3.35 (m, 1H), 3.22 (dd, J=13.3, 3.9 Hz, 1H), 2.75 (t, J=11.4 Hz, 1H), 2.27 (dt, J=19.2, 10.8 Hz, 1H), 1.99 (d, J=13.4 Hz, 1H), 1.84-1.60 (m, 6H), 1.31-1.11 (m, 5H), 0.97 (s, 3H), 0.95-0.86 (m, 7H). HRMS (ESI+, m/z): [M+H]⁺ calculated for C₂₆H₄₄N₂O₆S, 513.2993; found 513.2995.

Preparation of compounds of formula:

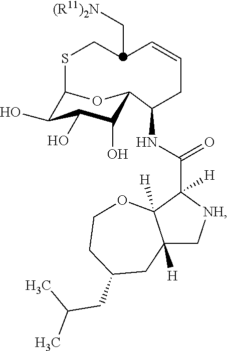

may be prepared by the following scheme:

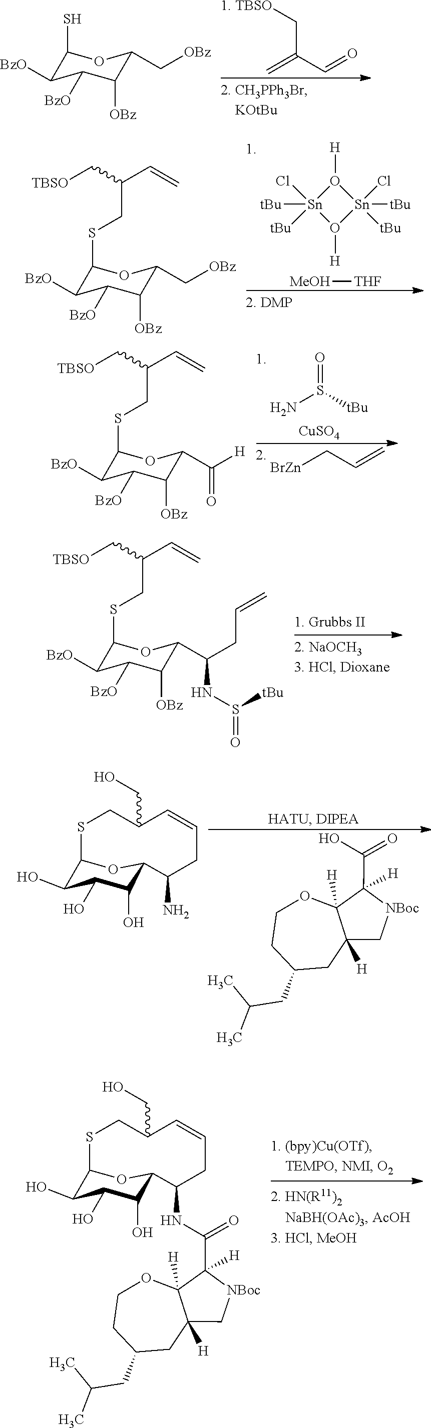

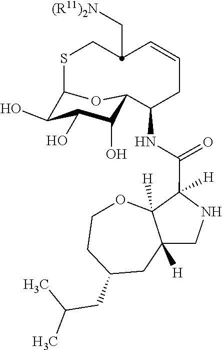

Preparation of compounds of formula:

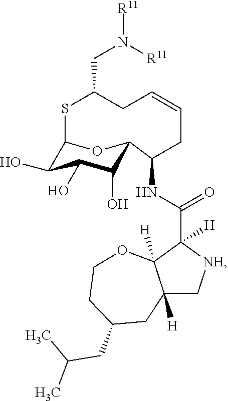

may be prepared by the following scheme:

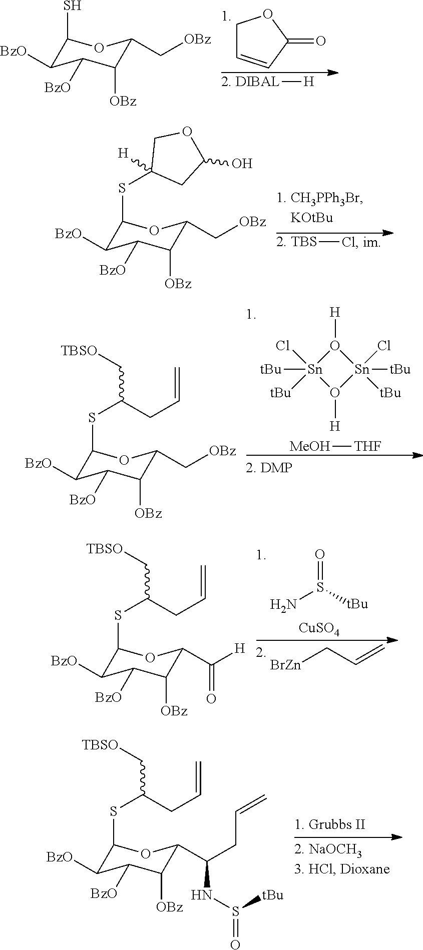

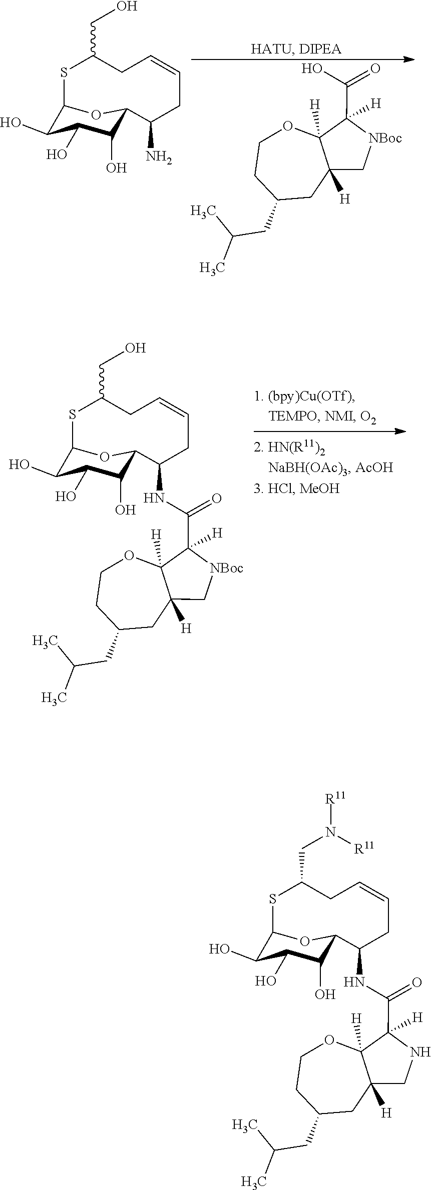

Additional exemplary compounds were prepared as described below.

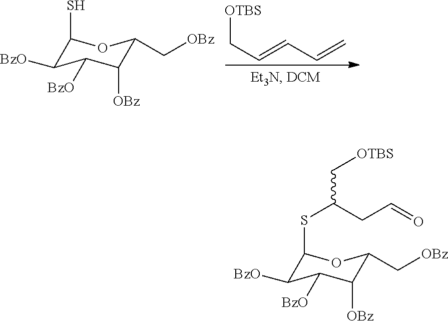

(2R,3S,4S,5R,6R)-2-((benzoyloxy)methyl)-6-((1-((tert-butyldimethylsilyl)oxy)-4-oxobutan-2-yl)thio)tetrahydro-2H-pyran-3,4,5-triyl tribenzoate (1:1 mixture of diastereomers)(A1): A 1000 mL 3-neck round bottom flask was charged with a solution of Et₃N(0.626 mL, 4.49 mmol) in CH₂Cl₂ (200 mL) and equipped with two addition funnels. A solution of (2R,3S,4S,5R,6R)-2-((benzoyloxy)methyl)-6-mercaptotetrahydro-2H-pyran-3,4,5-triyl tribenzoate (Doyle, L. M. et. al.; Org. Lett. 2017, 19, 5802-5805)(2.5 g, 4.08 mmol) in CH₂Cl₂ (200 mL) and (Z)-4-((tert-butyldimethylsilyl)oxy)but-2-enal (Ding, P. et al.; Org. Lett. 2004, 6, 11, 1805-1808)(0.818 g, 4.08 mmol) in CH₂Cl₂ (200 mL) were added dropwise simultaneously to the reaction mixture over 45 minutes at 23° C. After the addition was complete, the reaction mixture was stirred for an additional 60 min at 23° C. The reaction mixture was concentrated in vacuo and purified by flash chromatography (5-40% EtOAc in Hexanes) to afford product A1 (2.35 g, 4.08 mmol, 71% yield) as a white solid. ¹H NMR (400 MHZ, CDCl₃) δ 9.62 (s, 1H), 8.15-8.06 (m, 2H), 8.06-8.00 (m, 2H), 8.00-7.92 (m, 2H), 7.83-7.75 (m, 2H), 7.63 (t, J=7.4 Hz, 1H), 7.59-7.47 (m, 4H), 7.47-7.33 (m, 5H), 7.29-7.21 (m, 2H), 6.19 (d, J=5.4 Hz, 0.5H), 6.14 (d, J=4.6 Hz, 0.5H), 6.05 (dt, J=3.2, 1.6 Hz, 1H), 5.91-5.76 (m, 2H), 5.04 (t, J=6.2 Hz, 0.5H), 4.98 (t, J=6.4 Hz, 0.5H), 4.67 (dd, J=11.5, 7.0 Hz, 0.5H), 4.60 (dd, J=11.4, 6.7 Hz, 1H), 4.46 (td, J=10.7, 5.9 Hz, 1H), 3.82 (dd, J=10.2, 4.4 Hz, 0.5H), 3.75 (dd, J=10.1, 4.4 Hz, 0.5H), 3.66-3.53 (m, 1H), 3.53-3.38 (m, 1H), 2.92-2.75 (m, 1H), 2.69 (ddd, J=17.6, 6.5, 1.6 Hz, 0.5H), 2.62 (ddd, J=17.5, 7.3, 1.8 Hz, 0.5H), 0.85 (s, 4.5H), 0.80 (s, 4.5H), 0.01 (d, J=7.1 Hz, 3H), −0.05 (d, J=3.4 Hz, 3H). HRMS (ESI+, m/z): Calc'd for[C₄₄H₄₈O₁₁SSi+NH₄]⁺: 830.3025; found: 830.3019.

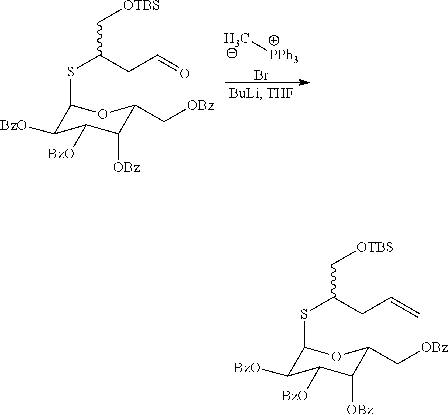

(2R,3S,4S,5R,6R)-2-((benzoyloxy)methyl)-6-((1-((tert-butyldimethylsilyl)oxy)pent-4-en-2-yl)thio)tetrahydro-2H-pyran-3,4,5-triyl tribenzoate (A2, 1:1 mixture of diastereomers): To a suspension of methyltriphenylphosphonium bromide (0.395 g, 1.107 mmol) in THF (18.45 mL) at 0° C., was added BuLi (0.410 ml, 2.7 M in hexanes, 1.107 mmol) dropwise. The resulting suspension was stirred for 30 min until the reaction became a canary-yellow solution, indicating the formation of methylene triphenylphosphorane. This solution was added dropwise via cannula into a solution of (2R,3S,4S,5R,6R)-2-((benzoyloxy)methyl)-6-((1-((tert-butyldimethylsilyl)oxy)-4-oxobutan-2-yl)thio)tetrahydro-2H-pyran-3,4,5-triyl tribenzoate (0.75 g, 0.923 mmol) in THF (10 mL) which was cooled to −78° C. The reaction was maintained at this temperature for an additional five minutes and then the reaction was diluted with ethyl ether (40 mL) and stirred at −78° C. for an additional five minutes. The reaction mixture was filtered through a pad of celite, concentrated in vacuo, and purified by flash chromatography (5-40% EtOAc in Hexanes) to afford product A2 (0.59 g, 0.74 mmol, 80% yield) as a white solid. ¹H NMR (400 MHZ, CDCl₃) δ 8.13-8.06 (m, 2H), 8.05-8.00 (m, 2H), 7.98 (ddd, J=8.5, 3.5, 1.3 Hz, 2H), 7.79 (ddd, J=8.4, 2.5, 1.3 Hz, 2H), 7.66-7.60 (m, 1H), 7.59-7.46 (m, 4H), 7.46-7.35 (m, 5H), 7.25 (td, J=7.9, 1.8 Hz, 2H), 6.11 (t, J=2.6 Hz, 0.5H), 6.08 (t, J=2.6 Hz, 0.5H), 6.06-6.02 (m, 1H), 5.90-5.87 (m, 1H), 5.87-5.83 (m, 1H), 5.83-5.72 (m, 0.5H), 5.72-5.61 (m, 0.5H), 5.12-4.98 (m, 2H), 4.99-4.87 (m, 1H), 4.61 (dd, J=7.0, 1.7 Hz, 0.5H), 4.58 (dd, J=7.0, 1.7 Hz, 0.5H), 4.43 (td, J=11.4, 5.8 Hz, 1H), 3.74 (dd, J=10.2, 4.9 Hz, 0.5H), 3.66 (dd, J=10.3, 5.2 Hz, 0.5H), 3.59 (app ddd, J=12.9, 10.3, 6.8 Hz, 1H), 3.00 (ddd, J=10.9, 6.4, 4.4 Hz, 0.5H), 2.97-2.91 (m, 0.5H), 2.58-2.43 (m, 1H), 2.37-2.20 (m, 1H), 0.87 (s, 4.5H), 0.81 (s, 4.5H), 0.03 (d, J=2.2 Hz, 3H), −0.04 (d, J=1.8 Hz, 3H). HRMS (ESI+, m/z): Calc'd for[C₄₅H₅₀O₁₀SSi+NH₄]⁺: 829.3232; found: 828.3222.

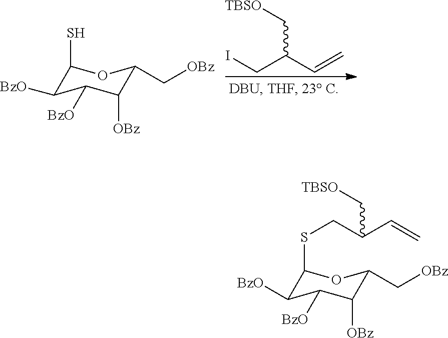

(2R,3S,4S,5R,6R)-2-((benzoyloxy)methyl)-6-((2-(((tert-butyldimethylsilyl)oxy)methyl) but-3-en-1-yl)thio)tetrahydro-2H-pyran-3,4,5-triyl tribenzoate A3 (1:1 mixture of diastereomers): To a solution of tert-butyl ((2-(iodomethyl) but-3-en-1-yl)oxy)dimethylsilane (1.57 g, 1.75 Eq, 4.80 mmol) in THF (3.4 mL) at 23° C., was added (2R,3S,4S,5R,6R)-2-((benzoyloxy)methyl)-6-mercaptotetrahydro-2H-pyran-3,4,5-triyl tribenzoate (2.80 g, 60% Wt, 1 Eq, 2.74 mmol) followed by DBU (417 mg, 0.41 mL, 1 Eq, 2.74 mmol). The reaction mixture was stirred for 5 min, then was concentrated in vacuo. The crude residue was purified by flash chromatography (0-50% EtOAc in Hexanes) to afford product A3 (1.1 g, 1.4 mmol, 49%). ¹H NMR (400 MHZ, CDCl₃) δ 8.12-8.05 (m, 2H), 8.02 (dt, J=8.4, 1.3 Hz, 2H), 7.98 (dt, J=8.3, 1.2 Hz, 2H), 7.82-7.76 (m, 2H), 7.66-7.58 (m, 1H), 7.58-7.45 (m, 5H), 7.45-7.35 (m, 6H), 7.28-7.21 (m, 4H), 6.06-5.98 (m, 2H), 5.91-5.82 (m, 2H), 5.73-5.56 (m, 1H), 5.09-4.93 (m, 3H), 4.60 (ddd, J=11.5, 7.2, 2.3 Hz, 1H), 4.44 (dd, J=11.4, 5.6 Hz, 1H), 3.57 (ddd, J=13.3, 9.9, 5.0 Hz, 1H), 3.45 (ddd, J=13.2, 9.9, 5.9 Hz, 1H), 2.86 (dd, J=12.7, 6.2 Hz, 0.5H), 2.73 (qd, J=13.0, 6.8 Hz, 1H), 2.57 (dd, J=12.6, 7.8 Hz, 0.5H), 2.47-2.34 (m, 1H), 0.84 (d, J=4.8 Hz, 9H), −0.05 (s, 6H). HRMS (ESI+, m/z): Calc'd for[C₄₅H₅₀O₁₀SSi+NH₄]⁺: 828.3238; found: 828.3238.

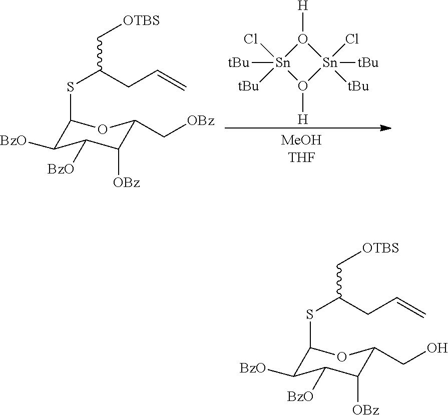

(2R,3R,4S,5S,6R)-2-((1-((tert-butyldimethylsilyl)oxy)pent-4-en-2-yl)thio)-6-(hydroxymethyl)tetrahydro-2H-pyran-3,4,5-triyl tribenzoate (3:2 mixture of diastereomers)(A4): (2R,3S,4S,5R,6R)-2-((benzoyloxy)methyl)-6-((1-((tert-butyldimethylsilyl)oxy)pent-4-en-2-yl)thio)tetrahydro-2H-pyran-3,4,5-triyl tribenzoate (940 mg, 1.159 mmol) was dissolved in MeOH (8.6 mL) and THF (2.9 mL). Dichlorotetrakis(1,1-dimethylethyl)di-μ-hydroxyditin (112 mg, 0.197 mmol) was added and the reaction was stirred at 66° C. for 2 days. The reaction was cooled to 23° C., concentrated in vacuo, and the crude residue was purified by flash chromatography (10-60% EtOAc in Hexanes) to afford product A4 (0.55 g, 0.78 mmol, 67% yield) and recovered starting material (120 mg, 12%). ¹H NMR (400 MHZ, CDCl₃) δ 8.16-8.06 (m, 2H), 8.02-7.94 (m, 2H), 7.81 (ddd, J=8.5, 2.1, 1.3 Hz, 2H), 7.68-7.59 (m, 1H), 7.57-7.48 (m, 3H), 7.46-7.35 (m, 3H), 7.28-7.20 (m, 2H), 6.06 (d, J=5.5 Hz, 0.4H), 6.02 (d, J=5.6 Hz, 0.6H), 5.93-5.67 (m, 4H), 5.17-5.05 (m, 1H), 5.02-4.89 (m, 1H), 4.76 (q, J=6.9 Hz, 1H), 3.83-3.71 (m, 2H), 3.71-3.56 (m, 2H), 2.92 (app dddd, J=12.7, 10.3, 7.5, 5.3 Hz, 1H), 2.61-2.50 (m, 1H), 2.27 (app dtd, J=14.8, 7.4, 4.5 Hz, 1H), 0.89 (s, 6H), 0.82 (s, 3H), 0.06 (d, J=4.0 Hz, 4H), −0.03 (d, J=4.8 Hz, 2H). HRMS (ESI+, m/z): Calc'd for[C₃₈H₄₆O₈SSi+Na]⁺: 729.2524; found: 729.2512.

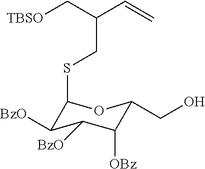

(2R,3R,4S,5S,6R)-2-((2-(((tert-butyldimethylsilyl)oxy)methyl) but-3-en-1-yl)thio)-6-(hydroxymethyl)tetrahydro-2H-pyran-3,4,5-triyl tribenzoate (A5)(1:1 mixture of diastereomers): Product A5 was prepared in a similar fashion to product A4 from (2R,3S,4S,5R,6R)-2-((benzoyloxy)methyl)-6-((2-(((tert-butyldimethylsilyl)oxy)methyl) but-3-en-1-yl)thio)tetrahydro-2H-pyran-3,4,5-triyl tribenzoate. ¹H NMR (400 MHZ, CDCl₃) δ 8.14-8.08 (m, 2H), 8.03-7.95 (m, 2H), 7.84-7.77 (m, 2H), 7.67-7.60 (m, 1H), 7.55-7.47 (m, 3H), 7.46-7.36 (m, 3H), 7.25 (t, J=7.8 Hz, 2H), 5.96 (t, J=5.2 Hz, 1H), 5.92-5.81 (m, 3H), 5.69 (dddd, J=17.1, 15.1, 10.5, 8.1 Hz, 1H), 5.15-4.98 (m, 2H), 4.72 (t, J=6.7 Hz, 1H), 3.85-3.71 (m, 1H), 3.64 (ddd, J=11.2, 10.0, 5.0 Hz, 2H), 3.56 (dd, J=10.0, 6.2 Hz, 0.5H), 3.49 (dd, J=9.9, 6.6 Hz, 0.5H), 2.85 (ddd, J=15.0, 12.9, 5.9 Hz, 1H), 2.66 (dd, J=13.0, 7.4 Hz, 0.5H), 2.58 (dd, J=12.8, 7.8 Hz, 0.5H), 2.53-2.40 (m, 2H), 0.87 (d, J=4.6 Hz, 9H), 0.07-−0.06 (m, 6H). HRMS (ESI+, m/z): Calc'd for[C₃₈H₄₆O₉SSi+H]⁺: 707.2705; found: 707.2699.

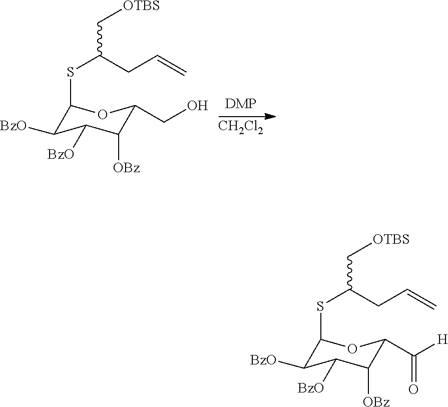

(2R,3R,4S,5R,6S)-2-((1-((tert-butyldimethylsilyl)oxy)pent-4-en-2-yl)thio)-6-formyltetrahydro-2H-pyran-3,4,5-triyl tribenzoate (A6, 3:2 mixture of diastereomers): To a solution of (2R,3R,4S,5S,6R)-2-((1-((tert-butyldimethylsilyl)oxy)pent-4-en-2-yl)thio)-6-(hydroxymethyl)tetrahydro-2H-pyran-3,4,5-triyl tribenzoate (550 mg, 1 Eq, 778 μmol) in CH₂Cl₂ (7.78 mL) was added Dess-Martin Periodinane (363 mg, 1.1 Eq, 856 μmol). The reaction was stirred for 1 hour. The reaction mixture was diluted with CH₂Cl₂ (10 mL) and quenched upon addition of sat. aq. NaHCO₃ (10 mL) and sat. aq. Na₂S₂O₃ (10 mL). The resulting biphasic mixture was stirred vigorously for 30 minutes, after which the biphasic mixture was separated. The aq. layer was extracted with CH₂Cl₂ (2×10 mL) and the combined organics were dried over sodium sulfate and concentrated in vacuo to afford the product A6 (0.55 g, 0.78 mmol, 99% yield). ¹H NMR (400 MHZ, CDCl₃) δ 9.65 (s, 1H), 8.03-7.94 (m, 4H), 7.83-7.75 (m, 2H), 7.63-7.55 (m, 1H), 7.56-7.49 (m, 1H), 7.49-7.42 (m, 3H), 7.42-7.36 (m, 2H), 7.30-7.22 (m, 2H), 6.28 (ddd, J=4.6, 3.0, 1.7 Hz, 1H), 6.21 (d, J=5.3 Hz, 0.4H), 6.19 (d, J=5.0 Hz, 0.6H), 5.89-5.66 (m, 3H), 5.21 (d, J=1.7 Hz, 0.4H), 5.18 (d, J=1.7 Hz, 0.6H), 5.11 (dd, J=17.1, 1.7 Hz, 0.4H), 5.06 (dd, J=10.1, 1.8 Hz, 0.4H), 5.00 (dd, J=17.1, 1.7 Hz, 0.6H), 4.97-4.93 (m, 0.6H), 3.76 (dd, J=10.3, 5.3 Hz, 0.6H), 3.70 (dd, J=10.3, 5.6 Hz, 0.4H), 3.66-3.60 (m, 1H), 2.97 (tt, J=7.1, 5.4 Hz, 1H), 2.62-2.47 (m, 1H), 2.35-2.20 (m, 1H), 0.89 (s, 6H), 0.83 (s, 3H), 0.05 (d, J=1.5 Hz, 4H), −0.02 (d, J=4.9 Hz, 2H). HRMS (ESI+, m/z): Calc'd for[C₃₈H₄₄O₉SSi+Na]⁺: 705.2548; found: 705.2539.

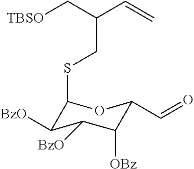

(2R,3R,4S,5R,6S)-2-((2-(((tert-butyldimethylsilyl)oxy)methyl) but-3-en-1-yl)thio)-6-formyltetrahydro-2H-pyran-3,4,5-triyl tribenzoate (A7 1:1 mixture of diastereomers): Product A7 was prepared in a similar fashion to product A6 from (2R,3R,4S,5S,6R)-2-((2-(((tert-butyldimethylsilyl)oxy)methyl) but-3-en-1-yl)thio)-6-(hydroxymethyl)tetrahydro-2H-pyran-3,4,5-triyl tribenzoate. ¹H NMR (400 MHZ, CDCl₃) δ 9.64 (s, 1H), 7.98 (ddt, J=8.5, 6.9, 1.7 Hz, 3H), 7.78 (dd, J=8.3, 1.4 Hz, 2H), 7.62-7.56 (m, 1H), 7.55-7.49 (m, 1H), 7.48-7.41 (m, 3H), 7.38 (t, J=7.8 Hz, 3H), 7.30-7.20 (m, 2H), 6.26 (dd, J=2.9, 1.5 Hz, 1H), 6.09 (t, J=4.3 Hz, 1H), 5.83-5.76 (m, 2H), 5.77-5.56 (m, 1H), 5.13 (t, J=1.5 Hz, 1H), 5.12-4.97 (m, 3H), 3.62 (ddd, J=9.9, 6.2, 4.8 Hz, 1H), 3.56-3.45 (m, 1H), 2.92 (dd, J=12.8, 5.5 Hz, 0.5H), 2.86 (dd, J=13.0, 6.1 Hz, 0.5H), 2.70 (dd, J=13.0, 7.6 Hz, 0.5H), 2.61 (dd, J=12.8, 8.1 Hz, 0.5H), 2.44 (d, J=6.6 Hz, 1H), 0.85 (d, J=7.1 Hz, 9H), 0.02-−0.05 (m, 6H). HRMS (ESI+, m/z): Calc'd for[C₃₈H₄₄O₉SSi+H]⁺: 705.2548; found: 705.2558.

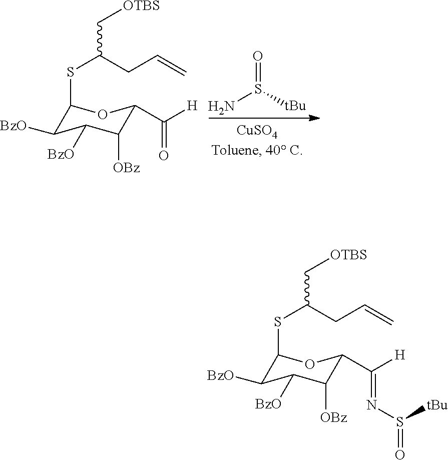

(2R,3R,4S,5S,6R)-2-((1-((tert-butyldimethylsilyl)oxy)pent-4-en-2-yl)thio)-6-((E)-(((R)-tert-butylsulfinyl)imino)methyl)tetrahydro-2H-pyran-3,4,5-triyl tribenzoate (A8, characterized as 9:1 mixture of diastereomers): To a solution of (2R,3R,4S,5R,6S)-2-((1-((tert-butyldimethylsilyl)oxy)pent-4-en-2-yl)thio)-6-formyltetrahydro-2H-pyran-3,4,5-triyl tribenzoate (540 mg, 1 Eq, 0.76 mmol) in PhCH₃ (0.6 mL) was added (R)-2-methylpropane-2-sulfinamide (186 mg, 2 Eq, 1.53 mmol) and CuSO₄ (183 mg, 1.5 Eq, 1.15 mmol). The reaction was stirred for 2 days at 40° C. The reaction was cooled to 23° C. and filtered through a celite pad. The celite pad was washed with CH₂Cl₂ (2×25 mL) and the combined organics were concentrated in vacuo. The crude residue was purified by flash chromatography (10-60% EtOAc in Hexanes) to afford product A8 (511 mg, 0.63 mmol, 82.5%). ¹H NMR (400 MHZ, CDCl₃) δ 8.03 (d, J=2.2 Hz, 1H), 7.98 (app ddd, J=8.4, 2.8, 1.3 Hz, 4H), 7.81-7.73 (m, 2H), 7.62-7.56 (m, 1H), 7.56-7.48 (m, 1H), 7.48-7.41 (m, 3H), 7.41-7.35 (m, 2H), 7.30-7.19 (m, 2H), 6.29 (dd, J=3.0, 1.5 Hz, 1H), 6.21 (d, J=5.2 Hz, 0.1H), 6.18 (d, J=5.1 Hz, 0.9H), 5.89-5.67 (m, 3H), 5.58 (t, J=1.9 Hz, 0.1H), 5.57 (t, J=1.9 Hz, 0.9H), 5.11 (dd, J=17.1, 1.7 Hz, 0.1H), 5.08-5.04 (m, 0.1H), 5.00 (dd, J=17.1, 1.8 Hz, 0.9H), 4.98-4.92 (m, 0.9H), 3.76 (dd, J=10.3, 5.2 Hz, 0.9H), 3.70 (dd, J=10.3, 5.5 Hz, 0.1H), 3.64 (dd, J=10.2, 6.9 Hz, 1H), 2.96 (app tt, J=7.1, 5.5 Hz, 1H), 2.62-2.48 (m, 1H), 2.39-2.25 (m, 1H), 0.99 (s, 0.9H) 0.98 (s, 8.1H), 0.89 (s, 8.1H), 0.83 (s, 0.9H), 0.05 (s, 5.4H), −0.02 (d, J=5.0 Hz, 0.6H). HRMS (ESI+, m/z): Calc'd for[C₃₂H₅₃NO₉S₂Si+H]+: 808.3004; found: 808.2986.

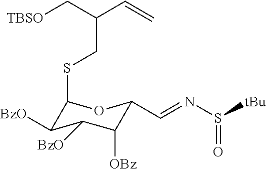

(2R,3R,4S,5S,6R)-2-((2-(((tert-butyldimethylsilyl)oxy)methyl) but-3-en-1-yl)thio)-6-((E)-(((R)-tert-butylsulfinyl)imino)methyl)tetrahydro-2H-pyran-3,4,5-triyl tribenzoate (A9 1:1 mixture of diastereomers): Product A9 was prepared in a similar fashion to product A8 from (2R,3R,4S,5R,6S)-2-((2-(((tert-butyldimethylsilyl)oxy)methyl) but-3-en-1-yl)thio)-6-formyltetrahydro-2H-pyran-3,4,5-triyl tribenzoate. ¹H NMR (400 MHZ, CDCl₃) δ 8.04 (dd, J=2.2, 1.2 Hz, 1H), 8.00-7.94 (m, 4H), 7.82-7.74 (m, 2H), 7.62-7.56 (m, 1H), 7.56-7.50 (m, 1H), 7.49-7.42 (m, 3H), 7.39 (t, J=7.8 Hz, 2H), 7.28-7.21 (m, 2H), 6.30 (dt, J=3.6, 1.9 Hz, 1H), 6.14-6.05 (m, 1H), 5.89-5.78 (m, 2H), 5.77-5.59 (m, 1H), 5.52 (q, J=1.9 Hz, 1H), 5.13-4.98 (m, 2H), 3.64 (td, J=9.7, 4.9 Hz, 1H), 3.53 (ddd, J=14.7, 10.0, 6.2 Hz, 1H), 2.91 (dd, J=12.8, 5.7 Hz, 0.5H), 2.84 (dd, J=13.1, 6.3 Hz, 0.5H), 2.70 (dd, J=13.1, 7.3 Hz, 0.5H), 2.61 (dd, J=12.8, 8.0 Hz, 0.5H), 2.45 (dh, J=13.0, 6.2 Hz, 1H), 0.98 (s, 9H), 0.87 (d, J=7.6 Hz, 9H), 0.10-−0.35 (m, 6H). HRMS (ESI+, m/z): Calc'd for[C₄₂H₅₃NO₉S₂Si+H]⁺: 808.3004; found: 808.3069.

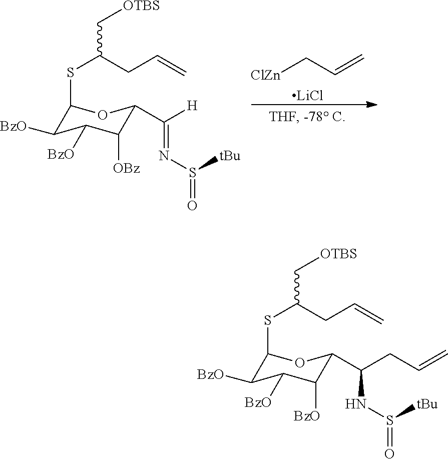

(2R,3R,4S,5S,6R)-2-((1-((tert-butyldimethylsilyl)oxy)pent-4-en-2-yl)thio)-6-((R)-1-(((R)-tert-butylsulfinyl)amino) but-3-en-1-yl)tetrahydro-2H-pyran-3,4,5-triyl tribenzoate (A10, 3:2 mixture of diastereomers): To a solution of (2R,3R,4S,5S,6R)-2-((1-((tert-butyldimethylsilyl)oxy)pent-4-en-2-yl)thio)-6-((E)-(((R)-tert-butylsulfinyl)imino)methyl)tetrahydro-2H-pyran-3,4,5-triyl tribenzoate (511 mg, 1 Eq, 632 μmol) in THF (6.3 mL) at −78° C. was added allylzinc lithium chloride (256 mg, 6.62 mL, 0.21 M solution in THF, 2.2 Eq, 1.39 mmol) dropwise over 15 minutes. Upon the complete addition of allylzinc, the reaction was quenched with sat. aq. NH₄Cl (10 mL). The reaction was warmed to 23° C. and was diluted with EtOAc (30 mL) and H₂O (10 mL). The layers of the resultant biphasic mixture were separated, and the aq. layer was extracted with EtOAc (2× 20 mL). The combined organics were dried over sodium sulfate and concentrated in vacuo. The crude residue was purified by flash chromatography (0-70% EtOAc in Hexanes) to afford product A10 (472 mg, 555 μmol, 87.8%) ¹H NMR (400 MHZ, CDCl₃) δ 8.08 (dd, J=8.2, 1.4 Hz, 2H), 8.01-7.92 (m, 2H), 7.80-7.68 (m, 2H), 7.68-7.58 (m, 1H), 7.58-7.46 (m, 3H), 7.46-7.33 (m, 3H), 7.24-7.18 (m, 2H), 6.13-6.08 (m, 1.4H), 6.07 (d, J=5.7 Hz, 0.6H), 5.95-5.62 (m, 4H), 5.27-5.07 (m, 2.8H), 4.97 (dd, J=17.0, 1.8 Hz, 0.6H), 4.92-4.86 (m, 0.6H), 4.75 (d, J=7.3 Hz, 0.4H), 4.69 (d, J=7.6 Hz, 0.6H), 3.83 (dd, J=10.1, 4.4 Hz, 0.6H), 3.78 (t, J=4.8 Hz, 1H), 3.67 (dd, J=10.3, 5.2 Hz, 0.4H), 3.62-3.54 (m, 2H), 3.00-2.88 (m, 1H), 2.67-2.48 (m, 3H), 2.35-2.21 (m, 1H), 1.11 (s, 6H), 1.09 (s, 3H), 0.89 (s, 6H), 0.82 (s, 3H), 0.07 (d, J=4.1 Hz, 4H), −0.03 (d, J=10.2 Hz, 2H). HRMS (ESI+, m/z): Calc'd for[C₄₅H₅₉NO₉S₂Si+H]⁺: 850.3473; found: 850.3465.

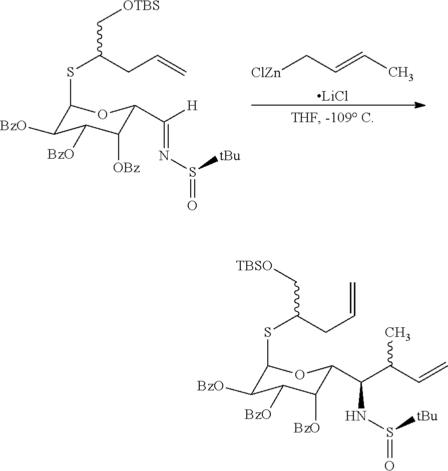

(2R,3R,4S,5S,6R)-2-((1-((tert-butyldimethylsilyl)oxy)pent-4-en-2-yl)thio)-6-((1R)-1-(((R)-tert-butylsulfinyl)amino)-2-methylbut-3-en-1-yl)tetrahydro-2H-pyran-3,4,5-triyl tribenzoate (A11 6:3:4:2 mixture of diastereomers): To a solution of (2R,3R,4S,5S,6R)-2-((1-((tert-butyldimethylsilyl)oxy)pent-4-en-2-yl)thio)-6-((E)-(((R)-tert-butylsulfinyl)imino)methyl)tetrahydro-2H-pyran-3,4,5-triyl tribenzoate (650 mg, 1 Eq, 804 μmol) in THF (8.04 mL) at −109° C. was added crotylzinc lithium chloride (191 mg, 3.22 mL, 0.30 molar, 1.2 Eq, 965 μmol) dropwise over 15 minutes. Upon the complete addition of allylzinc, the reaction was quenched with sat. aq. NH₄Cl (10 mL). The reaction was warmed to 23° C. and was diluted with EtOAc (30 mL) and H₂O (10 mL). The layers of the resultant biphasic mixture were separated, and the aq. layer was extracted with EtOAc (2×20 mL). The combined organics were dried over sodium sulfate and concentrated in vacuo. The crude residue containing product A11 (650 mg, 0.75 mmol, 94%) was used directly in the next step without additional purification. ¹H NMR (400 MHZ, CDCl₃) δ 8.15-8.05 (m, 2H), 8.01-7.91 (m, 2H), 7.79-7.71 (m, 2H), 7.65-7.58 (m, 1H), 7.54-7.46 (m, 3H), 7.46-7.33 (m, 3H), 7.25-7.18 (m, 2H), 6.28-6.18 (m, 1H), 6.14-5.93 (m, 1.6H), 5.89-5.59 (m, 3.4H), 5.25-5.04 (m, 2.6H), 5.02-4.81 (m, 1.4H), 4.75 (d, J=6.4 Hz, 0.2H), 4.69-4.61 (m, 0.4H), 4.58 (d, J=7.3 Hz, 0.4H), 3.83 (dd, J=10.0, 4.4 Hz, 0.6H), 3.73-3.43 (m, 3.4H), 3.04-2.84 (m, 1.6H), 2.70-2.47 (m, 1.4H), 2.36-2.22 (m, 1H), 1.13-1.11 (m, 6H), 1.08 (s, 1.8H), 1.07 (s, 1.2H), 0.90 (s, 2H), 0.89 (s, 4H), 0.81 (s, 3H), 0.10-0.03 (m, 4H), −0.04 (d, J=2.0 Hz, 1H), −0.07 (d, J=1.9 Hz, 1H).

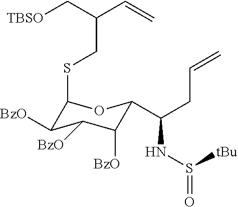

(2R,3R,4S,5S,6R)-2-((2-(((tert-butyldimethylsilyl)oxy)methyl) but-3-en-1-yl)thio)-6-((R)-1-(((R)-tert-butylsulfinyl)amino) but-3-en-1-yl)tetrahydro-2H-pyran-3,4,5-triyl tribenzoate (A12 1:1 mixture of diastereomers): Product A12 was prepared in a similar fashion to product A11 from (2R,3R,4S,5S,6R)-2-((2-(((tert-butyldimethylsilyl)oxy)methyl) but-3-en-1-yl)thio)-6-((E)-(((R)-tert-butylsulfinyl)imino)methyl)tetrahydro-2H-pyran-3,4,5-triyl tribenzoate and allylzinc lithium chloride. ¹H NMR (400 MHz, CDCl₃) δ 8.11-8.06 (m, 2H), 7.97 (dt, J=8.4, 1.2 Hz, 2H), 7.77-7.72 (m, 2H), 7.65-7.60 (m, 1H), 7.55-7.46 (m, 3H), 7.40 (dt, J=13.7, 7.6 Hz, 3H), 7.25-7.17 (m, 2H), 6.09 (d, J=3.2 Hz, 1H), 6.00 (t, J=5.2 Hz, 1H), 5.94-5.78 (m, 2H), 5.77-5.58 (m, 2H), 5.26-5.12 (m, 2H), 5.12-4.98 (m, 2H), 4.72 (dd, J=7.6, 5.1 Hz, 1H), 3.80 (d, J=4.3 Hz, 1H), 3.67-3.56 (m, 2H), 3.52 (ddd, J=9.9, 7.6, 6.1 Hz, 1H), 2.86 (dd, J=12.8, 5.7 Hz, 1H), 2.81 (dd, J=13.1, 6.5 Hz, 1H), 2.68 (dd, J=13.1, 6.8 Hz, 1H), 2.64-2.57 (m, 3H), 2.43 (dh, J=12.8, 6.1 Hz, 1H), 1.14 (d, J=2.9 Hz, 9H), 0.87 (s, 5H), 0.85 (s, 4H), 0.01 (s, 3H), −0.03 (s, 3H). HRMS (ESI+, m/z): Calc'd for[C₄₅H₅₉NO₉S₂Si+H]⁺: 850.3473 found: 850.3496.

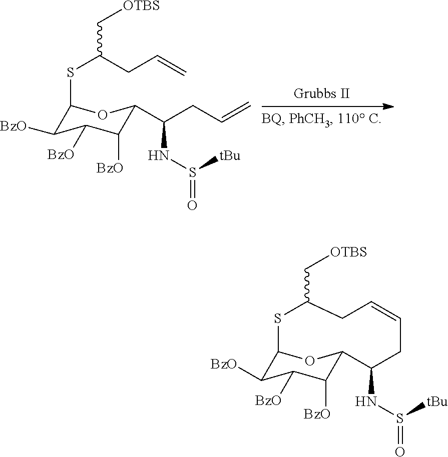

(1R,8R,9R,10S,11S,12R,Z)-3-(((tert-butyldimethylsilyl)oxy)methyl)-8-(((R)-tert-butylsulfinyl)amino)-13-oxa-2-thiabicyclo[7.3.1]tridec-5-ene-10,11,12-triyl tribenzoate (A13 7:3 mixture of diastereomers): A degassed solution of (2R,3R,4S,5S,6R)-2-((1-((tert-butyldimethylsilyl)oxy)pent-4-en-2-yl)thio)-6-((R)-1-(((R)-tert-butylsulfinyl)amino) but-3-en-1-yl)tetrahydro-2H-pyran-3,4,5-triyl tribenzoate (450 mg, 1 Eq, 529 μmol) in PhCH₃ (529 mL) was heated to reflux. A solution of Grubbs II (89.9 mg, 0.2 Eq, 106 μmol) and benzoquinone (22.9 mg, 0.4 Eq, 212 μmol) in PhCH₃ (1 mL) was added and the reaction mixture was refluxed for 15 minutes. The reaction was quenched upon addition of DMSO (0.75 ML, 20 Eq, 10.6 mmol) and the reaction was cooled to 0° C. The reaction was concentrated in vacuo and dissolved in EtOAc (100 mL). The EtOAc was washed with sat. aq. NaCl (1×50 mL). The combined aq. layers were extracted with EtOAc (50 mL). The combined organics were dried over sodium sulfate and concentrated in vacuo. The crude residue was purified by flash chromatography (0-70% EtOAc in Hexanes) to afford product A13 (370 mg, 0.45 mmol, 85.0%) as an off-white solid. ¹H NMR (400 MHZ, CDCl₃) δ 8.15-8.06 (m, 2H), 8.01-7.93 (m, 2H), 7.80-7.73 (m, 2H), 7.67-7.58 (m, 1H), 7.54-7.46 (m, 3H), 7.43-7.34 (m, 3H), 7.21 (t, J=7.5 Hz, 2H), 6.47 (d, J=4.7 Hz, 0.7H), 6.07 (d, J=5.8 Hz, 0.3H), 5.86-5.60 (m, 5H), 4.66 (d, J=9.8 Hz, 0.7H), 4.63 (d, J=10.0 Hz, 0.3H), 4.22 (t, J=4.1 Hz, 1H), 3.87 (app qd, J=10.2, 5.5 Hz, 1.4H), 3.79-3.65 (m, 0.6H), 3.61-3.51 (m, 0.7H), 3.45 (q, J=6.7 Hz, 0.3H), 3.05-2.74 (m, 3H), 2.62 (app d, J=14.4 Hz, 1H), 2.39 (t, J=13.7 Hz, 1H), 1.31 (br. s, 9H), 0.84 (s, 6.3H), 0.83 (s, 2.7H), 0.03 (s, 1H), 0.02 (s, 2H), 0.02 (s, 1H), −0.01 (s, 2H). HRMS (ESI+, m/z): Calc'd for[C₄₃H₅₅NO₉S₂Si+H]⁺: 823.3160; found: 822.3146

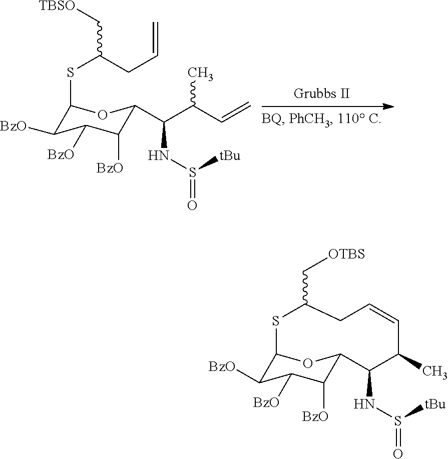

(1R,3S,7R,8R,9R,10S,11S,12R,Z)-3-(((tert-butyldimethylsilyl)oxy)methyl)-8-(((R)-tert-butylsulfinyl)amino)-7-methyl-13-oxa-2-thiabicyclo[7.3.1]tridec-5-ene-10,11,12-triyl tribenzoate (A14, inseparable from other diastereomers, minor components not reported): A degassed solution of (2R,3R,4S,5S,6R)-2-((1-((tert-butyldimethylsilyl)oxy)pent-4-en-2-yl)thio)-6-((1R)-1-(((R)-tert-butylsulfinyl)amino)-2-methylbut-3-en-1-yl)tetrahydro-2H-pyran-3,4,5-triyl tribenzoate (650 mg, 1 Eq, 752 μmol) in in PhCH₃ (375 mL) was heated to reflux. A solution of Grubbs II (128 mg, 0.2 Eq, 150 μmol) and benzoquinone (32.5 mg, 0.4 Eq, 301 μmol) in PhCH₃ (1 mL) was added and the reaction mixture was refluxed for 15 minutes. The reaction was quenched upon addition of DMSO (1.0 mL, 20 Eq, 15 mmol) and the reaction was cooled to 0° C. The reaction was concentrated in vacuo and dissolved in EtOAc (100 mL). The EtOAc was washed with sat. aq. NaCl (1×50 mL). The aq. layer was extracted with EtOAc (50 mL). The combined organics were dried over sodium sulfate and concentrated in vacuo. The crude residue was purified by flash chromatography (0-70% EtOAc in Hexanes) to afford product A14 (370 mg, 0.45 mmol, 85.0%) as an off-white solid. ¹H NMR (400 MHZ, CDCl₃) δ 8.15-8.01 (m, 2H), 8.00-7.88 (m, 2H), 7.83-7.72 (m, 2H), 7.66-7.55 (m, 1H), 7.55-7.45 (m, 3H), 7.45-7.31 (m, 3H), 7.25-7.16 (m, 2H), 6.46 (t, J=2.8 Hz, 1H), 6.20 (d, J=2.0 Hz, 1H), 6.13-5.51 (m, 4H), 4.75 (d, J=10.6 Hz, 1H), 3.93-3.66 (m, 2H), 3.48 (ddd, J=10.5, 7.2, 3.2 Hz, 1H), 3.31 (d, J=7.3 Hz, 1H), 3.14 (ddd, J=10.4, 6.9, 3.3 Hz, 1H), 2.96-2.67 (m, 2H), 2.50-2.33 (m, 1H), 1.27 (s, 9H), 1.11 (d, J=6.9 Hz, 3H), 0.85 (s, 9H), 0.02 (s, 3H), −0.01 (s, 3H). HRMS (ESI+, m/z): Calc'd for[C₄₄H₅₈NO₉S₂Si+H]⁺: 836.3317 found: 836.3365.

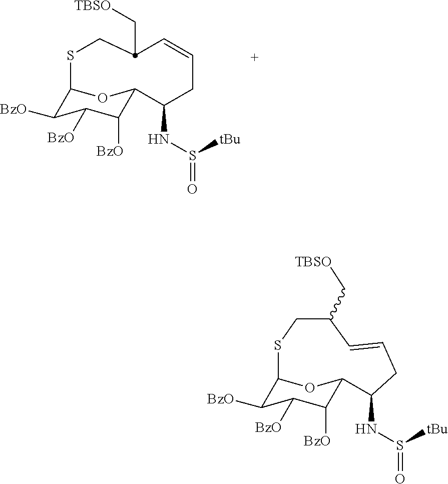

(1R,4S,8R,9R,10S,11S,12R,Z)-4-(((tert-butyldimethylsilyl)oxy)methyl)-8-(((R)-tert-butylsulfinyl)amino)-13-oxa-2-thiabicyclo[7.3.1]tridec-5-ene-10,11,12-triyl tribenzoate and (1R,8R,9R,10S,11S,12R,E)-4-(((tert-butyldimethylsilyl)oxy)methyl)-8—(((R)-tert-butylsulfinyl)amino)-13-oxa-2-thiabicyclo[7.3.1]tridec-5-ene-10,11,12-triyl tribenzoate (A15 as an inseparable mixture of isomers): Product A15 was prepared in a similar fashion to product A12 from (2R,3R,4S,5S,6R)-2-((2-(((tert-butyldimethylsilyl)oxy)methyl) but-3-en-1-yl)thio)-6-((R)-1-(((R)-tert-butylsulfinyl)amino) but-3-en-1-yl)tetrahydro-2H-pyran-3,4,5-triyl tribenzoate. ¹H NMR (400 MHz, CDCl₃) ¹H NMR (400 MHZ, CDCl₃) δ 8.16-8.02 (m, 2H), 8.02-7.89 (m, 2H), 7.85-7.70 (m, 2H), 7.68-7.58 (m, 1H), 7.55-7.45 (m, 3H), 7.45-7.34 (m, 3H), 7.24-7.13 (m, 2H), 6.11-5.93 (m, 1H), 5.89-5.06 (m, 5H), 4.83-4.43 (m, 1H), 4.28-3.10 (m, 5H), 2.95-2.09 (m, 4H), 1.36-1.26 (m, 9H), 1.00-0.79 (m, 9H), 0.17-−0.06 (m, 6H). HRMS (ESI+, m/z): Calc'd for[C₄₃H₅₆NO₉S₂Si+H]⁺: 822.3160 found: 822.3169.

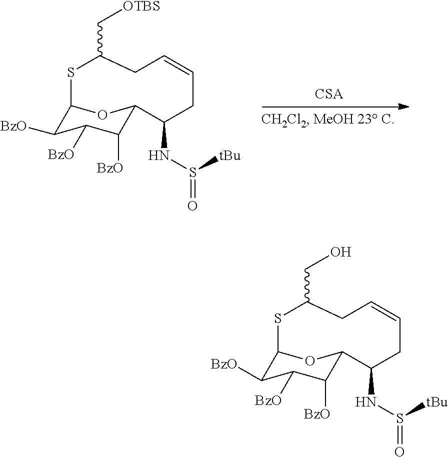

(1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate (A16, 7:3 mixture of diastereomers): To a solution of (1R,8R,9R,10S,11S,12R,Z)-3-(((tert-butyldimethylsilyl)oxy)methyl)-8-(((R)-tert-butylsulfinyl)amino)-13-oxa-2-thiabicyclo[7.3.1]tridec-5-ene-10,11,12-triyl tribenzoate (106 mg, 1 Eq, 129 μmol) in MeOH (0.64 mL) and CH₂Cl₂ (0.65 mL) at 23° C. was added camphorsulfonic acid (15.0 mg, 0.5 Eq, 64.5 μmol). The reaction was stirred at 23° C. for 30 min. The reaction mixture was diluted with sat. aq. NaHCO₃ (10 mL) and extracted with CH₂Cl₂ (2×10 mL). The combined organics were dried over sodium sulfate and concentrated in vacuo. The crude residue was purified by flash chromatography (0-70% EtOAc in Hexanes) to afford product A16 (72 mg, 0.10 mmol, 79%) as a white solid. ¹H NMR (400 MHZ, CD₃OD) δ 8.18-8.06 (m, 2H), 8.00-7.89 (m, 2H), 7.83-7.73 (m, 2H), 7.69-7.59 (m, 1H), 7.56-7.46 (m, 3H), 7.46-7.34 (m, 3H), 7.26-7.16 (m, 2H), 6.44 (d, J=6.0 Hz, 0.7H), 6.09 (d, J=5.8 Hz, 0.3H), 5.87-5.56 (m, 5H), 4.63 (t, J=9.5 Hz, 0.3H), 4.61 (t, J=9.7 Hz, 0.7H), 4.25 (t, J=4.9 Hz, 0.3H), 4.23 (d, J=4.4 Hz, 0.7H), 3.84-3.74 (m, 2H), 3.60-3.52 (m, 1H), 3.50 (q, J=5.8 Hz, 0.3H), 3.19-2.98 (m, 1H), 2.93-2.71 (m, 1.7H), 2.71-2.52 (m, 1H), 2.34 (d, J=14.3 Hz, 0.3H), 2.18 (d, J=13.9 Hz, 0.7H), 1.31 (d, J=1.6 Hz, 9H). HRMS (ESI+, m/z): Calc'd for[C₃₇H₄₂NO₉S₂+H]⁺: 708.2295 found: 708.318.

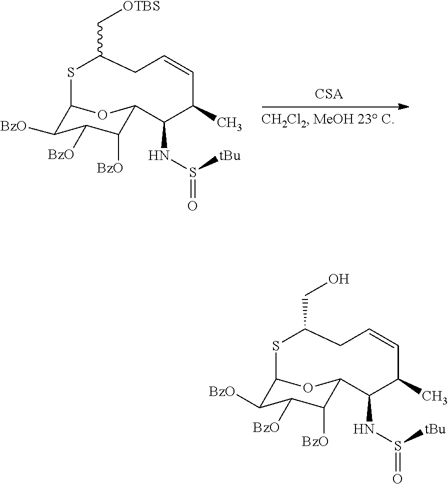

(1R,3S,7R,8R,9R,10S,11S,12R,Z)-8-(((R)-tert-butylsulfinyl)amino)-3-(hydroxymethyl)-7-methyl-13-oxa-2-thiabicyclo[7.3.1]tridec-5-ene-10,11,12-triyl tribenzoate A17: To a solution of (1R,7R,8R,9R,10S,11S,12R,Z)-3-(((tert-butyldimethylsilyl)oxy)methyl)-8-(((R)-tert-butylsulfinyl)amino)-7-methyl-13-oxa-2-thiabicyclo[7.3.1]tridec-5-ene-10,11,12-triyl tribenzoate (350 mg, 1 Eq, 0.41 mmol) in MeOH (2.0 mL) and CH₂Cl₂ (2.0 mL) at 23° C. was added camphorsulfonic acid (48.6.0 mg, 0.5 Eq, 0.2 mmol). The reaction was stirred at 23° C. for 30 min. The reaction mixture was diluted with sat. aq. NaHCO₃ (10 mL) and extracted with CH₂Cl₂ (2×10 mL). The combined organics were dried over sodium sulfate and concentrated in vacuo. The crude residue was purified by flash chromatography (0-70% EtOAc in Hexanes) to afford product A17 (117 mg, 0.16 mmol, 39%) as a white solid. ¹H NMR (400 MHZ, CDCl₃) δ 8.15-8.01 (m, 2H), 8.00-7.88 (m, 2H), 7.84-7.76 (m, 2H), 7.67-7.57 (m, 1H), 7.56-7.47 (m, 3H), 7.47-7.41 (m, 1H), 7.41-7.34 (m, 2H), 7.29-7.22 (m, 2H), 6.47 (d, J=6.2 Hz, 1H), 6.23 (d, J=3.1 Hz, 1H), 5.77-5.62 (m, 2H), 5.62-5.45 (m, 2H), 4.71 (d, J=10.2 Hz, 1H), 3.76 (d, J=7.3 Hz, 2H), 3.48 (ddd, J=10.6, 7.3, 3.3 Hz, 1H), 3.32 (d, J=7.3 Hz, 1H), 3.09 (ddt, J=11.7, 8.1, 4.1 Hz, 1H), 3.06-2.95 (m, 1H), 2.94-2.77 (m, 1H), 2.24-2.16 (m, 1H), 1.28 (s, 9H), 1.10 (d, J=6.9 Hz, 3H). HRMS (ESI+, m/z): Calc'd for[C₃₈H₄₃NO₉S₂+H]⁺: 722.2452 found: 722.2465.

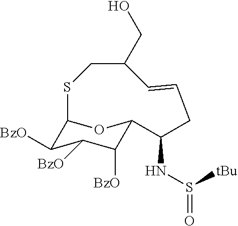

(1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate (A18 single diastereomer, unassigned): Product A18 was prepared in a similar fashion to product A17 from (1R,8R,9R,10S,11S,12R,E)-4-(((tert-butyldimethylsilyl)oxy)methyl)-8-(((R)-tert-butylsulfinyl) amino)-13-oxa-2-thiabicyclo[7.3.1]tridec-5-ene-10,11,12-triyl tribenzoate. ¹H NMR (400 MHZ, CDCl₃) δ 8.13-8.06 (m, 2H), 7.98-7.93 (m, 2H), 7.78-7.72 (m, 2H), 7.65-7.59 (m, 1H), 7.54-7.46 (m, 3H), 7.45-7.35 (m, 3H), 7.22 (t, J=7.8 Hz, 2H), 6.00 (d, J=3.4 Hz, 1H), 5.90 (td, J=14.9, 3.3 Hz, 2H), 5.83 (d, J=5.6 Hz, 1H), 5.74 (dd, J=10.8, 5.6 Hz, 1H), 5.65 (dd, J=10.8, 3.4 Hz, 1H), 5.18 (dd, J=14.9, 10.4 Hz, 1H), 4.78 (d, J=10.4 Hz, 1H), 4.17 (d, J=5.1 Hz, 1H), 3.62 (dt, J=11.5, 6.1 Hz, 1H), 3.59-3.51 (m, 2H), 3.22 (dd, J=14.6, 3.0 Hz, 1H), 2.93-2.81 (m, 1H), 2.61-2.42 (m, 2H), 2.32 (dd, J=14.6, 11.7 Hz, 1H), 1.31 (s, 9H). HRMS (ESI+, m/z): Calc'd for[C₃₇H₄₂NO₉S₂+H]⁺: 708.2995 found: 708.2302.

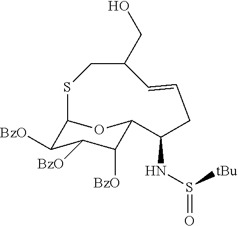

(1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate (A19 single diastereomer, unassigned, 4:1 mixture of rotamers): Product A19 was prepared in a similar fashion to product A17 from (1R,8R,9R,10S,11S,12R,E)-4-(((tert-butyldimethylsilyl)oxy)methyl)-8-(((R)-tert-butylsulfinyl)amino)-13-oxa-2-thiabicyclo[7.3.1]tridec-5-ene-10,11,12-triyl tribenzoate. ¹H NMR (400 MHZ, CDCl₃) δ 8.12-8.06 (m, 2H), 7.99-7.91 (m, 2H), 7.80-7.69 (m, 2H), 7.65-7.59 (m, 1H), 7.54-7.45 (m, 3H), 7.44-7.35 (m, 3H), 7.25-7.13 (m, 2H), 6.02-5.40 (m, 5H), 4.73-4.49 (m, 1H), 4.31-4.12 (m, 1H), 4.05-3.88 (m, 0.8H), 3.84 (dd, J=10.4, 8.3 Hz, 0.8H), 3.64-3.08 (m, 2.6H), 3.02-2.43 (m, 3.8H), 1.33-1.28 (m, 9H). HRMS (ESI+, m/z): Calc'd for[C₃₇H₄₂NO₉S₂+H]⁺: 708.2995 found: 708.2302

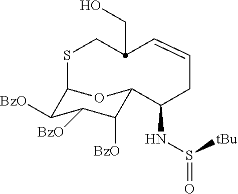

(1R,4S,8R,9R,10S,11S,12R,Z)-8-(((R)-tert-butylsulfinyl)amino)-4-(hydroxymethyl)-13-oxa-2-thiabicyclo[7.3.1]tridec-5-ene-10,11,12-triyl tribenzoate

(A20): Product A20 was prepared in a similar fashion to product A17 from (1R,4S,8R,9R,10S,11S,12R,Z)-4-(((tert-butyldimethylsilyl)oxy)methyl)-8-(((R)-tert-butylsulfinyl)amino)-13-oxa-2-thiabicyclo[7.3.1]tridec-5-ene-10,11,12-triyl tribenzoate. ¹H NMR (400 MHZ, CDCl₃) δ 8.15-8.09 (m, 2H), 7.99-7.95 (m, 2H), 7.79-7.74 (m, 2H), 7.66-7.60 (m, 1H), 7.56-7.47 (m, 3H), 7.44-7.36 (m, 3H), 7.24-7.17 (m, 2H), 6.05 (d, J=5.7 Hz, 1H), 5.90 (td, J=11.4, 4.9 Hz, 1H), 5.83 (d, J=3.2 Hz, 1H), 5.80 (dd, J=10.8, 5.6 Hz, 1H), 5.72 (dd, J=10.8, 3.5 Hz, 2H), 5.45 (t, J=11.0 Hz, 1H), 4.64 (d, J=9.9 Hz, 1H), 4.28 (d, J=4.0 Hz, 1H), 3.62-3.55 (m, 1H), 3.53 (dd, J=10.5, 5.3 Hz, 1H), 3.41 (dd, J=10.6, 7.1 Hz, 1H), 3.33 (dt, J=11.4, 5.4 Hz, 1H), 3.12 (dd, J=14.1, 4.0 Hz, 1H), 2.88-2.75 (m, 1H), 2.73-2.62 (m, 1H), 2.44 (dd, J=14.2, 12.3 Hz, 1H), 1.31 (s, 9H). HRMS (ESI+, m/z): Calc'd for[C₃₇H₄₂NO₉S₂+H]⁺: 708.2995 found: 708.2308.

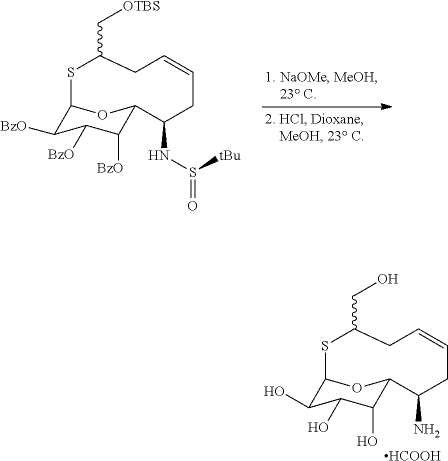

(1R,8R,9R,10R,11S,12R,Z)-8-amino-3-(hydroxymethyl)-13-oxa-2-thiabicyclo[7.3.1]tridec-5-ene-10,11,12-triol formate salt (A21,7:3 mixture of diastereomers): NaOMe in MeOH (0.5 M, 0.65 mL, 0.35 mmol, 10 equiv.) was added to (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate (29 mg, 0.035 mmol, 1.00 equiv.) and the resulting solution stirred for 1 h. HCl in dioxane (4.0 M, 0.25 mL, 1.0 mmol, 28.8 equiv.) was then added to the mixture, and the resulting white suspension stirred for 1 h. The reaction mixture was concentrated in vacuo and purified by prep-HPLC(1-30% MeCN in H₂O with 0.1% HCOOH over 30 minutes) to yield product A21 (9.5 mg, 0.033 mmol, 93% over 2 steps) as a white solid. ¹H NMR (400 MHZ, CD₃OD) δ 8.09 (s, 1H), 5.92-5.73 (m, 1H), 5.79 (d, J=6.1 Hz, 1H), 5.64-5.49 (m, 1H), 5.53 (d, J=5.8 Hz, 1H), 4.47 (dd, J=10.5, 1.2 Hz, 0.7H), 4.43 (dd, J=10.5, 1.1 Hz, 0.3H), 4.05 (app dt, J=10.1, 6.1 Hz, 2H), 3.90 (dd, J=11.6, 6.7 Hz, 0.7H), 3.85 (dd, J=3.4, 1.3 Hz, 1H), 3.78 (dd, J=11.5, 6.3 Hz, 0.7H), 3.76-3.69 (m, 0.6H), 3.69-3.62 (m, 1H), 3.52 (dd, J=10.2, 3.4 Hz, 0.7H), 3.47 (dd, J=10.2, 3.4 Hz, 0.3H), 3.08-2.77 (m, 3H), 2.39 (d, J=14.1 Hz, 0.3H), 2.31-2.24 (m, 0.7H), 2.23-2.08 (m, 1H). HRMS (ESI+, m/z): Calc'd for[C₁₂H₂₁NO₅S+H]⁺: 292.1213 found: 292.1210.

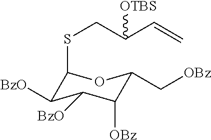

(2R,3S,4S,5R,6R)-2-((benzoyloxy)methyl)-6-((2-((tert-butyldimethylsilyl)oxy)but-3-en-1-yl)thio)tetrahydro-2H-pyran-3,4,5-triyl tribenzoate (A22; 1:1 mixture of diastereomers): Product A22 was prepared in a similar fashion as Product A3 from (2R,3S,4S,5R,6R)-2-((benzoyloxy)methyl)-6-mercaptotetrahydro-2H-pyran-3,4,5-triyl tribenzoate and ((1-bromobut-3-en-2-yl)oxy)(tert-butyl)dimethylsilane. ¹H NMR (400 MHZ, CDCl₃) δ 8.12-8.05 (m, 2H), 8.05-7.95 (m, 4H), 7.83-7.75 (m, 2H), 7.68-7.59 (m, 1H), 7.58-7.46 (m, 4H), 7.46-7.33 (m, 5H), 7.28-7.22 (m, 2H), 6.06-5.99 (m, 1H), 5.95-5.85 (m, 2H), 5.79-5.63 (m, 1H), 5.13 (app tt, J=17.4, 1.4 Hz, 1H), 5.08-4.95 (m, 2H), 4.60 (app ddd, J=11.5, 7.4, 5.3 Hz, 1H), 4.45 (app ddd, J=11.5, 10.2, 5.4 Hz, 1H), 4.29-4.16 (m, 1H), 2.78 (app ddd, J=13.4, 10.7, 6.1 Hz, 1H), 2.64 (dt, J=12.9, 6.6 Hz, 1H), 0.83 (d, J=2.8 Hz, 9H), 0.00 (app d, J=4.6 Hz, 3H), −0.03 (s, 3H). HRMS (ESI+, m/z): Calc'd for[C₄₄H₄₈O₁₀SSi+NH₄]⁺: 814.3070; found: 814.3094

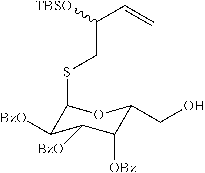

(2R,3R,4S,5S,6R)-2-((2-((tert-butyldimethylsilyl)oxy)but-3-en-1-yl)thio)-6-(hydroxymethyl)tetrahydro-2H-pyran-3,4,5-triyl tribenzoate (A23, 1:1 mixture of diastereomers): Product A23 was prepared in a similar fashion to product A4 from (2R,3S,4S,5R,6R)-2-((benzoyloxy)methyl)-6-((2-(((tert-butyldimethylsilyl)oxy)methyl) but-3-en-1-yl)thio)tetrahydro-2H-pyran-3,4,5-triyl tribenzoate. ¹H NMR (400 MHZ, CDCl₃) 8.14-8.09 (m, 2H), 8.05-7.96 (m, 3H), 7.84-7.75 (m, 2H), 7.65-7.57 (m, 1H), 7.26-7.21 (m, 2H) 7.44-7.35 (m, 3H), 7.24 (d, J=7.8 Hz, 2H), 6.06-5.64 (m, 5H), 5.26-4.99 (m, 2H), 4.72 (app td, J=6.8, 3.9 Hz, 1H), 4.25 (app dq, J=7.9, 6.1 Hz, 1H), 3.77 (app ddd, J=11.2, 6.6, 4.3 Hz, 1H), 3.71-3.55 (m, 1H), 2.84-2.58 (m, 2H), 0.85 (app d, J=5.4 Hz, 9H), 0.06 (app d, J=4.9 Hz, 3H), −0.00 (s, 3H). HRMS (ESI+, m/z): Calc'd for[C₃₇H₄₄O₉SSi+H]⁺: 710.2803; found: 710.2811.

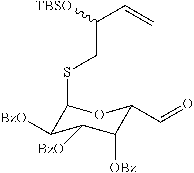

(2R,3R,4S,5R,6S)-2-((2-((tert-butyldimethylsilyl)oxy)but-3-en-1-yl)thio)-6-formyltetrahydro-2H-pyran-3,4,5-triyl tribenzoate (A25, 1:1 mixture of diastereomers): Product A25 was prepared in a similar fashion to product A6 from (2R,3R,4S,5S,6R)-2-((2-((tert-butyldimethylsilyl)oxy)but-3-en-1-yl)thio)-6-(hydroxymethyl)tetrahydro-2H-pyran-3,4,5-triyl tribenzoate. ¹H NMR (400 MHZ, CDCl₃) δ 9.66 (d, J=2.9 Hz, 1H), 8.02-7.94 (m, 4H), 7.81-7.75 (m, 2H), 7.66-7.57 (m, 2H), 7.55-7.35 (m, 5H), 7.30-7.22 (m, 2H), 6.28 (dd, J=3.2, 1.7 Hz, 1H), 6.11 (dd, J=10.0, 5.2 Hz, 1H), 5.92-5.72 (m, 3H), 5.24-4.97 (m, 3H), 4.30-4.19 (m, 1H), 2.83 (app ddd, J=14.4, 13.2, 6.2 Hz, 1H), 2.69 (app ddd, J=16.3, 13.1, 6.1 Hz, 1H), 0.85 (d, J=3.6 Hz, 9H), 0.03 (app d, J=6.5 Hz, 3H), −0.00 (app d, J=1.8 Hz, 3H). HRMS (ESI+, m/z): Calc'd for[C₃₇H₄₂O₉SSi+H]⁺: 691.2391; found: 691.2402

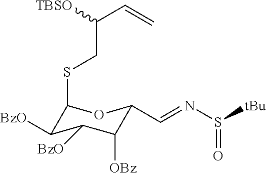

(2R,3R,4S,5S,6R)-2-((2-((tert-butyldimethylsilyl)oxy)but-3-en-1-yl)thio)-6-((E)-(((R)-tert-butylsulfinyl)imino)methyl)tetrahydro-2H-pyran-3,4,5-triyl tribenzoate (A26 1:1 mixture of diastereomers): Product A26 was prepared in a similar fashion to product A8 from (2R,3R,4S,5S,6R)-2-((2-((tert-butyldimethylsilyl)oxy)but-3-en-1-yl)thio)-6-(hydroxymethyl)tetrahydro-2H-pyran-3,4,5-triyl tribenzoate. ¹H NMR (400 MHZ, CDCl₃) δ 8.04 (dd, J=3.4, 2.3 Hz, 1H), 8.01-7.90 (m, 4H), 7.82-7.70 (m, 2H), 7.64-7.55 (m, 1H), 7.55-7.47 (m, 1H), 7.45 (t, J=7.7 Hz, 3H), 7.38 (t, J=7.7 Hz, 2H), 7.25 (t, J=7.8 Hz, 2H), 6.30 (td, J=3.7, 1.5 Hz, 1H), 6.11 (dd, J=11.4, 5.3 Hz, 1H), 5.92-5.75 (m, 3H), 5.51 (dt, J=5.3, 1.9 Hz, 1H), 5.18 (ddt, J=17.1, 10.1, 1.4 Hz, 1H), 5.08 (ddt, J=17.0, 10.4, 1.3 Hz, 1H), 4.25 (p, J=6.4 Hz, 1H), 2.81 (ddd, J=14.9, 13.1, 6.3 Hz, 1H), 2.67 (ddd, J=14.2, 13.1, 6.1 Hz, 1H), 0.98 (d, J=0.9 Hz, 9H), 0.85 (d, J=6.3 Hz, 9H), 0.06 (d, J=3.3 Hz, 3H), −0.01 (s, 3H). HRMS (ESI+, m/z): Calc'd for[C₄₁H₅₁NO₉S₂Si+H]⁺: 794.2842; found: 794.2852.

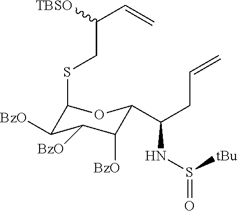

(2R,3R,4S,5S,6R)-2-((2-((tert-butyldimethylsilyl)oxy)but-3-en-1-yl)thio)-6-((R)-1-(((R)-tert-butylsulfinyl)amino) but-3-en-1-yl)tetrahydro-2H-pyran-3,4,5-triyl tribenzoate (A27 1:1 mixture of diastereomers): Product A27 was prepared in a similar fashion to product A10 from (2R,3R,4S,5S,6R)-2-((2-((tert-butyldimethylsilyl)oxy)but-3-en-1-yl)thio)-6-((E)-(((R)-tert-butylsulfinyl)imino)methyl)tetrahydro-2H-pyran-3,4,5-triyl tribenzoate and allylzinc lithium chloride. ¹H NMR (400 MHZ, CDCl₃) δ 8.12-8.04 (m, 2H), 8.03-7.90 (m, 2H), 7.79-7.70 (m, 2H), 7.66-7.58 (m, 1H), 7.57-7.47 (m, 3H), 7.46-7.33 (m, 3H), 7.22 (t, J=7.7 Hz, 2H), 6.10 (d, J=3.2 Hz, 1H), 6.02 (app dd, J=15.7, 5.7 Hz, 1H), 5.94-5.69 (m, 4H), 5.26-5.14 (m, 2H), 5.14-5.00 (m, 2H), 4.73 (d, J=7.4 Hz, 1H), 4.24 (dq, J=12.1, 6.2 Hz, 1H), 3.82 (d, J=4.1 Hz, 1H), 3.66-3.54 (m, 1H), 2.84-2.64 (m, 2H), 2.64-2.54 (m, 2H), 1.13 (d, J=7.0 Hz, 9H), 0.84 (d, J=4.8 Hz, 10H), 0.03 (app d, J=10.1 Hz, 3H), −0.02 (d, J=1.4 Hz, 3H). HRMS (ESI+, m/z): Calc'd for[C₄₄H₅₇NO₉S₂Si+H]⁺: 836.3317 found: 836.3362.

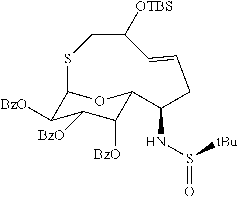

(1R,8R,9R,10S,11S,12R,E)-4-((tert-butyldimethylsilyl)oxy)-8-(((R)-tert-butylsulfinyl)amino)-13-oxa-2-thiabicyclo[7.3.1]tridec-5-ene-10,11,12-triyl tribenzoate (A28, single isomer reported): Product A28 was prepared in a similar fashion to product A12 from (2R,3R,4S,5S,6R)-2-((2-((tert-butyldimethylsilyl)oxy)but-3-en-1-yl)thio)-6-((R)-1-(((R)-tert-butylsulfinyl)amino) but-3-en-1-yl)tetrahydro-2H-pyran-3,4,5-triyl tribenzoate. ¹H NMR (400 MHZ, CDCl₃) δ 8.13-8.07 (m, 2H), 8.00-7.96 (m, 2H), 7.81-7.74 (m, 2H), 7.64-7.57 (m, 1H), 7.52-7.45 (m, 3H), 7.45-7.33 (m, 3H), 7.23-7.15 (m, 2H), 6.01-5.85 (m, 2H), 5.83-5.64 (m, 3H), 5.42-5.27 (m, 1H), 4.66 (d, J=9.9 Hz, 1H), 4.58 (dd, J=4.4, 2.6 Hz, 1H), 4.11 (d, J=4.8 Hz, 1H), 3.56 (dt, J=10.9, 5.7 Hz, 1H), 3.13 (dd, J=14.8, 3.9 Hz, 1H), 2.96 (d, J=14.3 Hz, 1H), 2.64 (dd, J=14.8, 1.6 Hz, 1H), 2.58-2.43 (m, 1H), 1.30 (s, 9H), 0.94 (s, 9H), 0.07 (s, 3H), 0.04 (s, 3H).

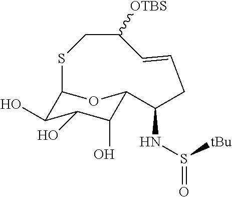 [SMILES: CC(C)(C)[S@@](=O)N[C@@H]1C/C=C/C(O[Si](C)(C)C(C)(C)C)CSC2OC1C(O)C(O)C2O]

N-((1R,8R,9R,10R,11S,12R,E)-4-((tert-butyldimethylsilyl)oxy)-10,11,12-trihydroxy-13-oxa-2-thiabicyclo[7.3.1]tridec-5-en-8-yl)-2-methylpropane-2-sulfinamide (A29, 3:2 mixture of diastereomers): The crude residue containing (1R,8R,9R,10S,11S,12R,Z)-4-((tert-butyldimethylsilyl)oxy)-8-(((R)-tert-butylsulfinyl)amino)-13-oxa-2-thiabicyclo[7.3.1]tridec-5-ene-10,11,12-triyl tribenzoate (110 mg, 1 Eq, 136 μmol) was dissolved in sodium methoxide (1.36 mL, 0.5 molar, 5 Eq, 681 μmol) in MeOH. The reaction maintained for 1 h at 23° C. and was neutralized upon addition of formic acid (77 μL, 15 Eq, 2.0 mmol). The reaction mixture was filtered (0.2 uM syringe filter) and purified by prep-HPLC(5-50% MeCN in H₂O with 0.1% HCOOH over 30 minutes) to afford product A29 (45 mg, 67%). ¹H NMR (400 MHZ, CDCl₃) δ 5.83 (dddd, J=15.0, 11.5, 3.6, 1.9 Hz, 0.6H), 5.57 (dd, J=16.3, 4.9 Hz, 0.4H), 5.37 (dd, J=14.9, 2.3 Hz, 0.6H), 5.33-5.27 (m, 0.4H), 5.17 (app dd, J=8.2, 5.9 Hz, 1H), 4.78-4.74 (m, 0.4H), 4.65 (hept, J=1.8 Hz, 0.6H), 4.32 (dd, J=10.7, 1.0 Hz, 0.6H), 4.13 (d, J=3.5 Hz, 1H), 4.12-4.09 (m, 0.4H), 4.06 (dd, J=10.2, 6.0 Hz, 0.4H), 4.00 (dd, J=10.3, 5.7 Hz, 0.6H), 3.75 (ddd, J=10.7, 5.8, 1.5 Hz, 0.6H), 3.50 (dd, J=10.3, 3.3 Hz, 0.4H), 3.43 (dd, J=10.3, 3.4 Hz, 0.6H), 3.30-3.28 (m, 0.4H), 3.28-3.22 (m, 0.4H), 3.15 (dd, J=14.9, 3.7 Hz, 0.6H), 2.75 (dd, J=14.8, 1.8 Hz, 0.6H), 2.69 (dd, J=11.8, 5.7 Hz, 0.4H), 2.55 (dd, J=13.8, 3.5 Hz, 0.4H), 2.47 (ddd, J=13.3, 11.5, 5.8 Hz, 0.6H), 2.42-2.34 (m, 0.6H), 1.98 (q, J=11.0 Hz, 0.4H), 1.26 (s, 9H), 0.91 (d, J=15.2 Hz, 9H), 0.12-−0.01 (m, 6H). HRMS (ESI+, m/z): Calc'd for[C₂₁H₄₁NO₆S₂Si+H]⁺: 496.2217 found: 496.2222.

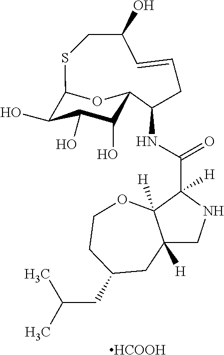 [SMILES: CC(C)C[C@@H]1CCO[C@@H]2[C@H](CN[C@@H]2C(=O)N[C@@H]2C/C=C/[C@H](O)CSC3O[C@H]2C(O)[C@@H](O)[C@H]3O)C1 ; "·HCOOH"]

(4S,5aS,8S,8aR)-4-isobutyl-N-((1R,4S,8R,9R,10R,11S,12R,E)-4,10,11,12-tetrahydroxy-13-oxa-2-thiabicyclo[7.3.1]tridec-5-en-8-yl) octahydro-2H-oxepino[2,3-c]pyrrole-8-carboxamide formate salt (FSA1608009 4:1 mixture of rotamers). Product FSA1608009 was prepared in a similar fashion to FSA1609033 from (R)—N-((1R,8R,9R,10R,11S,12R,E)-4-((tert-butyldimethylsilyl)oxy)-10,11,12-trihydroxy-13-oxa-2-thiabicyclo[7.3.1]tridec-5-en-8-yl)-2-methylpropane-2-sulfinamide. ¹H NMR (400 MHZ, CD₃OD) δ 8.47 (s, 2H), 5.73 (dddd, J=12.7, 10.5, 4.6, 1.9 Hz, 0.8H), 5.59 (dd, J=16.2, 5.1 Hz, 0.2H), 5.42 (dd, J=15.3, 2.2 Hz, 0.8H), 5.0-5.32 (m, 0.2H), 5.21 (d, J=5.7 Hz, 1H), 4.69 (br. s, 0.2H), 4.58 (dt, J=4.0, 2.0 Hz, 0.8H), 4.40-4.29 (m, 2.8H), 4.26 (d, J=11.2 Hz, 1H), 4.21 (d, J=8.5 Hz, 0.2H), 4.10-3.90 (m, 2H), 3.87-3.76 (m, 2H), 3.56 (dd, J=11.3, 7.4 Hz, 1H), 3.44 (dd, J=10.3, 3.4 Hz, 1H), 3.38 (d, J=13.9 Hz, 0.2H), 3.23 (dd, J=15.0, 3.8 Hz, 0.8H), 2.87 (t, J=11.8 Hz, 1H), 2.79 (dd, J=14.9, 1.8 Hz, 0.8H), 2.58 (dd, J=14.3, 3.9 Hz, 0.4H), 2.48-2.32 (m, 1.6H), 2.37-2.22 (m, 1H), 2.10-2.04 (m, 0.2H), 1.99 (d, J=3.1 Hz, 1H), 1.81-1.60 (m, H), 1.18 (td, J=6.8, 4.2 Hz, 2H), 1.03-0.91 (m, 1H), 0.89 (appt. d, J=6.6 Hz, 6H). HRMS (ESI-TOF) m/z: Calc'd for[C₂₄H₄₀N₂O₇S+H]⁺: 501.2629 Found: 501.2717.

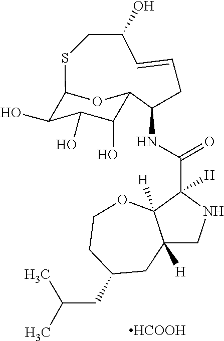 [SMILES: CC(C)C[C@@H]1CCO[C@@H]2[C@H](CN[C@@H]2C(=O)N[C@@H]2C/C=C/[C@@H](O)CSC3OC2C(O)C(O)C3O)C1 ; "·HCOOH"]

(4S,5aS,8S,8aR)-4-isobutyl-N-((1R,4R,8R,9R,10R,11S,12R,E)-4,10,11,12-tetrahydroxy-13-oxa-2-thiabicyclo[7.3.1]tridec-5-en-8-yl) octahydro-2H-oxepino[2,3-c]pyrrole-8-carboxamide formate salt (FSA1608016): Product FSA1608016 was prepared in a similar fashion to FSA1609033 from (R)—N-((1R,8R,9R,10R,11S,12R,E)-4-((tert-butyldimethylsilyl)oxy)-10,11,12-trihydroxy-13-oxa-2-thiabicyclo[7.3.1]tridec-5-en-8-yl)-2-methylpropane-2-sulfinamide. ¹H NMR (400 MHZ, CD₃OD) δ 8.57 (s, 1H), 5.71 (td, J=14.7, 4.0 Hz, 1H), 5.33 (dd, J=14.8, 9.7 Hz, 1H), 5.28 (d, J=5.6 Hz, 1H), 4.39-4.25 (m, 2H), 4.25-4.20 (m, 1H), 4.17 (d, J=10.8 Hz, 1H), 4.04-3.91 (m, 3H), 3.82 (d, J=3.4 Hz, 1H), 3.81-3.72 (m, 1H), 3.55-3.42 (m, 1H), 3.53-3.44 (m, 1H), 3.37 (dd, J=10.3, 3.3 Hz, 1H), 3.16 (dd, J=14.3, 3.9 Hz, 1H), 2.83-2.69 (m, 1H), 2.51 (dd, J=14.3, 10.8 Hz, 1H), 2.45-2.32 (m, 2H), 2.26 (s, 1H), 1.99 (d, J=12.9 Hz, 1H), 1.82-1.58 (m, 4H), 1.18 (h, J=6.9 Hz, 2H), 0.95 (d, J=11.8 Hz, 1H), 0.89 (appt. d, J=6.6 Hz, 6H). HRMS (ESI-TOF) m/z: Calc'd for[C₂₄H₄₀N₂O₇S+H]⁺: 501.2629 Found: 501.2635

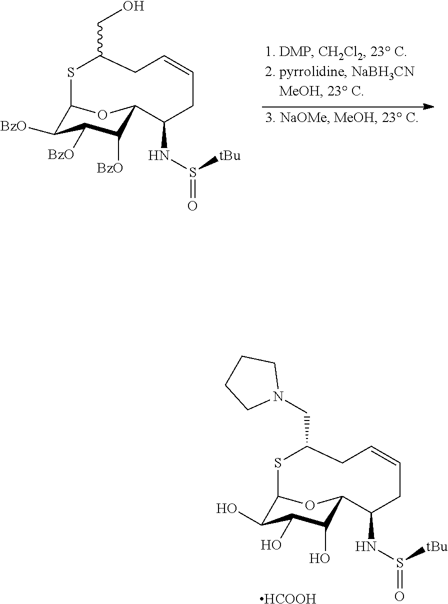

(R)-2-methyl-N-((1R,3S,8R,9R,10R,11S,12R,Z)-10,11,12-trihydroxy-3-(pyrrolidin-1-ylmethyl)-13-oxa-2-thiabicyclo[7.3.1]tridec-5-en-8-yl) propane-2-sulfinamide formate salt (B1): To a solution of (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate (6 mg, 1 Eq, 8 μmol) in CH₂Cl₂ (0.1 mL) at 23° C., was added Dess-Martin Periodinane (4 mg, 1.1 Eq, 9 μmol). The reaction was stirred for 1 hour. The reaction mixture was quenched upon addition of sat. aq. NaHCO₃ (0.5 mL), sat. aq. Na₂S₂O₃ (0.5 mL), and CH₂Cl₂ (0.5 mL). The biphasic reaction mixture was stirred vigorously for 30 minutes and the layers were separated. The aq. layer was extracted with DCM (2×0.5 mL) and the combined organics were dried over sodium sulfate and concentrated in vacuo to afford crude (1R,8R,9R,10S,11S,12R,Z)-8-(((R)-tert-butylsulfinyl)amino)-3-formyl-13-oxa-2-thiabicyclo[7.3.1]tridec-5-ene-10,11,12-triyl tribenzoate (6 mg, 1 Eq, 9 μmol).

To a solution of (1R,8R,9R,10S,11S,12R,Z)-8-(((R)-tert-butylsulfinyl)amino)-3-formyl-13-oxa-2-thiabicyclo[7.3.1]tridec-5-ene-10,11,12-triyl tribenzoate (6 mg, 1 Eq, 9 μmol) in MeOH (0.2 mL) at 23° C., was added pyrrolidine (0.6 mg, 0.7 μL, 1 Eq, 9 μmol) and sodium cyanoborohydride (0.5 mg, 1 Eq, 9 μmol). The resultant reaction mixture was maintained at 23° C. for 16 h. The reaction mixture was concentrated in vacuo and the crude residue containing (1R,8R,9R,10S,11S,12R,Z)-8-(((R)-tert-butylsulfinyl)amino)-3-(pyrrolidin-1-ylmethyl)-13-oxa-2-thiabicyclo[7.3.1]tridec-5-ene-10,11,12-triyl tribenzoate was dissolved in sodium methoxide in MeOH (95 μL, 0.5 molar, 6 Eq, 47 μmol). The reaction was maintained at 23° C. for 1 h and was neutralized upon addition of formic acid (1.1 mg, 0.89 μL, 3 Eq, 24 μmol). The reaction mixture was filtered (0.2 uM syringe filter) and purified by prep-HPLC(5-50% MeCN in H₂O with 0.1% HCOOH over 30 minutes) to afford product B1 (2.0 mg, 4.5 μmol, 57% over 3 steps).: ¹H NMR (400 MHZ, CD₃OD) δ 8 8.36 (s, 1H), 5.79 (d, J=6.0 Hz, 1H), 5.65 (app tdd, J=14.1, 11.3, 6.5 Hz, 2H), 5.38 (d, J=8.7 Hz, 1H), 4.26 (d, J=10.5 Hz, 1H), 4.14 (d, J=2.5 Hz, 1H), 4.12 (dd, J=9.9, 5.8 Hz, 1H), 3.82 (dd, J=14.2, 12.9 Hz, 1H), 3.79-3.68 (m, 1H), 3.68-3.32 (m, 5H), 3.47 (dd, J=10.1, 3.3 Hz, 1H), 3.34 (t, J=3.6 Hz, 1H), 2.94 (dt, J=13.5, 11.2 Hz, 1H), 2.72 (ddd, J=14.6, 11.1, 3.7 Hz, 1H), 2.14 (br. s, 4H), 2.09 (d, J=13.7 Hz, 1H), 1.98 (dd, J=13.3, 3.0 Hz, 1H), 1.26 (s, 9H). LRMS (ESI+, m/z): Calc'd for[C₂₀H₃₆N₂O₆S₂+H]⁺: 449.2 found: 449.2.

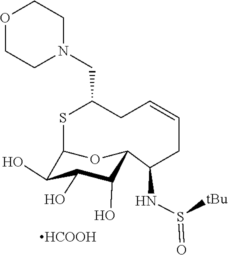

(R)-2-methyl-N-((1R,3S,8R,9R,10R,11S,12R,Z)-10,11,12-trihydroxy-3-(morpholinomethyl)-13-oxa-2-thiabicyclo[7.3.1]tridec-5-en-8-yl) propane-2-sulfinamide formate salt (B2): The product B2 was prepared in a similar fashion to product B1 from (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate and morpholine. ¹H NMR (400 MHz, CD₃OD) δ 8.27 (s, 1H), 5.74 (d, J=6.0 Hz, 1H), 5.66-5.57 (m, 2H), 5.35 (s, 1H), 4.28 (d, J=10.5 Hz, 1H), 4.13 (d, J=3.4 Hz, 1H), 4.09 (dd, J=10.2, 6.0 Hz, 1H), 3.84 (t, J=4.8 Hz, 4H), 3.78-3.65 (m, 1H), 3.46 (dd, J=10.2, 3.3 Hz, 1H), 3.30-3.20 (m, 2H), 3.14-3.06 (m, 1H), 3.00 (dt, J=10.0, 4.9 Hz, 4H), 2.88 (dt, J=13.7, 11.1 Hz, 1H), 2.79-2.65 (m, 1H), 2.21 (d, J=14.8 Hz, 1H), 1.96 (dt, J=14.3, 3.3 Hz, 1H), 1.26 (s, 9H). LRMS (ESI+, m/z): Calc'd for[C₂₀H₃₆N₂O₆S₂+H]⁺: 464.2 found: 464.2

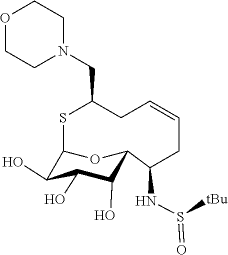

(S)-2-methyl-N-((1R,3S,8R,9R,10R,11S,12R,Z)-10,11,12-trihydroxy-3-(morpholinomethyl)-13-oxa-2-thiabicyclo[7.3.1]tridec-5-en-8-yl) propane-2-sulfinamide formate salt (B3): The product B3 was prepared in a similar fashion to A1 from (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate and morpholine. ¹H NMR (400 MHz, CD₃OD) ¹H NMR (400 MHZ, MeOD) δ 8.27 (s, 1H), 5.74-5.59 (m, 2H), 5.47 (d, J=5.9 Hz, 1H), 5.32 (d, J=8.6 Hz, 1H), 4.27 (d, J=10.6 Hz, 1H), 4.12 (d, J=3.3 Hz, 1H), 4.04 (dd, J=10.2, 5.9 Hz, 1H), 3.83-3.75 (m, 1H), 3.72 (t, J=4.7 Hz, 4H), 3.54 (d, J=6.3 Hz, 1H), 3.42 (dd, J=10.2, 3.3 Hz, 1H), 3.12-3.01 (m, 1H), 2.85-2.73 (m, 1H), 2.69-2.50 (m, 6H), 2.28 (d, J=13.8 Hz, 1H), 1.95 (d, J=13.3 Hz, 1H), 1.25 (s, 9H). LRMS (ESI+, m/z): Calc'd for[C₂₀H₃₆N₂O₆S₂+H]⁺: 464.2 found: 464.2.

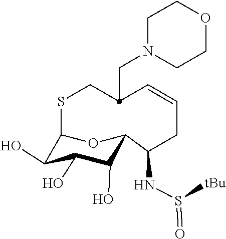

(R)-2-methyl-N-((1R,4S,8R,9R,10R,11S,12R,Z)-10,11,12-trihydroxy-4-(morpholinomethyl)-13-oxa-2-thiabicyclo[7.3.1]tridec-5-en-8-yl) propane-2-sulfinamide formate salt (B4): The product B4 was prepared in a similar fashion to product B1 from (1R,4S,8R,9R,10S,11S,12R,Z)-8-(((R)-tert-butylsulfinyl)amino)-4-(hydroxymethyl)-13-oxa-2-thiabicyclo[7.3.1]tridec-5-ene-10,11,12-triyl tribenzoate and morpholine. ¹H NMR (400 MHZ, CD₃OD) δ 8.30 (s, 2H), 5.72 (td, J=11.6, 5.0 Hz, 1H), 5.40 (d, J=5.6 Hz, 1H), 5.39-5.34 (m, 1H), 4.30 (d, J=10.6 Hz, 1H), 4.13 (d, J=3.3 Hz, 1H), 4.03 (dd, J=10.2, 5.9 Hz, 1H), 3.80-3.69 (m, 5H), 3.68-3.50 (m, 1H), 3.41 (dd, J=10.2, 3.3 Hz, 2H), 3.18 (dd, J=14.1, 4.1 Hz, 1H), 2.82-2.62 (m, 5H), 2.58 (dd, J=12.6, 6.3 Hz, 1H), 2.47 (dd, J=12.6, 7.7 Hz, 1H), 2.40 (dd, J=14.0, 11.9 Hz, 1H), 2.02 (dt, J=13.3, 4.1 Hz, 1H), 1.26 (s, 9H). HRMS (ESI+, m/z): Calc'd for[C₂₀H₃₇N₂O₆S₂+H]⁺: 465.2088 found: 465.2103.

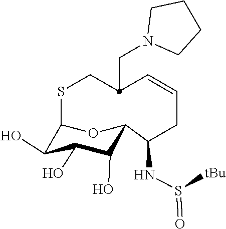

((R)-2-methyl-N-((1R,4S,8R,9R,10R,11S,12R,Z)-10,11,12-trihydroxy-4-(pyrrolidin-1-ylmethyl)-13-oxa-2-thiabicyclo[7.3.1]tridec-5-en-8-yl) propane-2-sulfinamide formate salt (B5): The product B5 was prepared in a similar fashion to product B1 from (1R,4S,8R,9R,10S,11S,12R,Z)-8-(((R)-tert-butylsulfinyl)amino)-4-(hydroxymethyl)-13-oxa-2-thiabicyclo[7.3.1]tridec-5-ene-10,11,12-triyl tribenzoate and pyrrolidine. ¹H NMR (400 MHZ, CD₃OD) ¹H NMR (400 MHZ, MeOD) δ 8.44 (s, 1H), 5.92 (td, J=11.6, 5.0 Hz, 1H), 5.46 (d, J=5.7 Hz, 1H), 5.44-5.37 (m, 1H), 4.26 (d, J=10.7 Hz, 1H), 4.13 (d, J=3.3 Hz, 1H), 4.05 (dd, J=10.2, 5.9 Hz, 1H), 3.86-3.71 (m, 1H), 3.54-3.44 (m, 1H), 3.40 (dd, J=10.2, 3.3 Hz, 1H), 3.37-3.02 (m, 7H), 2.74 (td, J=12.7, 3.7 Hz, 1H), 2.47 (dd, J=13.7, 11.9 Hz, 1H), 2.19-1.94 (m, 5H), 1.26 (s, 9H). HRMS (ESI+, m/z): Calc'd for[C₂₀H₃₆N₂O₅S₂+H]⁺: 449.2138 found: 449.2138

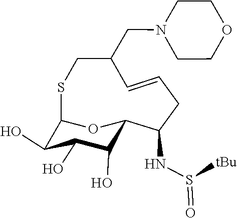 [SMILES: CC(C)(C)[S@@](=O)N[C@@H]1C/C=C/C(CN2CCOCC2)CSC2O[C@H]1[C@@H](O)C(O)[C@H]2O]

(R)-2-methyl-N-((1R,8R,9R,10R,11S,12R,E)-10,11,12-trihydroxy-4-(morpholinomethyl)-13-oxa-2-thiabicyclo[7.3.1]tridec-5-en-8-yl) propane-2-sulfinamide formate salt (B6, single diastereomer, unassigned): The product B6 was prepared in a similar fashion to product B1 from (1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate and morpholine. ¹H NMR (400 MHZ, CD₃OD) δ 8.29 (s, 1H), 5.75 (td, J=13.3, 2.8 Hz, 1H), 5.20 (d, J=5.7 Hz, 1H), 5.12 (dd, J=14.9, 10.3 Hz, 1H), 4.38 (d, J=10.8 Hz, 1H), 4.17 (d, J=3.3 Hz, 1H), 4.01 (dd, J=10.3, 5.7 Hz, 1H), 3.84-3.68 (m, 5H), 3.39 (dd, J=10.3, 3.4 Hz, 1H), 3.21 (dd, J=14.5, 2.9 Hz, 1H), 2.82 (br. s, 4H), 2.70 (br. s, 2H), 2.60-2.46 (m, 2H), 2.41-2.32 (m, 2H), 1.25 (s, 9H). HRMS (ESI+, m/z): Calc'd for[C₂₀H₃₇N₂O₆S₂+H]⁺: 465.2088 found: 465.2094.

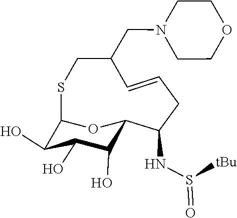 [SMILES: CC(C)(C)[S@@](=O)N[C@@H]1C/C=C/C(CN2CCOCC2)CSC2O[C@H]1[C@@H](O)C(O)[C@H]2O]

(R)-2-methyl-N-((1R,8R,9R,10R,11S,12R,E)-10,11,12-trihydroxy-4-(morpholinomethyl)-13-oxa-2-thiabicyclo[7.3.1]tridec-5-en-8-yl) propane-2-sulfinamide formate salt (B6, single diastereomer, 1:1 mixture of rotamers): The product B7 was prepared in a similar fashion to product B1 from (1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate and morpholine. ¹H NMR (400 MHZ, CD₃OD) δ 8.24 (s, 1H), 5.60-5.35 (m, 2H), 5.20 (app t, J=6.7 Hz, 1H), 4.23 (d, J=10.7 Hz, 0.5H), 4.17 (d, J=2.2, 0.5H), 4.13-4.06 (m, 1.5H), 4.01 (dd, J=10.2, 5.7 Hz, 0.5H), 3.72 (s, 5H), 3.51-3.37 (m, 1.5H), 2.95 (s, 0.5H), 2.83 (s, 1H), 2.79-2.39 (m, 8H), 2.33 (d, J=14.0 Hz, 0.5H), 2.08-1.95 (m, 0.5H), 1.26 (s, ₉H)·HRMS (ESI+, m/z): Calc'd for[C₂₀H₃₇N₂O₆S₂+H]⁺: 465.2088 found: 465.2106.

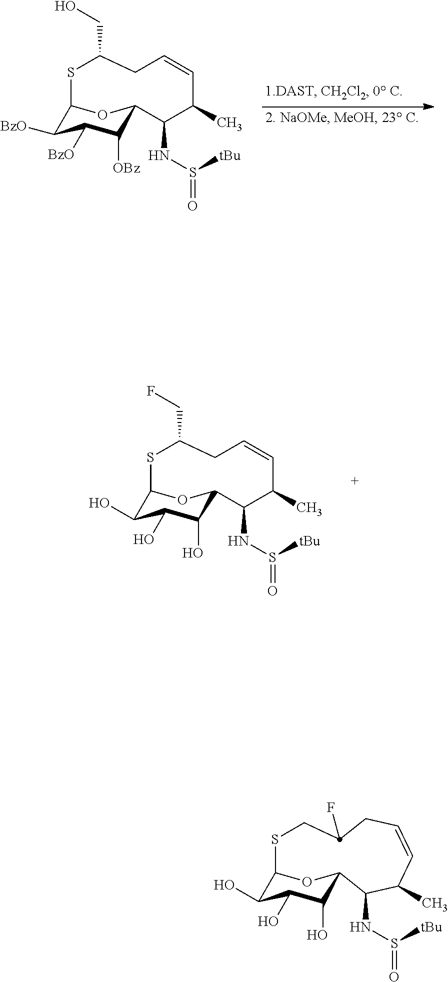

(R)—N-((1R,3S,7R,8R,9R,10R,11S,12R,Z)-3-(fluoromethyl)-10,11,12-trihydroxy-7-methyl-13-oxa-2-thiabicyclo[7.3.1]tridec-5-en-8-yl)-2-methylpropane-2-sulfinamide (C₁); (R)—N-((1R,4R,8R,9R,10R,11R,12S,13R,Z)-4-fluoro-11,12,13-trihydroxy-8-methyl-14-oxa-2-thiabicyclo[8.3.1]tetradec-6-en-9-yl)-2-methylpropane-2-sulfinamide (C₂): (1R,3S,7R,8R,9R,10S,11S,12R,Z)-8-(((R)-tert-butylsulfinyl)amino)-3-(hydroxymethyl)-7-methyl-13-oxa-2-thiabicyclo[7.3.1]tridec-5-ene-10,11,12-triyl tribenzoate (50 mg, 1 Eq, 69 μmol) was dissolved in CH₂Cl₂ (0.35 mL) at 0° C. and DAST (13 mg, 11 μL, 1.2 Eq, 83 μmol) was added. The resultant reaction was slowly warmed to 23° C. and stirred for 4 h. The reaction was quenched upon dilution with CH₂Cl₂ (5 mL) and the addition of H₂O (2 mL). The layers of the resultant biphasic mixture were separated and the organic later was washed with sat. aq. NaCl (1×3 mL) and dried over sodium sulfate. The organic layer was concentrated in vacuo and the crude residue was pushed forward to the next step without additional purification.

The crude residue containing (1R,3S,7R,8R,9R,10S,11S,12R,Z)-8-(((R)-tert-butylsulfinyl)amino)-3-(fluoromethyl)-7-methyl-13-oxa-2-thiabicyclo[7.3.1]tridec-5-ene-10,11,12-triyl tribenzoate and (1R,4R,8R,9R,10R,11S,12S,13R,Z)-9-(((R)-tert-butylsulfinyl)amino)-4-fluoro-8-methyl-14-oxa-2-thiabicyclo[8.3.1]tetradec-6-ene-11,12,13-triyl tribenzoate was added to was dissolved in sodium methoxide (15 mg, 0.55 mL, 0.5 molar, 10 Eq, 0.28 mmol) in MeOH at 23° C. The reaction was stirred at this temperature for 1 h and was neutralized upon addition of formic acid (19 mg, 16 μL, 15 Eq, 0.41 mmol). The reaction mixture was filtered (0.2 uM syringe filter) and purified by prep-HPLC(5-50% MeCN in H₂O with 0.1% HCOOH over 30 minutes) to afford separately product C₁ (4.0 mg, 9.7 μmol, 20% over 2 steps) and product C₂ (9.0 mg, 21 μmol, 40% over 2 steps) as colorless films.

Product C1: ¹H NMR (400 MHZ, CD₃OD) δ 5.78 (d, J=5.9 Hz, 1H), 5.50 (td, J=11.3, 3.5 Hz, 1H), 5.30 (td, J=10.9, 2.0 Hz, 1H), 4.76 (ddd, J=47.8, 9.3, 4.6 Hz, 1H), 4.62 (ddd, J=47.9, 9.3, 6.7 Hz, 1H), 4.37 (dd, J=10.6, 1.2 Hz, 1H), 4.21 (d, J=3.2 Hz, 1H), 4.00 (dd, J=10.3, 6.0 Hz, 1H), 3.54-3.45 (m, 2H), 3.14-2.91 (m, 3H), 2.30 (dq, J=13.4, 2.7 Hz, 1H), 1.30 (s, 9H), 1.03 (d, J=6.8 Hz, 3H). ¹⁹F NMR (376 MHZ, MeOD) 8-215.29. HRMS (ESI+, m/z): Calc'd for[C₁₇H₃₁FNO₅S₂+H]⁺: 412.1622 found: 412.1644.

Product C2: ¹H NMR (400 MHZ, CD₃OD) δ 5.59 (td, J=11.1, 3.3 Hz, 1H), 5.35 (d, J=5.6 Hz, 1H), 5.19 (tt, J=11.0, 2.6 Hz, 1H), 5.17 (d, J=8.9 Hz, 1H), 4.47 (ddddd, J=46.4, 11.1, 8.0, 3.2, 1.5 Hz, 1H), 4.13 (d, J=3.3 Hz, 1H), 4.02 (dd, J=10.5, 5.6 Hz, 1H), 3.91 (d, J=10.3 Hz, 1H), 3.57-3.41 (m, 2H), 3.35 (dd, J=10.6, 3.5 Hz, 1H), 3.14-2.88 (m, 2H), 2.65 (ddd, J=13.7, 11.1, 4.3 Hz, 1H), 2.39 (app ddhept, J=31.7, 15.4, 1.6 Hz, 1H), 1.30 (s, 9H), 1.00 (d, J=6.9 Hz, 3H). ¹⁹F NMR (376 MHZ, MeOD) 8-150.69. HRMS (ESI+, m/z): Calc'd for[C₁₇H₃₁FNO₅S₂+H]⁺: 412.1622 found: 412.1621

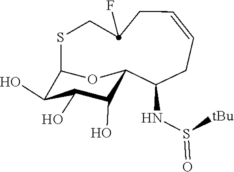 [SMILES: CC(C)(C)[S@@](=O)N[C@@H]1C/C=C\C[C@@H](F)CSC2O[C@H]1[C@@H](O)C(O)[C@H]2O]

(R)—N-((1R,4R,9R,10R,11R,12S,13R,Z)-4-fluoro-11,12,13-trihydroxy-14-oxa-2-thiabicyclo[8.3.1]tetradec-6-en-9-yl)-2-methylpropane-2-sulfinamide (C₃): The product C3 was prepared in a similar fashion to C2 from (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate. ¹H NMR (400 MHZ, CD₃OD) ¹H NMR (400 MHZ, MeOD) δ 5.62 (s, 1H), 5.47 (s, 2H), 5.34 (d, J=5.7 Hz, 1H), 4.66-4.36 (m, 1H), 4.10 (d, J=3.4 Hz, 1H), 4.03 (dd, J=10.5, 5.7 Hz, 1H), 3.94 (d, J=10.0 Hz, 1H), 3.71 (br. s, 1H), 3.42 (app t, J=12.2 Hz, 1H), 3.35 (dd, J=10.5, 3.3 Hz, 1H), 2.91 (br. s, 1H), 2.71 (br. s, 1H), 2.63 (ddd, J=13.4, 11.2, 5.1 Hz, 1H), 2.52 (br. s, 1H), 2.12 (br. s, 1H), 1.25 (s, 9H). LRMS (ESI+, m/z): Calc'd for[C₁₆H₂₈FNO₅S₂+H]⁺: 398.1 found: 398.1

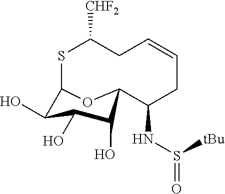 [SMILES: CC(C)(C)[S@@](=O)N[C@@H]1C/C=C\C[C@@H](C(F)F)SC2O[C@H]1C(O)[C@@H](O)[C@H]2O]

(R)—N-((1R,3S,8R,9R,10R,11S,12R,Z)-3-(difluoromethyl)-10,11,12-trihydroxy-13-oxa-2-thiabicyclo[7.3.1]tridec-5-en-8-yl)-2-methylpropane-2-sulfinamide (C4 4:1 mixture of diastereomers): Product C4 was prepared in a similar fashion to C1 from (1R,8R,9R,10S,11S,12R,Z)-8-(((R)-tert-butylsulfinyl)amino)-3-formyl-13-oxa-2-thiabicyclo[7.3.1]tridec-5-ene-10,11,12-triyl tribenzoate. ¹H NMR (400 MHZ, CD₃OD) δ 8 6.20 (td, J=56.4, 1.8 Hz, 0.8H), 6.06 (td, J=56.2, 3.6 Hz, 0.2H), 5.83 (d, J=6.0 Hz, 0.8H), 5.67 (tdd, J=11.3, 5.2, 2.0 Hz, 1.2H), 5.58 (td, J=11.2, 2.9 Hz, 0.8H), 5.52 (d, J=5.9 Hz, 0.2H), 5.37 (d, J=8.7 Hz, 1H), 4.28 (d, J=10.6 Hz, 0.8H), 4.25 (d, J=10.4 Hz, 0.2H), 4.14 (d, J=3.3 Hz, 1H), 4.08 (dd, J=10.4, 5.7 Hz, 0.2H), 4.03 (dd, J=10.2, 6.0 Hz, 0.8H), 3.80-3.70 (m, 1H), 3.48 (dd, J=10.3, 3.4 Hz, 0.8H), 3.43 (dd, J=10.2, 3.4 Hz, 0.2H), 3.21-3.01 (m, 2H), 2.76 (ddd, J=13.2, 11.4, 3.6 Hz, 1H), 2.41 (d, J=14.7 Hz, 0.2H), 2.32 (d, J=11.4 Hz, 0.8H), 2.02-1.96 (m, 1H), 1.26 (s, 9H). ¹⁹F NMR (376 MHZ, CD₃OD* denotes minor diastereomer when resolved) 8-110.83 (d, J=277.9 Hz), *−118.17 (d, J=281.0 Hz), *−119.55 (d, J=281.0 Hz), −123.82 (d, J=277.9 Hz).

LRMS (ESI+, m/z): Calc'd for[C₁₆H₂₇F₂N₂OS+H]⁺: 416.1 found: 416.1.

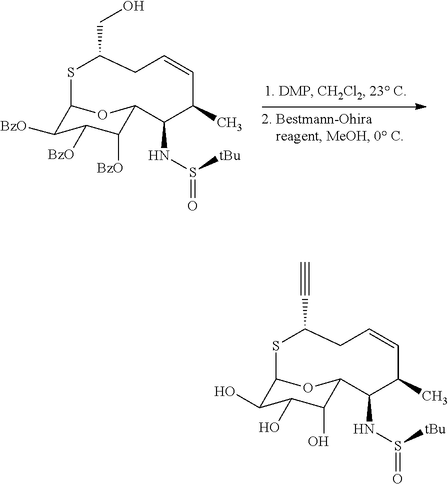

(R)—N-((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl)-2-methylpropane-2-sulfinamide (D1 4:1 mixture of diastereomers, major shown): To a solution of (1R,3S,7R,8R,9R,10S,11S,12R,Z)-8-(((R)-tert-butylsulfinyl)amino)-3-(hydroxymethyl)-7-methyl-13-oxa-2-thiabicyclo[7.3.1]tridec-5-ene-10,11,12-triyl tribenzoate (50 mg, 1 Eq, 69 μmol) in CH₂Cl₂ (0.35 mL) at 23° C. was added Dess-Martin Periodinane (32 mg, 1.1 Eq, 76 μmol). The resultant reaction was stirred at 23° C. for 1 hour. The reaction mixture was quenched upon addition of sat. aq. NaHCO₃ (0.2 mL) and sat. aq. Na₂S₂O₃ (0.2 mL) and the resultant biphasic mixture was stirred vigorously for 30 minutes. The layers were separated, and aq. layer was extracted with CH₂Cl₂ (2×1 mL) and the combined organics were dried over sodium sulfate and concentrated in vacuo to afford (1R,3S,7R,8R,9R,10S,11S,12R,Z)-8-(((R)-tert-butylsulfinyl)amino)-3-formyl-7-methyl-13-oxa-2-thiabicyclo[7.3.1]tridec-5-ene-10,11,12-triyl tribenzoate. The crude residue was used in the next step without additional purification.

To a solution of crude (1R,3S,7R,8R,9R,10S,11S,12R,Z)-8-(((R)-tert-butylsulfinyl)amino)-3-formyl-7-methyl-13-oxa-2-thiabicyclo[7.3.1]tridec-5-ene-10,11,12-triyl tribenzoate (50 mg, 1 Eq, 69 μmol) in MeOH (1.4 mL) at 0° C., was added Bestmann-Ohira Reagent (17 μL, 1.2 Eq, 83 μmol) and K₂CO₃ (29 mg, 3 Eq, 0.21 mmol). The reaction was warmed to 23° C. and stirred for 3 h. The reaction mixture was filtered (0.2 uM syringe filter) and purified by prep-HPLC(30-70% MeCN in H₂O with 0.1% HCOOH over 30 minutes) to afford product D1 (17.6 mg, 44 μmol, 63% over 2 steps). ¹H NMR (400 MHZ, CDCl₃): δ 5.87 (d, J=5.9 Hz, 0.8H), 5.78 (td, J=11.8, 3.3 Hz, 0.2H), 5.48 (d, J=5.9 Hz, 0.2H), 5.49-5.33 (m, 1.1H), 5.31 (td, J=11.0, 1.9 Hz, 0.8H), 4.32 (dd, J=10.5, 1.1 Hz, 0.2H), 4.27 (dd, J=10.5, 1.0 Hz, 0.8H), 4.21 (app td, J=3.3, 1.0 Hz, 1H), 4.08 (0.8 J=10.3, 6.0 Hz, 1H), 4.02 (dd, J=10.3, 5.9 Hz, 0.2H), 3.79 (ddd, J=12.4, 4.7, 2.5 Hz, 1H), 3.50 (dd, J=10.5, 3.4 Hz, 1H), 3.41 (dd, J=10.3, 3.5 Hz, 1H), 3.18-3.00 (m, 1H), 2.97 (dp, J=10.1, 3.3 Hz, 1H), 2.82 (d, J=2.4 Hz, 0.8H), 2.77 (d, J=2.5 Hz, 0.2H), 2.47-2.37 (m, 0.8H), 2.33-2.23 (m, 0.2H), 1.30 (d, J=1.8 Hz, 9H), 1.02 (app dd, J=6.8, 2.9 Hz, 3H). HRMS (ESI+, m/z): Calc'd for[C₁₈H₂₉NO₅S₂+H]⁺: 404.1560 found: 404.1562.

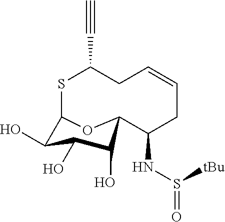 [SMILES: C#C[C@@H]1C/C=C\C[C@@H](N[S@](=O)C(C)(C)C)[C@H]2OC(S1)[C@H](O)[C@H](O)C2O]

(R)—N-((1R,3S,8R,9R,10R,11S,12R,Z)-3-ethynyl-10,11,12-trihydroxy-13-oxa-2-thiabicyclo[7.3.1]tridec-5-en-8-yl)-2-methylpropane-2-sulfinamide (D2, 4:1 mixture of diastereomers): Product D2 was prepared in a similar fashion to product D1 from (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate. ¹H NMR (400 MHZ, CD₃OD) δ 8 5.93-5.89 (m, 0.2H), 5.88 (d, J=6.0 Hz, 0.8H), 5.74-5.67 (m, 0.2H), 5.64 (tdd, J=11.4, 5.0, 1.9 Hz, 0.8H), 5.54 (td, J=11.5, 3.2 Hz, 0.8H), 5.47 (d, J=6.0 Hz, 0.2H), 4.27 (d, J=10.6 Hz, 0.2H), 4.22 (d, J=10.4 Hz, 0.8H), 4.14 (d, J=3.6 Hz, 1H), 4.09 (dd, J=10.3, 6.0 Hz, 0.8H), 4.04 (dd, J=10.3, 5.9 Hz, 0.2H), 3.80 (ddd, J=12.4, 4.6, 2.4 Hz, 1H), 3.74 (dt, J=10.6, 3.4 Hz, 1H), 3.39 (dd, J=10.2, 3.5 Hz, 1H), 3.20-3.14 (m, 0.2H), 3.08 (dt, J=13.8, 12.2 Hz, 0.8H), 2.83 (d, J=2.4 Hz, 0.8H), 2.78 (d, J=2.5 Hz, 0.2H), 2.79-2.67 (m, 1H), 2.44-2.33 (m, 0.8H), 2.26 (d, J=13.9 Hz, 0.2H), 1.95 (dt, J=13.4, 4.3 Hz, 1H), 1.26 (s, 9H). HRMS (ESI+, m/z): Calc'd for[C₁₇H₂₈NO₅S₂+H]⁺: 390.1403 found: 390.1305.

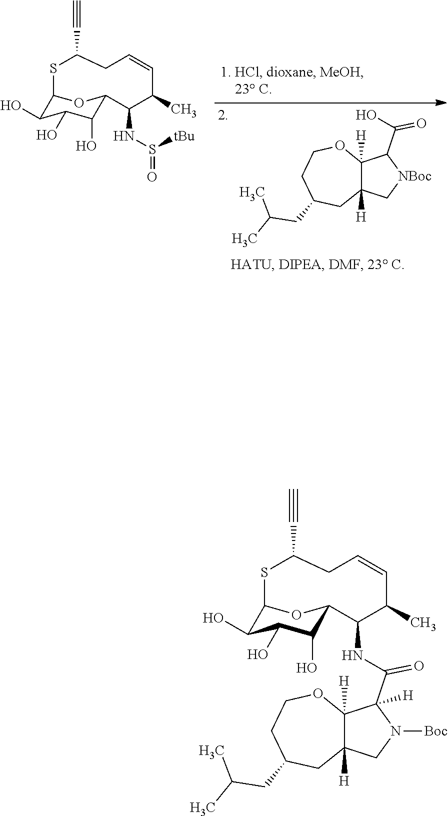 [SMILES: C#C[C@@H]1C/C=C\[C@@H](C)[C@@H](N[S@](=O)C(C)(C)C)C2OC(S1)C(O)C(O)C2O ; CC(C)C[C@@H]1CCO[C@H]2C(C(=O)O)N(C(=O)OC(C)(C)C)C[C@@H]2C1 ; C#C[C@@H]1C/C=C\[C@@H](C)[C@@H](NC(=O)[C@@H]2[C@@H]3OCC[C@@H](CC(C)C)C[C@H]3CN2C(=O)OC(C)(C)C)[C@H]2OC(S1)[C@H](O)[C@H](O)C2O]

tert-butyl (4S,5aS,8S,8aR)-8-(((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl) carbamoyl)-4-isobutyloctahydro-7H-oxepino[2,3-c]pyrrole-7-carboxylate (D3 4:1 mixture of diastereomers, 1:1 mixture of rotamers): (R)—N-((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl)-2-methylpropane-2-sulfinamide (17.6 mg, 1 Eq, 43.6 μmol) was dissolved in MeOH (0.3 mL) and a solution of hydrogen chloride in dioxane (200 μL, 4.0 M, 18.3 Eq) was added. The reaction mixture was maintained at rt for 45 min. The reaction was concentrated in vacuo to afford (1R,3S,7R,8R,9R,10R,11S,12R,Z)-8-amino-3-ethynyl-7-methyl-13-oxa-2-thiabicyclo[7.3.1]tridec-5-ene-10,11,12-triol hydrochloride as a colorless film.

To a solution of (4S,5aS,8aR)-7-(tert-butoxycarbonyl)-4-isobutyloctahydro-2H-oxepino[2,3-c]pyrrole-8-carboxylic acid (20.1 mg, 1.4 Eq, 58.8 μmol) and HATU (22 mg, 1.4 Eq, 59 μmol) in DMF (0.6 mL) was added DIPEA (19.0 mg, 25.6 μL, 3.5 Eq, 147 μmol). The reaction was stirred for 15 minutes and added to (1R,3S,7R,8R,9R,10R,11S,12R,Z)-8-amino-3-ethynyl-7-methyl-13-oxa-2-thiabicyclo[7.3.1]tridec-5-ene-10,11,12-triol hydrochloride (14.6 mg, 1 Eq, 43 μmol). The reaction was stirred for 16 hours. The reaction mixture was filtered (0.2 uM syringe filter) and purified by prep-HPLC(30-70% MeCN in H₂O with 0.1% HCOOH over 30 minutes) to afford product D3 (22 mg, 60% yield over 2 steps) as a white solid. ¹H NMR (400 MHZ, CD₃OD) δ 8.01-7.47 (m, 1H), 5.89 (app dd, J=6.0, 1.9 Hz, 0.8H), 5.81 (tt, J=12.1, 3.5 Hz, 0.2H), 5.63-5.39 (m, 2H), 4.50 (app dd, J=7.0, 6.5 Hz, 0.5H), 4.39 (app dd, J=8.2, 4.2 Hz, 0.5H), 4.25-3.98 (m, 4.2H), 3.98-3.85 (m, 2H), 3.85-3.64 (m, 2.8H), 3.44-3.32 (m, 1H), 3.23-2.95 (m, 2H), 2.89 (t, J=10.5 Hz, 1H), 2.83 (app t, J=2.5 Hz, 0.8H), 2.79 (app t, J=2.5 Hz, 0.2H), 2.59-2.35 (m, 1.8H), 2.34-2.25 (m, 0.2H), 2.05-1.86 (m, 1H), 1.81-1.54 (m, 4H), 1.45 (d, J=1.9 Hz, 9H), 1.15 (hept, J=7.0 Hz, 2H), 1.04 (app q, J=4.6 Hz, 3H), 0.99-0.91 (m, 1H), 0.88 (app d, J=6.5 Hz, 6H). HRMS (ESI+) m/z: Calc'd for[C₃₂H₅₀N₂O₈S+H]⁺: 623.3361 Found: 623.3363

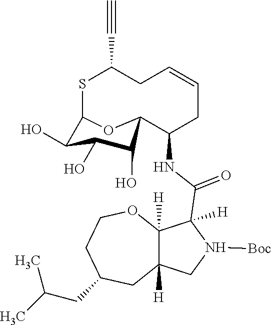 [SMILES: C#C[C@@H]1C/C=C\C[C@@H](NC(=O)[C@@H]2[C@@H]3OCC[C@@H](CC(C)C)C[C@H]3CN2C(=O)OC(C)(C)C)[C@H]2OC(S1)[C@H](O)[C@H](O)C2O]

tert-butyl (4S,5aS,8S,8aR)-8-(((1R,3S,8R,9R,10R,11S,12R,Z)-3-ethynyl-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (D4, 4:1 mixture of diastereomers, 1:1 mixture of rotamers): Product D4 was prepared in a similar fashion to product D3 from (R)—N-((1R,3S,8R,9R,10R,11S,12R,Z)-3-ethynyl-10,11,12-trihydroxy-13-oxa-2-thiabicyclo[7.3.1]tridec-5-en-8-yl)-2-methylpropane-2-sulfinamide. ¹H NMR (400 MHZ, CD₃OD) δ 8.00 (app dd, J=13.4, 8.0 Hz, 1H), 5.90 (dd, J=6.1, 1.9 Hz, 0.8H), 5.89-5.65 (m, 1H), 5.59-5.45 (m, 1H), 5.30 (d, J=5.2 Hz, 0.2H), 4.50-3.98 (m, 5H), 3.96-3.83 (m, 2H), 3.85-3.69 (m, 3H), 3.38 (app ddd, J=13.0, 6.8, 2.9 Hz, 1H), 3.24-3.11 (m, 0.2H), 3.10 (q, J=12.8 Hz, 0.8H), 2.89 (q, J=10.8 Hz, 1H), 2.83 (t, J=2.2 Hz, 0.8H), 2.78 (t, J=2.5 Hz, 0.2H), 2.71-2.53 (m, 1H), 2.50 (t, J=10.2 Hz, 1H), 2.39 (d, J=13.9 Hz, 0.8H), 2.26 (d, J=13.9 Hz, 0.2H), 2.08 (t, J=15.8 Hz, 1H), 1.94 (t, J=14.5 Hz, 1H), 1.76 (d, J=14.4 Hz, 1H), 1.64 (td, J=13.7, 8.1 Hz, 3H), 1.44 (app d, J=9.9 Hz, 9H), 1.15 (h, J=6.8 Hz, 2H), 0.99-0.91 (m, 1H), 0.88 (app d, J=6.5 Hz, 6H). LRMS (ESI+) m/z: Calc'd for[C₃₁H₄₂N₂O₈S+H]⁺: 609.3 Found: 609.3

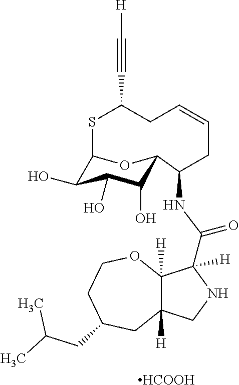 [SMILES: C#C[C@@H]1C/C=C\C[C@@H](NC(=O)[C@H]2NC[C@@H]3C[C@H](CC(C)C)CCO[C@H]32)[C@H]2OC(S1)[C@H](O)[C@H](O)C2O ; "·HCOOH"]

(4S,5aS,8S,8aR)—N-((1R,3S,8R,9R,10R,11S,12R,Z)-3-ethynyl-10,11,12-trihydroxy-13-oxa-2-thiabicyclo[7.3.1]tridec-5-en-8-yl)-4-isobutyloctahydro-2H-oxepino[2,3-c]pyrrole-8-carboxamide formate salt (FSA1607077; 4:1 mixture of diastereomers, major shown): tert-butyl (4S,5aS,8S,8aR)-8-(((1R,3S,8R,9R,10R,11S,12R,Z)-3-ethynyl-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (1.0 mg, 1.6 μmol) was dissolved in 1N HCl in MeOH (1 mL) at 23° C. and maintained at this temperature for 2 hours. The reaction mixture was concentrated in vacuo and purified by prep-HPLC(5-40% MeCN in H₂O with 0.1% HCOOH over 30 minutes) to afford FSA1607077. ¹H NMR (400 MHZ, CD₃OD) δ 8.35 (s, 1H), 5.95-5.87 (m, 0.2H), 5.91 (d, J=6.1 Hz, 0.8 H), 5.78-5.62 (m, 1H), 5.57 (td, J=11.5, 3.2 Hz, 0.8H), 5.50 (d, J=6.0 Hz, 0.2H), 4.39-4.28 (m, 3H), 4.26-4.19 (m, 0.2H), 4.22 (d, J=11.0 Hz, 0.8H), 4.10 (dd, J=10.2, 6.0 Hz, 0.8H), 4.04 (dd, J=10.9, 6.5 Hz, 0.2H), 3.99 (dd, J=11.6, 3.9 Hz, 1H), 3.87-3.74 (m, 3H), 3.59 (dd, J=11.3, 7.3 Hz, 1H), 3.39 (dd, J=10.3, 3.4 Hz, 1H), 3.24-3.11 (m, 0.2H), 3.10 (q, J=12.4 Hz, 0.8H), 2.89 (t, J=11.8 Hz, 1H), 2.85 (d, J=2.4 Hz, 0.8H), 2.80 (d, J=2.6 Hz, 0.2H), 2.65 (td, J=12.5, 3.8 Hz, 1H), 2.40 (d, J=14.2 Hz, 1H), 2.35-2.25 (m, 1H), 2.14-2.04 (m, 1H), 2.01 (d, J=13.7 Hz, 1H), 1.83-1.60 (m, 4H), 1.19 (td, J=6.9, 3.8 Hz, 2H), 1.01-0.92 (m, 1H), 0.90 (d, J=6.5 Hz, 6H). HRMS (ESI+, m/z): Calc'd for[C₂₆H₄₀N₂O₆S+H]⁺: 509.2680. Found: 509.2700.

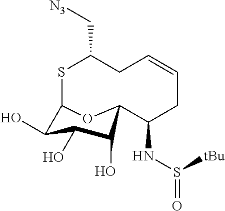 [SMILES: CC(C)(C)[S@@](=O)N[C@@H]1C/C=C\C[C@@H](CN=[N+]=[N-])SC2O[C@H]1C(O)[C@@H](O)[C@H]2O]

(R)—N-((1R,3S,8R,9R,10R,11S,12R,Z)-3-(azidomethyl)-10,11,12-trihydroxy-13-oxa-2-thiabicyclo[7.3.1]tridec-5-en-8-yl)-2-methylpropane-2-sulfinamide (E1 3:2 mixture of diastereomers): To a solution of (1R,8R,9R,10S,11S,12R,Z)-8-(((R)-tert-butylsulfinyl)amino)-3-(hydroxymethyl)-13-oxa-2-thiabicyclo[7.3.1]tridec-5-ene-10,11,12-triyl tribenzoate (45 mg, 1 Eq, 0.06 mmol) in CH₂Cl₂ (0.65 mL) at 23° C. was added Et₃N(11 uL, 1.2 Eq, 0.07 mmol) followed by methanesulfonyl chloride (5.9 μL, 1.2 Eq, 0.07 mmol). The resultant reaction was stirred at 23° C. for 1 hour and was then diluted with CH₂Cl₂ (2 mL). The reaction mixture was washed with sat. aq. NaCl (2 mL) and the organic layer was dried over sodium sulfate and concentrated in vacuo to afford crude (1R,8R,9R,10S,11S,12R,Z)-8-(((R)-tert-butylsulfinyl)amino)-3-(((methylsulfonyl)oxy)methyl)-13-oxa-2-thiabicyclo[7.3.1]tridec-5-ene-10,11,12-triyl tribenzoate. The crude residue was used directly in the next step without additional purification.

(1R,8R,9R,10S,11S,12R,Z)-8-(((R)-tert-butylsulfinyl)amino)-3-(((methylsulfonyl)oxy)methyl)-13-oxa-2-thiabicyclo[7.3.1]tridec-5-ene-10,11,12-triyl tribenzoate (50 mg, 1 Eq, 64 μmol) was dissolved in DMF (0.32 mL) and sodium azide (21 mg, 5 Eq, 0.32 mmol) was added and the reaction mixture was heated to 50° C. for 16 h. The reaction was cooled to 23° C. and treated with sodium methoxide (0.5 N in MeOH, 0.64 mL). The reaction was stirred for 30 minutes and concentrated under reduced pressure. The reaction mixture was filtered (0.2 uM syringe filter) and purified by prep-HPLC(30-70% MeCN in H₂O with 0.1% HCOOH over 30 minutes) to afford product E1 (19 mg, 50 μmol, 78% yield over 2 steps). ¹H NMR (400 MHZ, CD₃OD) δ 5.76 (d, J=6.0 Hz, 0.6H), 5.71-5.55 (m, 2H), 5.52 (d, J=5.9 Hz, 0.4H), 5.33 (app dd, J=8.6, 4.2 Hz, 1H), 4.29 (d, J=10.5 Hz, 0.6H), 4.25 (d, J=10.7 Hz, 0.4H), 4.13 (d, J=3.3 Hz, 1H), 4.05 (app ddd, J=10.3, 7.1, 5.9 Hz, 1H), 3.81 (dd, J=12.5, 6.3 Hz, 0.6H), 3.78-3.69 (m, 1H), 3.65 (dd, J=12.6, 7.1 Hz, 0.6H), 3.60-3.39 (m, 1.8H), 3.14 (td, J=13.2, 6.7 Hz, 0.4H), 3.06-2.84 (m, 1.6H), 2.76 (app ddt, J=14.0, 10.6, 7.9 Hz, 1H), 2.22 (app dt, J=11.5, 3.9 Hz, 1H), 1.95 (app dt, J=14.1, 3.4 Hz, 1H), 1.25 (s, 9H). HRMS (ESI+, m/z): Calc'd for[C₁₆H₂₈N₄O₅S₂+H]⁺: 421.1574 found: 421.1580.

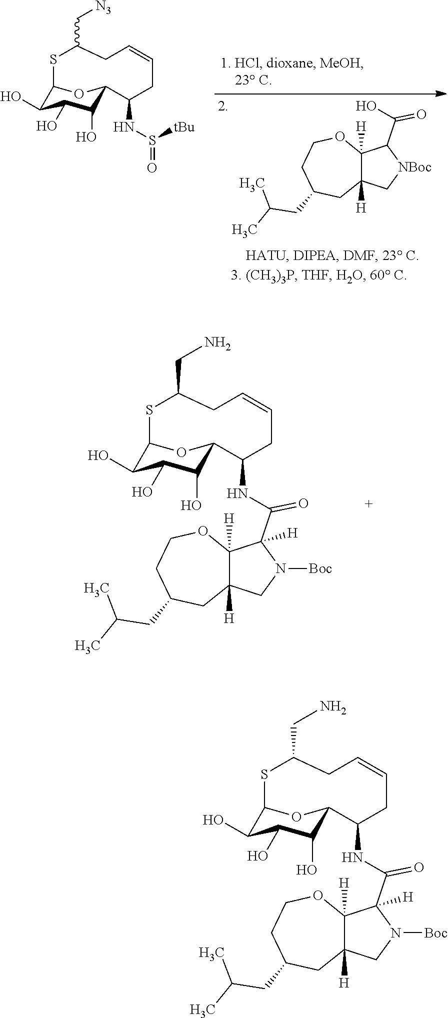 [SMILES: CC(C)(C)[S@@](=O)N[C@@H]1C/C=C\CC(CN=[N+]=[N-])SC2O[C@H]1C(O)[C@@H](O)[C@H]2O ; CC(C)C[C@@H]1CCO[C@H]2C(C(=O)O)N(C(=O)OC(C)(C)C)C[C@@H]2C1 ; CC(C)C[C@@H]1CCO[C@@H]2[C@@H](C1)CN(C(=O)OC(C)(C)C)[C@@H]2C(=O)N[C@@H]1C/C=C\C[C@H](CN)SC2O[C@H]1C(O)[C@@H](O)[C@H]2O ; CC(C)C[C@@H]1CCO[C@@H]2[C@@H](C1)CN(C(=O)OC(C)(C)C)[C@@H]2C(=O)N[C@@H]1C/C=C\C[C@@H](CN)SC2O[C@H]1C(O)[C@@H](O)[C@H]2O]

tert-butyl (4S,5aS,8S,8aR)-8-(((1R,3R,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate formate salt (E2 1:1 mixture of rotamers); tert-butyl (4S,5aS,8S,8aR)-8-(((1R,3S,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate formate salt (E3 1:1 mixture of rotamers): (R)—N-((1R,3S,8R,9R,10R,11S,12R,Z)-3-(azidomethyl)-10,11,12-trihydroxy-13-oxa-2-thiabicyclo[7.3.1]tridec-5-en-8-yl)-2-methylpropane-2-sulfinamide (24 mg, 1 Eq, 63 μmol) was dissolved in MeOH (0.3 mL) and a solution of hydrogen chloride in dioxane (200 μL, 4.0 M, 18.3) was added at 23° C. The reaction mixture was maintained at rt for 45 min at which point LCMS confirmed the reaction was complete. The reaction was concentrated in vacuo to afford (1R,8R,9R,10R,11S,12R,Z)-8-amino-3-(azidomethyl)-13-oxa-2-thiabicyclo[7.3.1]tridec-5-ene-10,11,12-triol hydrochloride as a colorless film.

To a solution of (4S,5aS,8aR)-7-(tert-butoxycarbonyl)-4-isobutyloctahydro-2H-oxepino[2,3-c]pyrrole-8-carboxylic acid (22 mg, 1 Eq, 63 μmol) and HATU (24 mg, 1.0 Eq, 63 μmol) in DMF (0.63 mL) at 23° C., was added DIPEA (28 μL, 2.5 Eq, 0.16 mmol). The reaction was stirred for 15 minutes at this temperature and was then added to (1R,8R,9R,10R,11S,12R,Z)-8-amino-3-(azidomethyl)-13-oxa-2-thiabicyclo[7.3.1]tridec-5-ene-10,11,12-triol hydrochloride (20 mg, 1 Eq, 63 μmol). The reaction was stirred for 16 hours at 23° C. The DMF was removed under a stream of argon and the crude residue was dissolved in CH₂Cl₂ (1 mL) and washed with H₂O (1 mL). The aq. layer was extracted with CH₂Cl₂ (1 mL) and the combined organics were dried over sodium sulfate and concentrated in vacuo to afford crude tert-butyl (4S,5aS,8aR)-8-(((1R,8R,9R,10R,11S,12R,Z)-3-(azidomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate. This crude residue was used in the next step without further purification.

To a solution of tert-butyl (4S,5aS,8aR)-8-(((1R,8R,9R,10R,11S,12R,Z)-3-(azidomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (30 mg, 1 Eq, 47 μmol) in THF (0.70 mL) and H₂O (0.23 mL) was added trimethylphosphine (59 μL, 1M in THF, 1.25 Eq, 59 μmol). The resulting reaction mixture was heated to 66° C. for 2 h. The reaction mixture was concentrated in vacuo. The crude residue was dissolved in methanol, filtered (0.2 uM syringe filter), and purified by prep-HPLC(30-70% MeCN in H₂O with 0.1% HCOOH over 30 minutes) to afford product separately product E2 and product E3.

Product E2: 1H NMR (400 MHZ, CD₃OD) δ 8 9.74 (s, 1H), 7.20 (dd, J=14.2, 9.2 Hz, 1H), 7.00 (t, J=12.0 Hz, 1H), 6.91 (d, J=5.5 Hz, 1H), 5.77 (d, J=8.1 Hz, 0.5H), 5.71 (d, J=8.2 Hz, 0.5H), 5.69-5.52 (m, 3H), 5.48 (td, J=10.2, 5.7 Hz, 1H), 5.37-5.25 (m, 2H), 5.20-5.09 (m, 2H), 4.94 (s, 1H), 4.80 (ddd, J=10.6, 7.5, 3.4 Hz, 1H), 4.66 (d, J=14.1 Hz, 1H), 4.53 (t, J=11.1 Hz, 1H), 4.28 (q, J=10.8 Hz, 1H), 4.15-3.99 (m, 1H), 3.98-3.83 (m, 1H), 3.63-3.44 (m, 2H), 3.34 (t, J=13.8 Hz, 1H), 3.21-2.96 (m, 4H), 2.85 (d, J=10.3 Hz, 10H), 2.55 (hept, J=7.0 Hz, 2H), 2.35 (d, J=19.7 Hz, 1H), 2.29 (app dd, J=6.5, 1.0 Hz, 6H). HRMS (ESI+, m/z): Calc'd for[C₃₀H₅₁N₃O₈S+H]⁺: 614.3470 found: 614.3482.

Product E3: ¹H NMR (400 MHZ, CD₃OD) δ 8.31 (s, 1H), 5.84-5.66 (m, 2H), 5.59 (t, J=11.3 Hz, 1H), 4.37 (d, J=8.1 Hz, 0.5H), 4.30 (d, J=8.2 Hz, 0.5H), 4.28-4.03 (m, 4H), 3.98-3.83 (m, 2H), 3.75 (ddt, J=12.3, 8.7, 4.1 Hz, 2H), 3.45 (dt, J=7.1, 3.2 Hz, 1H), 3.35-3.22 (m, 2H), 3.09 (d, J=11.9 Hz, 1H), 2.97 (q, J=11.8 Hz, 1H), 2.87 (q, J=10.6 Hz, 1H), 2.69-2.56 (m, 1H), 2.50 (p, J=10.8 Hz, 1H), 2.21-2.03 (m, 2H), 1.98-1.88 (m, 1H), 1.81-1.55 (m, 4H), 1.45 (app d, J=9.5 Hz, 9H), 1.15 (h, J=6.8 Hz, 2H), 0.99-0.91 (m, 1H), 0.91-0.84 (m, 6H). HRMS (ESI+, m/z): Calc'd for[C₃₀H₅₁N₃O₈S+H]⁺: 614.3470 found: 614.3474.

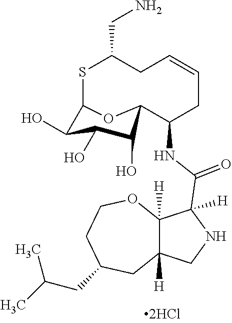 [SMILES: CC(C)C[C@@H]1CCO[C@@H]2[C@H](CN[C@@H]2C(=O)N[C@@H]2C/C=C\C[C@@H](CN)SC3O[C@H]2C(O)[C@@H](O)[C@H]3O)C1]

(4S,5aS,8S,8aR)—N-((1R,3S,8R,9R,10R,11S,12R,Z)-3-(aminomethyl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide hydrochloride salt (FSA1608083): tert-butyl (4S,5aS,8aR)-8-(((1R,3S,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (1.4 mg, 1 Eq, 2.3 μmol) formate salt was dissolved in 1N HCl in MeOH (1 mL) at 23° C. and maintained at this temperature for 2 hours. The reaction mixture was concentrated in vacuo to afford FSA1608083. ¹H NMR (400 MHZ, CD₃OD) δ 18 8.34 (d, J=8.5 Hz, 1H), 5.74 (d, J=6.0 Hz, 1H), 5.73-5.66 (m, 1H), 5.62 (td, J=11.1, 3.0 Hz, 1H), 4.43-4.31 (m, 3H), 4.31-4.25 (m, 1H), 4.11 (dd, J=10.1, 6.0 Hz, 1H), 4.01 (dt, J=12.0, 3.8 Hz, 1H), 3.84 (d, J=3.4 Hz, 1H), 3.83-3.76 (m, 1H), 3.62 (dd, J=11.0, 7.3 Hz, 1H), 3.48 (dd, J=10.2, 3.3 Hz, 1H), 3.33-3.24 (m, 2H), 3.19-3.06 (m, 1H), 3.02-2.93 (m, 1H), 2.91 (d, J=11.8 Hz, 1H), 2.69-2.50 (m, 1H), 2.37-2.23 (m, 1H), 2.18-2.06 (m, 2H), 2.03-1.97 (m, 1H), 1.84-1.58 (m, 4H), 1.19 (td, J=6.7, 4.2 Hz, 2H), 1.03-0.92 (m, 1H), 0.90 (d, J=6.5 Hz, 6H). HRMS (ESI+, m/z): Calc'd for[C₂₅H₄₃N₃O₆S+H]⁺: 514.2945 found: 514.2953.

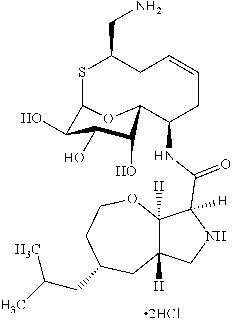 [SMILES: CC(C)C[C@@H]1CCO[C@@H]2[C@H](CN[C@@H]2C(=O)N[C@@H]2C/C=C\C[C@H](CN)SC3O[C@H]2C(O)[C@@H](O)[C@H]3O)C1]

(4S,5aS,8S,8aR)—N-((1R,3R,8R,9R,10R,11S,12R,Z)-3-(aminomethyl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide hydrochloride salt (FSA1608082): tert-butyl (4S,5aS,8aR)-8-(((1R,3R,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (1.4 mg, 1 Eq, 2.3 μmol) formate salt was dissolved in 1N HCl in MeOH (1 mL) at 23° C. and maintained at this temperature for 2 hours. The reaction mixture was concentrated in vacuo to afford FSA1608082. ¹H NMR (400 MHZ, CD₃OD) δ 5.77 (td, J=11.2, 4.9 Hz, 1H), 5.63 (td, J=12.5, 2.7 Hz, 1H), 5.53 (d, J=5.9 Hz, 1H), 4.44-4.33 (m, 3H), 4.27 (d, J=10.9 Hz, 1H), 4.09 (dd, J=10.2, 5.9 Hz, 1H), 4.01 (dt, J=12.0, 3.8 Hz, 1H), 3.90-3.75 (m, 2H), 3.68-3.51 (m, 2H), 3.41 (dd, J=10.2, 3.4 Hz, 1H), 3.33-3.22 (m, 2H), 3.13 (dd, J=13.5, 10.4 Hz, 1H), 2.92 (t, J=11.9 Hz, 1H), 2.68 (td, J=13.5, 3.7 Hz, 1H), 2.38-2.23 (m, 1H), 2.23-2.06 (m, 2H), 2.02 (d, J=14.1 Hz, 1H), 1.83-1.57 (m, 4H), 1.19 (td, J=6.8, 4.4 Hz, 2H), 1.05-0.92 (m, 1H), 0.90 (d, J=6.6 Hz, 6H). HRMS (ESI+, m/z): Calc'd for[C₂₅H₄₃N₃O₆S+H]⁺: 514.2945 found: 514.2950

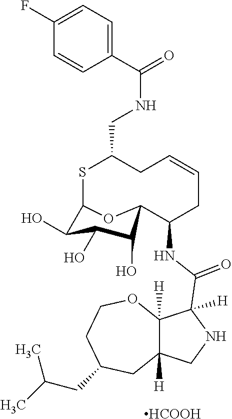 [SMILES: CC(C)C[C@@H]1CCO[C@@H]2[C@H](CN[C@@H]2C(=O)N[C@@H]2C/C=C\C[C@@H](CNC(=O)c3ccc(F)cc3)SC3O[C@H]2C(O)[C@@H](O)[C@H]3O)C1 ; "·HCOOH"]

(4S,5aS,8S,8aR)—N-((1R,3S,8R,9R,10R,11S,12R,Z)-3-((4-fluorobenzamido)methyl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide formate salt (FSA1608088): To a solution of 4-fluorobenzoic acid (0.41 mg, 1.2 Eq, 2.9 μmol) and HATU (1.1 mg, 1.2 Eq, 2.9 μmol) at 23° C., was added DIPEA (0.79 mg, 1.1 μL, 2.5 Eq, 6.1 μmol). The reaction mixture was stirred for 15 minutes and added to solid tert-butyl (4S,5aS,8aR)-8-(((1R,3S,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate (1.5 mg, 1 Eq, 2.4 μmol). The reaction was maintained at 23° C. for 2 h and was then diluted with EtOAc (1 mL) and the organic layer was washed with sat. aq. NaCl (2×1 mL). The organic layer was dried over sodium sulfate and concentrated in vacuo to afford tert-butyl (4S,5aS,8aR)-8-(((1R,3S,8R,9R,10R,11S,12R,Z)-3-((4-fluorobenzamido)methyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate in a crude residue. This residue was dissolved in 1N HCl in MeOH (1 mL) and maintained at 23° C. for 2 hours. The reaction mixture was concentrated in vacuo and purified by prep-HPLC(5-40% MeCN in H₂O with 0.1% HCOOH over 30 minutes) to afford FSA1608088. ¹H NMR (400 MHZ, CD₃OD) δ 8.45 (s, 1H), 7.99-7.77 (m, 2H), 7.31-7.05 (m, 2H), 5.80 (d, J=6.0 Hz, 1H), 5.63 (d, J=7.6 Hz, 2H), 4.38-4.21 (m, 4H), 4.09 (dd, J=10.2, 6.0 Hz, 1H), 3.99 (dt, J=11.7, 3.5 Hz, 1H), 3.84-3.74 (m, 3H), 3.66 (dd, J=13.8, 9.4 Hz, 1H), 3.60-3.50 (m, 1H), 3.50-3.43 (m, 1H), 3.18-3.04 (m, 1H), 3.01-2.89 (m, 1H), 2.89-2.75 (m, 1H), 2.67 (t, J=11.4 Hz, 1H), 2.29 (s, 1H), 2.19 (d, J=13.3 Hz, 1H), 2.09 (d, J=12.3 Hz, 1H), 2.00 (d, J=13.4 Hz, 1H), 1.83-1.59 (m, 4H), 1.18 (q, J=7.0 Hz, 2H), 1.01-0.91 (m, 1H), 0.89 (app d, J=6.6 Hz, 6H). ¹⁹F NMR (376 MHz, MeOD) 8-110.64. HRMS (ESI+, m/z): Calc'd for[C₃₂H₄₄FN₃O₇S+H]⁺: 636.3113 found: 636.3112

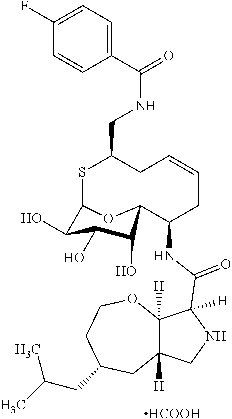 [SMILES: CC(C)C[C@@H]1CCO[C@@H]2[C@H](CN[C@@H]2C(=O)N[C@@H]2C/C=C\C[C@H](CNC(=O)c3ccc(F)cc3)SC3O[C@H]2C(O)[C@@H](O)[C@H]3O)C1 ; "·HCOOH"]

(4S,5aS,8S,8aR)—N-((1R,3R,8R,9R,10R,11S,12R,Z)-3-((4-fluorobenzamido)methyl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide formate salt (FSA1608089): The product FSA1608089 was prepared in a similar fashion to FSA1608088 from (4S,5aS,8aR)-8-(((1R,3R,8R,9R,10R,11S,12R,Z)-3-(aminomethyl)-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate. ¹H NMR (400 MHZ, CD₃OD) δ 8.47 (s, 1H), 7.96-7.81 (m, 2H), 7.19 (t, J=8.7 Hz, 2H), 5.81 (td, J=11.4, 2.5 Hz, 1H), 5.72 (td, J=11.0, 4.4 Hz, 1H), 5.49 (d, J=5.9 Hz, 1H), 4.42-4.21 (m, 4H), 4.12-3.95 (m, 2H), 3.85-3.76 (m, 2H), 3.70 (dd, J=11.1, 5.0 Hz, 1H), 3.65-3.51 (m, 3H), 3.43 (d, J=10.1 Hz, 1H), 3.22-3.08 (m, 1H), 2.84 (t, J=11.7 Hz, 1H), 2.70 (td, J=12.3, 2.9 Hz, 1H), 2.35-2.26 (m, 1H), 2.21 (d, J=14.3 Hz, 1H), 2.09 (d, J=12.9 Hz, 1H), 2.00 (d, J=13.2 Hz, 1H), 1.83-1.61 (m, 4H), 1.19 (h, J=7.8, 4.0 Hz, 2H), 1.00-0.92 (m, 1H), 0.92-0.83 (m, 6H). ¹⁹F NMR (376 MHz, MeOD) 8-110.57. HRMS (ESI+, m/z): Calc'd for[C₃₂H₄₄FN₃O₇S+H]⁺: 636.3113 found: 636.3114

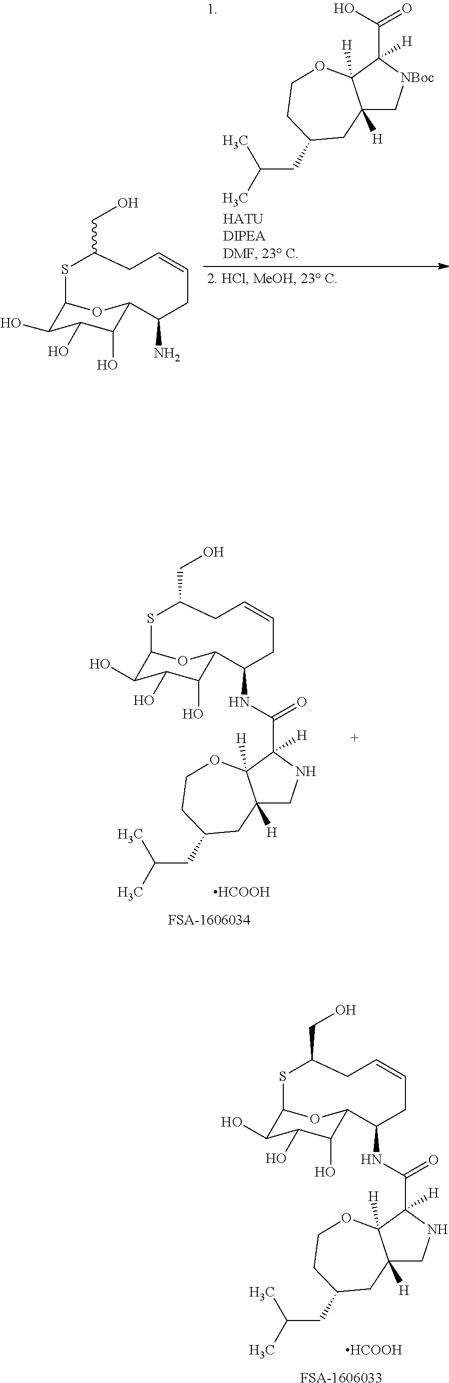

(4S,5aS,8S,8aR)-4-isobutyl-N-((1R,3R,8R,9R,10R,11S,12R,Z)-10,11,12-trihydroxy-3-(hydroxymethyl)-13-oxa-2-thiabicyclo[7.3.1]tridec-5-en-8-yl) octahydro-2H-oxepino[2,3-c]pyrrole-8-carboxamide hydrochloride salt (FSA1606033); (4S,5aS,8S,8aR)-4-isobutyl-N-((1R,3S,8R,9R,10R,11S,12R,Z)-10,11,12-trihydroxy-3-(hydroxymethyl)-13-oxa-2-thiabicyclo[7.3.1]tridec-5-en-8-yl) octahydro-2H-oxepino[2,3-c]pyrrole-8-carboxamide hydrochloride salt (FSA1606034): To a solution of (1R,8R,9R,10R,11S,12R,Z)-8-amino-3-(hydroxymethyl)-13-oxa-2-thiabicyclo[7.3.1]tridec-5-ene-10,11,12-triol formate (15 mg, 1 Eq, 44 μmol) and HATU (17 mg, 1.0 Eq, 44 μmol) in DMF (0.44 mL) at 23° C. was added DIPEA (14 mg, 19 μL, 2.5 Eq, 0.11 mmol). The reaction was stirred for 15 minutes and the resultant solution was added to solid (1R,8R,9R,10R,11S,12R,Z)-8-amino-3-(hydroxymethyl)-13-oxa-2-thiabicyclo[7.3.1]tridec-5-ene-10,11,12-triol (15 mg, 1.2 Eq, 51 μmol). The reaction was stirred for 16 hours at 23° C. and the purified directly by prep-HPLC(30-70% MeCN in H₂O with 0.1% HCOOH over 30 minutes) to afford tert-butyl (4S,5aS,8S,8aR)-4-isobutyl-8-(((1R,3S,8R,9R,10R,11S,12R,Z)-10,11,12-trihydroxy-3-(hydroxymethyl)-13-oxa-2-thiabicyclo[7.3.1]tridec-5-en-8-yl) carbamoyl) octahydro-7H-oxepino[2,3-c]pyrrole-7-carboxylate and tert-butyl (4S,5aS,8S,8aR)-4-isobutyl-8-(((1R,3R,8R,9R,10R,11S,12R,Z)-10,11,12-trihydroxy-3-(hydroxymethyl)-13-oxa-2-thiabicyclo[7.3.1]tridec-5-en-8-yl) carbamoyl) octahydro-7H-oxepino[2,3-c]pyrrole-7-carboxylate. These products were separately dissolved in HCl in MeOH (IN) at 23° C. The reactions were stirred for 2 hours and were then concentrated in vacuo to afford FSA1606033 and FSA1606034.

FSA1606033: 1H NMR (400 MHZ, CD₃OD) δ 5.77-5.60 (m, 2H), 5.51 (d, J=5.9 Hz, 1H), 4.35 (q, J=2.9 Hz, 3H), 4.31 (d, J=11.2 Hz, 1H), 4.05 (dd, J=10.2, 5.9 Hz, 1H), 4.00 (dt, J=11.9, 3.8 Hz, 1H), 3.84-3.76 (m, 2H), 3.75-3.58 (m, 3H), 3.43 (dd, J=10.3, 3.4 Hz, 1H), 3.38 (qd, J=6.8, 1.9 Hz, 1H), 3.13-2.99 (m, 1H), 2.91 (t, J=11.9 Hz, 1H), 2.78-2.62 (m, 1H), 2.43-2.24 (m, 2H), 2.13-1.98 (m, 2H), 1.83-1.60 (m, 5H), 1.19 (td, J=6.9, 3.4 Hz, 2H), 1.03-0.92 (m, 1H), 0.90 (dd, J=6.6, 1.1 Hz, 6H). HRMS (ESI+, m/z): Calc'd for[C₂₅H₄₂N₂O₇S+H]⁺: 515.2785 found: 515.2789.

FSA1606034: ¹H NMR (400 MHZ, CD₃OD) δ 8.36-8.25 (m, 1H), 5.76 (d, J=6.0 Hz, 1H), 5.69-5.58 (m, 3H), 4.40-4.29 (m, 5H), 4.02 (td, J=10.3, 5.0 Hz, 2H), 3.89 (dd, J=11.6, 6.5 Hz, 1H), 3.85-3.73 (m, 3H), 3.62 (dd, J=11.3, 7.3 Hz, 1H), 3.47 (dd, J=10.3, 3.4 Hz, 1H), 2.96-2.79 (m, 3H), 2.65 (t, J=11.2 Hz, 1H), 2.38-2.26 (m, 1H), 2.22 (d, J=10.1 Hz, 1H), 2.08 (d, J=11.9 Hz, 1H), 2.04-1.99 (m, 1H), 1.83-1.61 (m, 4H), 1.19 (td, J=6.7, 3.5 Hz, 2H), 1.03-0.92 (m, 1H), 0.90 (dd, J=6.6, 0.9 Hz, 6H). HRMS (ESI+, m/z): Calc'd for[C₂₅H₄₂N₂O₇S+H]⁺: 515.2785 found: 515.2789.

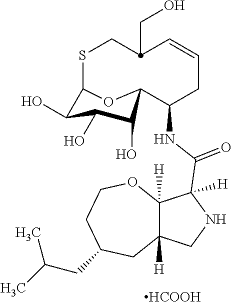 [SMILES: CC(C)C[C@@H]1CCO[C@@H]2[C@H](CN[C@@H]2C(=O)N[C@@H]2C/C=C\[C@@H](CO)CSC3O[C@H]2C(O)[C@@H](O)[C@H]3O)C1 ; "·HCOOH"]

(4S,5aS,8S,8aR)-4-isobutyl-N-((1R,4S,8R,9R,10R,11S,12R,Z)-10,11,12-trihydroxy-4-(hydroxymethyl)-13-oxa-2-thiabicyclo[7.3.1]tridec-5-en-8-yl) octahydro-2H-oxepino[2,3-c]pyrrole-8-carboxamide formate salt (FSA1608024): FSA1608024 was prepared in a similar fashion to FSA1606033 from (1R,4S,8R,9R,10S,11S,12R,Z)-8-(((R)-tert-butylsulfinyl)amino)-4-(hydroxymethyl)-13-oxa-2-thiabicyclo[7.3.1]tridec-5-ene-10,11,12-triyl tribenzoate. ¹H NMR (400 MHZ, CD₃OD) δ 8.38 (s, 1H), 5.73 (td, J=11.4, 5.1 Hz, 1H), 5.44 (d, J=5.9 Hz, 1H), 5.41 (t, J=11.0 Hz, 1H), 4.38-4.28 (m, 3H), 4.07-3.93 (m, 2H), 3.85-3.72 (m, 2H), 3.58 (dd, J=11.3, 7.1 Hz, 1H), 3.47-3.34 (m, 3H), 3.29-3.22 (m, 1H), 3.16 (dd, J=14.0, 4.1 Hz, 1H), 2.87 (t, J=11.8 Hz, 1H), 2.68 (t, J=12.4 Hz, 1H), 2.45 (dd, J=13.8, 12.2 Hz, 1H), 2.37-2.17 (m, 1H), 2.13 (dd, J=13.3, 5.1 Hz, 1H), 2.04-1.98 (m, 1H), 1.84-1.59 (m, 4H), 1.19 (td, J=6.9, 3.5 Hz, 2H), 1.01-0.91 (m, 1H), 0.90 (app d, J=6.6 Hz, 6H). HRMS (ESI+, m/z): Calc'd for[C₂₅H₄₂N₂O₇S+H]⁺: 515.2785 Found: 515.2792.

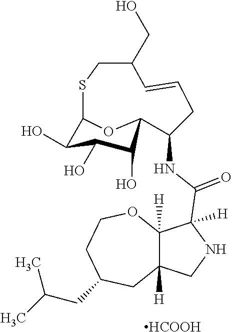 [SMILES: CC(C)C[C@@H]1CCO[C@@H]2[C@H](CN[C@@H]2C(=O)N[C@@H]2C/C=C/C(CO)CSC3O[C@H]2C(O)[C@@H](O)[C@H]3O)C1 ; "·HCOOH"]

(4S,5aS,8S,8aR)-4-isobutyl-N-((1R,8R,9R,10R,11S,12R,E)-10,11,12-trihydroxy-4-(hydroxymethyl)-13-oxa-2-thiabicyclo[7.3.1]tridec-5-en-8-yl) octahydro-2H-oxepino[2,3-c]pyrrole-8-carboxamide formate salt (FSA1608025A single diastereomer, unassigned): FSA1608025A was prepared in a similar fashion to FSA1606033 from (1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate. ¹H NMR (400 MHZ, CD₃OD) δ 8.34 (s, 1H), 5.67-5.53 (m, 1H), 5.24 (d, J=5.8 Hz, 1H), 5.14 (dd, J=14.9, 9.7 Hz, 1H), 4.39-4.24 (m, 4H), 4.08-3.92 (m, 3H), 3.86-3.72 (m, 2H), 3.58 (dd, J=11.3, 7.4 Hz, 1H), 3.52 (dd, J=10.8, 5.4 Hz, 1H), 3.49-3.33 (m, 2H), 3.29-3.22 (m, 1H), 2.89 (t, J=11.9 Hz, 1H), 2.44-2.21 (m, 4H), 2.00 (d, J=13.8 Hz, 1H), 1.82-1.60 (m, 4H), 1.18 (td, J=6.7, 3.7 Hz, 2H), 1.02-0.92 (m, 1H), 0.89 (app d, J=6.5 Hz, 6H). HRMS (ESI+, m/z): Calc'd for[C₂₅H₄₂N₂O₇S+H]⁺: 515.2785 Found: 515.2790.

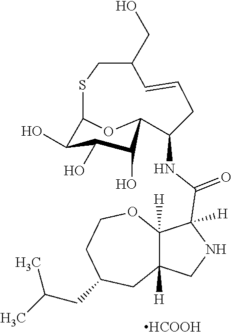 [SMILES: CC(C)C[C@@H]1CCO[C@@H]2[C@H](CN[C@@H]2C(=O)N[C@@H]2C/C=C/C(CO)CSC3O[C@H]2C(O)[C@@H](O)[C@H]3O)C1 ; "·HCOOH"]

(4S,5aS,8S,8aR)-4-isobutyl-N-((1R,8R,9R,10R,11S,12R,E)-10,11,12-trihydroxy-4-(hydroxymethyl)-13-oxa-2-thiabicyclo[7.3.1]tridec-5-en-8-yl) octahydro-2H-oxepino[2,3-c]pyrrole-8-carboxamide formate salt (FSA1608025B single diastereomer, unassigned): FSA1608025B was prepared in a similar fashion to FSA1606033 from (1R,8R,9R,10S,11S,12R,E)-8-(((R)-tert-butylsulfinyl)amino)-4-(hydroxymethyl)-13-oxa-2-thiabicyclo[7.3.1]tridec-5-ene-10,11,12-triyl tribenzoate. ¹H NMR (400 MHZ, CD₃OD) 8 8.29 (s, 1H), 5.47 (dd, J=15.7, 3.4 Hz, 1H), 5.39-5.26 (m, 1H), 5.22 (d, J=5.7 Hz, 1H), 4.41-4.24 (m, 3H), 4.21 (d, J=11.2 Hz, 1H), 4.12-3.87 (m, 3H), 3.85-3.69 (m, 3H), 3.59 (dd, J=11.3, 7.3 Hz, 1H), 3.50-3.35 (m, 2H), 2.89 (t, J=11.8 Hz, 1H), 2.67 (d, J=14.6 Hz, 3H), 2.39-2.24 (m, 2H), 2.00 (d, J=13.5 Hz, 1H), 1.86-1.57 (m, 4H), 1.19 (td, J=6.9, 3.7 Hz, 2H), 1.01-0.92 (m, 1H), 0.89 (app d, J=6.5 Hz, 6H). HRMS (ESI+, m/z): Calc'd for[C₂₅H₄₂N₂O₇S+H]⁺: 515.2785 Found: 515.2792.

FSA1608025A and FSA1608025B were each prepared as a single diastereomer. The stereocenter of the C-4 hydroxymethyl group was not assigned. However, one of FSA1608025A and FSA1608025B has the S-stereocenter at the C-4 site, and the other has the R-stereocenter at the C-4 site.

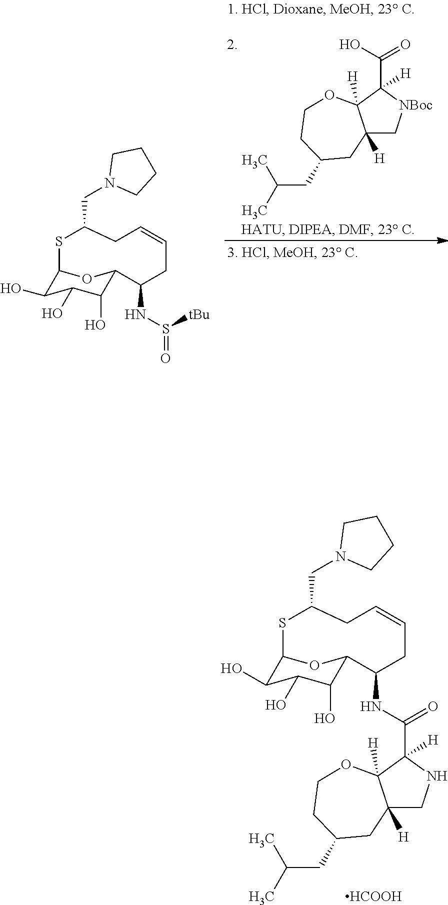 [SMILES: CC(C)(C)[S@@](=O)N[C@@H]1C/C=C\C[C@@H](CN2CCCC2)SC2OC1C(O)C(O)C2O ; CC(C)C[C@@H]1CCO[C@@H]2[C@@H](C1)CN(C(=O)OC(C)(C)C)[C@@H]2C(=O)O ; CC(C)C[C@@H]1CCO[C@@H]2[C@H](CN[C@@H]2C(=O)N[C@@H]2C/C=C\C[C@@H](CN3CCCC3)SC3OC2C(O)C(O)C3O)C1 ; "·HCOOH"]

(4S,5aS,8S,8aR)-4-isobutyl-N-((1R,3S,8R,9R,10R,11S,12R,Z)-10,11,12-trihydroxy-3-(pyrrolidin-1-ylmethyl)-13-oxa-2-thiabicyclo[7.3.1]tridec-5-en-8-yl) octahydro-2H-oxepino[2,3-c]pyrrole-8-carboxamide formate salt (FSA1606086): (R)-2-methyl-N-((1R,3S,8R,9R,10R,11S,12R,Z)-10,11,12-trihydroxy-3-(pyrrolidin-1-ylmethyl)-13-oxa-2-thiabicyclo[7.3.1]tridec-5-en-8-yl) propane-2-sulfinamide (2.0 mg, 1 Eq, 4.5 μmol) was dissolved in MeOH (300 μL) and 4M HCl in 1,4-Dioxane (75.0 μL) was added at 23° C. The reaction was maintained at 23° C. for 2 hours and concentrated in vacuo to afford crude (1R,3S,8R,9R,10R,11S,12R,Z)-8-amino-3-(pyrrolidin-1-ylmethyl)-13-oxa-2-thiabicyclo[7.3.1]tridec-5-ene-10,11,12-triol dihydrochloride.

To a solution of (4S,5aS,8aR)-7-(tert-butoxycarbonyl)-4-isobutyloctahydro-2H-oxepino[2,3-c]pyrrole-8-carboxylic acid (2.0 mg, 1.3 Eq, 6.0 μmol) and HATU (2.3 mg, 1.3 Eq, 6.0 μmol) in DMF (60 μL) at 23° C., was added DIPEA (2.7 mg, 3.7 μL, 4.5 Eq, 21 μmol). The reaction was stirred for 15 minutes and was added to solid (1R,3S,8R,9R,10R,11S,12R,Z)-8-amino-3-(pyrrolidin-1-ylmethyl)-13-oxa-2-thiabicyclo[7.3.1]tridec-5-ene-10,11,12-triol dihydrochloride (2.0 mg, 1 Eq, 4.5 μmol). The reaction was stirred at 23° C. for 16 h. The DMF was removed under a stream of argon and the crude residue was dissolved in CH₂Cl₂ (1 mL) and washed with H₂O (1 mL). The aq. layer was extracted with dichloromethane (1 mL) and the combined organics were dried over sodium sulfate and concentrated in vacuo. The crude residue was dissolved in HCl in MeOH (IN) at 23° C. The reaction was stirred for 2 hours. The reaction was concentrated in vacuo and purified by prep-HPLC(1-40% MeCN in H₂O with 0.1% HCOOH over 30 minutes) to afford FSA1606086. 1H NMR (400 MHZ, CD₃OD) δ 8.34 (app d, J=8.6 Hz, 1H), 5.84 (d, J=6.0 Hz, 1H), 5.77-5.57 (m, 2H), 4.43-4.31 (m, 3H), 4.27 (d, J=10.9 Hz, 1H), 4.13 (dd, J=10.1, 5.9 Hz, 1H), 4.01 (dt, J=11.7, 3.6 Hz, 1H), 3.88-3.75 (m, 5H), 3.73-3.65 (m, 1H), 3.61 (dd, J=11.3, 7.3 Hz, 1H), 3.48 (dd, J=10.1, 3.3 Hz, 1H), 3.41-3.33 (m, 2H), 3.27-3.19 (m, 01), 3.17-3.08 (m, 1H), 2.94 (q, J=13.4 Hz, 2H), 2.68-2.57 (m, 1H), 2.31 (s, 1H), 2.25-2.16 (m, 2H), 2.15-2.04 (m, 3H), 2.01 (d, J=13.3 Hz, 1H), 1.83-1.60 (m, 4H), 1.19 (td, J=6.9, 3.9 Hz, 2H), 1.06-0.92 (m, 1H), 0.90 (app d, J=6.6 Hz, 6H). HRMS (ESI+, m/z): Calc'd for[C₂₉H₄₉N₃O₆S+H]⁺: 568.3415 found: 568.3415.

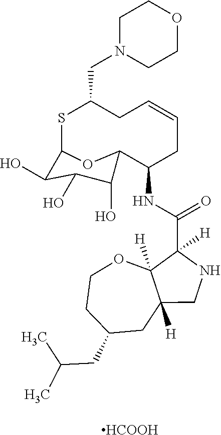 [SMILES: CC(C)C[C@@H]1CCO[C@@H]2[C@H](CN[C@@H]2C(=O)N[C@@H]2C/C=C\C[C@@H](CN3CCOCC3)SC3OC2C(O)C(O)C3O)C1 ; "·HCOOH"]

(4S,5aS,8S,8aR)-4-isobutyl-N-((1R,3S,8R,9R,10R,11S,12R,Z)-10,11,12-trihydroxy-3-(morpholinomethyl)-13-oxa-2-thiabicyclo[7.3.1]tridec-5-en-8-yl) octahydro-2H-oxepino[2,3-c]pyrrole-8-carboxamide formate salt (FSA1607019): FSA1607019 was prepared in a similar fashion to FSA1606086 from (R)-2-methyl-N-((1R,3S,8R,9R,10R,11S,12R,Z)-10,11,12-trihydroxy-3-(morpholinomethyl)-13-oxa-2-thiabicyclo[7.3.1]tridec-5-en-8-yl) propane-2-sulfinamide formate salt. ¹H NMR (400 MHZ, CD₃OD) δ 8.42 (s, 2H), 5.71 (d, J=6.0 Hz, 1H), 5.71-5.56 (m, 2H), 4.39-4.25 (m, 4H), 4.05 (dd, J=10.2, 6.0 Hz, 1H), 4.00 (dt, J=12.1, 3.8 Hz, 1H), 3.85-3.77 (m, 2H), 3.74 (t, J=4.7 Hz, 4H), 3.57 (dd, J=11.2, 7.3 Hz, 1H), 3.45 (dd, J=10.2, 3.3 Hz, 1H), 3.11 (dtt, J=11.2, 7.0, 3.5 Hz, 1H), 2.97 (dd, J=12.9, 6.9 Hz, 1H), 2.92-2.80 (m, 3H), 2.75 (dt, J=9.8, 4.5 Hz, 2H), 2.64 (dt, J=12.5, 4.7 Hz, 3H), 2.38-2.23 (m, 2H), 2.08 (dd, J=13.1, 3.1 Hz, 1H), 2.00 (d, J=13.6 Hz, 1H), 1.82-1.60 (m, 5H), 1.19 (td, J=6.8, 3.9 Hz, 2H), 1.01-0.91 (m, 1H), 0.89 (d, J=0.9 Hz, 6H). HRMS (ESI+, m/z): Calc'd for[C₂₉H₄₉N₃O₇S+H]⁺: 584.3364 found: 584.3364.

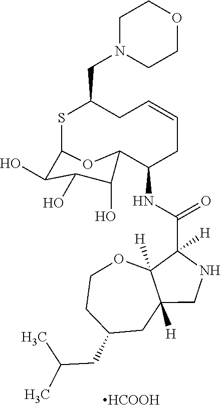 [SMILES: CC(C)C[C@@H]1CCO[C@@H]2[C@H](CN[C@@H]2C(=O)N[C@@H]2C/C=C\C[C@H](CN3CCOCC3)SC3OC2C(O)C(O)C3O)C1 ; "·HCOOH"]

(4S,5aS,8S,8aR)-4-isobutyl-N-((1R,3R,8R,9R,10R,11S,12R,Z)-10,11,12-trihydroxy-3-(morpholinomethyl)-13-oxa-2-thiabicyclo[7.3.1]tridec-5-en-8-yl) octahydro-2H-oxepino[2,3-c]pyrrole-8-carboxamide formate salt (FSA1607020): FSA1607020 was prepared in a similar fashion to FSA1606086 from(S)-2-methyl-N-((1R,3S,8R,9R,10R,11S,12R,Z)-10,11,12-trihydroxy-3-(morpholinomethyl)-13-oxa-2-thiabicyclo[7.3.1]tridec-5-en-8-yl) propane-2-sulfinamide formate salt. ¹H NMR (400 MHZ, CD₃OD) δ 8.53 (s, 1H), 5.68 (d, J=8.8 Hz, 2H), 5.49 (d, J=5.8 Hz, 1H), 4.35-4.19 (m, 3H), 4.09-4.00 (m, 2H), 3.96 (d, J=11.8 Hz, 1H), 3.86-3.73 (m, 2H), 3.69 (t, J=4.7 Hz, 4H), 3.52 (s, 1H), 3.41 (t, J=9.0 Hz, 2H), 3.11-3.01 (m, 1H), 2.75-2.59 (m, 2H), 2.57-2.43 (m, 2H), 2.31 (d, J=13.6 Hz, 1H), 2.21 (t, J=10.7 Hz, 1H), 2.06 (d, J=13.3 Hz, 1H), 1.98 (d, J=12.6 Hz, 1H), 1.78-1.60 (m, 4H), 1.16 (p, J=6.6 Hz, 2H), 0.89 (d, J=6.5 Hz, 7H). HRMS (ESI+, m/z): Calc'd for[C₂₉H₄₉N₃O₇S+H]⁺: 584.3364 found: 584.3364

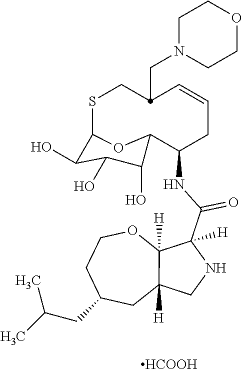 [SMILES: CC(C)C[C@@H]1CCO[C@@H]2[C@H](CN[C@@H]2C(=O)N[C@@H]2C/C=C\[C@@H](CN3CCOCC3)CSC3OC2C(O)C(O)C3O)C1 ; "·HCOOH"]

(4S,5aS,8S,8aR)-4-isobutyl-N-((1R,4S,8R,9R,10R,11S,12R,Z)-10,11,12-trihydroxy-4-(morpholinomethyl)-13-oxa-2-thiabicyclo[7.3.1]tridec-5-en-8-yl) octahydro-2H-oxepino[2,3-c]pyrrole-8-carboxamide formate salt (FSA1608036): FSA1608036 was prepared in a similar fashion to FSA1606086 from (R)-2-methyl-N-((1R,4S,8R,9R,10R,11S,12R,Z)-10,11,12-trihydroxy-4-(morpholinomethyl)-13-oxa-2-thiabicyclo[7.3.1]tridec-5-en-8-yl) propane-2-sulfinamide formate salt. ¹H NMR (400 MHZ, CD₃OD) δ 8.36 (s, 2H), 5.71 (td, J=11.3, 5.2 Hz, 1H), 5.42 (d, J=5.9 Hz, 2H), 5.38 (t, J=10.8 Hz, 1H), 4.33 (s, 4H), 4.03 (dd, J=10.2, 5.9 Hz, 1H), 4.02-3.97 (m, 1H), 3.86-3.75 (m, 2H), 3.75-3.65 (m, 4H), 3.58 (dd, J=11.2, 7.3 Hz, 1H), 3.41 (dd, J=10.1, 3.3 Hz, 1H), 3.36 (d, J=8.7 Hz, 1H), 3.21 (dd, J=14.1, 4.0 Hz, 1H), 2.96-2.81 (m, 1H), 2.68-2.25 (m, 9H), 2.17-2.09 (m, 1H), 2.03-1.96 (m, 1H), 1.85-1.56 (m, 4H), 1.19 (td, J=6.8, 3.7 Hz, 2H), 1.00-0.91 (m, 1H), 0.90 (app d, J=6.5 Hz, 6H). HRMS (ESI+, m/z): Calc'd for[C₂₉H₄₉N₃O₇S+H]⁺: 584.3364 found: 584.3390

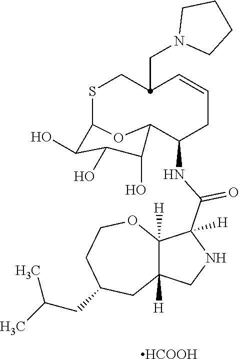 [SMILES: CC(C)C[C@@H]1CCO[C@@H]2[C@H](CN[C@@H]2C(=O)N[C@@H]2C/C=C\[C@@H](CN3CCCC3)CSC3OC2C(O)C(O)C3O)C1 ; "·HCOOH"]

(4S,5aS,8S,8aR)-4-isobutyl-N-((1R,4S,8R,9R,10R,11S,12R,Z)-10,11,12-trihydroxy-4-(pyrrolidin-1-ylmethyl)-13-oxa-2-thiabicyclo[7.3.1]tridec-5-en-8-yl) octahydro-2H-oxepino[2,3-c]pyrrole-8-carboxamide formate salt (FSA1608037): FSA1608037 was prepared in a similar fashion to FSA1606086 from ((R)-2-methyl-N-((1R,4S,8R,9R,10R,11S,12R,Z)-10,11,12-trihydroxy-4-(pyrrolidin-1-ylmethyl)-13-oxa-2-thiabicyclo[7.3.1]tridec-5-en-8-yl) propane-2-sulfinamide formate salt. ¹H NMR (400 MHZ, CD₃OD) δ 8.15 (s, 2H), 5.90 (td, J=11.6, 5.0 Hz, 1H), 5.44 (d, J=5.9 Hz, 1H), 5.39 (t, J=10.9 Hz, 1H), 4.45 (d, J=8.8 Hz, 1H), 4.41-4.31 (m, 2H), 4.25 (d, J=11.0 Hz, 1H), 4.04 (dd, J=10.2, 5.9 Hz, 1H), 3.96 (dt, J=12.2, 3.8 Hz, 1H), 3.84 (d, J=3.3 Hz, 1H), 3.75 (td, J=10.8, 4.0 Hz, 1H), 3.63 (dd, J=11.1, 7.3 Hz, 1H), 3.48 (d, J=10.8 Hz, 6H), 3.38 (dd, J=10.2, 3.1 Hz, 1H), 3.21 (dd, J=12.6, 4.2 Hz, 1H), 3.15-3.04 (m, 2H), 2.96 (t, J=13.8 Hz, 1H), 2.61 (td, J=12.7, 3.6 Hz, 1H), 2.47 (dd, J=13.9, 11.8 Hz, 1H), 2.39-2.19 (m, 2H), 2.06 (s, 4H), 2.00 (d, J=13.2 Hz, 1H), 1.79-1.56 (m, 4H), 1.18 (h, J=7.0 Hz, 2H), 1.05-0.92 (m, 1H), 0.89 (app dd, J=6.5, 1.4 Hz, 6H). HRMS (ESI+, m/z): Calc'd for[C₂₉H₄₉N₃O₆S+H]⁺: 568.3415 found: 568.3418.

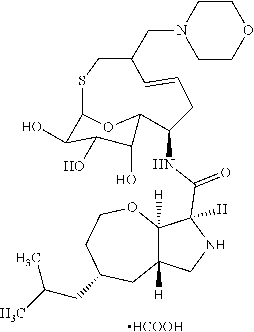 [SMILES: CC(C)C[C@@H]1CCO[C@@H]2[C@H](CN[C@@H]2C(=O)N[C@@H]2C/C=C/C(CN3CCOCC3)CSC3OC2C(O)C(O)C3O)C1 ; "·HCOOH"]

(4S,5aS,8S,8aR)-4-isobutyl-N-((1R,8R,9R,10R,11S,12R,E)-10,11,12-trihydroxy-4-(morpholinomethyl)-13-oxa-2-thiabicyclo[7.3.1]tridec-5-en-8-yl) octahydro-2H-oxepino[2,3-c]pyrrole-8-carboxamide formate salt (FSA1608056 single diastereomer, unassigned): FSA1608056 was prepared in a similar fashion to FSA1606086 from (R)-2-methyl-N-((1R,8R,9R,10R,11S,12R,E)-10,11,12-trihydroxy-4-(morpholinomethyl)-13-oxa-2-thiabicyclo[7.3.1]tridec-5-en-8-yl) propane-2-sulfinamide formate salt. ¹H NMR (400 MHZ, CD₃OD) δ 8.30 (s, 2H), 5.64 (td, J=12.3, 3.2 Hz, 1H), 5.22 (d, J=5.7 Hz, 1H), 5.12 (dd, J=14.8, 10.2 Hz, 1H), 4.43-4.26 (m, 4H), 4.01 (dd, J=10.3, 5.8 Hz, 1H), 3.98-3.93 (m, 1H), 3.83 (d, J=3.3 Hz, 1H), 3.80-3.69 (m, 6H), 3.60 (dd, J=11.2, 7.4 Hz, 1H), 3.36 (dd, J=10.2, 2.7 Hz, 1H), 3.21 (dd, J=14.5, 2.7 Hz, 1H), 2.92 (t, J=11.8 Hz, 1H), 2.83-2.66 (m, 4H), 2.63 (d, J=7.3 Hz, 2H), 2.55-2.44 (m, 1H), 2.44-2.25 (m, 4H), 2.05-1.95 (m, 1H), 1.82-1.58 (m, 4H), 1.18 (td, J=6.8, 3.9 Hz, 2H), 1.00-0.92 (m, 1H), 0.89 (app d, J=6.4, Hz, 6H). HRMS (ESI+, m/z): Calc'd for[C₂₉H₄₉N₃O₇S+H]⁺: 584.3364 found: 584.3364.

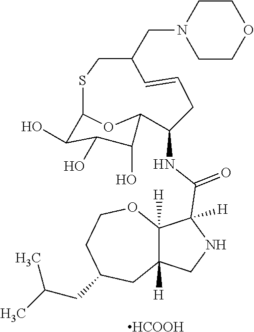 [SMILES: CC(C)C[C@@H]1CCO[C@@H]2[C@H](CN[C@@H]2C(=O)N[C@@H]2C/C=C/C(CN3CCOCC3)CSC3OC2C(O)C(O)C3O)C1 ; "·HCOOH"]

(4S,5aS,8S,8aR)-4-isobutyl-N-((1R,8R,9R,10R,11S,12R,E)-10,11,12-trihydroxy-4-(morpholinomethyl)-13-oxa-2-thiabicyclo[7.3.1]tridec-5-en-8-yl) octahydro-2H-oxepino[2,3-c]pyrrole-8-carboxamide formate salt (FSA1608057 single diastereomer, unassigned): FSA1608057 was prepared in a similar fashion to FSA1606086 from (R)-2-methyl-N-((1R,8R,9R,10R,11S,12R,E)-10,11,12-trihydroxy-4-(morpholinomethyl)-13-oxa-2-thiabicyclo[7.3.1]tridec-5-en-8-yl) propane-2-sulfinamide formate salt. ¹H NMR (400 MHZ, CD₃OD) δ 8.36 (s, 2H), 5.60-5.33 (m, 2H), 5.22 (d, J=5.7 Hz, 1H), 4.31 (s, 3H), 4.21 (d, J=11.2 Hz, 1H), 4.08-3.88 (m, 2H), 3.79 (d, J=11.7 Hz, 2H), 3.68 (s, 4H), 3.62-3.52 (m, 1H), 3.52-3.37 (m, 2H), 2.94-2.76 (m, 2H), 2.75-2.19 (m, 9H), 2.02 (s, 1H), 1.86-1.55 (m, 4H), 1.19 (s, 2H), 1.00-0.91 (m, 1H), 0.90 (d, J=6.5 Hz, 6H). HRMS (ESI+, m/z): Calc'd for[C₂₉H₄₉N₃O₇S+H]⁺: 584.3364 found: 584.3364.

FSA1608056 and FSA1608057 were each prepared as a single diastereomer. The stereocenter of the C-4 morpholinomethyl group was not assigned. However, one of FSA1608025A and FSA1608025B has the S-stereocenter at the C-4 site, and the other has the R-stereocenter at the C-4 site.

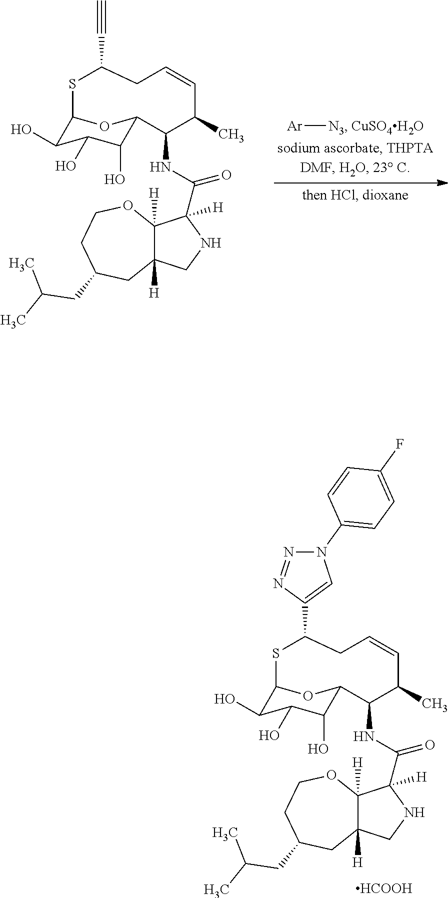

(4S,5aS,8S,8aR)—N-((1R,3S,7R,8R,9R,10R,11S,12R,Z)-3-(1-(4-fluorophenyl)-1H-1,2,3-triazol-4-yl)-10,11,12-trihydroxy-7-methyl-13-oxa-2-thiabicyclo[7.3.1]tridec-5-en-8-yl)-4-isobutyloctahydro-2H-oxepino[2,3-c]pyrrole-8-carboxamide formate salt (FSA1609037): To a degassed solution of sodium ascorbate (96 μL, 0.1 M in H₂O 1.5 Eq, 9.6 μmol) at 23° C. was added a degassed solution of copper (II) sulfate (0.21 mg, 13 μL, 0.1 M in H₂O, 0.2 Eq, 1.3 μmol). The resultant suspension was maintained at 23° C. for 5 min. A degassed solution of tris-hydroxypropyltriazolylmethylamine (32 μL, 0.1 M in H₂O, 0.5 Eq, 3.2 μmol) was then added to the suspension. The resultant solution was added to a degassed solution of tert-butyl (4S,5aS,8aR)-8-(((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl) carbamoyl)-4-isobutyloctahydro-7H-oxepino[2,3-c]pyrrole-7-carboxylate (4.0 mg, 1 Eq, 6.4 μmol) and 1-azido-4-fluorobenzene (0.97 mg, 1.1 Eq, 7.1 μmol) in DMF (0.13 mL). The resultant reaction mixture was stirred at 23° C. for 16 h. The reaction was quenched upon addition of 4N HCl in dioxane (0.2 mL) and stirred at 23° C. for an additional 2 h. The reaction mixture was concentrated in vacuo, diluted with MeOH (0.5 mL), filtered (0.2 uM syringe filter), and purified by prep-HPLC(5-50% MeCN in H₂O with 0.1% HCOOH over 30 minutes) to afford FSA1609037. 1H NMR (400 MHZ, CD₃OD) δ 8.56 (s, 1H), 8.50 (s, 1H), 8.07-7.82 (m, 2H), 7.42-7.21 (m, 2H), 5.67 (td, J=11.6, 3.4 Hz, 1H), 5.50 (d, J=5.9 Hz, 1H), 5.46 (td, J=10.9, 1.2 Hz, 1H), 4.41 (dd, J=12.8, 4.1 Hz, 1H), 4.31 (dd, J=10.9, 3.5 Hz, 1H), 4.29-4.14 (m, 3H), 3.99 (dq, J=9.9, 4.8 Hz, 2H), 3.87-3.80 (m, 1H), 3.79 (d, J=3.5 Hz, 1H), 3.54 (p, J=13.0 Hz, 1H), 3.47 (dd, J=9.6, 6.0 Hz, 1H), 3.41 (dd, J=10.3, 3.4 Hz, 1H), 3.19 (ddd, J=10.7, 6.9, 3.6 Hz, 1H), 2.73 (t, J=11.4 Hz, 1H), 2.63-2.55 (m, 1H), 2.31-2.18 (m, 1H), 1.99 (d, J=13.4 Hz, 1H), 1.84-1.59 (m, 4H), 1.19 (h, J=6.9 Hz, 2H), 1.07 (d, J=6.8 Hz, 3H), 0.99-0.92 (m, 1H), 0.90 (d, J=6.5 Hz, 6H). ¹⁹F NMR (376 MHZ, MeOD) δ-114.69. HRMS (ESI+, m/z): Calc'd for[C₃₃H₄₆FN₅O₆S+H]⁺: 660.3226 found: 660.3221.

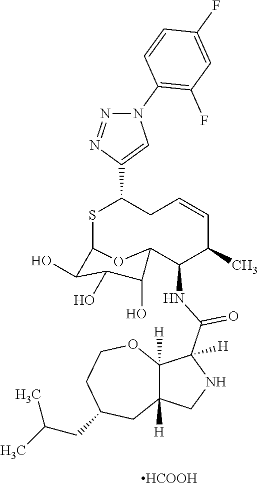 [SMILES: CC(C)C[C@@H]1CCO[C@@H]2[C@H](CN[C@@H]2C(=O)N[C@H]2C3OC(S[C@H](c4cn(-c5ccc(F)cc5F)nn4)C/C=C\[C@H]2C)C(O)C(O)C3O)C1 ; "·HCOOH"]

(4S,5aS,8S,8aR)—N-((1R,3S,7R,8R,9R,10R,11S,12R,Z)-3-(1-(2,4-difluorophenyl)-1H-1,2,3-triazol-4-yl)-10,11,12-trihydroxy-7-methyl-13-oxa-2-thiabicyclo[7.3.1]tridec-5-en-8-yl)-4-isobutyloctahydro-2H-oxepino[2,3-c]pyrrole-8-carboxamide formate salt FSA1609052 (4:1 mixture of diastereomers): The product FSA1609052 was prepared in a similar fashion to FSA1609037 from tert-butyl (4S,5aS,8aR)-8-(((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl) carbamoyl)-4-isobutyloctahydro-7H-oxepino[2,3-c]pyrrole-7-carboxylate and 1-azido-2,4-difluorobenzene. ¹H NMR (400 MHZ, CD₃OD) δ 8.52 (s, 1H), 8.41 (d, J=2.2 Hz, 0.8 H), 8.31 (d, J=2.2 Hz, 0.2H), 7.89 (tt, J=8.8, 6.0 Hz, 1H), 7.36 (ddt, J=11.8, 8.7, 3.0 Hz, 1H), 7.24 (dddd, J=9.1, 7.8, 2.8, 1.5 Hz, 1H), 5.74-5.60 (m, 1.2H), 5.53 (d, J=6.0 Hz, 0.8H), 5.47 (td, J=11.1, 2.0 Hz, 1H), 4.87-4.79 (m, 0.2H), 4.43 (dd, J=12.8, 4.1 Hz, 0.8H), 4.35-4.16 (m, 4H), 4.11 (dd, J=10.3, 5.8 Hz, 0.2H), 4.05-3.92 (m, 1.8H), 3.87-3.75 (m, 2H), 3.54 (q, J=12.6 Hz, 1H), 3.52-3.42 (m, 2H), 3.41 (dd, J=10.3, 3.4 Hz, 1H), 3.18 (ddd, J=10.8, 7.1, 3.7 Hz, 1H), 2.74 (t, J=11.4 Hz, 1H), 2.65 (d, J=13.3 Hz, 0.2H), 2.63-2.54 (m, 0.8H), 2.31-2.10 (m, 1H), 1.99 (d, J=13.3 Hz, 1H), 1.87-1.54 (m, 4H), 1.19 (td, J=6.8, 4.0 Hz, 2H), 1.06 (d, J=7.3 Hz, 3H), 1.00-0.92 (m, 1H), 0.89 (app d, J=6.6 Hz, 6H). ¹⁹F NMR (376 MHZ, MeOD, *denotes minor diastereomer where resolved)) 8*−108.97 (d, J=7.6 Hz), −109.13 (d, J=7.7 Hz), −120.58 (d, J=7.7 Hz), *−120.91 (d, J=8.2 Hz). HRMS (ESI+, m/z): Calc'd for[C₃₃H₄₅F₂N₅O₆S+H]⁺: 678.3131 found: 678.3134.

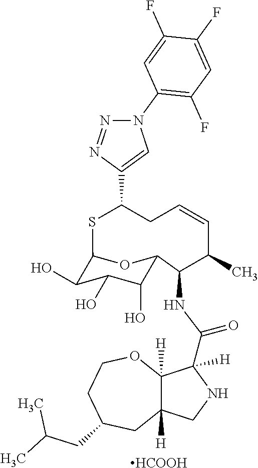 [SMILES: CC(C)C[C@@H]1CCO[C@@H]2[C@H](CN[C@@H]2C(=O)N[C@H]2C3OC(S[C@H](c4cn(-c5cc(F)c(F)cc5F)nn4)C/C=C\[C@H]2C)C(O)C(O)C3O)C1 ; "·HCOOH"]

(4S,5aS,8S,8aR)-4-isobutyl-N-((1R,3S,7R,8R,9R,10R,11S,12R,Z)-10,11,12-trihydroxy-7-methyl-3-(1-(2,4,5-trifluorophenyl)-1H-1,2,3-triazol-4-yl)-13-oxa-2-thiabicyclo[7.3.1]tridec-5-en-8-yl) octahydro-2H-oxepino[2,3-c]pyrrole-8-carboxamide formate salt (FSA1609051; 4:1 mixture of diastereomers): The product FSA1609051 was prepared in a similar fashion to FSA1609037 from tert-butyl (4S,5aS,8aR)-8-(((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl) carbamoyl)-4-isobutyloctahydro-7H-oxepino[2,3-c]pyrrole-7-carboxylate and 1-azido-2,4,5-trifluorobenzene. ¹H NMR (400 MHZ, CD₃OD) δ 8 8.52 (s, 1H), 8.45 (d, J=2.3 Hz, 0.8H), 8.35 (d, J=2.3 Hz, 0.8H), 8.01-7.88 (m, 1H), 7.67-7.52 (m, 1H), 5.74-5.62 (m, 1.2H), 5.52 (d, J=6.0 Hz, 0.8H), 5.51-5.40 (m, 1H), 4.87-4.78 (m, 0.2H), 4.42 (dd, J=12.9, 4.1 Hz, 0.8H), 4.34-4.20 (m, 3H), 4.16 (d, J=8.5 Hz, 1H), 4.11 (dd, J=10.2, 5.9 Hz, 0.2H), 4.05-3.93 (m, 1.8H), 3.86-3.75 (m, 2H), 3.55 (q, J=12.6 Hz, 1H), 3.50-3.37 (m, 2H), 3.22-3.06 (m, 1H), 2.71 (t, J=11.3 Hz, 1H), 2.67-2.62 (m, 0.2H), 2.62-2.50 (m, 0.8H), 2.32-2.14 (m, 1H), 1.99 (d, J=12.9 Hz, 1H), 1.83-1.60 (m, 4H), 1.19 (dt, J=11.4, 7.1 Hz, 2H), 1.06 (d, J=7.4 Hz, 3H), 0.91 (app d, J=6.6 Hz, 6H). ¹⁹F NMR (376 MHZ, MeOD, *denotes minor diastereomer where resolved) 8-125.58 (dd, J=14.3, 4.4 Hz), *−125.92 (dd, J=14.5, 4.2 Hz), *−133.51 (dd, J=21.1, 4.4 Hz), −133.64 (dd, J=21.2, 4.3 Hz), *−141.90 (d, J=20.9 Hz), −141.96 (dd, J=21.4, 14.6 Hz). HRMS (ESI+, m/z): Calc'd for[C₃₃H₄₄F₃N₅O₆S+H]⁺: 696.3037 found: 696.3047

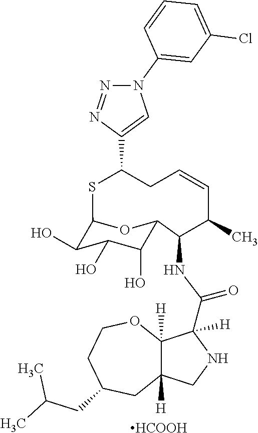 [SMILES: CC(C)C[C@@H]1CCO[C@@H]2[C@H](CN[C@@H]2C(=O)N[C@H]2C3OC(S[C@H](c4cn(-c5cccc(Cl)c5)nn4)C/C=C\[C@H]2C)C(O)C(O)C3O)C1 ; "·HCOOH"]

(4S,5aS,8S,8aR)—N-((1R,3S,7R,8R,9R,10R,11S,12R,Z)-3-(1-(3-chlorophenyl)-1H-1,2,3-triazol-4-yl)-10,11,12-trihydroxy-7-methyl-13-oxa-2-thiabicyclo[7.3.1]tridec-5-en-8-yl)-4-isobutyloctahydro-2H-oxepino[2,3-c]pyrrole-8-carboxamide formate salt (FSA1609015; 7:2:1 mixture of isomers): The product FSA1609015 was prepared in a similar fashion to FSA1609037 from tert-butyl (4S,5aS,8aR)-8-(((1R,3S,7R,8R,9R,10R,11S,12R,Z)-3-ethynyl-10,11,12-trihydroxy-7-methyl-13-oxa-2-thiabicyclo[7.3.1]tridec-5-en-8-yl) carbamoyl)-4-isobutyloctahydro-7H-oxepino[2,3-c]pyrrole-7-carboxylate and 1-azido-3-chlorobenzene. ¹H NMR (400 MHZ, CD₃OD) δ 8.64 (s, 0.7H), 8.58 (s, 0.2H), 8.53 (s, 0.1H), 8.51 (s, 1H), 7.99 (t, J=2.1 Hz, 0.7H), 7.97 (t, J=2.0 Hz, 0.1H), 7.95 (t, J=2.0 Hz, 0.2H), 7.86 (ddd, J=8.2, 2.2, 1.1 Hz, 0.7H), 7.84-7.81 (m, 0.3H), 7.58 (td, J=8.1, 3.0 Hz, 1H), 7.51 (ddd, J=8.1, 2.1, 1.1 Hz, 1H), 5.89-5.61 (m, 1H), 5.59-5.38 (m, 2H), 4.85-4.77 (m, 0.2H), 4.41 (dd, J=12.9, 4.1 Hz, 0.7H), 4.35-3.91 (m, 6.1H), 3.87-3.75 (m, 1.8H), 3.65 (t, J=9.1 Hz, 0.2H), 3.52 (q, J=13.3 Hz, 0.7H), 3.49-3.35 (m, 2.3H), 3.19 (ddd, J=10.8, 6.9, 3.6 Hz, 1H), 2.95-2.85 (m, 0.2H), 2.71 (t, J=11.2 Hz, 1H), 2.67-2.55 (m, 0.8H), 2.49-2.38 (m, 0.2H)2.35-2.15 (m, 1H), 1.98 (d, J=14.1 Hz, 1H), 1.84-1.59 (m, 4H), 1.25-1.12 (m, 2H), 1.13 (d, J=6.5 Hz, 0.6H), 1.07 (d, J=6.8 Hz, 2.1H), 1.05 (d, J=6.8 Hz, 0.2H), 0.99-0.83 (m, 7H). HRMS (ESI+, m/z): Calc'd for[C₃₃H₄₆ClN₅O₆S+H]⁺: 676.2930 found: 676.2936

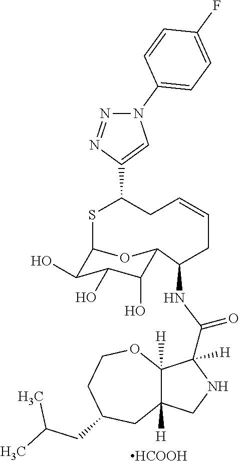 [SMILES: CC(C)C[C@@H]1CCO[C@@H]2[C@H](CN[C@@H]2C(=O)N[C@@H]2C/C=C\C[C@@H](c3cn(-c4ccc(F)cc4)nn3)SC3OC2C(O)C(O)C3O)C1 ; "·HCOOH"]

(4S,5aS,8S,8aR)—N-((1R,3S,8R,9R,10R,11S,12R,Z)-3-(1-(4-fluorophenyl)-1H-1,2,3-triazol-4-yl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide formate salt (FSA1608021; 4:1 mixture of diastereomers): The product FSA1608021 was prepared in a similar fashion to FSA1609037 from tert-butyl (4S,5aS,8S,8aR)-8-(((1R,3S,8R,9R,10R,11S,12R,Z)-3-ethynyl-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate and 1-azido-4-fluorobenzene. ¹H NMR (400 MHZ, CD₃OD) δ 8.56 (s, 1H), 8.45 (s, 1H), 7.90 (tdd, J=9.1, 4.6, 2.2 Hz, 2H), 7.34 (ddd, J=10.0, 8.2, 3.1 Hz, 2H), 5.87-5.72 (m, 2H), 5.70 (d, J=5.8 Hz, 0.2H), 5.50 (d, J=6.0 Hz, 0.8H), 4.50-4.23 (m, 5H), 4.12 (dd, J=10.3, 5.9 Hz, 0.2H), 4.08-3.93 (m, 1.8H), 3.90-3.74 (m, 2H), 3.63-3.51 (m, 2H), 3.44 (dd, J=10.3, 3.4 Hz, 1H), 2.93-2.71 (m, 2H), 2.64 (d, J=14.0 Hz, 0.2H), 2.57 (dd, J=13.6, 3.9 Hz, 0.2H), 2.38-2.24 (m, 1H), 2.14 (d, J=12.8 Hz, 1H), 2.01 (d, J=13.4 Hz, 1H), 1.87-1.57 (m, 4H), 1.19 (tt, J=10.6, 5.1 Hz, 2H), 1.03-0.92 (m, 1H), 0.92-0.85 (m, 6H). ¹⁹F NMR (376 MHZ, MeOD, *denotes minor diastereomer where resolved) δ*−114.55,-114.66. HRMS (ESI+, m/z): Calc'd for[C₃₂H₄₄FN₅O₆S+H]⁺: 646.3069 found: 646.3070.

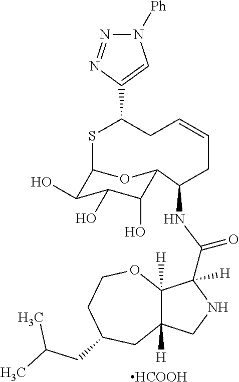 [SMILES: CC(C)C[C@@H]1CCO[C@@H]2[C@H](CN[C@@H]2C(=O)N[C@@H]2C/C=C\C[C@@H](c3cn(-c4ccccc4)nn3)SC3OC2C(O)C(O)C3O)C1 ; "·HCOOH"]

(4S,5aS,8S,8aR)-4-isobutyl-N-((1R,3S,8R,9R,10R,11S,12R,Z)-10,11,12-trihydroxy-3-(1-phenyl-1H-1,2,3-triazol-4-yl)-13-oxa-2-thiabicyclo[7.3.1]tridec-5-en-8-yl) octahydro-2H-oxepino[2,3-c]pyrrole-8-carboxamide formate salt (FSA1607075): The product FSA1607075 was prepared in a similar fashion to FSA1609037 from tert-butyl (4S,5aS,8S,8aR)-8-(((1R,3S,8R,9R,10R,11S,12R,Z)-3-ethynyl-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate and azidobenzene. ¹H NMR (400 MHz, CD₃OD) δ 8.59 (s, 1H), 8.47 (s, 1H), 7.88 (dd, J=7.4, 1.0 Hz, 2H), 7.59 (dd, J=8.7, 7.0 Hz, 2H), 7.54-7.46 (m, 1H), 5.76 (d, J=10.2 Hz, 2H), 5.50 (d, J=6.0 Hz, 1H), 4.43 (dd, J=12.8, 4.1 Hz, 1H), 4.39-4.28 (m, 3H), 4.24 (d, J=8.8 Hz, 1H), 4.05-3.95 (m, 2H), 3.87-3.79 (m, 2H), 3.62-3.49 (m, 2H), 3.44 (d, J=10.3 Hz, 1H), 2.87-2.75 (m, 2H), 2.58 (dd, J=13.3, 3.4 Hz, 1H), 2.39-2.23 (m, 1H), 2.14 (d, J=13.0 Hz, 1H), 2.01 (d, J=13.3 Hz, 1H), 1.85-1.60 (m, 4H), 1.19 (td, J=6.7, 3.7 Hz, 2H), 1.01-0.92 (m, 1H), 0.90 (d, J=6.5 Hz, 6H). HRMS (ESI+, m/z): Calc'd for[C₃₂H₄₅N₅O₆S+H]⁺: 628.3163 Found: 628.3103.

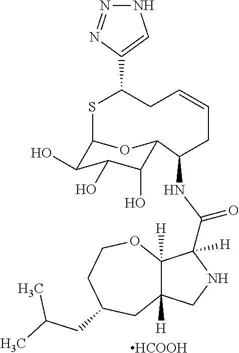 [SMILES: CC(C)C[C@@H]1CCO[C@@H]2[C@H](CN[C@@H]2C(=O)N[C@@H]2C/C=C\C[C@@H](c3c[nH]nn3)SC3OC2C(O)C(O)C3O)C1 ; "·HCOOH"]

(4S,5aS,8S,8aR)-4-isobutyl-N-((1R,3S,8R,9R,10R,11S,12R,Z)-10,11,12-trihydroxy-3-(1H-1,2,3-triazol-4-yl)-13-oxa-2-thiabicyclo[7.3.1]tridec-5-en-8-yl) octahydro-2H-oxepino[2,3-c]pyrrole-8-carboxamide formate salt (FSA1607090): The product FSA1607090 was prepared in a similar fashion to FSA1609037 from tert-butyl (4S,5aS,8S,8aR)-8-(((1R,3S,8R,9R,10R,11S,12R,Z)-3-ethynyl-10,11,12-trihydroxy-13-oxa-2-thiabicyclo[7.3.1]tridec-5-en-8-yl) carbamoyl)-4-isobutyloctahydro-7H-oxepino[2,3-c]pyrrole-7-carboxylate and trimethylsilylazide. ¹H NMR (400 MHZ, CD₃OD) δ 8.55-8.34 (m, 1H), 7.84 (s, 1H), 5.73 (d, J=9.4 Hz, 2H), 5.37 (d, J=6.1 Hz, 1H), 4.58 (s, 2H), 4.39 (dd, J=12.9, 4.0 Hz, 2H), 4.32 (t, J=10.5 Hz, 2H), 4.25 (d, J=9.0 Hz, 1H), 4.01 (dt, J=11.9, 3.6 Hz, 1H), 3.96 (dd, J=10.3, 6.1 Hz, 1H), 3.87-3.77 (m, 2H), 3.57-3.45 (m, 2H), 3.41 (dd, J=10.3, 3.2 Hz, 1H), 2.85-2.72 (m, 1H), 2.50 (dd, J=13.3, 4.1 Hz, OH), 2.35-2.24 (m, 1H), 2.12 (d, J=13.0 Hz, 1H), 2.00 (d, J=13.4 Hz, 1H), 1.82-1.61 (m, 4H), 1.19 (td, J=6.7, 3.8 Hz, 2H), 1.00-0.91 (m, 1H), 0.90 (d, J=6.5 Hz, 6H). HRMS (ESI+, m/z): Calc'd for[C₂₆H₄₁N₅O₆S+H]⁺: 552.2850 Found: 552.2846

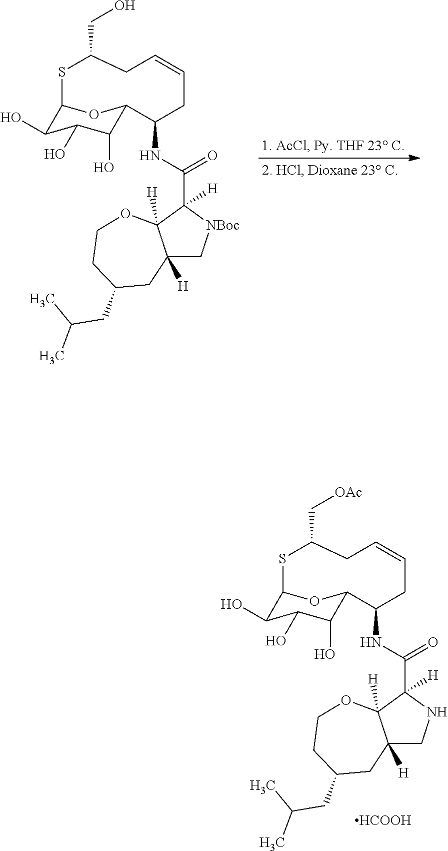 [SMILES: CC(C)C[C@@H]1CCO[C@@H]2[C@@H](C1)CN(C(=O)OC(C)(C)C)[C@@H]2C(=O)N[C@@H]1C/C=C\C[C@@H](CO)SC2OC1C(O)C(O)C2O ; CC(=O)OC[C@@H]1C/C=C\C[C@@H](NC(=O)[C@H]2NC[C@@H]3C[C@H](CC(C)C)CCO[C@H]32)C2OC(S1)C(O)C(O)C2O ; "·HCOOH"]

((1R,3S,8R,9R,10R,11S,12R,Z)-10,11,12-trihydroxy-8-((4S,5aS,8S,8aR)-4-isobutyloctahydro-2H-oxepino[2,3-c]pyrrole-8-carboxamido)-13-oxa-2-thiabicyclo[7.3.1]tridec-5-en-3-yl)methyl acetate formate salt (FSA1607032): To a solution of tert-butyl (4S,5aS,8aR)-4-isobutyl-8-(((1R,3S,8R,9R,10R,11S,12R,Z)-10,11,12-trihydroxy-3-(hydroxymethyl)-13-oxa-2-thiabicyclo[7.3.1]tridec-5-en-8-yl) carbamoyl) octahydro-7H-oxepino[2,3-c]pyrrole-7-carboxylate (2.2 mg, 1 Eq, 3.6 μmol) in THF (0.05 mL) at 23° C., was added pyridine (0.29 μL, 1 Eq, 3.6 μmol) and acetyl chloride (0.25 μL, 1 Eq, 3.6 μmol). The reaction mixture was maintained at 23° C. for 3 h and concentrated in vacuo afford a crude mixture of acylated products. The crude residue was dissolved in HCl in dioxane (4 N, 1 mL) and maintained at 23° C. for 2 hours. The reaction mixture was concentrated in vacuo and purified by prep-HPLC(5-40% MeCN in H₂O with 0.1% HCOOH over 30 minutes) to afford FSA1607032. ¹H NMR (400 MHZ, CD₃OD) δ 8.53 (s, 1H), 5.78 (d, J=6.0 Hz, 1H), 5.73-5.55 (m, 2H), 4.55 (dd, J=11.6, 6.8 Hz, 1H), 4.39-4.19 (m, 4H), 4.03 (dd, J=10.0, 5.6 Hz, 2H), 3.96 (d, J=11.8 Hz, 1H), 3.86-3.72 (m, 2H), 3.45 (dd, J=10.2, 3.2 Hz, 1H), 3.39 (dd, J=10.9, 7.3 Hz, 1H), 3.20-3.05 (m, 1H), 2.93 (q, J=12.5 Hz, 1H), 2.65 (q, J=10.8 Hz, 2H), 2.20 (d, J=12.9 Hz, 2H), 2.10-2.03 (m, 1H), 2.08 (s, 3H), 1.98 (d, J=13.3 Hz, 1H), 1.83-1.56 (m, 4H), 1.17 (q, J=6.2 Hz, 2H), 0.89 (app d, J=6.5 Hz, 6H). HRMS (ESI+, m/z): Calc'd for[C₂₇H₄₄N₂O₆S+H]⁺: 557.2891 found: 557.2908.

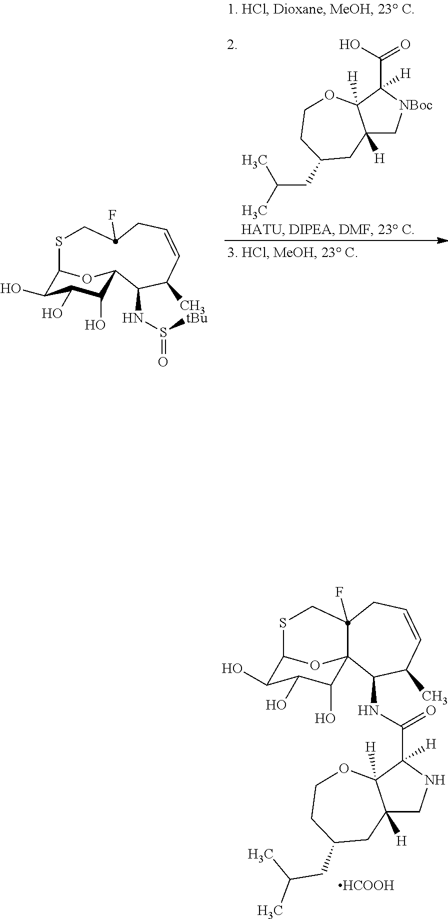

(4S,5aS,8S,8aR)—N-((1R,4R,8R,9R,10R,11R,12S,13R,Z)-4-fluoro-11,12,13-trihydroxy-8-methyl-14-oxa-2-thiabicyclo[8.3.1]tetradec-6-en-9-yl)-4-isobutyloctahydro-2H-oxepino[2,3-c]pyrrole-8-carboxamide formate salt (FSA1609033): To a solution of (R)—N-((1R,4R,8R,9R,10R,11R,12S,13R,Z)-4-fluoro-11,12,13-trihydroxy-8-methyl-14-oxa-2-thiabicyclo[8.3.1]tetradec-6-en-9-yl)-2-methylpropane-2-sulfinamide (9 mg, 1 Eq, 0.02 mmol) in MeOH (0.3 mL) at 23° C., was added HCl (4 N in dioxane, 200 μL). The reaction was maintained at 23° C. for 45 min and then concentrated in vacuo to afford crude (1R,4R,8R,9R,10R,11R,12S,13R,Z)-9-amino-4-fluoro-8-methyl-14-oxa-2-thiabicyclo[8.3.1]tetradec-6-ene-11,12,13-triol hydrochloride (8 mg, 0.02 mmol) as a colorless film.

To a solution of (4S,5aS,8aR)-7-(tert-butoxycarbonyl)-4-isobutyloctahydro-2H-oxepino[2,3-c]pyrrole-8-carboxylic acid (7.9 mg, 1 Eq, 23 μmol) and HATU (8.8 mg, 1.0 Eq, 23 μmol) in DMF (0.23 mL) at 23° C., was added DIPEA (7.5 mg, 10 μL, 2.5 Eq, 58 μmol). The resultant reaction was stirred for 15 minutes and added to solid (1R,4R,8R,9R,10R,11R,12S,13R,Z)-9-amino-4-fluoro-8-methyl-14-oxa-2-thiabicyclo[8.3.1]tetradec-6-ene-11,12,13-triol hydrochloride (8 mg, 1Eq, 23 μmol). The resultant reaction mixture was stirred at 23° C. for 16 h. The reaction was quenched upon addition of MeOH (0.4 mL) and 4N HCl in dioxane (0.2 mL). The reaction was stirred at 23° C. for an additional 2 h. The reaction mixture was concentrated in vacuo, diluted with MeOH (0.5 mL), filtered (0.2 uM syringe filter), and purified by prep-HPLC(5-50% MeCN in H₂O with 0.1% HCOOH over 30 minutes) to afford FSA1609033. ¹H NMR (400 MHZ, CD₃OD) δ 8.40 (s, 1H), 5.64 (td, J=11.1, 3.1 Hz, 1H), 5.39 (d, J=5.6 Hz, 1H), 5.28 (tt, J=11.3, 2.1 Hz, 1H), 4.60-4.39 (m, 1H), 4.39-4.26 (m, 3H), 4.04 (dd, J=10.5, 5.6 Hz, 1H), 3.99 (dt, J=12.0, 4.0 Hz, 1H), 3.87-3.68 (m, 3H), 3.59 (t, J=7.4 Hz, 1H), 3.48 (ddd, J=13.8, 9.8, 3.3 Hz, 1H), 3.33 (d, J=3.3 Hz, 1H), 3.19-3.03 (m, 2H), 2.87 (t, J=11.5 Hz, 1H), 2.67 (ddd, J=13.7, 11.1, 4.7 Hz, 1H), 2.49-2.31 (m, 1H), 2.29 (d, J=11.1 Hz, 1H), 2.00 (d, J=13.2 Hz, 1H), 1.86-1.59 (m, 4H), 1.19 (td, J=6.8, 3.4 Hz, 2H), 1.03 (d, J=6.9 Hz, 3H), 0.96 (d, J=11.9 Hz, 1H), 0.89 (app d, J=6.6 Hz, 6H). ¹⁹F NMR (376 MHZ, MeOD) δ-150.99. HRMS (ESI+, m/z): Calc'd for[C₂₆H₄₃FNO₆S+H]⁺: 531.2899 found: 531.2904

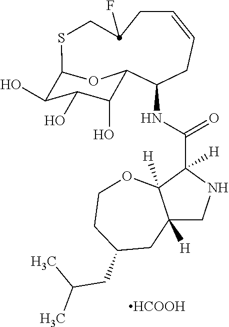 [SMILES: CC(C)C[C@@H]1CCO[C@@H]2[C@H](CN[C@@H]2C(=O)N[C@@H]2C/C=C\C[C@@H](F)CSC3OC2C(O)C(O)C3O)C1 ; "·HCOOH"]

(4S,5aS,8S,8aR)—N-((1R,4R,9R,10R,11R,12S,13R,Z)-4-fluoro-11,12,13-trihydroxy-14-oxa-2-thiabicyclo[8.3.1]tetradec-6-en-9-yl)-4-isobutyloctahydro-2H-oxepino[2,3-c]pyrrole-8-carboxamide formate salt (FSA1607022): The product FSA1607022 was prepared in a similar fashion to FSA1609033 from (R)—N-((1R,4R,9R,10R,11R,12S,13R,Z)-4-fluoro-11,12,13-trihydroxy-14-oxa-2-thiabicyclo[8.3.1]tetradec-6-en-9-yl)-2-methylpropane-2-sulfinamide. ¹H NMR (400 MHZ, CD₃OD) δ 8.47 (s, 1H), 5.72-5.59 (m, 1H), 5.59-5.49 (m, 1H), 5.37 (d, J=5.8 Hz, 1H), 4.66-4.44 (m, 1H), 4.36-4.26 (m, 1H), 4.30 (t, J=9.2 Hz, 1H), 4.21 (d, J=4.6 Hz, 1H), 4.04 (dd, J=10.5, 5.7 Hz, 1H), 4.00-3.89 (m, 2H), 3.83 (d, J=2.9 Hz, 1H), 3.78 (ddd, J=13.6, 10.8, 3.2 Hz, 1H), 3.52 (t, J=9.4 Hz, 1H), 3.43 (t, J=11.7 Hz, 1H), 3.35-3.32 (m, 1H), 2.97 (br. s, 1H), 2.80 (t, J=11.7 Hz, 1H), 2.65 (ddd, J=13.5, 11.2, 5.3 Hz, 1H), 2.70-2.40 (m, 2H), 2.36-2.10 (m, 2H), 1.99 (d, J=13.3 Hz, 1H), 1.82-1.58 (m, 4H), 1.18 (td, J=6.8, 4.0 Hz, 2H), 0.99-0.91 (m, 1H), 0.89 (app d, J=5.6 Hz, 6H). HRMS (ESI+, m/z): Calc'd for[C₂₅H₄₂FN₂O₆S+H]⁺: 517.2742 found: 517.2744

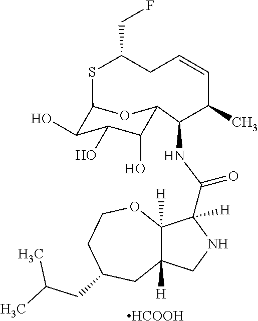 [SMILES: CC(C)C[C@@H]1CCO[C@@H]2[C@H](CN[C@@H]2C(=O)N[C@H]2C3OC(S[C@H](CF)C/C=C\[C@H]2C)C(O)C(O)C3O)C1 ; "·HCOOH"]

(4S,5aS,8S,8aR)—N-((1R,3S,7R,8R,9R,10R,11S,12R,Z)-3-(fluoromethyl)-10,11,12-trihydroxy-7-methyl-13-oxa-2-thiabicyclo[7.3.1]tridec-5-en-8-yl)-4-isobutyloctahydro-2H-oxepino[2,3-c]pyrrole-8-carboxamide formate salt (FSA1609034): The product FSA1609034 was prepared in a similar fashion to FSA1609033 from (R)—N-((1R,3S,7R,8R,9R,10R,11S,12R,Z)-3-(fluoromethyl)-10,11,12-trihydroxy-7-methyl-13-oxa-2-thiabicyclo[7.3.1]tridec-5-en-8-yl)-2-methylpropane-2-sulfinamide. ¹H NMR (400 MHZ, CD₃OD) δ 8.41 (s, 1H), 5.81 (d, J=5.9 Hz, 1H), 5.55 (td, J=11.0, 3.5 Hz, 1H), 5.39 (td, J=11.1, 1.9 Hz, 1H), 4.78 (ddd, J=47.8, 9.1, 4.7 Hz, 1H), 4.70 (ddd, J=47.9, 9.3, 6.6 Hz, 1H),4.08-3.95 (m, 2H), 3.82 (ddd, J=11.5, 10.1, 3.7 Hz, 1H), 3.76 (d, J=3.4 Hz, 1H), 3.60 (dd, J=11.3, 7.5 Hz, 1H), 3.45 (dd, J=10.3, 3.4 Hz, 1H), 3.16-2.94 (m, 3H), 2.89 (t, J=11.7 Hz, 1H), 2.39-2.21 (m, 2H), 2.00 (dt, J=12.9, 3.2 Hz, 1H), 1.84-1.61 (m, 4H), 1.19 (td, J=6.8, 3.4 Hz, 2H), 1.05 (d, J=6.9 Hz, 3H), 1.02-0.91 (m, 1H), 0.90 (app d, J=6.6 Hz, 7H). ¹⁹F NMR (376 MHZ, MeOD) 8-215.48 (d, J=6.2 Hz). HRMS (ESI+, m/z): Calc'd for[C₂₆H₄₄FN₂O₆S+H]⁺: 531.2899 found: 531.2894

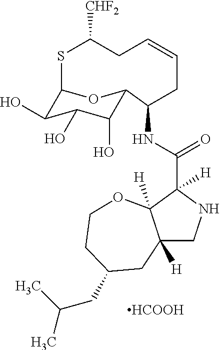 [SMILES: CC(C)C[C@@H]1CCO[C@@H]2[C@H](CN[C@@H]2C(=O)N[C@@H]2C/C=C\C[C@@H](C(F)F)SC3OC2C(O)C(O)C3O)C1 ; "·HCOOH"]

(4S,5aS,8S,8aR)—N-((1R,3S,8R,9R,10R,11S,12R,Z)-3-(difluoromethyl)-10,11,12-trihydroxy-13-oxa-2-thiabicyclo[7.3.1]tridec-5-en-8-yl)-4-isobutyloctahydro-2H-oxepino[2,3-c]pyrrole-8-carboxamide formate salt (FSA1607044, 4:1 mixture of diastereomers): The product FSA1607044 was prepared in a similar fashion to FSA1609033 from (R)—N-((1R,3S,8R,9R,10R,11S,12R,Z)-3-(difluoromethyl)-10,11,12-trihydroxy-13-oxa-2-thiabicyclo[7.3.1]tridec-5-en-8-yl)-2-methylpropane-2-sulfinamide. ¹H NMR (400 MHZ, CD₃OD) δ 8 6.19 (td, J=56.7, 1.4 Hz, 0.8H 6.07 (td, J=55.7, 3.6 Hz, 0.2H), 5.86 (d, J=6.0 Hz, 0.8H), 5.72 (tdd, J=11.4, 5.3, 1.9 Hz, 1.2H), 5.61 (td, J=10.7, 3.2 Hz, 0.8H), 5.55 (d, J=5.9 Hz, 0.2H), 4.40-4.21 (m, 4H), 4.08 (dd, J=10.3, 5.8 Hz, 0.2H), 4.03 (dd, J=10.4, 6.1 Hz, 0.8H), 4.02-3.95 (m, 1H), 3.82 (d, J=3.4 Hz, 1H), 3.81-3.76 (m, 1H), 3.55 (dd, J=11.2, 7.3 Hz, 1H), 3.48 (dd, J=10.3, 3.3 Hz, 0.8H), 3.43 (dd, J=10.2, 3.4 Hz, 0.2H), 3.24-3.01 (m, 2H), 2.84 (t, J=11.7 Hz, 1H), 2.66 (td, J=12.5, 3.5 Hz, 1H), 2.43 (d, J=14.4 Hz, 0.2H), 2.34 (d, J=12.0 Hz, 0.8H), 2.37-2.22 (m, 1H), 2.17-2.07 (m, 1H), 2.00 (d, J=13.4 Hz, 1H), 1.84-1.58 (m, 4H), 1.19 (td, J=6.8, 3.8 Hz, 2H), 1.01-0.92 (m, 1H), 0.90 (app d, J=6.6 Hz, 6H). ¹⁹F NMR (376 MHZ, CD₃OD, *denotes minor diastereomer when resolved) 8-110.97 (d, J=278.0 Hz), *−118.16 (d, J=281.2 Hz), *−119.45 (d, J=281.4 Hz), −123.69 (d, J=278.1 Hz). HRMS (ESI+, m/z): Calc'd for[C₂₅H₄₀F₂N₂O₆S₂+H]⁺: 535.2648 found: 535.2637.

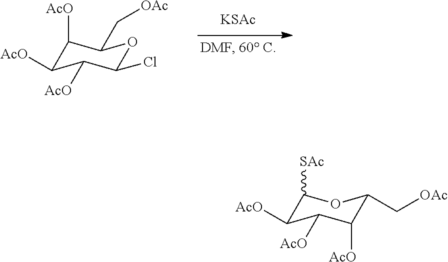

(R)—N-((1R,4R,8R,9R,10R,11R,12S,13R,Z)-4-fluoro-11,12,13-trihydroxy-8-methyl-14-oxa-2-thiabicyclo[8.3.1]tetradec-6-en-9-yl)-2-methylpropane-2-sulfinamide (F1): A solution of (2R,3S,4S,5R,6S)-2-(acetoxymethyl)-6-chlorotetrahydro-2H-pyran-3,4,5-triyl triacetate (4.45 g, 1 Eq, 12.1 mmol) in DMF (24.3 mL) was degassed and heated to 60° C. Potassium thioacetate (4.16 g, 3.0 Eq, 36.4 mmol) was added and the reaction was maintained at this temperature for 30 min. The reaction was cooled to 23° C. and diluted with EtOAc (125 mL) and washed with sat. aq. NaCl (3×100 mL). The combined organics were dried over sodium sulfate and concentrated in vacuo. The crude residue was purified by flash chromatography (5-20% EtOAc in Hexanes) to afford product F1 (4 g, ˜60% purity, ˜50% yield, inseparable mixture with beta anomer) as light orange oil. ¹H NMR (400 MHZ, CDCl₃) δ 6.24 (d, J=5.5 Hz, 1H), 5.45 (dd, J=11.0, 5.5 Hz, 1H), 5.41 (dd, J=3.4, 1.3 Hz, 1H), 5.02 (dd, J=11.0, 3.3 Hz, 1H), 4.15 (td, J=6.5, 1.3 Hz, 1H), 4.12-4.00 (m, 2H), 2.40 (s, 3H), 2.13 (s, 3H), 2.00 (s, 3H), 2.00 (s, 3H), 1.97 (s, 3H). HRMS (ESI+, m/z): Calc'd for[C₁₆H₂₂O₁₀S+NH₄]⁺: 424.1272 found::424.1272

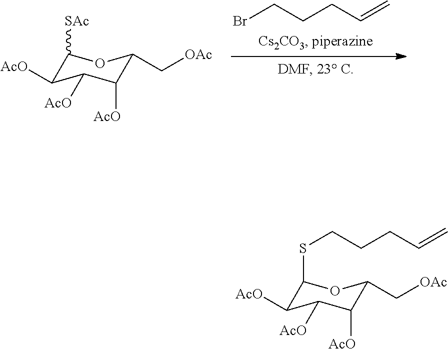

(2R,3S,4S,5R,6R)-2-(acetoxymethyl)-6-(pent-4-en-1-ylthio)tetrahydro-2H-pyran-3,4,5-triyl triacetate (F2): To a degassed solution of (2R,3S,4S,5R)-2-(acetoxymethyl)-6-(acetylthio)tetrahydro-2H-pyran-3,4,5-triyl triacetate (1.2 g, 1 Eq, 3.0 mmol) and 5-bromopent-1-ene (1.3 g, 3 Eq, 8.9 mmol) in DMF (9.8 mL) at 23° C., was added piperazine (0.31 g, 1.2 Eq, 3.5 mmol) and cesium carbonate (1.9 g, 2 Eq, 5.9 mmol). The resultant reaction was stirred for 10 min at 23° C. and was then diluted with H₂O (100 mL) and extracted with EtOAc (2×100 mL). The combined organics were washed sequentially with 2M HCl (100 mL), H₂O (100 mL), sat. aq. NaHCO₃ (100 mL), and sat. aq. NaCl (100 mL). The combined organics were dried over sodium sulfate and concentrated in vacuo. The crude residue was purified by flash chromatography (5-20% EtOAc in Hexanes) to afford product F2 (365 mg, 0.84 mmol, 28% yield) as a yellow oil. ¹H NMR (400 MHZ, CDCl₃) δ 5.83-5.73 (m, 1H), 5.72 (d, J=5.2 Hz, 1H), 5.44 (dd, J=3.2, 1.3 Hz, 1H), 5.23 (qd, J=10.9, 4.2 Hz, 2H), 5.08-4.89 (m, 2H), 4.58 (td, J=6.6, 1.4 Hz, 1H), 4.10 (dd, J=6.5, 1.9 Hz, 2H), 2.64-2.45 (m, 2H), 2.18-2.11 (m, 5H), 2.07 (s, 3H), 2.04 (s, 3H), 1.98 (s, 3H), 1.77-1.64 (m, 2H). HRMS (ESI+, m/z): Calc'd for[C₁₉H₂₈O₉S+NH₄]⁺: 450.1792 found: 450.1797.

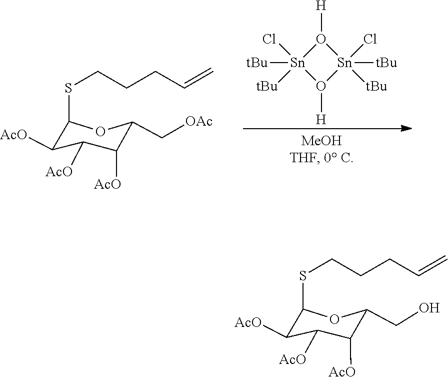

(2R,3S,4S,5R,6R)-2-(hydroxymethyl)-6-(pent-4-en-1-ylthio)tetrahydro-2H-pyran-3,4,5-triyl triacetate (F3): To a solution of (2R,3S,4S,5R,6R)-2-(acetoxymethyl)-6-(pent-4-en-1-ylthio)tetrahydro-2H-pyran-3,4,5-triyl triacetate (365 mg, 1 Eq, 844 μmol) in MeOH (6.75 mL) and THF (1.69 mL) at 0° C., was added dichlorotetrakis(1,1-dimethylethyl)di-μ-hydroxyditin (43.4 mg, 0.09 Eq, 76.0 μmol). The reaction was maintained at 0° C. for 6 hours and concentrated under reduced pressure. The crude residue was purified by flash chromatography (0-60% EtOAc in Hexanes) to afford F3 (127 mg, 325 μmol, 38.5%) as a colorless oil. ¹H NMR (400 MHZ, CDCl₃) δ 5.81-5.72 (m, 1H), 5.71 (d, J=5.1 Hz, 1H), 5.42 (d, J=2.5 Hz, 1H), 5.32-5.19 (m, 2H), 5.07-4.92 (m, 2H), 4.42 (t, J=6.5 Hz, 1H), 3.65 (dd, J=11.7, 6.8 Hz, 1H), 3.51 (dd, J=11.7, 6.2 Hz, 1H), 2.63-2.40 (m, 2H), 2.15 (s, 3H), 2.15-2.10 (m, 2H), 2.07 (s, 3H), 2.00 (s, 3H), 1.67 (ddp, J=10.8, 7.1, 3.6 Hz, 2H). HRMS (ESI+, m/z): Calc'd for[C₁₇H₂₆O₈S+NH₄]⁺: 408.1687 found: 408.1686

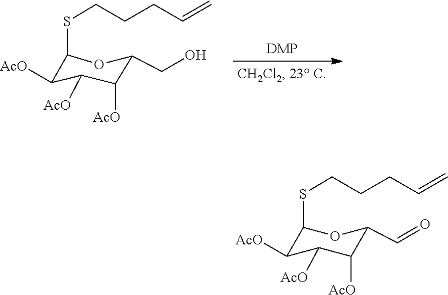 [SMILES: C=CCCCSC1OC(CO)C(OC(C)=O)C(OC(C)=O)C1OC(C)=O ; C=CCCCSC1OC(C=O)C(OC(C)=O)C(OC(C)=O)C1OC(C)=O]

(2S,3R,4S,5R,6R)-2-formyl-6-(pent-4-en-1-ylthio)tetrahydro-2H-pyran-3,4,5-triyl triacetate (F4): To a solution of (2R,3S,4S,5R,6R)-2-(hydroxymethyl)-6-(pent-4-en-1-ylthio)tetrahydro-2H-pyran-3,4,5-triyl triacetate (127 mg, 1 Eq, 0.325 mmol) in CH₂Cl₂ (3.25 mL), was added Dess-Martin Periodinane (221 mg, 1.6 Eq, 0.52 mmol). The reaction was stirred for 1 hour. The reaction mixture was quenched upon addition of sat. aq. NaHCO₃ (7.5 mL), sat. aq. Na₂S₂O₃ (7.5 mL) and CH₂Cl₂ (10 mL). The biphasic mixture was stirred vigorously for 30 min and the layers were then separated. The aq. layer was extracted with CH₂Cl₂ (2×10 mL) and the combined organics were dried over sodium sulfate and concentrated in vacuo to afford product F4 (104 mg, 0.27 mmol, 82% yield) as a colorless oil. ¹H NMR (400 MHZ, CDCl₃) δ 9.52 (d, J=0.8 Hz, 1H), 5.86 (d, J=5.3 Hz, 1H), 5.81 (dd, J=3.4, 1.7 Hz, 1H), 5.80-5.69 (m, 1H), 5.34-5.24 (m, 1H), 5.20 (dd, J=10.8, 3.3 Hz, 1H), 5.10-4.96 (m, 2H), 4.83 (d, J=1.7 Hz, 1H), 2.72-2.33 (m, 2H), 2.19-2.11 (m, 2H), 2.09 (s, 3H), 2.07 (s, 3H), 2.00 (s, 3H), 1.71 (ddq, J=14.2, 7.2, 3.1 Hz, 2H). HRMS (ESI+, m/z): Calc'd for[C₁₇H₂₄O₈S+H]⁺: 389.1265 found: 389.1275

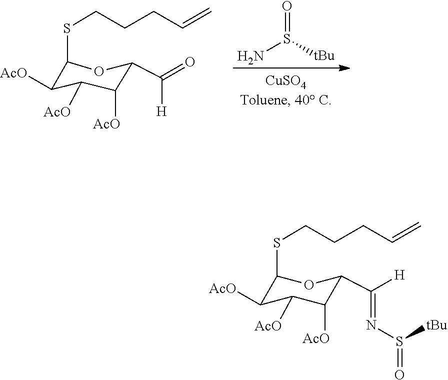 [SMILES: C=CCCCSC1OC(C=O)C(OC(C)=O)C(OC(C)=O)C1OC(C)=O ; CC(C)(C)[S@](N)=O ; C=CCCCSC1OC(/C=N/[S@](=O)C(C)(C)C)C(OC(C)=O)C(OC(C)=O)C1OC(C)=O]

(2R,3S,4S,5R,6R)-2-((E)-(((R)-tert-butylsulfinyl)imino)methyl)-6-(pent-4-en-1-ylthio)tetrahydro-2H-pyran-3,4,5-triyl triacetate (F5): To a solution of (2S,3R,4S,5R,6R)-2-formyl-6-(pent-4-en-1-ylthio)tetrahydro-2H-pyran-3,4,5-triyl tribenzoate (104 mg, 1 Eq, 0.18 mmol) in PhCH₃ (660 μl) was added (R)-2-methylpropane-2-sulfinamide (43.9 mg, 2 Eq, 0.36 mmol) and copper sulfate (43.3 mg, 1.5 Eq, 0.27 mmol). The reaction was stirred for 16 h at 40° C. and filtered through a pad of celite. The celite was washed with CH₂Cl₂ (2×25 mL) and the combined organics were concentrated under reduced pressure. The crude residue was purified by flash chromatography (0-60% EtOAc in Hexanes) to afford F5 (78 mg, 0.12 mmol, 64%) as a colorless oil. ¹H NMR (400 MHZ, CDCl₃) δ 7.94 (d, J=2.5 Hz, 1H), 5.87 (d, J=5.5 Hz, 1H), 5.81 (dt, J=2.6, 1.3 Hz, 1H), 5.80-5.70 (m, 1H), 5.33 (dd, J=10.8, 5.5 Hz, 1H), 5.29-5.23 (m, 2H), 5.09-4.98 (m, 2H), 2.68-2.46 (m, 2H), 2.15 (qd, J=7.0, 1.6 Hz, 2H), 2.11 (s, 3H), 2.09 (s, 3H), 2.01 (s, 3H), 1.78-1.63 (m, 2H), 1.18 (s, 9H). HRMS (ESI+, m/z): Calc'd for[C₂₁H₃₃NO: S₂+H]⁺: 492.1720 found: 492.1719.

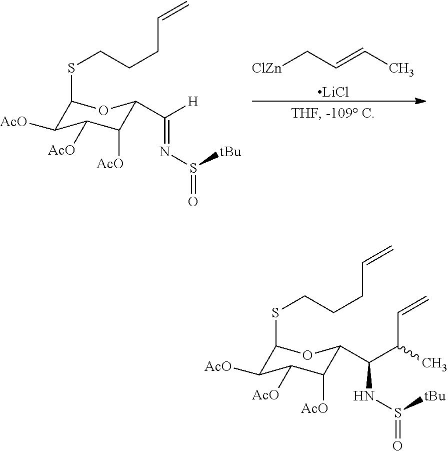

(2R,3S,4S,5R,6R)-2-((1R,2R)-1-(((R)-tert-butylsulfinyl)amino)-2-methylbut-3-en-1-yl)-6-(pent-4-en-1-ylthio)tetrahydro-2H-pyran-3,4,5-triyl triacetate (F6, 7:3 mixture of diastereomers): To a solution of (2R,3S,4S,5R,6R)-2-((E)-(((R)-tert-butylsulfinyl)imino)methyl)-6-(pent-4-en-1-ylthio)tetrahydro-2H-pyran-3,4,5-triyl triacetate (78 mg, 1 Eq, 0.16 mmol) in THF (1.6 mL) at −109° C., was added a solution of crotylzinc lithium chloride (0.71 mL, 0.27 M in THF, 1.2 Eq, 0.19 mmol) which was cooled to −109° C. The reaction was maintained at −109° C. for 5 min and sat. aq. NH₄Cl (10 mL) was added. The reaction was warmed to 23° C. and was diluted with EtOAc (30 mL) and H₂O (10 mL).

The layers were separated, and the aq. layer was extracted with EtOAc (2×20 mL). The combined organics were dried over sodium sulfate and concentrated in vacuo to afford product F6 (83 mg, 0.15 mmol, 96%). ¹H NMR (400 MHZ, CDCl₃) δ 5.90-5.69 (m, 3H), 5.63 (ddd, J=4.4, 3.0, 1.1 Hz, 1H), 5.21 (app ddd, J=11.1, 5.8, 2.9 Hz, 1H), 5.14 (dt, J=10.6, 1.5 Hz, 0.7H), 5.12-4.95 (m, 4.3H), 4.36 (d, J=8.0 Hz, 0.3H), 4.29 (d, J=8.6 Hz, 0.7H), 3.49 (ddd, J=8.1, 6.0, 4.4 Hz, 0.3H), 3.41 (ddd, J=8.5, 7.4, 2.9 Hz, 1H), 3.26 (d, J=7.4 Hz, 0.7H), 2.74 (ddd, J=9.7, 4.8, 2.0 Hz, 0.7H), 2.64-2.46 (m, 2H), 2.16 (s, 3H), 2.15-2.08 (m, 2H), 2.06 (s, 3H), 1.98 (s, 2H), 1.98 (s, 1H), 1.75-1.59 (m, 2H), 1.20 (s, 3H), 1.19 (s, 6H), 1.09 (d, J=7.0 Hz, 2H), 1.03 (d, J=6.8 Hz, 1H). HRMS (ESI+, m/z): Calc'd for[C₂₅H₄₁NO: S₂+H]⁺: 548.2346 found: 548.2351.

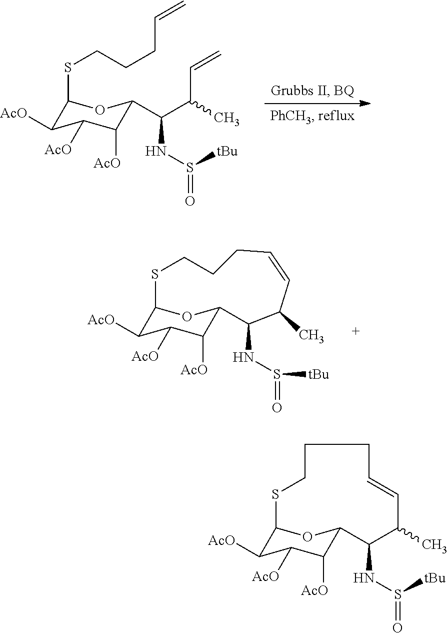

(1R,8R,9R,10R,11S,12S,13R,Z)-9-(((R)-tert-butylsulfinyl)amino)-8-methyl-14-oxa-2-thiabicyclo[8.3.1]tetradec-6-ene-11,12,13-triyl triacetate and (1R,9R,10R,11S,12S,13R,E)-9-(((R)-tert-butylsulfinyl)amino)-8-methyl-14-oxa-2-thiabicyclo[8.3.1]tetradec-6-ene-11,12,13-triyl triacetate (F7, inseparable 3:6:1 mixture of isomers): A degassed solution of (2R,3S,4S,5R,6R)-2-((1R,2R)-1-(((R)-tert-butylsulfinyl)amino)-2-methylbut-3-en-1-yl)-6-(pent-4-en-1-ylthio)tetrahydro-2H-pyran-3,4,5-triyl triacetate (83 mg, 1 Eq, 0.15 mmol) in PhCH₃ (76 mL) was heated to reflux. A solution of Grubbs II (26 mg, 0.2 Eq, 30 μmol) and benzoquinone (6.6 mg, 0.4 Eq, 61 μmol) in PhCH₃ (1 mL) was added and the solution was refluxed for 15 min. The reaction was quenched upon addition of DMSO (0.24 g, 0.22 mL, 20 Eq, 3.0 mmol) and the reaction was cooled to 0° C. The reaction was concentrated in vacuo pressure and the crude residue was dissolved in EtOAc (15 mL). The organic layer was washed with sat. aq. NaCl (15 mL), the layers were separated, and the aq. fraction was extracted with EtOAc (15 mL). The combined organics were dried over sodium sulfate and concentrated in vacuo. The crude residue was purified by flash chromatography (5-20% EtOAc in Hexanes) to afford product F7 (67 mg, 0.13 mmol, 85%). ¹H NMR (400 MHZ, CDCl₃) δ 5.68-5.35 (m, 4H), 5.15 (dd, J=10.9, 6.0 Hz, 1H), 4.97 (ddd, J=12.8, 11.1, 3.2 Hz, 1H), 4.43 (d, J=9.0 Hz, 0.3H), 4.39 (d, J=7.1 Hz, 0.6H), 4.04 (d, J=9.8 Hz, 0.1H), 3.57-3.48 (m, 0.6H), 3.42 (d, J=4.8 Hz, 0.3H), 3.37 (ddd, J=14.0, 4.8, 2.5 Hz, 0.1H), 3.28 (d, J=4.9 Hz, 0.6H), 3.22-3.00 (m, 1.4H), 2.77-2.64 (m, 0.9H), 2.45-2.21 (m, 2.4H), 2.18 (s, 1.8H), 2.17 (s, 0.9H), 2.16 (s, 0.3H), 2.18-2.06 (m, 0.6H), 2.07 (s, 0.9H), 2.06 (s, 1.8H), 2.04 (s, 0.3H), 1.98 (s, 1.2H), 1.97 (s, 1.8H), 1.95-1.82 (m, 1H), 1.24-1.18 (m, 9H), 1.16 (d, J=6.8 Hz, 0.9H), 1.07 (d, J=6.9 Hz, 1.8H), 1.02 (d, J=7.0 Hz, 0.3H). HRMS (ESI+, m/z): Calc'd for[C23H37NO: S₂+H]⁺: 520.2033 found: 520.2042.

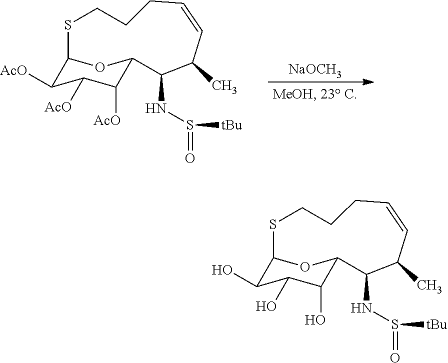 [SMILES: CC(=O)OC1C2OC(C(OC(C)=O)C1OC(C)=O)[C@H](N[S@](=O)C(C)(C)C)[C@H](C)/C=C\CCCS2 ; "NaOCH3" ; C[C@@H]1/C=C\CCCSC2OC(C(O)C(O)C2O)[C@@H]1N[S@](=O)C(C)(C)C]

(R)-2-methyl-N-((1R,8R,9R,10R,11R,12S,13R,Z)-11,12,13-trihydroxy-8-methyl-14-oxa-2-thiabicyclo[8.3.1]tetradec-6-en-9-yl) propane-2-sulfinamide (F8): The mixture containing (1R,8R,9R,10R,11S,12S,13R,Z)-9-(((R)-tert-butylsulfinyl)amino)-8-methyl-14-oxa-2-thiabicyclo[8.3.1]tetradec-6-ene-11,12,13-triyl triacetate (59 mg, 1 Eq, 0.11 mmol), was dissolved in sodium methoxide (1.1 mL, 0.5 M in MeOH, 5 Eq, 0.57 mmol) at 23° C. The reaction was maintained at 23° C. for 1 h and was neutralized upon the addition of formic acid (64 μL, 15 Eq, 1.7 mmol). The reaction mixture was filtered (0.2 uM syringe filter) and purified by prep-HPLC(40-100% MeCN in H₂O with 0.1% HCOOH over 30 minutes) to afford product F8 (6 mg, 20 μM, 10% yield) as a colorless film. ¹H NMR (400 MHZ, CD₃OD) δ 5.47 (td, J=10.9, 3.3 Hz, 1H), 5.24 (d, J=5.7 Hz, 1H), 5.20 (d, J=9.1 Hz, 1H), 5.12 (td, J=11.3, 2.3 Hz, 1H), 4.14 (d, J=3.5 Hz, 1H), 4.04 (d, J=10.3 Hz, 1H), 4.00 (dd, J=10.5, 5.7 Hz, 1H), 3.50 (td, J=9.6, 2.7 Hz, 1H), 3.43-3.33 (m, 1H), 3.35 (dd, J=10.5, 3.6 Hz, 1H), 3.11 (dqd, J=9.8, 6.8, 2.8 Hz, 1H), 2.47-2.28 (m, 2H), 2.25-2.06 (m, 2H), 1.50-1.38 (m, 1H), 1.31 (s, 9H), 0.98 (d, J=6.9 Hz, 3H). HRMS (ESI+, m/z): Calc'd for[C₁₇H₃₁NO: S₂+H]⁺: 394.1716 found: 394.1725.

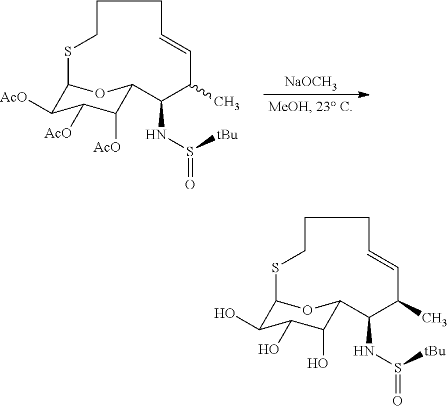 [SMILES: CC(=O)OC1C2OC(C(OC(C)=O)C1OC(C)=O)[C@H](N[S@](=O)C(C)(C)C)C(C)/C=C/CCCS2 ; "NaOCH3" ; C[C@@H]1/C=C/CCCSC2OC(C(O)C(O)C2O)[C@@H]1N[S@](=O)C(C)(C)C]

(R)-2-methyl-N-((1R,8R,9R,10R,11R,12S,13R,E)-11,12,13-trihydroxy-8-methyl-14-oxa-2-thiabicyclo[8.3.1]tetradec-6-en-9-yl) propane-2-sulfinamide (F9, isolated as >10:1 mixture of isomers, major reported): The mixture containing (1R,8R,9R,10R,11S,12S,13R,Z)-9-(((R)-tert-butylsulfinyl)amino)-8-methyl-14-oxa-2-thiabicyclo[8.3.1]tetradec-6-ene-11,12,13-triyl triacetate (59 mg, 1 Eq, 0.11 mmol), was dissolved in sodium methoxide (1.1 mL, 0.5 M in MeOH, 5 Eq, 0.57 mmol) at 23° C. The reaction was maintained at 23° C. for 1 h and was neutralized upon the addition of formic acid (64 μL, 15 Eq, 1.7 mmol). The reaction mixture was filtered (0.2 uM syringe filter) and purified by prep-HPLC(40-100% MeCN in H₂O with 0.1% HCOOH over 30 minutes) to afford product F9 (20 mg, 51 uM, 44% yield) as a colorless film. ¹H NMR (400 MHZ, CD₃OD) δ 5.54-5.50 (m, 2H), 5.22 (d, J=5.8 Hz, 1H), 4.26 (dd, J=7.6, 1.2 Hz, 1H), 4.06-4.03 (m, 1H), 3.99 (dd, J=10.3, 5.8 Hz, 1H), 3.56 (dd, J=7.6, 4.1 Hz, 1H), 3.40 (dd, J=10.3, 3.3 Hz, 1H), 3.09 (ddd, J=12.3, 6.2, 2.7 Hz, 1H), 2.79-2.67 (m, 1H), 2.46 (ddt, J=12.2, 8.2, 2.2 Hz, 1H), 2.37-2.26 (m, 1H), 2.23-2.13 (m, 1H), 1.99-1.88 (m, 1H), 1.60-1.49 (m, 1H), 1.29 (s, 9H), 1.03 (d, J=7.0 Hz, 3H). HRMS (ESI+, m/z): Calc'd for[C₁₇H₃₁NO₈S₂+H]⁺: 394.1716 found: 394.1729

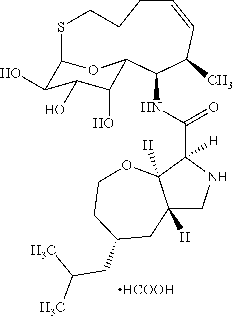 [SMILES: CC(C)C[C@@H]1CCO[C@@H]2[C@H](CN[C@@H]2C(=O)N[C@H]2C3OC(SCCC/C=C\[C@H]2C)C(O)C(O)C3O)C1 ; "·HCOOH"]

(4S,5aS,8S,8aR)-4-isobutyl-N-((1R,8R,9R,10R,11R,12S,13R,Z)-11,12,13-trihydroxy-8-methyl-14-oxa-2-thiabicyclo[8.3.1]tetradec-6-en-9-yl) octahydro-2H-oxepino[2,3-c]pyrrole-8-carboxamide formate salt (FSA1609096): The product FSA1609096 was prepared in a similar fashion to FSA1609033 from (R)-2-methyl-N-((1R,8R,9R,10R,11R,12S,13R,Z)-11,12,13-trihydroxy-8-methyl-14-oxa-2-thiabicyclo[8.3.1]tetradec-6-en-9-yl) propane-2-sulfinamide. ¹H NMR (400 MHZ, CD₃OD) δ 8 8.52 (s, 1H), 5.50 (td, J=10.8, 2.8 Hz, 1H), 5.26 (d, J=5.7 Hz, 1H), 5.19 (td, J=11.0, 1.7 Hz, 1H), 4.29 (dd, J=10.6, 3.0 Hz, 1H), 4.24 (t, J=9.1 Hz, 1H), 4.14 (d, J=8.7 Hz, 1H), 4.02 (dd, J=10.4, 5.7 Hz, 1H), 3.96 (dt, J=12.1, 3.8 Hz, 1H), 3.89 (dd, J=10.6, 1.0 Hz, 1H), 3.84-3.79 (m, 1H), 3.78 (d, J=3.6 Hz, 1H), 3.44 (dd, J=10.9, 7.5 Hz, 1H), 3.38 (dt, J=14.4, 3.5 Hz, 1H), 3.18 (ddd, J=10.6, 7.0, 3.2 Hz, 1H), 2.70 (t, J=11.3 Hz, 1H), 2.51-2.40 (m, 1H), 2.36 (t, J=13.3 Hz, 1H), 2.29-2.09 (m, 3H), 1.97 (d, J=13.4 Hz, 1H), 1.83-1.59 (m, 4H), 1.51-1.40 (m, 1H), 1.25-1.11 (m, 2H), 1.00 (d, J=6.9 Hz, 3H), 1.05-0.88 (m, 2H), 0.89 (d, J=6.6 Hz, 6H). HRMS (ESI+, m/z): Calc'd for[C₂₆H₄₄NO₆S+H]⁺: 513.2993 found: 513.2993

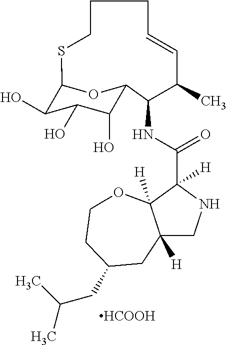 [SMILES: CC(C)C[C@@H]1CCO[C@@H]2[C@H](CN[C@@H]2C(=O)N[C@H]2C3OC(SCCC/C=C/[C@H]2C)C(O)C(O)C3O)C1 ; "·HCOOH"]

(4S,5aS,8S,8aR)-4-isobutyl-N-((1R,8R,9R,10R,11R,12S,13R,E)-11,12,13-trihydroxy-8-methyl-14-oxa-2-thiabicyclo[8.3.1]tetradec-6-en-9-yl) octahydro-2H-oxepino[2,3-c]pyrrole-8-carboxamide formate salt (FSA1609095): The product FSA1609095 was prepared in a similar fashion to FSA1609033 from (R)-2-methyl-N-((1R,8R,9R,10R,11R,12S,13R,E)-11,12,13-trihydroxy-8-methyl-14-oxa-2-thiabicyclo[8.3.1]tetradec-6-en-9-yl) propane-2-sulfinamide. ¹H NMR (400 MHZ, CD₃OD) δ 8 5.60-5.45 (m, 2H), 5.24 (d, J=5.8 Hz, 1H), 4.44 (d, J=8.9 Hz, 1H), 4.42-4.30 (m, 2H), 4.07 (dd, J=10.2, 1.0 Hz, 1H), 4.03-3.94 (m, 2H), 3.84-3.71 (m, 2H), 3.63 (dd, J=11.3, 7.4 Hz, 1H), 3.34 (d, J=3.4 Hz, 2H), 3.17 (dt, J=12.3, 4.0 Hz, 1H), 2.93 (t, J=11.8 Hz, 1H), 2.69-2.59 (m, 1H), 2.57 (td, J=12.6, 3.8 Hz, 1H), 2.36-2.21 (m, 3H), 2.05-1.89 (m, 2H), 1.82-1.58 (m, 5H), 1.19 (td, J=6.8, 3.5 Hz, 2H), 1.04 (d, J=7.2 Hz, 3H), 1.01-0.93 (m, 1H), 0.89 (d, J=6.4 Hz, 6H). HRMS (ESI+, m/z): Calc'd for[C₂₆H₄₄NO₆S+H]⁺: 513.2993 found: 513.2996.

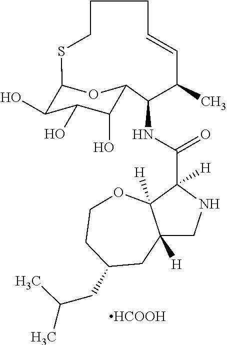 [SMILES: CC(C)C[C@@H]1CCO[C@@H]2[C@H](CN[C@@H]2C(=O)N[C@H]2C3OC(SCCC/C=C/[C@H]2C)C(O)C(O)C3O)C1 ; "·HCOOH"]

(4S,5aS,8S,8aR)-4-isobutyl-N-((1R,8S,9R,10R,11R,12S,13R,E)-11,12,13-trihydroxy-8-methyl-14-oxa-2-thiabicyclo[8.3.1]tetradec-6-en-9-yl) octahydro-2H-oxepino[2,3-c]pyrrole-8-carboxamide formate salt (FSA1609094): The product FSA1609094 was prepared in a similar fashion to FSA1609033 from (R)-2-methyl-N-((1R,8S,9R,10R,11R,12S,13R,E)-11,12,13-trihydroxy-8-methyl-14-oxa-2-thiabicyclo[8.3.1]tetradec-6-en-9-yl) propane-2-sulfinamide. ¹H NMR (400 MHZ, CD₃OD) δ 8 8.46 (s, 1H), 5.54 (ddd, J=14.6, 9.2, 5.2 Hz, 1H), 5.29 (dd, J=15.1, 9.4 Hz, 1H), 5.26 (d, J=5.9 Hz, 1H), 4.29 (t, J=9.2 Hz, 1H), 4.18 (dd, J=11.0, 8.5 Hz, 2H), 4.00-3.93 (m, 2H), 3.85 (d, J=3.1 Hz, 1H), 3.84-3.75 (m, 1H), 3.71 (t, J=9.0 Hz, 1H), 3.54-3.46 (m, 1H), 3.30-3.27 (m, 1H), 3.16 (ddd, J=12.7, 8.0, 1.4 Hz, 1H), 2.80 (t, J=11.4 Hz, 1H), 2.44 (ddd, J=12.9, 8.8, 1.5 Hz, 1H), 2.36-2.20 (m, 3H), 2.11 (q, J=9.7 Hz, 1H), 2.04-1.95 (m, 1H), 1.94 (s, 1H), 1.82-1.59 (m, 4H), 1.52 (q, J=11.2 Hz, 1H), 1.18 (td, J=6.8, 4.5 Hz, 2H), 1.04 (d, J=6.6 Hz, 3H), 0.99-0.91 (m, 1H), 0.91-0.84 (m, 6H). HRMS (ESI+, m/z): Calc'd for[C₂₆H₄₄NO₆S+H]⁺: 513.2993 found: 513.2996

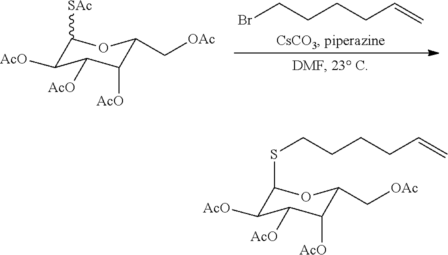

(2R,3S,4S,5R,6R)-2-(acetoxymethyl)-6-(hex-5-en-1-ylthio)tetrahydro-2H-pyran-3,4,5-triyl triacetate (G1): To a degassed solution of (2R,3S,4S,5R)-2-(acetoxymethyl)-6-(acetylthio)tetrahydro-2H-pyran-3,4,5-triyl triacetate (F1, 0.90 g, 1 Eq, 2.2 mmol) and 6-bromohex-1-ene (720 mg, 2 Eq, 4.4 mmol) in DMF (9.6 mL) at 23° C., was added piperazine (0.23 g, 1.2 Eq, 2.7 mmol) and cesium carbonate (1.44 g, 2 Eq, 4.4 mmol). The resultant reaction was stirred for 10 min at 23° C. and was then diluted with H₂O (100 mL) and extracted with EtOAc (2×100 mL). The combined organics were washed with 2M HCl (100 mL), H₂O (100 mL), sat. aq. NaHCO₃ (100 mL), and sat. aq. NaCl (100 mL). The combined organics were dried over sodium sulfate and concentrated in vacuo. The crude residue was purified by flash chromatography (5-20% EtOAc in Hexanes) to afford product G1 (350 mg, 0.78 mmol, 35% yield) as an orange oil. ¹H NMR (400 MHZ, CDCl₃) δ 5.77 (ddt, J=16.9, 10.1, 6.7 Hz, 1H), 5.70 (d, J=5.2 Hz, 1H), 5.43 (dd, J=3.1, 1.3 Hz, 1H), 5.25 (dd, J=10.8, 5.2 Hz, 1H), 5.20 (dd, J=10.9, 3.1 Hz, 1H), 5.03-4.89 (m, 2H), 4.57 (td, J=6.5, 1.3 Hz, 1H), 4.16-4.02 (m, 2H), 2.68-2.36 (m, 2H), 2.13 (s, 3H), 2.06 (s, 3H), 2.08-2.01 (m, 2H), 2.03 (s, 3H), 1.97 (s, 3H), 1.67-1.54 (m, 2H), 1.51-1.41 (m, 2H). HRMS (ESI+, m/z): Calc'd for[C₂₀H₃₀O₉S+NH₄]⁺: 464.1949 found: 464.1966.

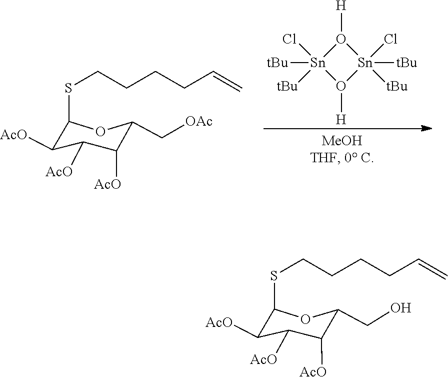

(2R,3R,4S,5S,6R)-2-(hex-5-en-1-ylthio)-6-(hydroxymethyl)tetrahydro-2H-pyran-3,4,5-triyl triacetate (G2): To a solution of (2R,3S,4S,5R,6R)-2-(acetoxymethyl)-6-(hex-5-en-1-ylthio)tetrahydro-2H-pyran-3,4,5-triyl triacetate (250 mg, 1 Eq, 560 μmol) in MeOH (1.49 mL) and THF (0.75 mL) at 0° C., was added dichlorotetrakis(1,1-dimethylethyl)di-μ-hydroxyditin (38.4 mg, 0.12 Eq, 67.2 μmol). The reaction mixture was maintained at this temperature for 7 hours. The reaction mixture was concentrated in vacuo, and the crude residue was purified by flash chromatography (0-60% EtOAc in Hexanes) to afford G2 (200 mg, 0.49 mmol, 88%) as a light orange oil. ¹H NMR (400 MHZ, CDCl₃) δ 5.76 (ddt, J=16.9, 10.1, 6.6 Hz, 1H), 5.70 (d, J=5.2 Hz, 1H), 5.42 (dd, J=3.1, 1.2 Hz, 1H), 5.27 (dd, J=10.9, 5.2 Hz, 1H), 5.22 (dd, J=10.8, 3.1 Hz, 1H), 4.98 (dq, J=17.6, 2.0 Hz, 1H), 4.96-4.91 (m, 1H), 4.42 (t, J=6.5 Hz, 1H), 3.64 (dd, J=11.6, 6.8 Hz, 1H), 3.52 (dd, J=11.6, 6.2 Hz, 1H), 2.61-2.41 (m, 2H), 2.14 (s, 3H), 2.06 (s, 3H), 2.05-2.00 (m, 2H), 1.99 (s, 3H), 1.65-1.52 (m, 2H), 1.50-1.39 (m, 2H). HRMS (ESI+, m/z): Calc'd for[C₁₈H₁₈O₈S+NH₄]⁺: 422.1834, found: 422.1841.

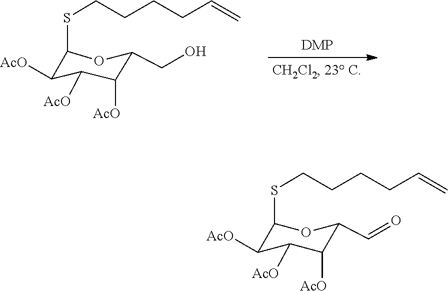 [SMILES: C=CCCCCSC1OC(CO)C(OC(C)=O)C(OC(C)=O)C1OC(C)=O ; C=CCCCCSC1OC(C=O)C(OC(C)=O)C(OC(C)=O)C1OC(C)=O]

(2S,3R,4S,5R,6R)-2-formyl-6-(hex-5-en-1-ylthio)tetrahydro-2H-pyran-3,4,5-triyl triacetate (G3): To a solution of (2R,3R,4S,5S,6R)-2-(hex-5-en-1-ylthio)-6-(hydroxymethyl)tetrahydro-2H-pyran-3,4,5-triyl triacetate (240 mg, 1 Eq, 0.59 mmol) in CH₂Cl₂ (6.0 mL) was added, Dess-Martin Periodinane (277 mg, 1.1 Eq, 0.65 mmol). The reaction was stirred for 1 hour. The reaction mixture was quenched upon addition of sat. aq. NaHCO₃ (7.5 mL), sat. aq. Na₂S₂O₃ (7.5 mL) and CH₂Cl₂ (10 mL). The biphasic mixture was stirred vigorously for 30 min and the layers were then separated. The aq. layer was extracted with CH₂Cl₂ (2×10 mL) and the combined organics were dried over sodium sulfate and concentrated in vacuo to afford product G3 (230 mg, 0.57 mmol, 96% yield) as a colorless oil. ¹H NMR (400 MHZ, CDCl₃) δ 9.50 (s, 1H), 5.84 (d, J=5.5 Hz, 1H), 5.80 (dd, J=3.4, 1.8 Hz, 1H), 5.79-5.68 (m, 1H), 5.27 (dd, J=10.8, 5.5 Hz, 1H), 5.18 (dd, J=10.8, 3.3 Hz, 1H), 5.04-4.91 (m, 2H), 4.82 (d, J=1.6 Hz, 1H), 2.67-2.46 (m, 2H), 2.07 (d, J=0.6 Hz, 3H), 2.06 (s, 3H), 2.02 (d, J=6.7 Hz, 2H), 1.99 (d, J=0.6 Hz, 3H), 1.65-1.54 (m, 2H), 1.45 (pd, J=7.4, 2.7 Hz, 2H). HRMS (ESI+, m/z): Calc'd for[C₁₈H₂₆O₈S+H]⁺: 403.1421 found: 403.1421

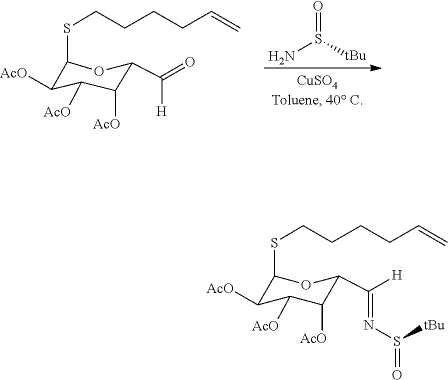 [SMILES: C=CCCCCSC1OC(C=O)C(OC(C)=O)C(OC(C)=O)C1OC(C)=O ; CC(C)(C)[S@](N)=O ; C=CCCCCSC1OC(/C=N/[S@](=O)C(C)(C)C)C(OC(C)=O)C(OC(C)=O)C1OC(C)=O]

(2R,3S,4S,5R,6R)-2-((E)-(((R)-tert-butylsulfinyl)imino)methyl)-6-(hex-5-en-1-ylthio)tetrahydro-2H-pyran-3,4,5-triyl triacetate (G4): To a solution of (2S,3R,4S,5R,6R)-2-formyl-6-(hex-5-en-1-ylthio)tetrahydro-2H-pyran-3,4,5-triyl tribenzoate (220 mg, 1 Eq, 0.37 mmol) in PhCH₃ (1.25 mL) was added (R)-2-methylpropane-2-sulfinamide (90.6 mg, 2 Eq, 0.75 mmol) and CuSO₄ (89.5 mg, 1.5 Eq, 0.56 mmol). The reaction was stirred for 12 h at 40° C. and filtered through a pad of celite. The celite was washed with CH₂Cl₂ (2×25 mL) and the combined organics were concentrated under reduced pressure. The crude residue was purified by flash chromatography (0-100% EtOAc in Hexanes) to afford G4 (230 mg, 0.33 mmol, 89% yield) as a colorless oil. ¹H NMR (400 MHz, CDCl₃) δ 7.91 (d, J=2.5 Hz, 1H), 5.83 (d, J=5.5 Hz, 1H), 5.77 (dd, J=3.1, 1.4 Hz, 1H), 5.81-5.70 (m, 1H), 5.30 (dd, J=10.8, 5.5 Hz, 1H), 5.26-5.19 (m, 2H), 5.03-4.91 (m, 2H), 2.63-2.46 (m, 2H), 2.07 (s, 3H), 2.06 (s, 3H), 2.05-2.00 (m, 2H), 1.97 (s, 3H), 1.65-1.55 (m, 2H), 1.45 (dtd, J=15.1, 7.5, 2.8 Hz, 2H), 1.14 (s, 9H). HRMS (ESI+, m/z): Calc'd for[C₂₂H₃₆NO: S₂+H]⁺: 506.1877 found: 506.1879.

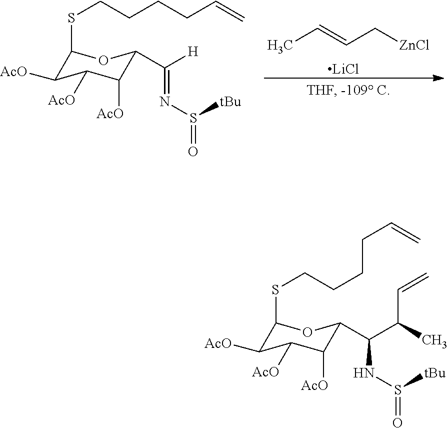

(2R,3S,4S,5R,6R)-2-((1R,2R)-1-(((R)-tert-butylsulfinyl)amino)-2-methylbut-3-en-1-yl)-6-(hex-5-en-1-ylthio)tetrahydro-2H-pyran-3,4,5-triyl triacetate (G5, as a 7:3 mixture of diastereomers): To a solution of (2R,3S,4S,5R,6R)-2-((E)-(((R)-tert-butylsulfinyl)imino)methyl)-6-(hex-5-en-1-ylthio)tetrahydro-2H-pyran-3,4,5-triyl triacetate (230 mg, 1 Eq, 455 μmol) in THF (4.55 mL) at −109° C. was added (E)-but-2-en-1-ylzinc (II) lithium chloride (180 mg, 3.37 mL, 0.27 molar, 2 Eq, 910 μmol) that was also cooled to −109° C. The reaction was maintained at this temperature for 10 minutes and sat. aq. NH₄Cl (5 mL) was added. The reaction was warmed to 23° C. and was diluted with 30 mL EtOAc and 15 mL water. The layers were separated, and the aq. layer was extracted with EtOAc (2×20 mL). The combined organics were dried over sodium sulfate and concentrated in vacuo to afford G5 (250 mg, 0.45 mmol, 97% yield) as a colorless oil. ¹H NMR (400 MHZ, CDCl₃) 8 5.95-5.67 (m, 3H), 5.63 (d, J=3.3 Hz, 1H), 5.21 (app ddd, J=11.1, 5.7, 2.4 Hz, 1H), 5.14 (dt, J=10.7, 1.4 Hz, 0.7H), 5.11-5.04 (m, 2.3H), 5.03-4.92 (m, 2H), 4.37 (d, J=8.2 Hz, 0.3H), 4.29 (d, J=8.7 Hz, 0.7H), 3.48 (ddd, J=7.9, 5.9, 4.4 Hz, 0.3H), 3.40 (app ddd, J=8.6, 6.6, 2.8 Hz, 1H), 3.23 (d, J=7.4 Hz, 0.7H), 2.80-2.69 (m, 0.7H), 2.64-2.48 (m, 2.3H), 2.16 (s, 3H), 2.06 (s, 3H), 2.10-2.00 (m, 2H), 1.98 (s, 3H), 1.66-1.53 (m, 2H), 1.52-1.41 (m, 2H), 1.19 (d, J=2.1 Hz, 9H), 1.09 (d, J=7.0 Hz, 2.1H), 1.03 (d, J=6.7 Hz, 0.9H). HRMS (ESI+, m/z): Calc'd for[C₂₆H₄₃NO: S₂+H]⁺: 562.2503 found: 562.2502

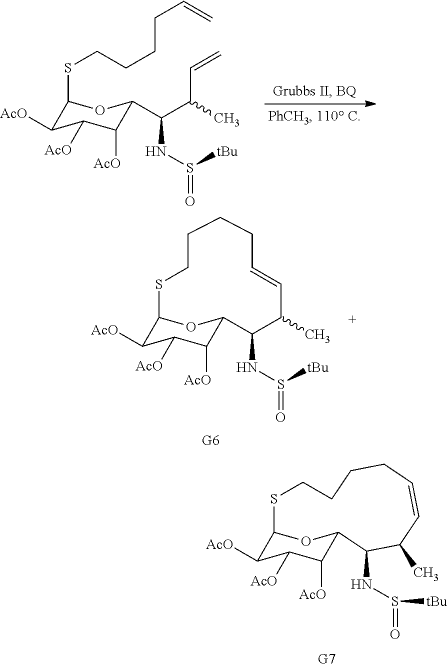

(1R,10R,11R,12S,13S,14R,E)-10-(((R)-tert-butylsulfinyl)amino)-9-methyl-15-oxa-2-thiabicyclo[9.3.1]pentadec-7-ene-12,13,14-triyl triacetate (G6, 3:1 Mixture of diastereomers); (1R,10R,11R,12S,13S,14R,Z)-10-(((R)-tert-butylsulfinyl)amino)-9-methyl-15-oxa-2-thiabicyclo[9.3.1]pentadec-7-ene-12,13,14-triyl triacetate (G7): A degassed solution of (2R,3S,4S,5R,6R)-2-((1R,2R)-1-(((R)-tert-butylsulfinyl)amino)-2-methylbut-3-en-1-yl)-6-(hex-5-en-1-ylthio)tetrahydro-2H-pyran-3,4,5-triyl triacetate (250 mg, 1 Eq, 445 μmol) in PhCH₃ (223 mL) was heated to reflux. A solution of Grubbs II (77.7 mg, 0.2 Eq, 89.0 μmol) and benzoquinone (19.2 mg, 0.4 Eq, 178 μmol) in PhCH₃ (1 mL) was added and the solution was refluxed for a further 15 minutes. The reaction was quenched upon addition of DMSO (632 μL, 20 Eq, 8.90 mmol) and the reaction was cooled 0° C. The reaction was concentrated in vacuo and the crude residue was dissolved in EtOAc (100 mL). The organic layer was washed with sat. aq. NaCl (1×50 mL). The organic fraction was dried over sodium sulfate and concentrated in vacuo to afford a crude residue that was purified by flash chromatography (0%-80% EtOAc in Hexanes) to afford G6 (154 mg, 289 μmol, 64.8%) and G7 (15 mg, 28 μmol, 6.3%).

Product G6: 1H NMR (400 MHZ, CDCl₃) δ 5.71-5.46 (m, 4H), 5.30-5.18 (m, 1.25H), 5.14 (dd, J=11.0, 2.9 Hz, 0.75H), 4.40 (d, J=9.2 Hz, 0.25H), 4.31 (d, J=9.8 Hz, 0.75H), 3.49-3.37 (m, 1H), 3.28 (dd, J=9.5, 4.1 Hz, 0.25H), 3.18 (d, J=6.2 Hz, 0.75H), 2.83 (s, 0.25H), 2.76-2.63 (m, 2.75H), 2.18 (s, 0.75H), 2.16 (s, 2.25H), 2.10 (app dq, J=7.4, 3.3 Hz, 2H), 2.05 (s, 3H), 1.97 (s, 3H), 1.87-1.76 (m, 1H), 1.74-1.65 (m, 1H), 1.39 (app. dp, J=10.3, 6.9 Hz, 2H), 1.23 (s, 6.75H), 1.21 (s, 2.25H), 1.21-1.18 (m, 0.75H), 1.06 (d, J=7.1 Hz, 2.25H). HRMS (ESI+, m/z): Calc'd for[C₂₄H₃₉NO₈S₂+H]⁺: 534.2190 found: 534.2186

Product G7: HRMS (ESI+, m/z): Calc'd for[C₂₄H₃₉NO: S₂+H]⁺: 534.2190 found: 534.2213.

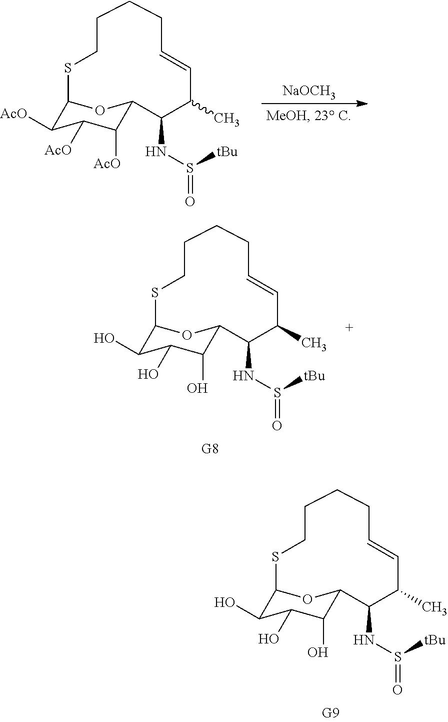

(R)-2-methyl-N-((1R,9R,10R,11R,12R,13S,14R,E)-12,13,14-trihydroxy-9-methyl-15-oxa-2-thiabicyclo[9.3.1]pentadec-7-en-10-yl) propane-2-sulfinamide (G8); (R)-2-methyl-N-((1R,9S,10R,11R,12R,13S,14R,E)-12,13,14-trihydroxy-9-methyl-15-oxa-2-thiabicyclo[9.3.1]pentadec-7-en-10-yl) propane-2-sulfinamide (G9): (1R,10R,11R,12S,13S,14R,E)-10-(((R)-tert-butylsulfinyl)amino)-9-methyl-15-oxa-2-thiabicyclo[9.3.1]pentadec-7-ene-12,13,14-triyl triacetate (150 mg, 1 Eq, 281 μmol) was dissolved in a solution of sodium methoxide (60.7 mg, 2.25 mL, 0.5 molar, 4 Eq, 1.12 mmol) in MeOH at 23° C. The reaction mixture was maintained at 23° C. for 30 min. The reaction was then diluted with MeOH (0.5 mL) and filtered (0.2 μM syringe filter) and purified by prep-HPLC(5-50% MeCN in H₂O with 0.1% HCOOH over 30 minutes) to afford separately product G8 (65 mg, 0.16 mmol, 57% yield) and G9 (8.5 mg, 21 μmol, 7.2% yield).

G8: ¹H NMR (400 MHZ, CD₃OD) δ 5.61 (ddd, J=15.0, 10.3, 4.6 Hz, 1H), 5.45 (dd, J=15.1, 9.1 Hz, 1H), 5.31-5.23 (m, 2H), 4.22 (d, J=2.2 Hz, 1H), 4.20 (dd, J=11.3, 1.4 Hz, 1H), 4.03 (dd, J=10.2, 5.4 Hz, 1H), 3.65 (dd, J=10.2, 3.6 Hz, 1H), 3.67-3.57 (m, 1H), 2.84 (ddd, J=11.1, 9.5, 6.8 Hz, 1H), 2.71-2.59 (m, 2H), 2.23-2.12 (m, 1H), 2.12-1.98 (m, 1H), 1.95-1.74 (m, 2H), 1.49-1.34 (m, 2H), 1.31 (s, 9H), 1.02 (d, J=7.1 Hz, 3H).

G9: ¹H NMR (400 MHZ, CD₃OD) δ 5.68 (dd, J=15.5, 5.8 Hz, 1H), 5.46 (dtd, J=15.1, 7.2, 1.3 Hz, 1H), 5.28 (d, J=5.5 Hz, 1H), 4.20 (d, J=9.3 Hz, 1H), 4.12 (d, J=3.4 Hz, 1H), 4.05 (dd, J=10.2, 5.5 Hz, 1H), 3.61 (dd, J=10.2, 3.3 Hz, 1H), 3.42 (dd, J=9.3, 6.0 Hz, 1H), 2.74-2.57 (m, 2H), 2.34 (h, J=6.7 Hz, 1H), 2.23-2.13 (m, 1H), 2.13-2.02 (m, 1H), 1.79 (dtd, J=17.1, 9.2, 4.4 Hz, 1H), 1.72-1.59 (m, 1H), 1.59-1.44 (m, 2H), 1.28 (s, 9H), 1.16 (d, J=6.9 Hz, 3H). HRMS (ESI+, m/z): Calc'd for[C₁₈H₃₃NO₅S₂+H]⁺: 408.1873 found: 408.1873. HRMS (ESI+, m/z): Calc'd for[C₁₈H₃₃NO₅S₂+H]⁺: 408.1873 found: 408.1873.

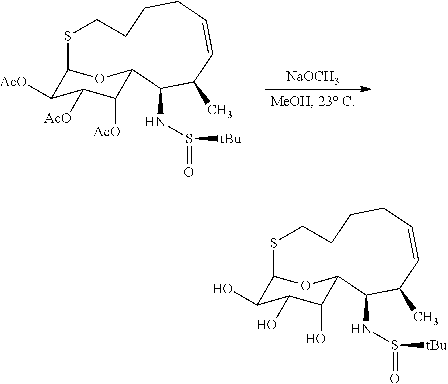 [SMILES: CC(=O)OC1C2OC(C(OC(C)=O)C1OC(C)=O)[C@H](N[S@](=O)C(C)(C)C)[C@H](C)/C=C\CCCCS2 ; "NaOCH3" ; C[C@@H]1/C=C\CCCCSC2OC(C(O)C(O)C2O)[C@@H]1N[S@](=O)C(C)(C)C]

(R)-2-methyl-N-((1R,9R,10R,11R,12R,13S,14R,Z)-12,13,14-trihydroxy-9-methyl-15-oxa-2-thiabicyclo[9.3.1]pentadec-7-en-10-yl) propane-2-sulfinamide (G10): (1R,9R,10R,11R,12S,13S,14R,Z)-10-(((R)-tert-butylsulfinyl)amino)-9-methyl-15-oxa-2-thiabicyclo[9.3.1]pentadec-7-ene-12,13,14-triyl triacetate (7.5 mg, 1 Eq, 14 μmol) was dissolved in a solution of sodium methanolate (0.76 mg, 1 Eq, 14 μmol) in MeOH at 23° C. The reaction mixture was maintained at 23° C. for 30 min. The reaction was then diluted with MeOH (0.5 mL) and filtered (0.2 uM syringe filter) and purified by prep-HPLC(5-50% MeCN in H₂O with 0.1% HCOOH over 30 minutes) to afford G10 (2.5 mg, 6.1 μmol, 44%). ¹H NMR (400 MHZ, CD₃OD) δ 5.47-5.37 (m, 2H), 5.36 (d, J=5.3 Hz, 1H), 4.12 (d, J=3.5 Hz, 1H), 4.07 (dd, J=10.2, 5.5 Hz, 1H), 3.97 (d, J=10.1 Hz, 1H), 3.66 (dd, J=10.2, 3.5 Hz, 1H), 3.55-3.41 (m, 1H), 3.09 (p, J=7.6 Hz, 1H), 2.62-2.36 (m, 3H), 1.89 (d, J=14.0 Hz, 1H), 1.75-1.56 (m, 2H), 1.56-1.42 (m, 2H), 1.32 (s, 9H), 0.99 (d, J=7.0 Hz, 3H). HRMS (ESI+, m/z): Calc'd for[C₁₈H₃₃NO₅S₂+H]⁺: 408.1873 found: 408.1885

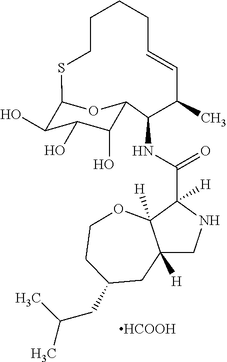 [SMILES: CC(C)C[C@@H]1CCO[C@@H]2[C@H](CN[C@@H]2C(=O)N[C@H]2C3OC(SCCCC/C=C/[C@H]2C)C(O)C(O)C3O)C1 ; "·HCOOH"]

(4S,5aS,8S,8aR)-4-isobutyl-N-((1R,9R,10R,11R,12R,13S,14R,E)-12,13,14-trihydroxy-9-methyl-15-oxa-2-thiabicyclo[9.3.1]pentadec-7-en-10-yl) octahydro-2H-oxepino[2,3-c]pyrrole-8-carboxamide (FSA1610016): Product FSA1610016 was prepared in a similar fashion to FSA1609033 from (R)-2-methyl-N-((1R,9R,10R,11R,12R,13S,14R,E)-12,13,14-trihydroxy-9-methyl-15-oxa-2-thiabicyclo[9.3.1]pentadec-7-en-10-yl) propane-2-sulfinamide. ¹H NMR (400 MHZ, CD₃OD) δ 8.53 (s, 1H), 5.67 (ddd, J=14.9, 10.4, 4.5 Hz, 1H), 5.52 (dd, J=15.2, 9.6 Hz, 1H), 5.27 (d, J=5.4 Hz, 1H), 4.35 (dd, J=10.6, 3.5 Hz, 1H), 4.21 (t, J=9.1 Hz, 1H), 4.11-3.99 (m, 3H), 3.94 (dt, J=12.2, 3.8 Hz, 1H), 3.84-3.72 (m, 2H), 3.58 (dd, J=10.2, 3.6 Hz, 1H), 3.38 (dd, J=10.7, 7.2 Hz, 1H), 2.91-2.78 (m, 1H), 2.72 (ddd, J=9.9, 7.0, 3.4 Hz, 1H), 2.69-2.56 (m, 2H), 2.27-2.14 (m, 2H), 2.08 (td, J=9.7, 3.7 Hz, 1H), 2.01-1.93 (m, 1H), 1.93-1.78 (m, 2H), 1.78-1.59 (m, 4H), 1.41 (q, J=9.5 Hz, 2H), 1.17 (h, J=6.8 Hz, 2H), 1.02 (d, J=7.2 Hz, 3H), 0.93 (d, J=11.7 Hz, 1H), 0.89 (d, J=6.5 Hz, 6H). HRMS (ESI+, m/z): Calc'd for[C₂₇H₄₆N₂O₅S+H]⁺: 527.3149 found: 527.3161.

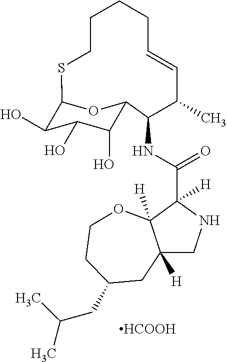 [SMILES: CC(C)C[C@@H]1CCO[C@@H]2[C@H](CN[C@@H]2C(=O)N[C@H]2C3OC(SCCCC/C=C/[C@@H]2C)C(O)C(O)C3O)C1 ; "·HCOOH"]

(4S,5aS,8S,8aR)-4-isobutyl-N-((1R,9S,10R,11R,12R,13S,14R,E)-12,13,14-trihydroxy-9-methyl-15-oxa-2-thiabicyclo[9.3.1]pentadec-7-en-10-yl) octahydro-2H-oxepino[2,3-c]pyrrole-8-carboxamide (FSA1610014): Product FSA1610014 was prepared in a similar fashion to FSA1609033 from (R)-2-methyl-N-((1R,9S,10R,11R,12R,13S,14R,E)-12,13,14-trihydroxy-9-methyl-15-oxa-2-thiabicyclo[9.3.1]pentadec-7-en-10-yl) propane-2-sulfinamide. ¹H NMR (400 MHZ, CD₃OD) δ 8.53 (s, 1H), 5.63 (dd, J=15.3, 6.8 Hz, 1H), 5.48-5.36 (m, 1H), 5.33 (d, J=5.6 Hz, 1H), 4.26 (t, J=9.2 Hz, 1H), 4.14-4.07 (m, 2H), 4.07-3.90 (m, 3H), 3.87 (dd, J=3.3, 1.1 Hz, 1H), 3.77 (ddd, J=11.9, 10.0, 3.1 Hz, 1H), 3.60 (dd, J=10.2, 3.3 Hz, 1H), 3.42 (dd, J=10.7, 7.3 Hz, 1H), 2.72 (t, J=11.3 Hz, 1H), 2.68-2.55 (m, 2H), 2.42 (h, J=6.9 Hz, 1H), 2.32-2.13 (m, 2H), 2.11-2.00 (m, 1H), 2.00-1.93 (m, 1H), 1.82-1.61 (m, 5H), 1.61-1.50 (m, 3H), 1.17 (td, J=6.8, 4.7 Hz, 2H), 1.12 (d, J=6.9 Hz, 3H), 0.96-0.90 (m, 1H), 0.89 (d, J=6.5 Hz, 6H). HRMS (ESI+, m/z): Calc'd for[C₂₇H₄₆N₂O₅S+H]⁺: 527.3149 found: 527.3154

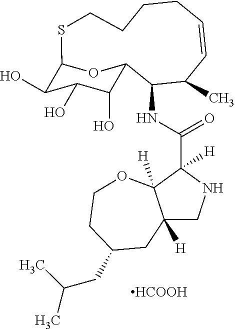 [SMILES: CC(C)C[C@@H]1CCO[C@@H]2[C@H](CN[C@@H]2C(=O)N[C@H]2C3OC(SCCCC/C=C\[C@H]2C)C(O)C(O)C3O)C1 ; "·HCOOH"]

(4S,5aS,8S,8aR)-4-isobutyl-N-((1R,9R,10R,11R,12R,13S,14R,Z)-12,13,14-trihydroxy-9-methyl-15-oxa-2-thiabicyclo[9.3.1]pentadec-7-en-10-yl) octahydro-2H-oxepino[2,3-c]pyrrole-8-carboxamide (FSA1610015): Product FSA1610015 was prepared in a similar fashion to FSA1609033 from (R)-2-methyl-N-((1R,9R,10R,11R,12R,13S,14R,Z)-12,13,14-trihydroxy-9-methyl-15-oxa-2-thiabicyclo[9.3.1]pentadec-7-en-10-yl) propane-2-sulfinamide. ¹H NMR (400 MHZ, CD₃OD) 8 5.51-5.39 (m, 2H), 5.37 (d, J=5.5 Hz, 1H), 4.30 (dd, J=10.5, 1.7 Hz, 1H), 4.25 (t, J=9.1 Hz, 1H), 4.16 (d, J=8.8 Hz, 1H), 4.08 (dd, J=10.2, 5.6 Hz, 1H), 3.97 (dt, J=12.2, 3.9 Hz, 1H), 3.90 (d, J=10.6 Hz, 1H), 3.85-3.75 (m, 2H), 3.61 (dd, J=10.2, 3.4 Hz, 1H), 3.46 (dd, J=10.8, 7.4 Hz, 1H), 3.21-3.10 (m, 1H), 2.73 (t, J=11.3 Hz, 1H), 2.67-2.53 (m, 2H), 2.53-2.41 (m, 1H), 2.24 (p, J=8.8 Hz, 1H), 2.03-1.94 (m, 1H), 1.92-1.80 (m, 1H), 1.80-1.46 (m, 8H), 1.26-1.12 (m, 2H), 1.01 (d, J=7.0 Hz, 3H), 0.97-0.91 (m, 1H), 0.89 (d, J=6.5 Hz, 6H). HRMS (ESI+, m/z): Calc'd for[C₂₇H₄₆N₂O₅S+H]⁺: 527.3149 found: 527.3156.

### Biological Assays

Minimum inhibitory concentrations (MICs) were determined by the broth microdilution method following the Clinical and Laboratory Standards Institute (CLSI) guidelines. Test organisms consisted of standard reference strains from the American Type Culture Collection (ATCC; Manassa, VA, USA) and clinical isolates obtained from JMI Laboratories Inc. (North Liberty, IA, USA), Micromyx repository (MMX; Kalamazoo, MI, USA), and Massachusetts Eye and Ear Hospital (Boston, USA). Before the start of the MIC experiment, standard and test compound stock solutions were prepared in dimethyl sulfoxide (DMSO, Aldrich D2650) at a stock concentration of 2,560 μg/mL. The compound concentration range typically employed for each experiment was 64-0.06 μg/mL (2.5% final DMSO concentration). Iboxamycin was used as a comparator in all experiments. All bacterial strains were sub-cultured on blood agar plates (tryptic soy agar with 5% sheep blood, Hardy Diagnostics) and incubated overnight at 35° C. Organisms were suspended in cation-adjusted Mueller-Hinton broth (CaMHB, BD 212322) and optical density was adjusted to 0.5 McFarland standard. Suspensions were further diluted to obtain a final inoculum of 5×10⁵ CFU/mL for broth microdilution experiments. The minimum concentration of compound required to inhibit visible bacterial growth after 24 h of incubation was recorded as the MIC in μg/mL. MICs for P. aeruginosa were recorded in cation-adjusted Mueller-Hinton broth which had been pre-treated with Chelex 100 ion exchange resin according to procedures reported in Ito et al. Antimicrob Agents Chemother (2016) 60 (12), 7396-7401. The tables below show that compounds of the disclosure possess potent antibacterial activity against both Gram-positive and Gram-negative strains.

### MIC(μg/mL) Of Compounds Against Gram-Positive and Gram-Negative Strains

| | Species | Strain Description | FSA1503054 | FSA1503055 | FSA1503058 |
|---|---|---|---|---|---|
| Gram+ | S. aureus | HAV#001, ATCC 29213 | 0.125 | 0.5 | 0.125 |
| | S. aureus | HAV#031, c-ermA | NT | NT | NT |
| | S. aureus | HAV#016, MRSA | 0.125 | 0.5 | 0.125 |
| | S. aureus | HAV#202, ATCC 700699 | 8 | 32 | 4 |
| | S. pneumoniae | HAV#003, ATCC 49619 | ≤0.03 | 0.25 | ≤0.03 |
| | S. pyogenes | HAV#004, ATCC 19615 | ≤0.03 | 0.125 | ≤0.03 |
| | E. faecium | HAV#318, VRE; clinical | 16 | 4 | 2 |
| Gram− | E. coli | HAV#006, ATCC 25922 | 16 | >64 | 16 |
| | E. coli | HAV#038, ΔTolC | 1 | 16 | 1 |
| | E. coli | HAV#040, LptD | 4 | 64 | 4 |
| | K. oxytoca | HAV#450; Clinical | 32 | >64 | 32 |

| | Species | Strain Description | FSA1507013 | FSA1508003 | FSA1507066 | FSA1508001 | FSA1507068 |
|---|---|---|---|---|---|---|---|
| Gram+ | S. aureus | HAV#001, ATCC 29213 | 0.125 | 0.125 | ≤0.06 | 0.25 | ≤0.06 |
| | S. aureus | HAV#031, c-ermA | 4 | 2 | 0.5 | 0.5 | 0.5 |
| | S. aureus | HAV#216, cfr | NT | 8 | 0.5 | 1 | 2 |
| | S. pyogenes | HAV#033, c-ermB | 0.5 | 2 | 0.125 | 0.25 | 0.25 |
| | E. faecalis | HAV#005, ATCC 29212 | 0.125 | 0.125 | ≤0.06 | 0.125 | 0.125 |
| | E. faecium | HAV#318, VRE; clinical | NT | 1 | 0.25 | 0.25 | 0.5 |
| Gram− | E. coli | HAV#006, ATCC 25922 | 2 | 4 | 4 | 8 | 4 |
| | E. coli | HAV#038, ΔTolC | 0.125 | 0.25 | 0.125 | 0.25 | 0.5 |
| | E. coli | HAV#040, LptD | 0.5 | 0.25 | 0.25 | 0.25 | 0.5 |
| | K. pneumoniae | HAV#402, FQ-R | 8 | 16 | 16 | 64 | 8 |
| | K. oxytoca | HAV#450; Clinical | 2 | 8 | 4 | 16 | 8 |
| | A. baumanii | HAV#010, ATCC 19606 | 2 | 2 | 2 | 2 | 4 |
| | P. aeruginosa | HAV#009, ATCC 27853 | 64 | NT | NT | NT | NT |

| | Species | Strain Description | FSA1504066A | FSA1504066B | FSA1504068 | FSA1503085 |
|---|---|---|---|---|---|---|
| Gram+ | S. aureus | HAV#001, ATCC 29213 | ≤0.06 | 0.5 | 0.5 | 0.25 |
| | S. aureus | HAV#031, c-ermA | 4 | 32 | >64 | 32 |
| | S. aureus | HAV#216, cfr | >64 | >64 | >64 | 8 |
| | S. pyogenes | HAV#033, c-ermB | NT | NT | NT | NT |
| | E. faecalis | HAV#005, ATCC 29212 | NT | NT | 2 | 0.25 |
| | E. faecium | HAV#318, VRE; clinical | 1 | 4 | NT | NT |
| Gram− | E. coli | HAV#006, ATCC 25922 | 32 | 64 | 64 | 64 |
| | E. coli | HAV#038, ΔTolC | 1 | 32 | 32 | 4 |
| | E. coli | HAV#040, LptD | 2 | 64 | 64 | 8 |
| | K. pneumoniae | HAV#402, FQ-R | 32 | 64 | >64 | >64 |
| | K. oxytoca | HAV#450; Clinical | NT | NT | NT | NT |
| | A. baumanii | HAV#010, ATCC 19606 | 8 | 64 | >64 | 64 |
| | P. aeruginosa | HAV#009, ATCC 27853 | NT | NT | NT | NT |

| | Strain | | | | | | |
|---|---|---|---|---|---|---|---|
| Species | Description | FSA1607077 | FSA1607090 | FSA1607075 | FSA1608021 | FSA1609015 | FSA1609037 |
| S. aureus | ATCC 29213 | ≤0.125 | 8 | 2 | 0.5 | 1 | 0.5 |
| S. aureus | MMX 3035 | 4 | 32 | 4 | 2 | 2 | 0.5 |
| S. aureus | Clinical (cfr) | 16 | >64 | 8 | 4 | 2 | 1 |
| S. pneumoniae | c-ermB | ND | ND | ND | ND | 0.25 | ND |
| E. faecalis | ATCC 29212 | ≤0.125 | 32 | 2 | 0.5 | 2 | 0.25 |
| E. faecium | JMI-990536 | 2 | 16 | 2 | 0.5 | 0.5 | ND |
| E. coli | ATCC 25922 | 4 | >64 | 4 | 1 | 8 | 1 |
| E. coli | ΔtolC | 1 | 4 | 2 | 1 | 2 | 0.5 |
| E. coli | lptD mutant | 1 | 8 | 2 | 0.25 | 2 | 0.5 |
| K. pneumoniae | JMI-991193 | 16 | >64 | 32 | 4 | 32 | 16 |
| P. aeruginosa | ATCC 27853 | ND | ND | ND | ND | 16 | ND |
| A. baumannii | ATCC 19606 | 8 | >64 | 8 | 4 | 8 | ND |

| | Strain | | | | | | |
|---|---|---|---|---|---|---|---|
| Species | Description | FSA1609052 | FSA1609051 | FSA1606033 | FSA1606034 | FSA1608082 | FSA1608083 |
| S. aureus | ATCC 29213 | 0.25 | 0.25 | 1 | 1 | 1 | 1 |
| S. aureus | MMX 3035 | 0.5 | 0.5 | 16 | 64 | 16 | 16 |
| S. aureus | Clinical (cfr) | 0.5 | 0.5 | 32 | >64 | 16 | 32 |
| S. pneumoniae | c-ermB | ND | ND | 4 | 16 | ND | ND |
| E. faecalis | ATCC 29212 | 0.25 | 0.25 | 1 | 2 | 2 | 1 |
| E. faecium | JMI-990536 | 0.5 | 0.5 | 16 | >64 | ND | ND |
| E. coli | ATCC 25922 | 2 | 4 | 16 | 64 | 32 | 16 |
| E. coli | Clinical (armA) | ND | ND | ND | ND | 64 | 64 |
| E. coli | ΔtolC | 0.125 | 0.25 | 1 | 2 | 2 | 2 |
| E. coli | ΔlptD mutant | 0.125 | 0.5 | 4 | 8 | 1 | 1 |
| K. pneumoniae | JMI-991193 | 4 | 8 | >64 | >64 | >64 | >64 |
| P. aeruginosa | ATCC 27853 | ND | ND | ND | ND | 16 | 8 |
| A. baumannii | ATCC 19606 | 1 | 2 | 64 | >64 | ND | ND |

| | Strain | | | | | | |
|---|---|---|---|---|---|---|---|
| Species | Description | FSA1608088 | FSA1608089 | FSA1607032 | FSA1607044 | FSA1609034 | FSA1606086 |
| S. aureus | ATCC 29213 | 1 | 2 | 0.5 | 0.125 | ≤0.06 | 16 |
| S. aureus | MMX 3035 | 8 | 8 | 4 | 2 | 0.25 | 64 |
| S. aureus | Clinical (cfr) | 8 | 8 | 16 | 16 | 0.25 | >64 |
| E. faecalis | ATCC 29212 | 1 | 1 | 0.5 | 0.125 | ≤0.06 | 16 |
| E. faecium | JMI-990536 | ND | ND | 2 | 1 | ND | 16 |
| E. coli | ATCC 25922 | 8 | 16 | 8 | 8 | 1 | 64 |
| E. coli | Clinical (armA) | 16 | 64 | ND | ND | 2 | ND |
| E. coli | ΔtolC | 2 | 2 | 0.25 | 0.5 | ≤0.06 | 2 |
| E. coli | lptD mutant | 0.5 | 1 | 2 | 2 | 0.25 | 4 |
| K. pneumoniae | JMI-991193 | 32 | >64 | 64 | 16 | 2 | >64 |
| P. aeruginosa | ATCC 27853 | 32 | 32 | ND | ND | 2 | |
| A. baumannii | ATCC 19606 | ND | ND | 32 | 8 | ND | >64 |

| | Strain | | | | | | |
|---|---|---|---|---|---|---|---|
| Species | Description | FSA1607019 | FSA1607020 | FSA1608009 | FSA1608016 | FSA1608024 | FSA1608025A |
| S. aureus | ATCC 29213 | 4 | 4 | 32 | 8 | 4 | 4 |
| S. aureus | MMX 3035 | 8 | 16 | >64 | >64 | 64 | 64 |
| S. aureus | Clinical (cfr) | 16 | 16 | >64 | >64 | 64 | 64 |
| E. faecalis | ATCC 29212 | 2 | 4 | >64 | 8 | 2 | 4 |
| E. faecium | JMI-990536 | 4 | 16 | >64 | 8 | 2 | 8 |
| E. coli | ATCC 25922 | 4 | 8 | >64 | 64 | 16 | 32 |
| E. coli | ΔtolC | 0.25 | 0.25 | >64 | ND | ND | ND |
| E. coli | ΔlptD mutant | 0.5 | 1 | >64 | 8 | 4 | 8 |
| K. pneumoniae | JMI-991193 | >64 | >64 | >64 | >64 | >64 | >64 |
| A. baumannii | ATCC 19606 | 32 | 32 | >64 | >64 | >64 | >64 |

| | Strain | | | | | | |
|---|---|---|---|---|---|---|---|
| Species | Description | FSA1608025B | FSA1608057 | FSA1608056 | FSA1608037 | FSA1608036 | FSA1609094 |
| S. aureus | ATCC 29213 | 32 | 4 | 4 | 32 | 0.5 | 0.25 |
| S. aureus | MMX 3035 | >64 | >64 | >64 | >64 | 16 | >32 |
| S. aureus | Clinical (cfr) | >64 | >64 | >64 | >64 | 8 | >32 |
| E. faecalis | ATCC 29212 | 32 | 2 | 4 | 16 | 1 | 2 |
| E. faecium | JMI-990536 | 32 | ND | ND | 16 | 1 | >32 |
| E. coli | ATCC 25922 | >64 | >64 | 64 | >64 | 1 | >32 |
| E. coli | Clinical (armA) | ND | >64 | 64 | ND | ND | ND |
| E. coli | ΔtolC | >64 | 16 | 16 | ND | 0.25 | 32 |
| E. coli | ΔlptD mutant | >64 | 32 | 4 | 4 | ND | 32 |
| K. pneumoniae | JMI-991193 | >64 | >64 | >64 | >64 | 8 | >32 |
| P. aeruginosa | ATCC 27853 | ND | 32 | 32 | ND | ND | ND |
| A. baumannii | ATCC 19606 | >64 | ND | ND | >64 | 16 | >32 |

| | Strain | | | | | | | |
|---|---|---|---|---|---|---|---|---|
| Species | Description | FSA1609033 | FSA1607022 | FSA1609096 | FSA1609095 | FSA1610015 | FSA1610016 | FSA1610014 |
| S. aureus | ATCC 29213 | ≤0.06 | ≤0.06 | ≤0.06 | ≤0.06 | 0.125 | 0.125 | 0.5 |
| S. aureus | MMX 3035 | 0.25 | 4 | 0.25 | 2 | 8 | 16 | 32 |
| S. aureus | Clinical (cfr) | 0.25 | 4 | 0.25 | 8 | 2 | 16 | 32 |
| S. pneumoniae | c-ermB | ≤0.03 | ND | ND | ND | 4 | 8 | 16 |
| E. faecalis | ATCC 29212 | ≤0.06 | ≤0.06 | ≤0.06 | 0.125 | 0.25 | 0.25 | 2 |
| E. faecium | JMI-990536 | ≤0.06 | 1 | 0.125 | 2 | 1 | 4 | 32 |
| E. coli | ATCC 25922 | 1 | 2 | 2 | 8 | 8 | 16 | >32 |
| E. coli | Clinical (armA) | 4 | ND | ND | ND | ND | ND | ND |
| E. coli | ΔtolC | ≤0.06 | 0.25 | 0.25 | 4 | ND | ND | ND |
| E. coli | ΔlptD mutant | 0.125 | 0.25 | 0.125 | 0.5 | 1 | 2 | 16 |
| K. pneumoniae | JMI-991193 | 2 | 8 | 8 | 16 | 32 | 32 | >32 |
| P. aeruginosa | ATCC 27853 | 1 | ND | ND | ND | 4 | 4 | 32 |
| A. baumannii | ATCC 19606 | 1 | 2 | 1 | 4 | ND | ND | ND |
| H. influenzae | ATCC 49247 | ≤0.06 | ND | ND | ND | ≤0.06 | ≤0.06 | 0.25 |
| M. catarrhalis | ATCC 25238 | ND | ND | ND | ND | 1 | 2 | 16 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 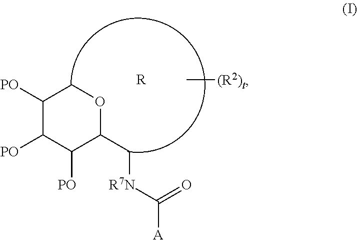
or a pharmaceutically acceptable salt thereof, wherein:
each occurrence of P is independently hydrogen or a protecting group;
A is substituted or unsubstituted heteroaliphatic, substituted or unsubstituted heteroaryl, or substituted or unsubstituted heteroaralkyl;
R⁷ is hydrogen or unsubstituted alkyl;
R is heterocyclyl;
each occurrence of R² is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0-12; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted carbocyclylalkyl, substituted or unsubstituted heterocyclyl, substituted or unsubstituted aryl, substituted or unsubstituted aralkyl, substituted or unsubstituted heteroaryl, substituted or unsubstituted hetaralkyl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring.

2. The compound of claim 1, or a pharmaceutically acceptable salt thereof, wherein:
A is substituted or unsubstituted heteroaliphatic.

3. The compound of claim 1 or 2, or a pharmaceutically acceptable salt thereof, wherein:
A is substituted or unsubstituted heterocyclyl.

4. The compound of any of claims 1-3, or a pharmaceutically acceptable salt thereof, wherein:
A is substituted or unsubstituted 4-8 membered monocyclic heterocyclyl, or substituted or unsubstituted fused bicyclic heterocyclyl.

5. The compound of any of claims 1-4, or a pharmaceutically acceptable salt thereof, wherein:
A is substituted or unsubstituted 4-8 membered monocyclic heterocyclyl.

6. The compound of any of claims 1-5, or a pharmaceutically acceptable salt thereof, wherein:
A is of formula: 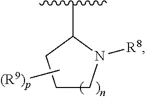
wherein:
n is 1, 2, or 3;
R⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaliphatic, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —S(O)₂R^{A}, or a nitrogen protecting group;
Each occurrence of R⁹ is independently, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; and
p is 0-4.

7. The compound of claim 6, or a pharmaceutically acceptable salt thereof, wherein:
A is of formula:

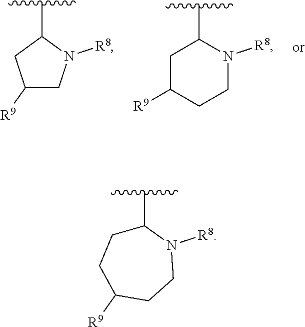

8. The compound of any of claims 1-4, or a pharmaceutically acceptable salt thereof, wherein:
A is substituted or unsubstituted fused bicyclic heterocyclyl.

9. The compound of any of claims 1-4 or 8, or a pharmaceutically acceptable salt thereof, wherein:
A is of formula: 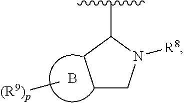
wherein:
B is carbocyclyl or heterocyclyl;
R⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroaliphatic, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —S(O)₂R^{A}, or a nitrogen protecting group;
each occurrence of R⁹ is independently, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted aralkyl, substituted or unsubstituted heteroaryl, substituted or unsubstituted heteroaralkyl, substituted or unsubstituted heteroaliphatic, —OR^{A}, —N(R^{A})₂, —SR^{A}, —CN, —SCN, —C(═NR^{A})R^{A}, —C(═NR^{A})OR^{A}, C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}; or two R⁹ groups are joined to form a substituted or unsubstituted heterocyclyl ring, or a substituted or unsubstituted carbocyclyl ring; and
p is 0-4.

10. The compound of claim 9, or a pharmaceutically acceptable salt thereof, wherein:
A is of formula: 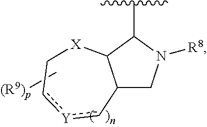
wherein:
X is O, S, NR⁹ or C(R⁹)₂;
Y is O, S, N, NR⁹, C(R⁹)₂, CR⁹, CH₂, or CH;
n is 0 or 1;
p is 0-4; and [symbol]
represents a single or double bond.

11. The compound of claim 10, or a pharmaceutically acceptable salt thereof, wherein:
A is of formula:

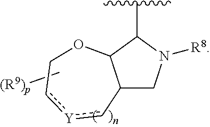

12. The compound of claim 10 or 11, or a pharmaceutically acceptable salt thereof, wherein:
A is of formula:

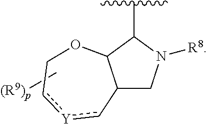

13. The compound of any of claims 10-12, or a pharmaceutically acceptable salt thereof, wherein:
A is of formula:

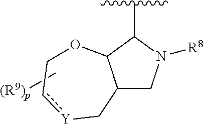

14. The compound of any of claims 9-13, or a pharmaceutically acceptable salt thereof, wherein:
A is of formula:

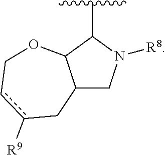

15. The compound of any of claims 9-14, or a pharmaceutically acceptable salt thereof, wherein:
A is of formula:

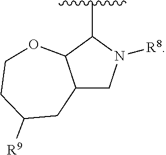

16. The compound of any of claims 9-15, or a pharmaceutically acceptable salt thereof, wherein:
A is of formula:

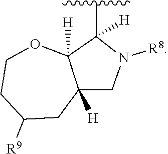

17. The compound of any of claims 9-16, or a pharmaceutically acceptable salt thereof, wherein:
A is of formula:

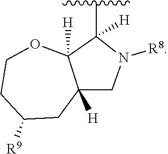

18. The compound of any of claims 9-17, or a pharmaceutically acceptable salt thereof, wherein:
A is of formula:

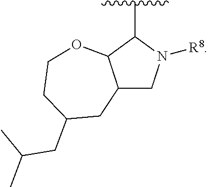

19. The compound of any of claims 1-4 or 8-18, or a pharmaceutically acceptable salt thereof, wherein:
A is of formula:

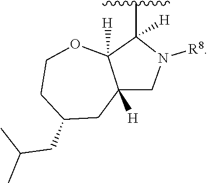

20. The compound of any of claims 1-19, or a pharmaceutically acceptable salt thereof, wherein:
R⁷ is hydrogen.

21. The compound of any of claims 6-20, or a pharmaceutically acceptable salt thereof, wherein:
R⁸ is hydrogen, substituted or unsubstituted alkyl, substituted or unsubstituted carbocyclyl, or —C(═O)R^{A}.

22. The compound of any of claims 6-21, or a pharmaceutically acceptable salt thereof, wherein:
R⁸ is hydrogen or substituted or unsubstituted alkyl.

23. The compound of any of claims 6-22, or a pharmaceutically acceptable salt thereof, wherein:
R⁸ is hydrogen or methyl.

24. The compound of any of claims 6-23, or a pharmaceutically acceptable salt thereof, wherein:
R⁸ is hydrogen.

25. The compound of any of claims 6-23, or a pharmaceutically acceptable salt thereof, wherein:
R⁸ is methyl.

26. The compound of any of claims 6-25, or a pharmaceutically acceptable salt thereof, wherein:
R⁹ is substituted or unsubstituted alkyl.

27. The compound of any of claims 6-26, or a pharmaceutically acceptable salt thereof, wherein:
R⁹ is substituted or unsubstituted C₁₋₆ alkyl.

28. The compound of any of claims 6-26, or a pharmaceutically acceptable salt thereof, wherein:
R⁹ is —CH₃, —CH₂CH₃, —CH₂CH₂CH₃, —CH₂CH₂CH₂CH₃, —CH(CH₃)₂, —CH₂CH(CH₃)₂, or —CH(CH₃)₃.

29. The compound of any of claims 6-28, or a pharmaceutically acceptable salt thereof, wherein:
R⁹ is —CH₂CH(CH₃)₂.

30. The compound of any of claims 1-29, or a pharmaceutically acceptable salt thereof, wherein:
each P is hydrogen.

31. The compound of any of claims 1-30, or a pharmaceutically acceptable salt thereof, wherein:
R is 6-14 membered heterocyclyl.

32. The compound of any of claims 1-31, or a pharmaceutically acceptable salt thereof, wherein:
R is 8-12 membered heterocyclyl.

33. The compound of any of claims 1-32, or a pharmaceutically acceptable salt thereof, wherein:
R is 9-12 membered heterocyclyl.

34. The compound of any of claims 1-33, or a pharmaceutically acceptable salt thereof, wherein:
R is 9-11 membered heterocyclyl.

35. The compound of any of claims 1-34, or a pharmaceutically acceptable salt thereof, wherein:
R is 11-membered heterocyclyl.

36. The compound of any of claims 1-34, or a pharmaceutically acceptable salt thereof, wherein:
R is 10-membered heterocyclyl.

37. The compound of any of claims 1-36, or a pharmaceutically acceptable salt thereof, wherein:
R comprises at least one unsaturated bond in its ring.

38. The compound of any of claims 1-37, or a pharmaceutically acceptable salt thereof, wherein:
R comprises at least one unsaturated carbon-carbon bond in its ring.

39. The compound of any of claims 1-38, or a pharmaceutically acceptable salt thereof, wherein:
R comprises at least one heteroatom in addition to the oxygen in its ring.

40. The compound of any of claims 1-39, or a pharmaceutically acceptable salt thereof, wherein:
R comprises at least one sulfur atom in its ring.

41. The compound of any of claims 1-40, or a pharmaceutically acceptable salt thereof, wherein:
R comprises one sulfur atom in its ring.

42. The compound of any of claims 1-40, or a pharmaceutically acceptable salt thereof, wherein R is a heterocyclyl of formula:
wherein: R¹ is —NR²—, —O—, or —S—.

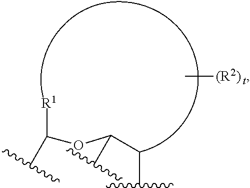

43. The compound of any of claims 1-42, or a pharmaceutically acceptable salt thereof, wherein R is a heterocyclyl of formula:

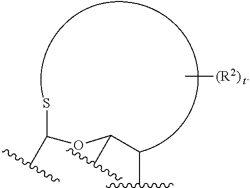

44. The compound of any of claims 1-43, or a pharmaceutically acceptable salt thereof, wherein R is a heterocyclyl of formula: 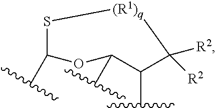
wherein: each R¹ is independently a bond, —CR²R²—, CR², C, N, —NR², —O—, or S; and q is 3-6.

45. The compound of any of claims 1-43, or a pharmaceutically acceptable salt thereof, wherein R is a heterocyclyl of formula: 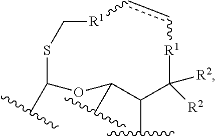
wherein:
each R¹ is independently a bond or —CR²R²; and [symbol]
represents a single or double bond.

46. The compound of any of claims 1-43, or a pharmaceutically acceptable salt thereof, wherein R is a heterocyclyl of formula: 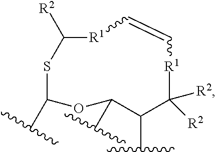
wherein:
each R¹ is independently a bond or —CR²R²—.

47. The compound of any of claims 1-43, or a pharmaceutically acceptable salt thereof, wherein R is a heterocyclyl of formula: 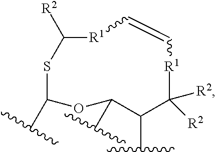
wherein:
each R¹ is independently a bond or —CR²R²—.

48. The compound of any of claims 1-43, or a pharmaceutically acceptable salt thereof, wherein R is a heterocyclyl of formula: 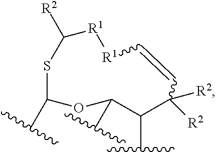
wherein:
each R¹ is independently a bond or —CR²R²—.

49. The compound of any of claims 1-43, or a pharmaceutically acceptable salt thereof, wherein R is a heterocyclyl of formula: 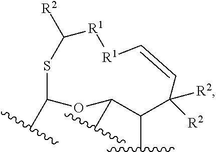
wherein:
each R¹ is independently a bond or —CR²R²—.

50. The compound of any of claims 1-43, or a pharmaceutically acceptable salt thereof, wherein R is a heterocyclyl of formula:

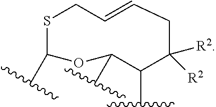

51. The compound of any of claims 1-43, or a pharmaceutically acceptable salt thereof, wherein R is a heterocyclyl of formula:

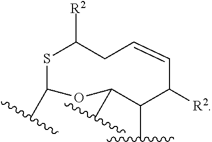

52. The compound of any of claims 1-43, or a pharmaceutically acceptable salt thereof, wherein R is a heterocyclyl of formula:

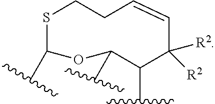

53. The compound of any of claims 1-43, or a pharmaceutically acceptable salt thereof, wherein R is a heterocyclyl of formula:

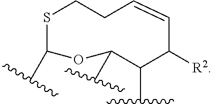

54. The compound of any of claims 1-43, or a pharmaceutically acceptable salt thereof, wherein R is a heterocyclyl of formula:

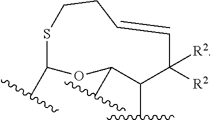

55. The compound of any of claims 1-43, or a pharmaceutically acceptable salt thereof, wherein R is a heterocyclyl of formula:

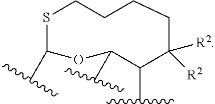

56. The compound of any of claims 1-43, or a pharmaceutically acceptable salt thereof, wherein R is a heterocyclyl of formula:

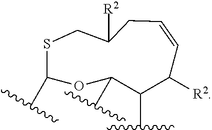

57. The compound of any of claims 1-56, or a pharmaceutically acceptable salt thereof, wherein:
each occurrence of R² is, independently, hydrogen, halogen, substituted or unsubstituted alkyl, substituted or unsubstituted alkenyl, substituted or unsubstituted alkynyl, substituted or unsubstituted heteroalkyl, substituted or unsubstituted aryl, substituted or unsubstituted heteroaryl, —OR^{A}, —N(R^{A})₂, —SR^{A}, or —CN; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring.

58. The compound of any of claims 1-57, or a pharmaceutically acceptable salt thereof, wherein:
each occurrence of R² is, independently, hydrogen, halogen, substituted or unsubstituted alkyl, substituted or unsubstituted alkenyl, substituted or unsubstituted alkynyl, substituted or unsubstituted heteroalkyl, a nitrogen protecting group when attached to a nitrogen atom, —OR^{A}, —N(R^{A})₂, —SR^{A}, or —CN; or two R² groups are joined to form a substituted or unsubstituted carbocyclyl ring, a substituted or unsubstituted aryl ring, a substituted or unsubstituted heterocyclyl ring, or a substituted or unsubstituted heteroaryl ring.

59. The compound of any of claims 1-58, or a pharmaceutically acceptable salt thereof, wherein:
each occurrence of R² is, independently, hydrogen, halogen, substituted or unsubstituted alkyl, —OR^{A}, —N(R^{A})₂, or —SR^{A}; or two R² groups are joined to form a substituted or unsubstituted carbocyclyl ring, a substituted or unsubstituted aryl ring, or a substituted or unsubstituted heterocyclyl ring.

60. The compound of any of claims 1-59, or a pharmaceutically acceptable salt thereof, wherein:
each occurrence of R² is, independently, halogen, substituted or unsubstituted alkyl, or —OR^{A}; or two R² groups are joined to form a substituted or unsubstituted carbocyclyl ring, a substituted or unsubstituted aryl ring, or a substituted or unsubstituted heterocyclyl ring.

61. The compound of any of claims 1-60, or a pharmaceutically acceptable salt thereof, wherein:
each occurrence of R² is, independently, halogen, substituted or unsubstituted alkyl, or —OR^{A}.

62. The compound of any of claims 1-57, or a pharmaceutically acceptable salt thereof, wherein:
each occurrence of R² is, independently, hydrogen, halogen, substituted or unsubstituted alkyl, substituted or unsubstituted alkynyl, substituted or unsubstituted aryl, or substituted or unsubstituted heteroaryl.

63. The compound of any of claims 1-57, or a pharmaceutically acceptable salt thereof, wherein:
each occurrence of R² is, independently, hydrogen, halogen, substituted or unsubstituted alkyl, substituted or unsubstituted aryl, or substituted or unsubstituted heteroaryl.

64. The compound of any of claims 1-57, or a pharmaceutically acceptable salt thereof, wherein:
each occurrence of R² is, independently, hydrogen, halogen, substituted or unsubstituted alkyl, or substituted or unsubstituted heteroaryl.

65. The compound of any of claims 1-57, or a pharmaceutically acceptable salt thereof, wherein:
each occurrence of R² is, independently, halogen, substituted or unsubstituted alkyl, or substituted or unsubstituted heteroaryl.

66. The compound of any of claims 1-65, or a pharmaceutically acceptable salt thereof, wherein:
each occurrence of R² is, independently, substituted or unsubstituted alkyl.

67. The compound of any of claims 1-66, or a pharmaceutically acceptable salt thereof, wherein:
each occurrence of R² is, independently, unsubstituted alkyl.

68. The compound of any of claims 1-67, or a pharmaceutically acceptable salt thereof, wherein:
t is 0-4.

69. The compound of any of claims 1-68, or a pharmaceutically acceptable salt thereof, wherein:
t is 0-2.

70. The compound of any of claims 1-69 or a pharmaceutically acceptable salt thereof, wherein:
t is 0 or 1.

71. The compound of any of claims 1-70, or a pharmaceutically acceptable salt thereof, wherein:
t is 0.

72. The compound of any of claims 1-69, or a pharmaceutically acceptable salt thereof, wherein:
t is 1 or 2.

73. The compound of claim 1, wherein the compound is of Formula (I-a): 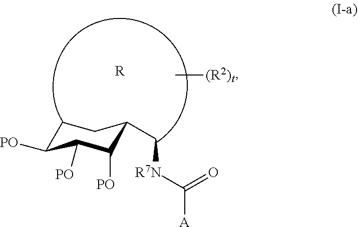
or a pharmaceutically acceptable salt thereof.

74. The compound of claim 1, wherein the compound is of Formula (I-b): 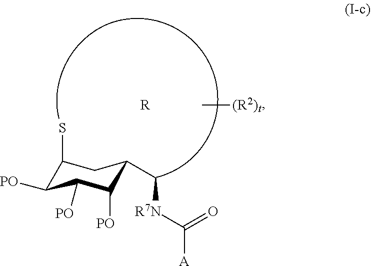
or a pharmaceutically acceptable salt thereof, wherein:
R¹ is —NR²—, —O—, or —S—.

75. The compound of claim 1, wherein the compound is of Formula (I-c): 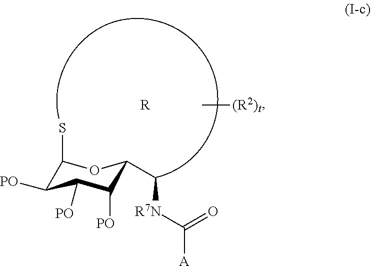
or a pharmaceutically acceptable salt thereof.

76. The compound of claim 1, wherein the compound is of Formula (I-d): 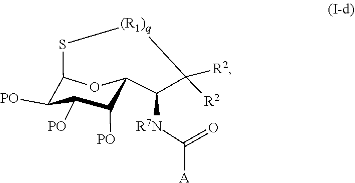
or a pharmaceutically acceptable salt thereof, wherein:
each R¹ is independently a bond, —CR²R², CR², C, N, —NR², —O—, or S; and q is 3-6.

77. The compound of claim 1, wherein the compound is of Formula (I-e): 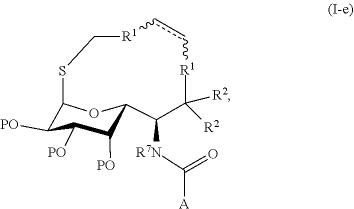
or a pharmaceutically acceptable salt thereof, wherein:
each R¹ is independently a bond or —CR²R²—; and [symbol]
represents a single or double bond.

78. The compound of claim 1, wherein the compound is of Formula (I-e-1): 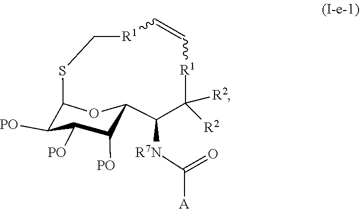
or a pharmaceutically acceptable salt thereof, wherein:
each R¹ is independently a bond or —CR²R²—.

79. The compound of claim 1, wherein the compound is of Formula (I-f): 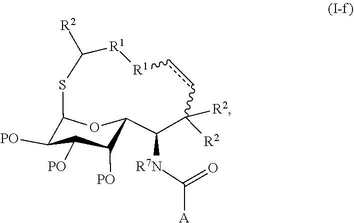
or a pharmaceutically acceptable salt thereof, wherein:
each R¹ is independently a bond or —CR²R²—; and [symbol]
represents a single or double bond.

80. The compound of claim 1, wherein the compound is of Formula (I-f-1): 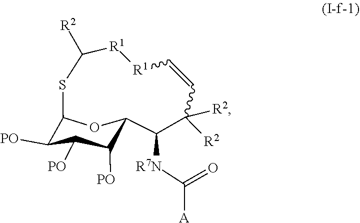
or a pharmaceutically acceptable salt thereof, wherein:
each R¹ is independently a bond or —CR²R²—.

81. The compound of claim 1, wherein the compound is of Formula (I-f-2): 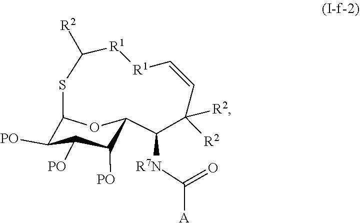
or a pharmaceutically acceptable salt thereof, wherein:
each R¹ is independently a bond or —CR²R²—.

82. The compound of claim 1, wherein the compound is of Formula (I-g): 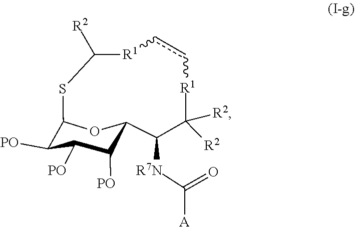
or a pharmaceutically acceptable salt thereof, wherein:
each R¹ is independently a bond or —CR²R²—; and [symbol]
represents a single or double bond.

83. The compound of claim 1, wherein the compound is of Formula (I-g-1): 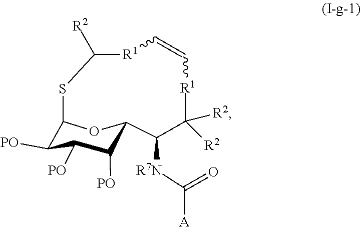
or a pharmaceutically acceptable salt thereof, wherein:
each R¹ is independently a bond or —CR²R²—.

84. The compound of claim 1, wherein the compound is of Formula (I-g-2): 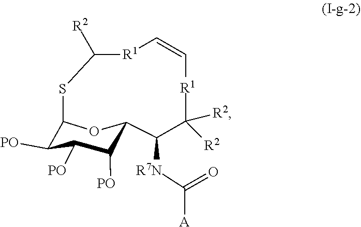
or a pharmaceutically acceptable salt thereof, wherein:
each R¹ is independently a bond or —CR²R²—.

85. The compound of claim 1, wherein the compound is of Formula (I-h): 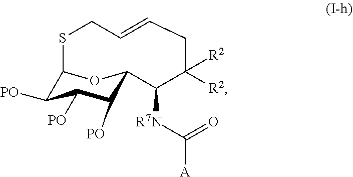
or a pharmaceutically acceptable salt thereof.

86. The compound of claim 1, wherein the compound is of Formula (I-h-1): 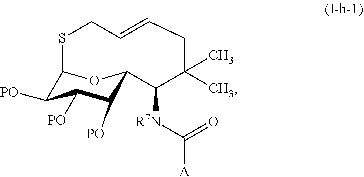
or a pharmaceutically acceptable salt thereof.

87. The compound of claim 1, wherein the compound is of Formula (I-h-2): 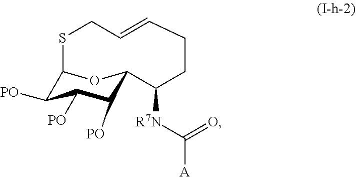
or a pharmaceutically acceptable salt thereof.

88. The compound of claim 1, wherein the compound is of Formula (I-i): 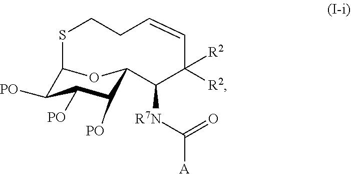
or a pharmaceutically acceptable salt thereof.

89. The compound of claim 1, wherein the compound is of Formula (I-i-1): 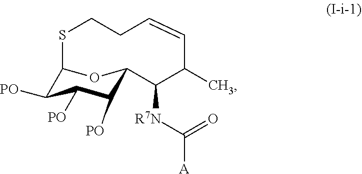
or a pharmaceutically acceptable salt thereof.

90. The compound of claim 1, wherein the compound is of Formula (I-i-2): 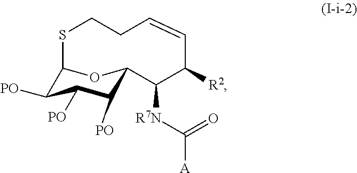
or a pharmaceutically acceptable salt thereof.

91. The compound of claim 1, wherein the compound is of Formula (I-i-8): 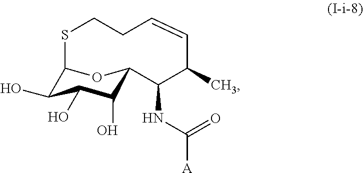
or a pharmaceutically acceptable salt thereof.

92. The compound of claim 1, wherein the compound is of Formula (I-j): 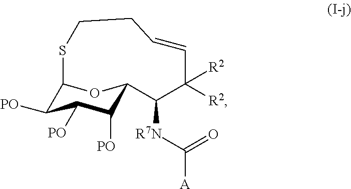
or a pharmaceutically acceptable salt thereof.

93. The compound of claim 1, wherein the compound is of Formula (I-j-1): 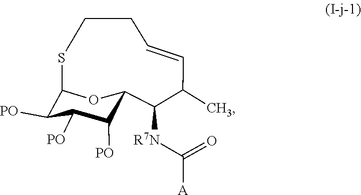
or a pharmaceutically acceptable salt thereof.

94. The compound of claim 1, wherein the compound is of Formula (I-k): 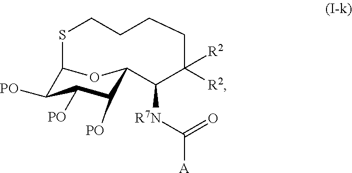
or a pharmaceutically acceptable salt thereof.

95. The compound of claim 1, wherein the compound is of Formula (I-k-1): 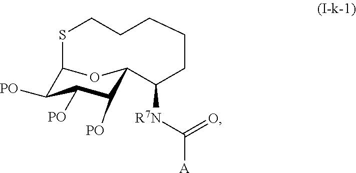
or a pharmaceutically acceptable salt thereof.

96. The compound of claim 1, wherein the compound is of Formula (I-I): 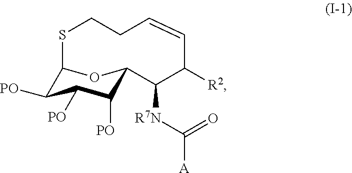
or a pharmaceutically acceptable salt thereof.

97. The compound of claim 1, wherein the compound is of Formula (I-1-4): 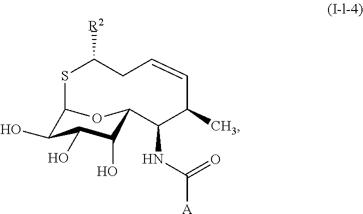
or a pharmaceutically acceptable salt thereof.

98. The compound of claim 1, wherein the compound is of Formula (I-m-1): 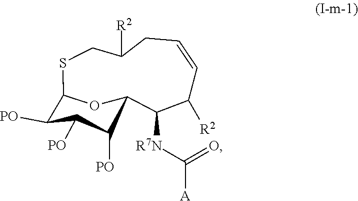
or a pharmaceutically acceptable salt thereof.

99. The compound of claim 1, wherein the compound is of Formula (I-m-5): 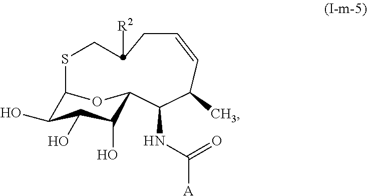
or a pharmaceutically acceptable salt thereof.

100. The compound of claim 1, wherein the compound is of Formula (I-n-1): 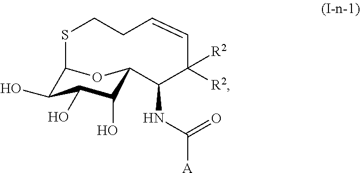
or a pharmaceutically acceptable salt thereof.

101. The compound of claim 9, wherein the compound is of Formula (I-n-2): 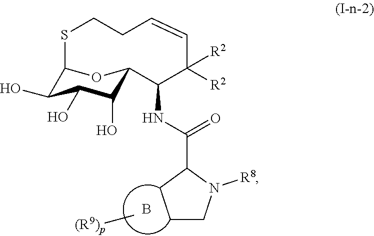
or a pharmaceutically acceptable salt thereof.

102. The compound of claim 10, wherein the compound is of Formula (I-n-4): 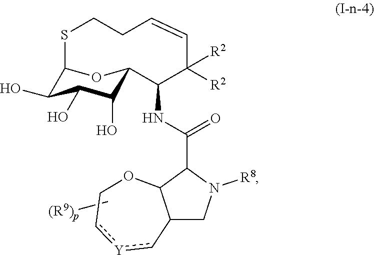
or a pharmaceutically acceptable salt thereof.

103. The compound of claim 9, wherein the compound is of Formula (I-n-8): 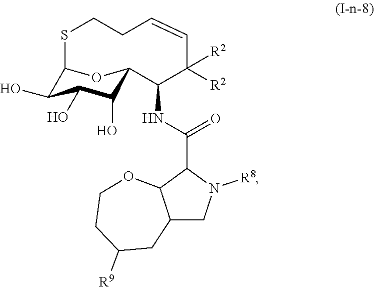
or a pharmaceutically acceptable salt thereof.

104. The compound of claim 1, wherein the compound is of Formula (I-n-11): 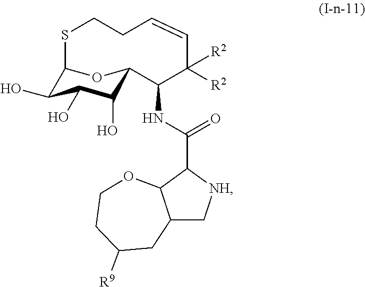
or a pharmaceutically acceptable salt thereof.

105. The compound of claim 1, or a pharmaceutically acceptable salt thereof, wherein the compound is: 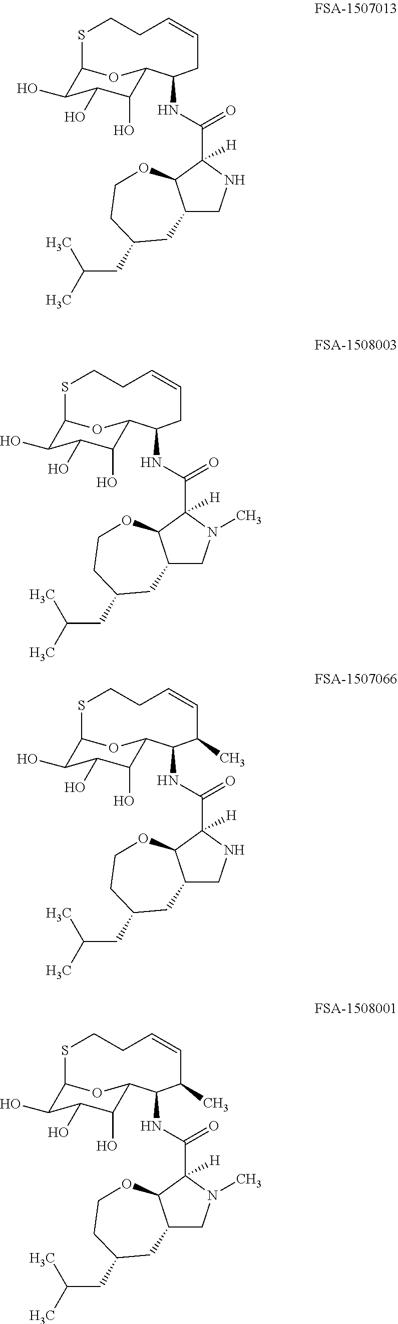 [SMILES: CC(C)C[C@@H]1CCO[C@@H]2[C@H](CN[C@@H]2C(=O)N[C@@H]2C/C=C\CCSC3OC2C(O)C(O)C3O)C1 ; CC(C)C[C@@H]1CCO[C@@H]2[C@@H](C1)CN(C)[C@@H]2C(=O)N[C@@H]1C/C=C\CCSC2OC1C(O)C(O)C2O ; CC(C)C[C@@H]1CCO[C@@H]2[C@H](CN[C@@H]2C(=O)N[C@H]2C3OC(SCC/C=C\[C@H]2C)C(O)C(O)C3O)C1 ; CC(C)C[C@@H]1CCO[C@@H]2[C@@H](C1)CN(C)[C@@H]2C(=O)N[C@H]1C2OC(SCC/C=C\[C@H]1C)C(O)C(O)C2O] 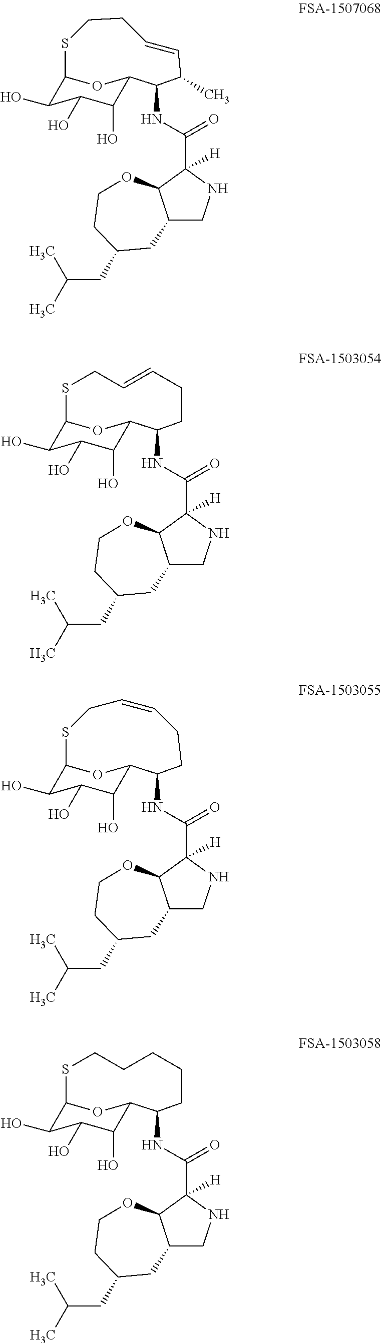 [SMILES: CC(C)C[C@@H]1CCO[C@@H]2[C@H](CN[C@@H]2C(=O)N[C@H]2C3OC(SCC/C=C/[C@@H]2C)C(O)C(O)C3O)C1 ; CC(C)C[C@@H]1CCO[C@@H]2[C@H](CN[C@@H]2C(=O)N[C@@H]2CC/C=C/CSC3OC2C(O)C(O)C3O)C1 ; CC(C)C[C@@H]1CCO[C@@H]2[C@H](CN[C@@H]2C(=O)N[C@@H]2CC/C=C\CSC3OC2C(O)C(O)C3O)C1 ; CC(C)C[C@@H]1CCO[C@@H]2[C@H](CN[C@@H]2C(=O)N[C@@H]2CCCCCSC3OC2C(O)C(O)C3O)C1] 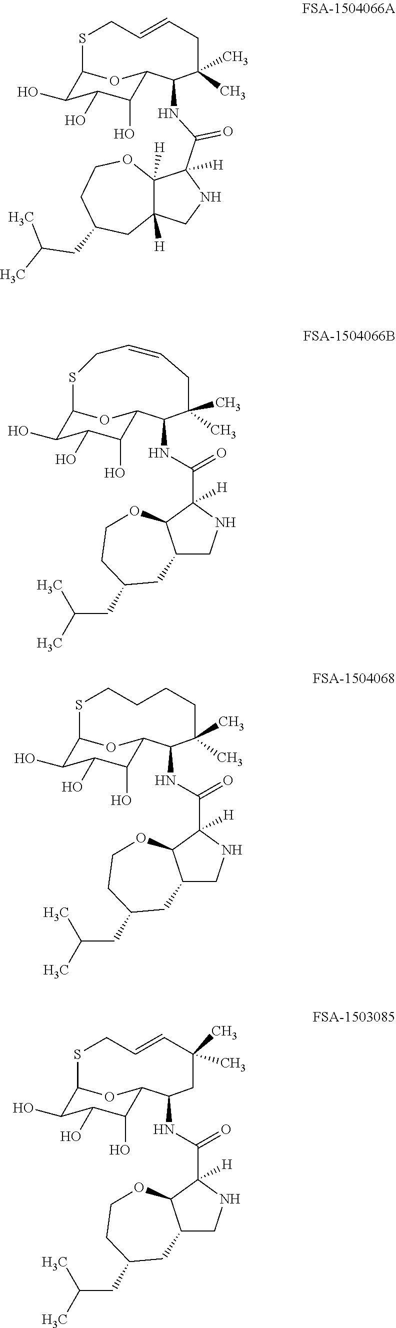 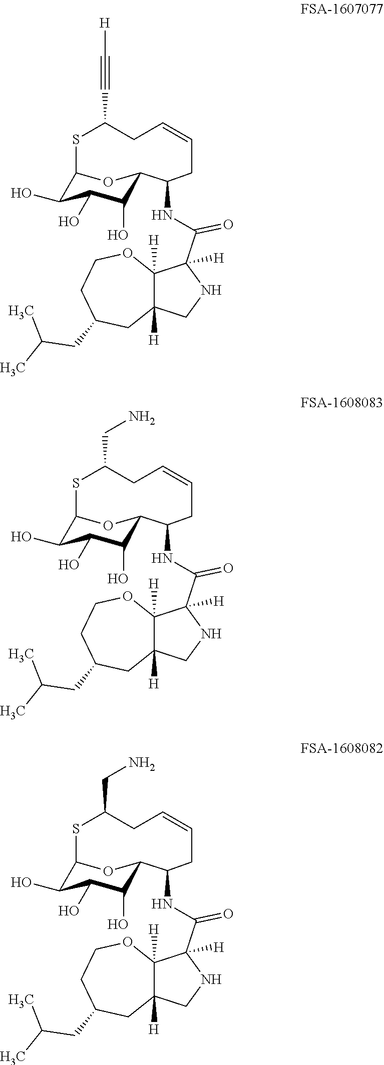 [SMILES: C#C[C@@H]1C/C=C\C[C@@H](NC(=O)[C@H]2NC[C@@H]3C[C@H](CC(C)C)CCO[C@H]32)[C@H]2OC(S1)[C@H](O)[C@H](O)C2O ; CC(C)C[C@@H]1CCO[C@@H]2[C@H](CN[C@@H]2C(=O)N[C@@H]2C/C=C\C[C@@H](CN)SC3O[C@H]2C(O)[C@@H](O)[C@H]3O)C1 ; CC(C)C[C@@H]1CCO[C@@H]2[C@H](CN[C@@H]2C(=O)N[C@@H]2C/C=C\C[C@H](CN)SC3O[C@H]2C(O)[C@@H](O)[C@H]3O)C1] 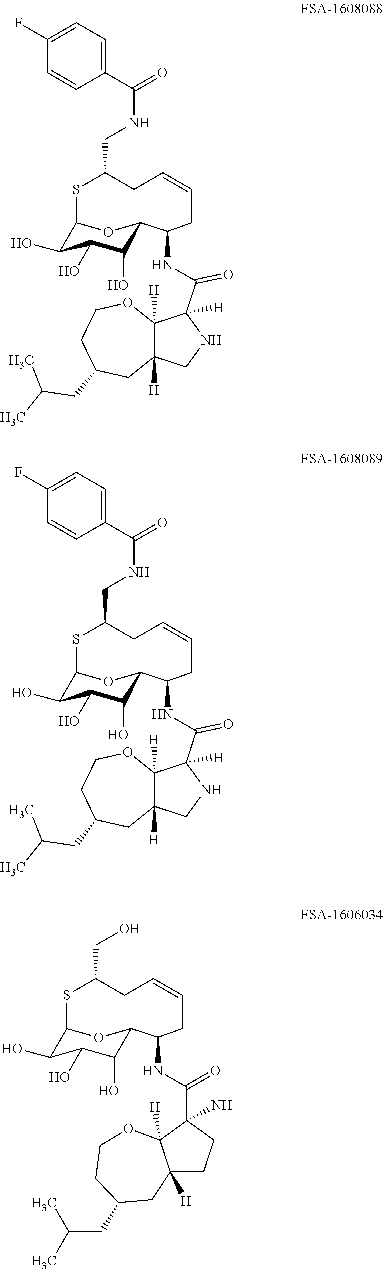 [SMILES: CC(C)C[C@@H]1CCO[C@@H]2[C@H](CN[C@@H]2C(=O)N[C@@H]2C/C=C\C[C@@H](CNC(=O)c3ccc(F)cc3)SC3O[C@H]2C(O)[C@@H](O)[C@H]3O)C1 ; CC(C)C[C@@H]1CCO[C@@H]2[C@H](CN[C@@H]2C(=O)N[C@@H]2C/C=C\C[C@H](CNC(=O)c3ccc(F)cc3)SC3O[C@H]2C(O)[C@@H](O)[C@H]3O)C1 ; CC(C)C[C@@H]1CCO[C@@H]2[C@H](CC[C@]2(N)C(=O)N[C@@H]2C/C=C\C[C@@H](CO)SC3OC2C(O)C(O)C3O)C1] 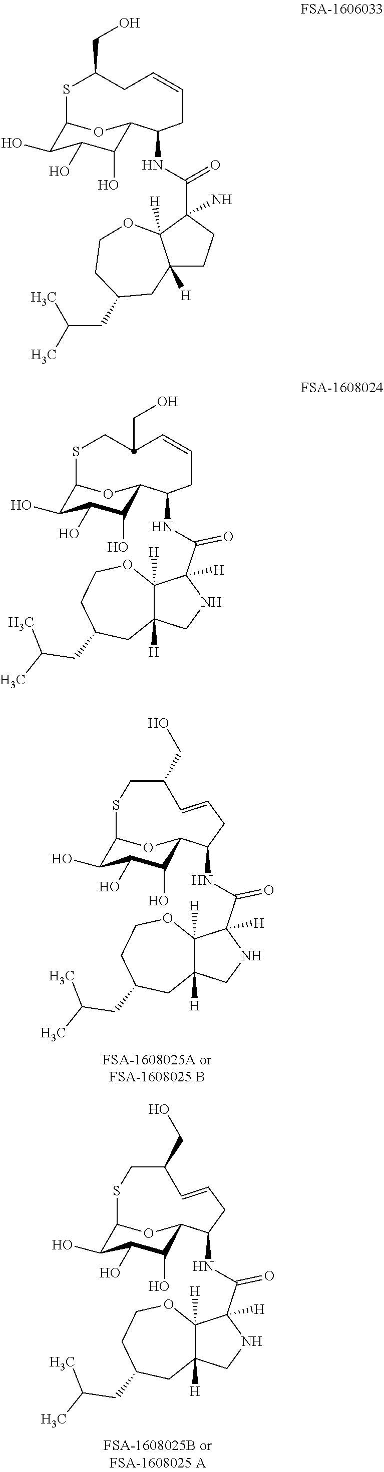 [SMILES: CC(C)C[C@@H]1CCO[C@@H]2[C@H](CC[C@]2(N)C(=O)N[C@@H]2C/C=C\C[C@H](CO)SC3OC2C(O)C(O)C3O)C1 ; CC(C)C[C@@H]1CCO[C@@H]2[C@H](CN[C@@H]2C(=O)N[C@@H]2C/C=C\[C@@H](CO)CSC3O[C@H]2C(O)[C@@H](O)[C@H]3O)C1 ; CC(C)C[C@@H]1CCO[C@@H]2[C@H](CN[C@@H]2C(=O)N[C@@H]2C/C=C/[C@@H](CO)CSC3O[C@H]2C(O)[C@@H](O)[C@H]3O)C1 ; CC(C)C[C@@H]1CCO[C@@H]2[C@H](CN[C@@H]2C(=O)N[C@@H]2C/C=C/[C@H](CO)CSC3O[C@H]2C(O)[C@@H](O)[C@H]3O)C1] 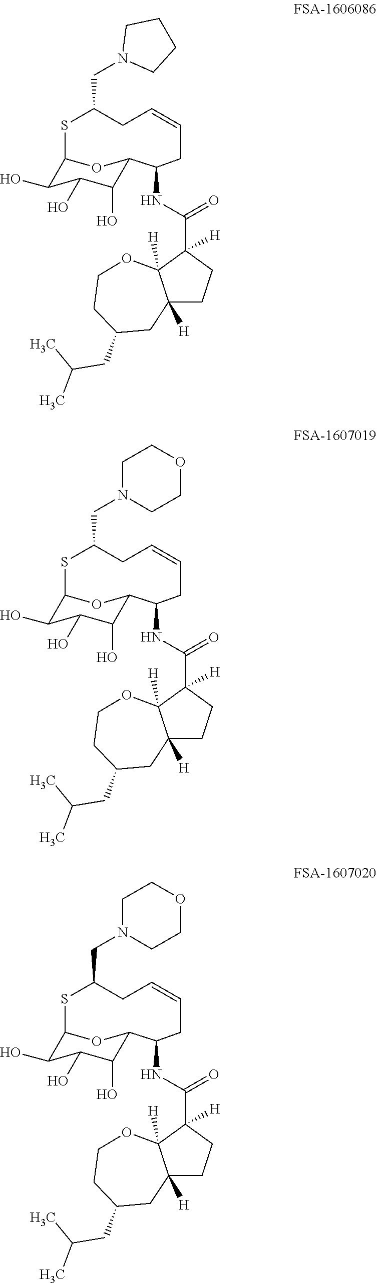 [SMILES: CC(C)C[C@@H]1CCO[C@@H]2[C@H](CC[C@@H]2C(=O)N[C@@H]2C/C=C\C[C@@H](CN3CCCC3)SC3OC2C(O)C(O)C3O)C1 ; CC(C)C[C@@H]1CCO[C@@H]2[C@H](CC[C@@H]2C(=O)N[C@@H]2C/C=C\C[C@@H](CN3CCOCC3)SC3OC2C(O)C(O)C3O)C1 ; CC(C)C[C@@H]1CCO[C@@H]2[C@H](CC[C@@H]2C(=O)N[C@@H]2C/C=C\C[C@H](CN3CCOCC3)SC3OC2C(O)C(O)C3O)C1] 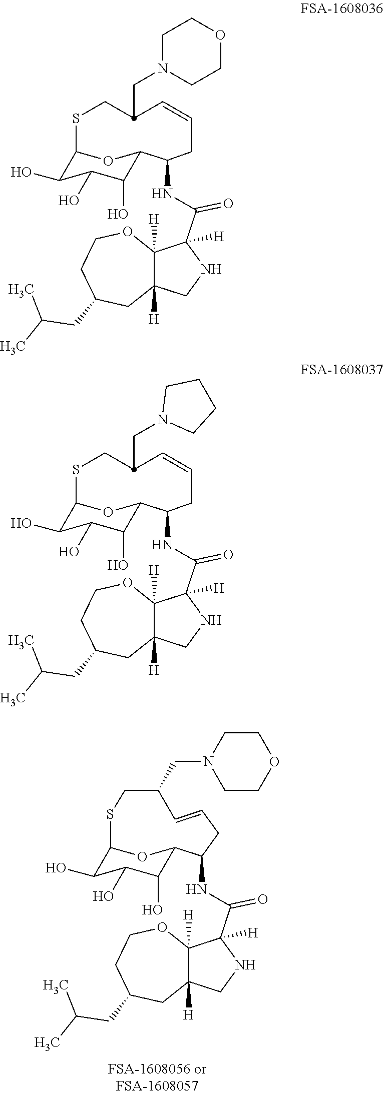 [SMILES: CC(C)C[C@@H]1CCO[C@@H]2[C@H](CN[C@@H]2C(=O)N[C@@H]2C/C=C\[C@@H](CN3CCOCC3)CSC3OC2C(O)C(O)C3O)C1 ; CC(C)C[C@@H]1CCO[C@@H]2[C@H](CN[C@@H]2C(=O)N[C@@H]2C/C=C\[C@@H](CN3CCCC3)CSC3OC2C(O)C(O)C3O)C1 ; CC(C)C[C@@H]1CCO[C@@H]2[C@H](CN[C@@H]2C(=O)N[C@@H]2C/C=C/[C@@H](CN3CCOCC3)CSC3OC2C(O)C(O)C3O)C1] 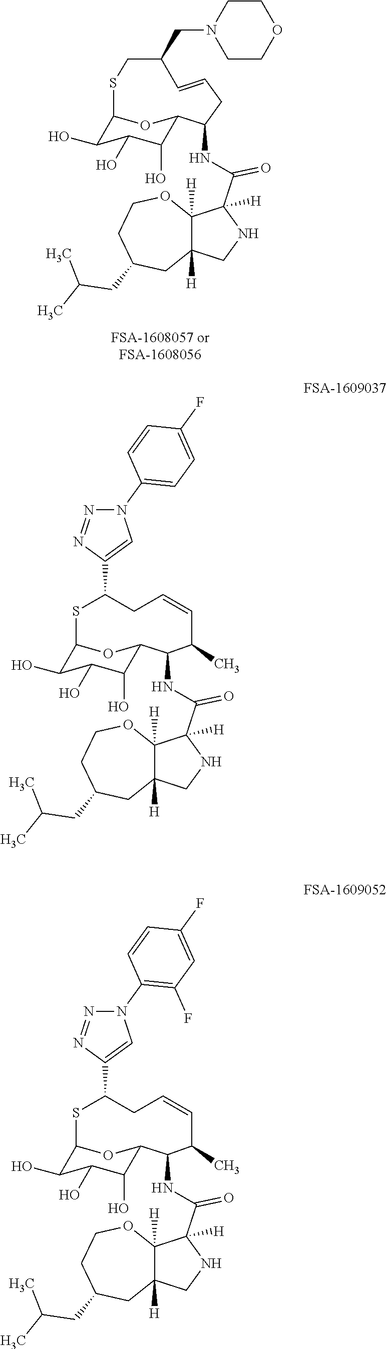 [SMILES: CC(C)C[C@@H]1CCO[C@@H]2[C@H](CN[C@@H]2C(=O)N[C@@H]2C/C=C/[C@H](CN3CCOCC3)CSC3OC2C(O)C(O)C3O)C1 ; CC(C)C[C@@H]1CCO[C@@H]2[C@H](CN[C@@H]2C(=O)N[C@H]2C3OC(S[C@H](c4cn(-c5ccc(F)cc5)nn4)C/C=C\[C@H]2C)C(O)C(O)C3O)C1 ; CC(C)C[C@@H]1CCO[C@@H]2[C@H](CN[C@@H]2C(=O)N[C@H]2C3OC(S[C@H](c4cn(-c5ccc(F)cc5F)nn4)C/C=C\[C@H]2C)C(O)C(O)C3O)C1] 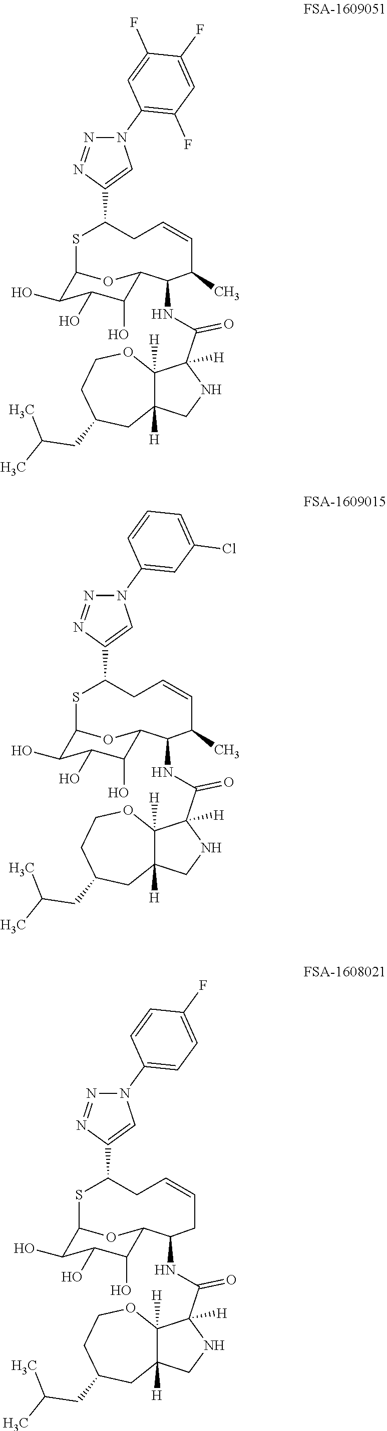 [SMILES: CC(C)C[C@@H]1CCO[C@@H]2[C@H](CN[C@@H]2C(=O)N[C@H]2C3OC(S[C@H](c4cn(-c5cc(F)c(F)cc5F)nn4)C/C=C\[C@H]2C)C(O)C(O)C3O)C1 ; CC(C)C[C@@H]1CCO[C@@H]2[C@H](CN[C@@H]2C(=O)N[C@H]2C3OC(S[C@H](c4cn(-c5cccc(Cl)c5)nn4)C/C=C\[C@H]2C)C(O)C(O)C3O)C1 ; CC(C)C[C@@H]1CCO[C@@H]2[C@H](CN[C@@H]2C(=O)N[C@@H]2C/C=C\C[C@@H](c3cn(-c4ccc(F)cc4)nn3)SC3OC2C(O)C(O)C3O)C1] 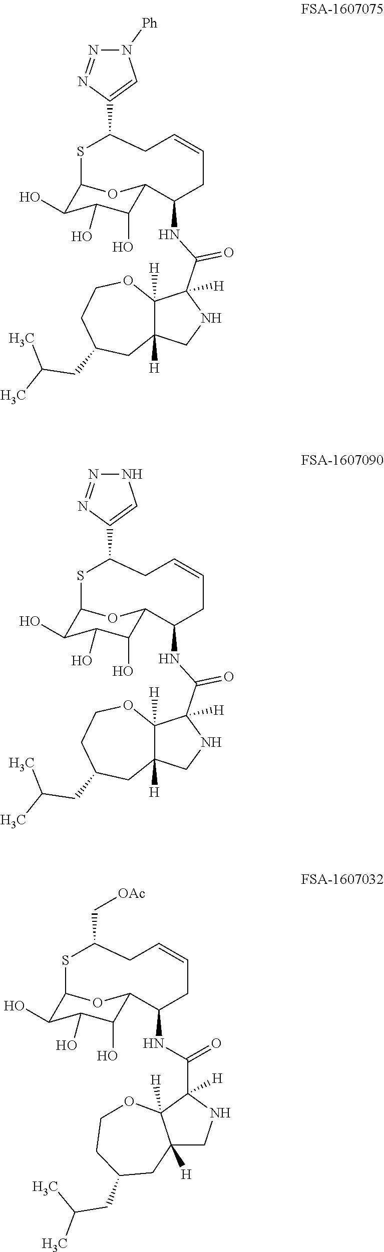 [SMILES: CC(C)C[C@@H]1CCO[C@@H]2[C@H](CN[C@@H]2C(=O)N[C@@H]2C/C=C\C[C@@H](c3cn(-c4ccccc4)nn3)SC3OC2C(O)C(O)C3O)C1 ; CC(C)C[C@@H]1CCO[C@@H]2[C@H](CN[C@@H]2C(=O)N[C@@H]2C/C=C\C[C@@H](c3c[nH]nn3)SC3OC2C(O)C(O)C3O)C1 ; CC(=O)OC[C@@H]1C/C=C\C[C@@H](NC(=O)[C@H]2NC[C@@H]3C[C@H](CC(C)C)CCO[C@H]32)C2OC(S1)C(O)C(O)C2O] 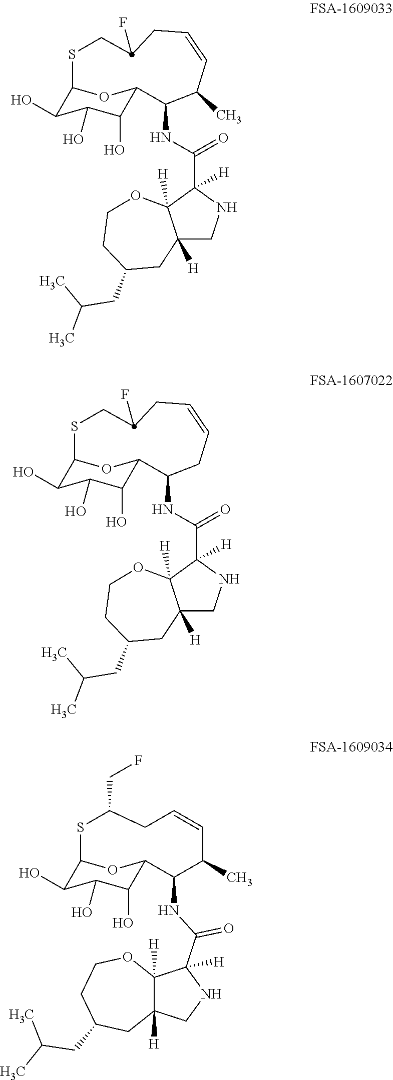 [SMILES: CC(C)C[C@@H]1CCO[C@@H]2[C@H](CN[C@@H]2C(=O)N[C@H]2C3OC(SC[C@H](F)C/C=C\[C@H]2C)C(O)C(O)C3O)C1 ; CC(C)C[C@@H]1CCO[C@@H]2[C@H](CN[C@@H]2C(=O)N[C@@H]2C/C=C\C[C@@H](F)CSC3OC2C(O)C(O)C3O)C1 ; CC(C)C[C@@H]1CCO[C@@H]2[C@H](CN[C@@H]2C(=O)N[C@H]2C3OC(S[C@H](CF)C/C=C\[C@H]2C)C(O)C(O)C3O)C1] 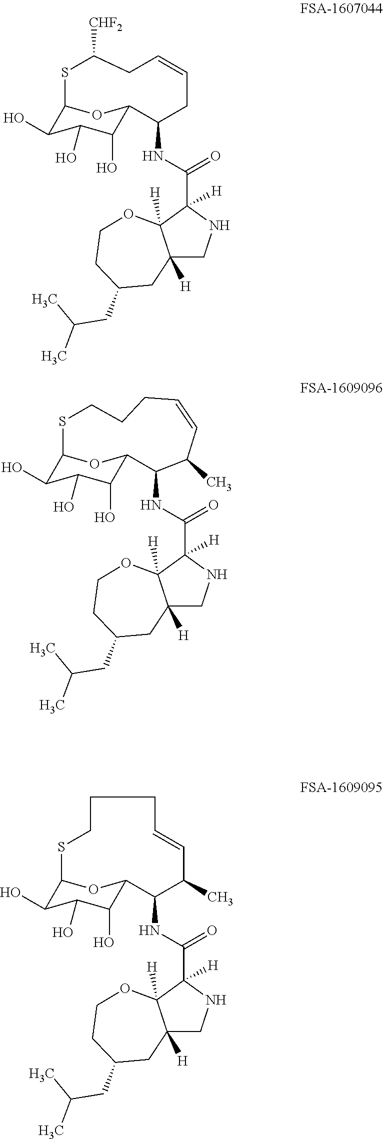 [SMILES: CC(C)C[C@@H]1CCO[C@@H]2[C@H](CN[C@@H]2C(=O)N[C@@H]2C/C=C\C[C@@H](C(F)F)SC3OC2C(O)C(O)C3O)C1 ; CC(C)C[C@@H]1CCO[C@@H]2[C@H](CN[C@@H]2C(=O)N[C@H]2C3OC(SCCC/C=C\[C@H]2C)C(O)C(O)C3O)C1 ; CC(C)C[C@@H]1CCO[C@@H]2[C@H](CN[C@@H]2C(=O)N[C@H]2C3OC(SCCC/C=C/[C@H]2C)C(O)C(O)C3O)C1] 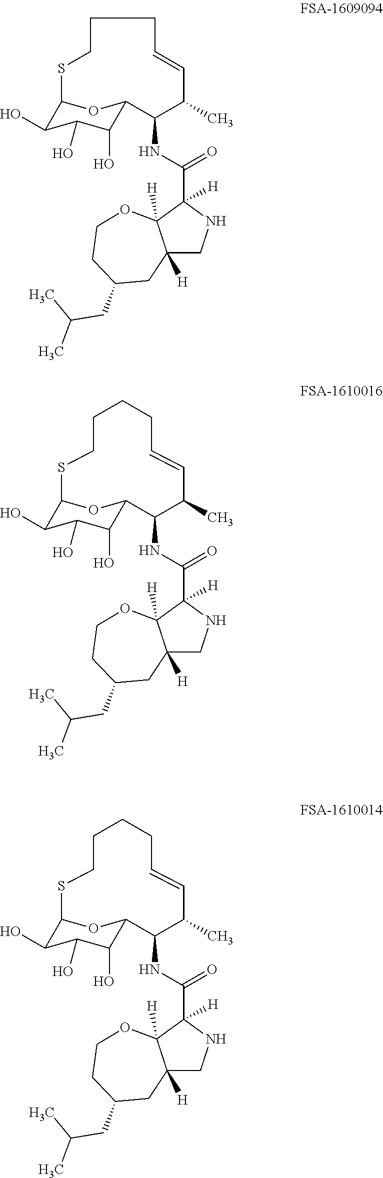 [SMILES: CC(C)C[C@@H]1CCO[C@@H]2[C@H](CN[C@@H]2C(=O)N[C@H]2C3OC(SCCC/C=C/[C@@H]2C)C(O)C(O)C3O)C1 ; CC(C)C[C@@H]1CCO[C@@H]2[C@H](CN[C@@H]2C(=O)N[C@H]2C3OC(SCCCC/C=C/[C@H]2C)C(O)C(O)C3O)C1 ; CC(C)C[C@@H]1CCO[C@@H]2[C@H](CN[C@@H]2C(=O)N[C@H]2C3OC(SCCCC/C=C/[C@@H]2C)C(O)C(O)C3O)C1] 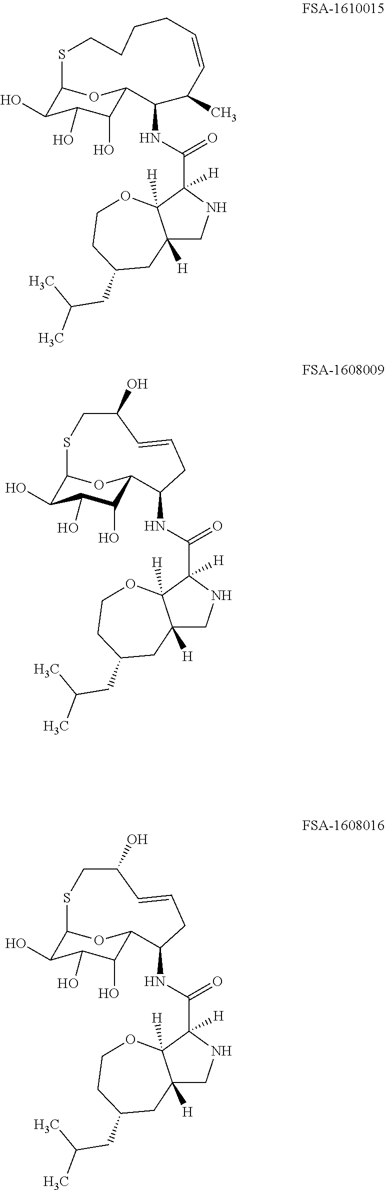 [SMILES: CC(C)C[C@@H]1CCO[C@@H]2[C@H](CN[C@@H]2C(=O)N[C@H]2C3OC(SCCCC/C=C\[C@H]2C)C(O)C(O)C3O)C1 ; CC(C)C[C@@H]1CCO[C@@H]2[C@H](CN[C@@H]2C(=O)N[C@@H]2C/C=C/[C@H](O)CSC3O[C@H]2C(O)[C@@H](O)[C@H]3O)C1 ; CC(C)C[C@@H]1CCO[C@@H]2[C@H](CN[C@@H]2C(=O)N[C@@H]2C/C=C/[C@@H](O)CSC3OC2C(O)C(O)C3O)C1]
or pharmaceutically acceptable salt thereof.

106. A pharmaceutical composition comprising a compound of any of claims 1-105, or a pharmaceutically acceptable salt thereof, and a pharmaceutically acceptable excipient.

107. The pharmaceutical composition of claim 106, comprising a therapeutically effective amount of the compound, or a pharmaceutically acceptable salt thereof.

108. A kit comprising a compound of any of claims 1-105, or pharmaceutically acceptable salt thereof, or a pharmaceutical composition of claim 106 or 107, and instructions for administering the compound or pharmaceutical composition to a subject in need thereof.

109. A method of treating an infectious disease comprising administering an effective amount of a compound of any of claims 1-105, or pharmaceutically acceptable salt thereof, or a pharmaceutical composition of claim 106 or 107, to a subject in need thereof.

110. The method of claim 109, wherein the infectious disease is a bacterial infection.

111. The method of claim 110, wherein the bacterial infection is an infection caused by a Gram positive bacteria.

112. The method of claim 110, wherein the bacterial infection is an infection caused by a Gram negative bacteria.

113. The method of claim 110, wherein the bacterial infection is a Staphylococcus infection, a Streptococcus infection, an Enterococcus infection, an Acenitobacter infection, a Clostridium infection, a Bacterioides infection, an Escherichia infection, a Pseudomonas infection, a Neisseria infection, a Klebsiella infection, or a Haemophilus infection.

114. The method of claim 110, wherein the bacterial infection is a C. difficile infection or a B. fragilis infection.

115. The method of claim 109, wherein the infectious disease is a parasitic infection.

116. A method of killing a microorganism comprising contacting the microorganism with an effective amount of a compound of any of claims 1-105, or pharmaceutically acceptable salt thereof, or a pharmaceutical composition of claim 106 or 107, to a subject in need thereof.

117. A method of inhibiting the growth of a microorganism comprising contacting the microorganism with an effective amount of a compound of any of claims 1-105, or pharmaceutically acceptable salt thereof, or a pharmaceutical composition of claim 106 or 107, to a subject in need thereof.
